(19) United States
(12) Patent Application Publication
(10) Pub. No.: US 2021/0221784 A1
(43) Pub. Date: Jul. 22, 2021

(54) COMPOUNDS AND COMPOSITIONS FOR THE TREATMENT OF PAIN

(71) Applicants: Université de Strasbourg, Strasbourg (FR); Centre National de la Recherche Scientifique, Paris (FR)

(72) Inventors: Martine SCHMITT, Strasbourg (FR); Jacques BRICARD, Illkirch-Graffenstaden (FR); Frédéric SIMONIN, Illkirch-Graffenstaden (FR); Jean-Jacques BOURGUIGNON, Illkirch-Graffenstaden (FR); Frédéric BIHEL, Fegerscheim (FR); Khadija ELHABAZI, Illkirch-Graffenstaden (FR)

(73) Assignees: Université de Strasbourg, Strasbourg (FR); Centre National de la Recherche Scientifique, Paris (FR)

(21) Appl. No.: 16/967,386

(22) PCT Filed: Feb. 5, 2019
(86) PCT No.: PCT/EP2019/052810

§ 371 Date: Aug. 4, 2020

(30) Foreign Application Priority Data
Feb. 5, 2018 (EP) 18305117.6

(51) Int. Cl. C07D 401/04; C07D 213/73

(52) U.S. Cl. CPC C07D 401/04; C07D 213/73

(57) ABSTRACT

The invention relates to compounds, pyridine derivatives, and pharmaceutical 10 compositions containing same for use in the treatment of pain. It also relates to specific compounds, compositions comprising the same and uses thereof, in particular in the treatment of pain.

The invention relates to compounds, pyridine derivatives, and pharmaceutical compositions containing same for use in the treatment of pain. It also relates to specific compounds, compositions comprising the same and uses thereof, in particular in the treatment of pain;

### BACKGROUND OF THE INVENTION

The treatment of pain, in particular chronic pain, is a major public health issue. The use of opiate analgesics (such as morphine or fentanyl) constitutes an effective treatment for acute pain. However, their repeated and prolonged use leads to a loss of effectiveness (tolerance), followed by hypersensitivity to pain (hyperalgesia), making such treatments complex and delicate for the treatment of chronic pain.

The invention describes a novel series of compounds, derivatives of pyridine, that have high affinity for neuropeptide FF (NPFF) receptors, in particular NPFF1 and NPFF2 receptors, which are involved in the modulation of nociceptive signals. In 2006 in PNAS (Simonin et al., PNAS (2006) 103, 2, 466-71), the dipeptide RF9 (referred to as N^{α}-adamantan-1-yl-L-Arg-L-Phe-NH₂ acetate in WO02/24192) was described as being the first nanomolar NPFF receptor antagonist. Administered in vivo in the rat, RF9 showed antihyperalgesic activity, reversing hyperalgesia induced by the repeated administration of opiate analgesics. Similar results were later observed in mice (Elhabazi, K. et al. British Journal of Pharmacology, 2012, 165, 2:424-35)

Opiate analgesics are at present the treatment of choice for moderate or severe pain. For many patients, notably those suffering from advanced cancer, the treatment of pain requires strong, repeated doses of opiates such as morphine or fentanyl. The clinical effectiveness and tolerability of such treatments are, however, qualified by two phenomena induced by the use of opiates. The first is the tolerance effect, which is characterized by a shortening of action duration and a reduction in analgesia intensity. The clinical result is a growing need to increase the doses of opiates in order to maintain the same analgesic effect, uncorrelated with a progression of the disease. The second problem, related to repeated administration of strong doses of opiates, is known as opioid-induced hyperalgesia (OIH). Indeed, prolonged administration of opiates leads to a paradoxical increase in pain, unrelated to the initial nociceptive stimulus.

It has been suggested that such hyperalgesia would be the cause of tolerance. Tolerance would indeed be apparent since the analgesic effect characteristic of each daily dose remains constant; it would thus be the development of hypersensitivity to pain which would give the impression of a decrease in the effects of the opiate. It would thus not be the opiate that would have lost its effectiveness, but the individual who would have become hypersensitive to pain.

These two phenomena have been widely documented in both animal and human studies. Overall, they have been observed after administration of all types of opiates, regardless of the routes of administration or the doses used.

Furthermore, the administration of high doses of opiates leads to a certain number of side effects such as nausea, constipation, sedation and respiratory deficiencies (e.g.: delayed respiratory depression).

Currently, several strategies for mitigating these opiate-induced effects of tolerance and hyperalgesia are under investigation:

1) One of the most commonly used clinical strategies consists of combining opiates with adjuvants such as anticonvulsants or antidepressants, particularly in the treatment of neuropathic pain. In spite of some effectiveness, these additives involve numerous side effects, notably cardiac risks.

2) The rotation of opiates is also used as an alternative strategy, supported by the fact that different opiates have different affinities for each receptor, and that tolerance develops independently for each receptor. However, very few results have been described, and this strategy is the subject of much discussion.

3) NMDA receptor antagonists are known to block calcium channels, which leads in man or animals to a reduction in opiate-induced hyperalgesia as well as to a delay in tolerance effects. However, the clinical use of ketamine as an NMDA receptor antagonist involves a broad spectrum of side effects in man, notably hallucinations.

Although a certain amount of success has been reported, no strategy at present effectively blocks the effects of hyperalgesia and tolerance related to the repeated use of opiates. Consequently, the search for alternative strategies is necessary, notably in the field of neuropathic or cancer pain. Indeed, in the context of these pathologies, the treatments currently used are relatively ineffective and involve the use of high doses of opiates, leading to many particularly disabling side effects. Consequently, a major therapeutic issue relates to the development of novel drugs that act on novel therapeutic targets involved in the modulation of pain.

Research is currently under way for therapies that improve the use of opioid analgesics in mammals, in particular human mammals, in particular during the long-term use or the single administration of high doses, as is the case during surgical procedures.

Among the anti-opiate systems responsible for the loss of effectiveness of opiate analgesics and the appearance of hyperalgesia, NPFF receptors appear to be relevant targets. The design of drugs that inhibit the action of these receptors will make it possible to restore the long-term effectiveness of opiate analgesics while preventing the appearance of opiate-induced hyperalgesia.

In that context, a first patent application published under the number WO02/24192 described Arg-Phe dipeptide derivatives which provided proof of this concept in vivo. In particular, a single administration of Arg-Phe dipeptide derivatives in the rat blocks hyperalgesia induced by administration of fentanyl, an opiate analgesic that acts as a p receptor agonist and is typically used in a hospital setting.

Sampirtine and derivatives thereof have already been described in U.S. Pat. No. 4,851,420. They are described as analgesic and antipyretic agents. WO94/14780 describes pyridine derivatives as NO synthase inhibitors which are more particularly suitable for use as analgesics, chronic neurodegenratie diseases and chronic pain.

### SUMMARY OF THE INVENTION

The present invention describes a family of compounds whose therapeutic use could enable better treatment of postoperative pain or of chronic pain accompanying certain pathologies such as diabetes, cancer, inflammatory disease (rheumatoid arthritis, for example) or neuropathy. These types of pain are regarded as severe and particularly disabling.

The compounds of the present invention are pyridine derivatives that are powerful NPFF1 and/or NPFF2 receptor ligands. Certain compounds show selectivity for NPFF1 or NPFF2.

More particularly, in mice, compounds of the invention prevent long lasting hyperalgesia induced by fentanyl, and prevent the development of hyperalgesia and the development of analgesic tolerance associated with chronic morphine administration, through NPFF1 receptor blockade.

The selectivity of this compound for NPFF1 receptors, located in the supraspinal region, shows the involvement of these receptors in the control of OIH. The advantage of said derivative, as a representative of this novel family of NPFF receptor ligands, is due to the fact that, in contrast to dipeptides represented by RF9, this compound shows highly satisfactory in vivo activity after oral administration of a low dose of 1 mg/kg. Moreover, its effectiveness by oral route was confirmed in a dose-dependent manner.

Furthermore, the study of said compounds shows that an NPFF receptor ligand has an intrinsic effect on hyperalgesia induced by postsurgical, inflammatory or neuropathic pain and improve morphine analgesic effect in these pain models.

Thus, the present invention describes a novel type of NPFF receptor ligand compounds whose administration in a mammal, for example by oral or subcutaneous route, opposes hyperalgesic effects and analgesic tolerance induced by administration of opiate analgesics. Furthermore, the compounds of the invention improve analgesic effect of opiates in different models of pain. The therapeutic prospects envisaged consist notably of co-administration of these compounds with opiate analgesics in the context of the treatment of postoperative pain, but also for the treatment of severe chronic pain caused by inflammation, neuropathy, cancer, diabetes or drugs. Furthermore, the effect of the compounds according to the invention on hypersensitivity to pain makes it possible to also envisage the administration of said compounds alone in the context of the prophylactic treatment of pain.

An object of the invention thus relates to compounds and pharmaceutical compositions comprising the same for use in the treatment of pain, more particularly chronic pain. In particular, the compounds and compositions according to the invention prevent the development of hyperalgesia and the development of analgesic tolerance associated with chronic opiate (such as morphine) administration. Moreover, the compounds and compositions according to the invention decrease hyperalgesic effects and analgesic tolerance induced by administration of opiate analgesics. Furthermore, the compounds and compositions according to the invention improve analgesic effect of opiates in the treatment of pain.

Thus, the compounds and the pharmaceutical compositions according to the invention may be used in the treatment of postoperative pain or of severe chronic pain caused by inflammation, neuropathy, cancer, diabetes or drugs.

The invention also describes a method for treating pain in a subject, comprising the administration to said subject of an effective amount of a compound according to the invention.

The invention also relates to specific compounds, notably as drugs, and to a method for preparing same. The invention also relates to pharmaceutical compositions comprising said specific compounds in a pharmaceutically acceptable carrier.

### LEGENDS TO THE FIGURES

FIG. 1: Effect of compound 1j (mentioned as cpd 1j) on hyperalgesia induced by fentanyl in mice. On day 0, a single dose of compound 1j (5 mg/kg, p.o.) solubilised in 0.5% Tween 80 (A, B) or 10% Kolliphor EL (C, D) was administrated to mice 35 min before fentanyl injections (4×60 μg/kg; 15 min interval; s.c.). Nociceptive responses were measured by using the tail immersion test (48° C.) every 1 h after the last fentanyl injection until return to baseline and once daily from d1 to d4. E, F: Increasing doses of compound 1j or vehicle were administrated at d0 to mice (0.2, 1 and 5 mg/kg, sc.) and 20 min later, animals received four consecutive fentanyl injections (60 μg/kg; 15 min interval; s.c.). Hyperalgesia indexes (HI) (panels B, D and F) were calculated from d1 to d4 as detailed in methods. Data are expressed as mean±S.E.M, n=6-10. ***p<0.001 by Fisher's test as compared with the vehicle+saline group. +++p<0.01 by Fisher's test as compared with the fentanyl pre-treated vehicle group.

FIG. 2: Effect of compound 1j on hyperalgesia and tolerance induced by morphine. A. From d0 to d7, mice received daily oral treatment of R1359 (5 mg/kg) or vehicle 35 min prior to morphine (10 mg/kg; s.c.) or saline. Basal nociceptive latencies were measured once daily before treatment (d1 to d7), using tail immersion test (48° C.). On day 0 and day 8, the analgesic effect of the morphine (5 mg/kg; s.c.) combined or not with compound 1j was monitored during 4 h using the tail immersion test at 48° C. B: Comparison of hyperalgesia index values calculated from d1 to d7 between tested groups. C: Comparison of the peak of analgesia reached at d0 and at d8 for both groups of morphine+vehicle and morphine+compound 1j. Data are expressed as mean±S.E.M, n=8-10. ***p<0.001 by Fisher's test as compared with the vehicle+saline group. +++p<0.001 by Fisher's test as compared with the morphine pre-treated vehicle group. ^{oo}p<0.01, ^{ooo}p<0.001 by Paired t-test as compared with the peak of analgesia value of the same group at d0. μμ p<0.01 by Unpaired t-test as compared with morphine pre-treated vehicle group.

FIG. 3: Effect of compound 1j alone or in combination with morphine on incisional pain. A: Mice that were subjected to a plantar incision at d0 were treated daily from d1 to d6 with compound 1j (5 mg/kg, po) or vehicle 35 min before injection of morphine (2.5 mg/kg, sc.) or saline. Mechanical nociceptive threshold was measured daily 30 min after the sc. injection of morphine with Von Frey filaments. Mechanical nociceptive threshold of the animals was also measured at d15 to check if they returned to normal mechanical sensitivity. B: Comparison of allodynia index values calculated from d1 to d6 between the tested groups. Data are expressed as mean±S.E.M, n=7-9. ++p<0.01 by Fisher's test as compared with the morphine pre-treated vehicle group.

FIG. 4: Effect of compound 1j alone or in combination with morphine on neuropathic pain. A: Mice who were subjected to CCI at d0 were treated daily from d11 to d21 with compound 1j (5 mg/kg, p.o.) or vehicle 35 min before injection of morphine (3 mg/kg, sc.) or saline. Mechanical nociceptive thresholds were measured daily 30 min after the sc. injection of morphine using Von Frey filaments. At d11 post-CCI, mice were subjected before any treatment to a pre-test using Von Frey filaments to verify the development of neuropathic pain. B: Comparison of allodynia index values calculated from d11 to d21between the tested groups. Data are expressed as mean±S.E.M, n=8-11. *p<0.05 by Fisher's test as compared with the vehicle+saline group. ++p<0.01 by Fisher's test as compared with the morphine pre-treated vehicle group.

FIG. 5: Effect of compound 1j on morphine-induced hyperalgesia and analgesic tolerance model in NPFF1R knockout mice. A: Comparison of the basal nociceptive values between NPFF1R KO mice and their littermates WT. B: From d0 to d7, KO and WT mice received daily oral treatment of R1359 (5 mg/kg) or vehicle 35 min prior to morphine (10 mg/kg; s.c.) or saline injection. Basal nociceptive latencies were measured once daily before treatment (d1 to d7), using tail immersion test (48° C.). On day 0 and day 8, the analgesic effect of the morphine (5 mg/kg; s.c.) combined or not with compound 1j was monitored during 4 h using the tail immersion test at 48° C. C: Comparison of hyperalgesia index values calculated from d1 to d7 between KO and WT mice treated with morphine in combination or not with compound 1j. C: Comparison of the peak of analgesia reached at d0 and at d8 for both groups of KO and WT animals treated either with morphine+vehicle or morphine+compound 1j. Data are expressed as mean±S.E.M, n=8-11. && p<0.01 by Unpaired t-test as compared with WT group. *p<0.05 by Fisher's test as compared with the vehicle+morphine WT group. +p<0.05 by Fisher's test as compared with the morphine-pre-treated vehicle WT group. ^{ooo}p<0.001 by Paired t-test as compared with the peak of analgesia value of the same group at d0. μp<0.05 by Unpaired t-test as compared with morphine pre-treated vehicle WT group at d0.

FIG. 6: Dose-response effect of compound 1c (mentioned as cpd 1c) on hyperalgesia induced by fentanyl. A. Increasing doses of compound 1c or vehicle were administrated at d0 to mice (0.2, 1 and 5 mg/kg, sc.) and 20 min later, animals received four consecutive fentanyl injections (60 μg/kg; 15 min interval; s.c.). Nociceptive responses were measured by using the tail immersion test (48° C.) every 1 h after the last fentanyl injection until return to baseline and once daily from d1 to d4. B: Comparison of hyperalgesia index values calculated from d1 to d4 between the tested groups. Data are expressed as mean±S.E.M, n=6-12. ++p<0.01 by Fisher's test as compared with the fentanyl pre-treated vehicle group.

FIG. 7: Effect of different doses of compound 1j (A) and compound 1c (B) on the inhibition of the forskolin-stimulated cAMP production by NPVF (alias RFRP-3) in HEK-293 cells expressing hNPFF1R.

### DETAILED DESCRIPTION OF THE INVENTION

The compounds according to the invention for a use in the treatment of pain have the following general formula (I):

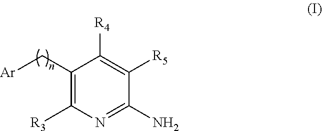

where:
Ar is a carbocyclyl, heterocyclyl, aryl or heteroaryl ring, said ring can optionally be substituted by one or more groups selected from a halogen atom, a (C₁-C₁₀)alkyl group, a cyano group (—CN), a carbocycle, aryl, heterocycle, —C(O)R, —C(O)₂R, —C(O)NRR′, —CONHOR, —CONHSO₂R, —NRR′, —N(R)C(O)R′, —N(R)NR′R″, —N(R)C(O)₂R′, —N(R)C(O)NR′R″, —N(R)S(O)₂R′, —OR, —SR, —S(O)R, —S(O₂)R, —S(O)NRR′, or —S(O)₂NRR′, R, R′, and R″ being independently H, (C₁-C₁₀)alkyl, carbocycle, aryl, heterocycle, heteroaryl, (C₁-C₁₀)alkylcarbocycle, (C₁-C₁₀)alkylaryl, (C₁-C₁₀)alkylheterocycle, (C₁-C₁₀)alkylheteroaryl, (C₁-C₁₀)alkoxycarbocycle, (C₁-C₁₀)alkoxyaryl, (C₁-C₁₀)alkoxyheterocycle), or (C₁-C₁₀)alkoxyheteroaryl group, or R and R′ or R′ and R″ may form a 5-10 membered ring, said 5-10 membered ring is optionally substituted by at least one —OH, halogen, (C₁-C₁₀)alkyl, or (C₁-C₁₀)alkyloxy; said substituent can be further substituted by at least one group selected from a halogen atom, an hydroxyl group, a (C₁-C₁₀)alkyl group, a (C₁-C₁₀)alkoxy group and aryl group;
n is 0, 1, 2, or 3;
R₃ represents an hydrogen atom, halogen atom, NRR′, (C₁-C₁₀)alkyl, or (C₁-C₁₀)alkoxy group;
R₄ represents an hydrogen atom, halogen, NRR′, (C₁-C₁₀)alkyl, or (C₁-C₁₀)alkoxy group;
R₅ represents an hydrogen atom, halogen, NRR′, (C₁-C₁₀)alkyl, or (C₁-C₁₀)alkoxy group;
where R and R′, identical or different, are as defined above;
or any salt thereof.

According to a particular embodiment, the compounds described in U.S. Pat. No. 4,851,420 and WO94/14780 are excluded from the invention.

According a specific embodiment, the excluded compounds according to the invention are the compounds selected in the group consisting of 2,6-diamino-3-(2,4,5-trichlorophenyl)pyridine, 2,6-diamino-3-(phenyl)pyridine, 2,6-diamino-3-(4-methoxyphenyl)pyridine, 2,6-diamino-3-(3,4-dimethoxyphenylphenyl)pyridine, 2,6-diamino-3-(naphtalen-2-yl)pyridine, and 2,6-diamino-3-(3,5-dichlorophenyl)pyridine.

### Definitions

As used herein, the term “about” will be understood by a person of ordinary skill in the art and will vary to some extent on the context in which it is used. If there are uses of the term which are not clear to persons of ordinary skill in the art given the context in which it is used, “about” will mean up to plus or minus 10% of the particular term.

According to the invention, the term “comprise(s)” or “comprising” (and other comparable terms, e.g., “containing,” and “including”) is “open-ended” and can be generally interpreted such that all of the specifically mentioned features and any optional, additional and unspecified features are included. According to specific embodiments, it can also be interpreted as the phrase “consisting essentially of” where the specified features and any optional, additional and unspecified features that do not materially affect the basic and novel characteristic(s) of the claimed invention are included or the phrase “consisting of” where only the specified features are included, unless otherwise stated.

The terms mentioned herein with prefixes such as for example C₁-C₃, C₁-C₆ or C₂-C₆ can also be used with lower numbers of carbon atoms such as C₁-C₂, C₁-C₅, or C₂-C₅. If, for example, the term C₁-C₃ is used, it means that the corresponding hydrocarbon chain may comprise from 1 to 3 carbon atoms, especially 1, 2 or 3 carbon atoms. If, for example, the term C₁-C₆ is used, it means that the corresponding hydrocarbon chain may comprise from 1 to 6 carbon atoms, especially 1, 2, 3, 4, 5 or 6 carbon atoms. If, for example, the term C₂-C₆ is used, it means that the corresponding hydrocarbon chain may comprise from 2 to 6 carbon atoms, especially 2, 3, 4, 5 or 6 carbon atoms.

According to the invention, the term “(C₁-C₁₀)alkyl” designates a saturated or unsaturated hydrocarbonated group, linear, branched or cyclic, having from 1 to 10, preferably from 1 to 8, from 1 to 6 or from 1 to 4, carbon atoms. Among the saturated alkyl group, one can cite methyl, ethyl, propyl, isopropyl, cyclopropyl, butyl, isobutyl, tert-butyl, cyclobutyl, pentyl, cyclopentyl, neopentyl, n-hexyl. The alkyl term also designates an alkyl group having both linear and cyclic hydrocarbonated group, such as —CH₃(C₃H₅). The unsaturated alkyl group can be an alkenyl group or an alkynyl group. The term “alkenyl” refers to an unsaturated, linear, branched or cyclic aliphatic group comprising at least one carbon-carbon double bound. The term “(C₂-C₆)alkenyl more specifically means ethenyl, propenyl, isopropenyl, butenyl, isobutenyl, pentenyl, or hexenyl.

The term “alkynyl” refers to an unsaturated, linear branched or cyclic aliphatic group comprising at least one carbon-carbon triple bound. The term “(C₂-C₆)alkynyl more specifically means ethynyl, propynyl, butynyl, pentynyl, isopentynyl, or hexynyl.

The alkyl group can be substituted by at least one halogen atom or NRR′ group (R and R′ being as defined above, and are more particularly and independently hydrogen atom or a (C₁-C₁₀)alkyl group as defined above). In that context, when halogenated, the alkyl group can be more particularly CF₃ or CH₂CF₃.

The alkyl group can be interrupted by at least one heteroatom or a group, such as oxygen, sulfur atom, NR group, —C(O)NR— or —N(R)C(O)—, where R is as defined above, and it includes more particularly hydrogen atom or a (C₁-C₁₀)alkyl group as defined above, to form, respectively, an ether, thioether, amine, carboxamine or amide bond within the alkyl chain or within a cycle to form a heterocycle. When the alkyl group is an ether group, it can be —O(CH2)mOCH3, where m is an integer from 1 to 6, such as 1, 2 or 3.

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C3-6 carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl. In a preferred embodiment, the “carbocycle” is a cyclopentyl or a cyclohexyl.

As used herein, “heterocycle” or “heterocyclyl” means a non-aromatic cyclic ring or ring system containing at least one heteroatom in the ring backbone. Heterocycles may be joined together in a fused, bridged or spiro-connected fashion. Heterocycles may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, tetrahydrofuranyl, tetrahydropyranyl, tetrahydrothiophenyl, tetrahydrothiopyranyl, tetrahydro-1,4-thiazinyl, and thiamorpholinyl. A “(heterocyclyl)alkyl” is a heterocyclyl group connected, as a substituent, via an alkylene group. Examples include, but are not limited to, piperidinylethyl, or imidazolinylmethyl.

The term alkoxy refers to an alkyl chain linked to the rest of the compound by means of an oxygen atom (ether linkage). The alkyl chain corresponds to the definition given above, including the interrupted or substituted alkyl as defined above. As examples, one can cite the methoxy, trifluoromethoxy, ethoxy, n-propyloxy, isopropyloxy, n-butoxy, iso-butoxy, tert-butoxy, sec-butoxy, hexyloxy radicals. The alkoxy group can be an amino(C₁-C₁₀)alkoxy group. An amino(C₁-C₁₀)alkoxy group refers to an alkoxy chain terminated by an amino group (—NH₂) and linked to the rest of the molecule by an oxygen atom.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

The term “aryl” corresponds to a mono- or bi-cyclic aromatic hydrocarbons having from 6 to 12 carbon atoms. For instance, the term “aryl” includes phenyl or naphthyl. In a preferred embodiment, the aryl is a phenyl.

The term “heteroaryl” as used herein corresponds to an aromatic, mono- or poly-cyclic group comprising between 5 and 14 atoms and comprising at least one heteroatom such as nitrogen, oxygen or sulphur atom. Examples of such mono- and poly-cyclic heteroaryl group may be: pyridinyl, thiazolyl, thienyl, furanyl, pyrrolyl, pyrazolyl, imidazolyl, triazolyl, tetrazolyl, benzofuranyl, triazinyl, isothiazolyl, isoxazolyl, pyrazinyl, pyridazinyl, pyrimidinyl, furazanyl, imidazolidinyl, imidazolinyl, pyrazolidinyl, pyrazolinyl, oxazolidinyl, dihydropyridyl, pyrimidinyl, s-triazinyl, oxazolyl, or thiofuranyl.

In a preferred embodiment, the heteroaryl group is a thienyl, a furanyl, a benzofuranyl, a pyridinyl, a pyrazolyl, a pyrazinyl, or a thiazolyl.

The term 5-10 membered ring of R and R′ or R′ and R″ includes heterocycle or heteroaryl groups as defined above having 5 to 10 ring members, preferably 5-7 ring members.

The terms (C₁-C₁₀)alkylcarbocycle, (C₁-C₁₀)alkoxycarbocycle, (C₁-C₁₀)alkylaryl, (C₁-C₁₀)alkoxyaryl, (C₁-C₁₀)alkylheterocycle), (C₁-C₁₀)alkoxyheterocycle), (C₁-C₁₀)alkylheteroaryl, and (C₁-C₁₀)alkoxyheteroaryl refer to carbocycle, aryl, heterocycle or heteroaryl substituted by alkyl or alkoxy group, respectively.

The aryl and heteroaryl groups can be attached to the rest of the compound by an alkyl group as defined above, they are thus referred to as aralkyl (or an aryl(C₁-C₁₀)alkyl group) or heteroaralkyl groups, respectively.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

The herein described specific or preferred embodiments can be combined to each other whenever it is chemically feasible. For instance, specifically described embodiments relative to Ar definitions can be combined with specifically described embodiments relative to n, R3, R4 and/or R5.

### Compounds and Uses Thereof

According to a specific embodiment, the compounds of the invention are of formula (I) where Ar is an aryl, preferably a phenyl group, said group is optionally substituted as specified above, more specifically by one or more groups selected from a halogen atom, a cyano group, a (C₁-C₁₀)alkyl group, an aryl group, or a —OR, R being as defined above, preferably R being H or (C₁-C₁₀)alkyl.

According to a particular embodiment A, the compounds of the invention are of formula (I) where Ar is 1-naphtyl, said naphtyl being optionally substituted as defined above. According to this particular embodiment, at least one of, or more particularly all, the following features are fulfilled:

n is 0,

R₃ is an (C₁-C₁₀)alkyl group, such as ethyl, or NRR′, such as NH2,

the 1-naphtyl is unsubstituted or substituted by at least one group selected from a halogen atom, a cyano group, a (C₁-C₁₀)alkyl group, —OR, or —NRR′, where R and R′ are as defined above,

R₄ represents an hydrogen atom, and

R₅ represents an hydrogen atom.

According to another embodiment B, the compounds of the invention are of formula (I) where Ar is a carbocyclyl or an heteroaryl, preferably a furanyl, benzofuranyl, a pyrazolyl (preferably 4-pyrazolyl) or a pyridinyl (preferably 3-pyridyl or 4-pyridyl) group, said Ar group can optionally be substituted as specified above, more specifically by one or more groups selected from a halogen atom, a (C₁-C₁₀)alkyl group, an aryl group, a —OR, R being as defined above, preferably R being H, (C₁-C₁₀)alkyl, or a —NRR′ group, R and R′ being as defined above, preferably R and R′ are independently H, (C₁-C₁₀)alkyl, or heterocycle. According to this particular embodiment, at least one of, or more particularly all, the following features are fulfilled:

n is 0,

R₃ is an (C₁-C₁₀)alkyl group, such as ethyl, or NRR′, such as NH2,

R₄ represents an hydrogen atom, and

R₅ represents an hydrogen atom.

According to another embodiment C, the compounds of the invention are of formula (I) where Ar is an heterocycle, optionally substituted as defined above, and R3 represents an halogen atom, NRR′, (C₁-C₁₀)alkyl, (C₁-C₁₀)alkoxy group. According to this particular embodiment, n is preferably 1.

According to a specific embodiment, the compounds of the invention are of formula (I) where R₄ represents H, an halogen atom, an alkyl group (such as CH₃ or CF₃), an akoxy group (such as OCH₃, OCH₂CF₃, O(CH₂)₂CF₃), O(CH₂)₂NH₂). According to a preferred embodiment, R₄ represents H.

According to another specific embodiment, the compounds of the invention are of formula (I) where R₅ represents H, an halogen atom or an alkyl group (such as CH₃ or CF₃). According to a preferred embodiment, R₅ represents H

According to a more specific embodiment, the compounds of the invention are of formula (I) where R₄ and R₅ both represent an hydrogen atom.

According to a specific embodiment, the compounds of the invention are of formula (I) where R₃ is NH₂, an halogen atom, such as Cl or F, a (C₁-C₄)alkyl (such as methyl or ethyl), CF₃, (C₁-C₄)alkoxy group (such as methoxy, ethoxy, OCH₂CF₃. O(CH₂)₂NH₂), an ether group (such as methoxymethyl), NRR′, where R and R′ are as defined above, preferably R is H and R′ is (C₁-C₁₀)alkyl (more particularly methyl, n-butyl, ethyl, isopropyl), optionally substituted by an aryl (such as phenyl), by an alkoxy (such as methoxy), or by an heterocycle (such as piperidine), R′ can also be an an heterocycle (such as piperidine), or alternatively R and R′ can form together an heterocycle with the nitrogen to which they are attached, such as piperidine.

According to a specific embodiment, the compounds of the invention are of formula (I) where R₃ is NH₂.

According to a specific embodiment, the compounds of the invention are of formula (I) where n is 1. When n is 1, R₃ is NH₂ and R₄ and R₅ are hydrogen atoms, then Ar is preferably an aryl and more preferably a phenyl group, said phenyl group is more particularly substituted with only one or two chlorine atoms (i.e. the phenyl group is substituted by one or two chlorine atoms, only), where preferably at least one of said chlorine atom is on position 2 or 3 or 4, more preferably only one chlorine on position 2 or two chlorine atoms on positions 2 and 4.

According to another specific embodiment, the compounds of the invention are of formula (I) where n is 0. In a more particular embodiment, n is 0 and Ar is substituted at least on position 2 (the substituents being as defined above).

According to a specific embodiment, the compounds of the invention are compounds of formula (II):

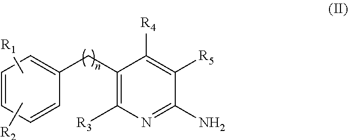

where:
n is 0, 1 or 2, and preferably n is 0;
R₃, R₄ and R₅ are as defined above, and
R₁ and R₂ are independently hydrogen atoms or the substituents of Ar are as defined above.

The compounds of formula (II) are, in a preferred embodiment, of formula (II) where:

R₁ represents a halogen atom, a (C₁-C₁₀)alkyl group, a cyano group (—CN), an aryl(C₁-C₁₀)alkyl group, carbocycle, aryl, heterocycle, —C(O)R, —C(O)₂R, —C(O)NRR′, —CONHOR, —CONHSO₂R, —NRR′, —N(R)C(O)R′, —N(R)NR′R″, —N(R)C(O)₂R′, —N(R)C(O)NR′R″, —N(R)S(O)₂R′, —OR, —SR, —S(O)R, —S(O₂)R, —S(O)NRR′, or —S(O)₂NRR′, R, R′, and R″ being independently H, (C₁-C₁₀)alkyl, carbocycle, aryl, aralkyl, heterocycle, heteroaryl, (C₁-C₁₀)alkylcarbocycle, (C₁-C₁₀)alkylaryl, (C₁-C₁₀)alkylheterocycle), (C₁-C₁₀)alkylheteroaryl, (C₁-C₁₀)alkoxycarbocycle, (C₁-C₁₀)alkoxyaryl, (C₁-C₁₀)alkoxyheterocycle), or (C₁-C₁₀)alkoxyheteroaryl group, or R and R′ or R′ and R″ may form a 5-10 membered ring, said 5-10 membered ring is optionally substituted by at least one —OH, halogen, (C₁-C₁₀)alkyl, or (C₁-C₁₀)alkyloxy; said R₁ group can be further substituted by at least one group selected from a halogen atom, an hydroxyl group, a (C₁-C₁₀)alkyl group, a (C₁-C₁₀)alkoxy group and aryl group; and/or R₂ represents an hydrogen atom, a halogen atom, a (C₁-C₁₀)alkyl group, —C(O)R, —C(O)₂R, —C(O)NRR′, —CONHOR, —CONHSO₂R, —NRR′, —N(R)C(O)R′, —N(R)NR′R″, —N(R)C(O)₂R′, —N(R)C(O)NR′R″, —N(R)S(O)₂R′, —OR, —SR, —S(O)R, —S(O₂)R, —S(O)NRR′, or —S(O)₂NRR′, R, R′, and R″ being independently H, (C₁-C₁₀)alkyl, carbocycle, aryl, heterocycle, heteroaryl, (C₁-C₁₀)alkylcarbocycle, (C₁-C₁₀)alkylaryl, (C₁-C₁₀)alkylheterocycle), (C₁-C₁₀)alkylheteroaryl, (C₁-C₁₀)alkoxycarbocycle, (C₁-C₁₀)alkoxyaryl, (C₁-C₁₀)alkoxyheterocycle), or (C₁-C₁₀)alkoxyheteroaryl group, or R and R′ or R′ and R″ may form a 5-10 membered ring, said 5-10 membered ring is optionally substituted by at least one —OH, halogen, (C₁-C₁₀)alkyl, or (C₁-C₁₀)alkyloxy; said R₂ group can be further substituted by at least one group selected from a halogen atom, an hydroxyl group, a (C₁-C₁₀)alkyl group, and a (C₁-C₁₀)alkoxy group.

More preferably, R₂ is H and R₁ represents a halogen atom, a (C₁-C₁₀)alkyl group, or —OR, and most preferably n is 0. Even more preferably, R₁ is on position 2 of the phenyl group of formula (II).

According to another specific embodiment, the compounds of the invention are compounds of formula (III):

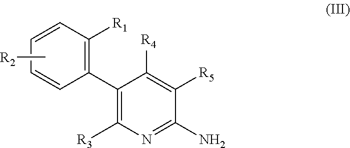

where R₃, R₄ and R₅ are as defined above, including preferred embodiments as identified above, and
R₁ represents a halogen atom, a (C₁-C₁₀)alkyl group, a cyano group (—CN), an aryl(C₁-C₁₀)alkyl group, carbocycle, aryl, heterocycle, —C(O)R, —C(O)₂R, —C(O)NRR′, —CONHOR, —CONHSO₂R, —NRR′, —N(R)C(O)R′, —N(R)NR′R″, —N(R)C(O)₂R′, —N(R)C(O)NR′R″, —N(R)S(O)₂R′, —OR, —SR, —S(O)R, —S(O₂)R, —S(O)NRR′, or —S(O)₂NRR′, R, R′, and R″ being independently H, (C₁-C₁₀)alkyl, carbocycle, aryl, aralkyl, heterocycle, heteroaryl, (C₁-C₁₀)alkylcarbocycle, (C₁-C₁₀)alkylaryl, (C₁-C₁₀)alkylheterocycle), (C₁-C₁₀)alkylheteroaryl, (C₁-C₁₀)alkoxycarbocycle, (C₁-C₁₀)alkoxyaryl, (C₁-C₁₀)alkoxyheterocycle), or (C₁-C₁₀)alkoxyheteroaryl group, or R and R′ or R′ and R″ may form a 5-10 membered ring, said 5-10 membered ring is optionally substituted by at least one —OH, halogen, (C₁-C₁₀)alkyl, or (C₁-C₁₀)alkyloxy; said R₁ group can be further substituted by at least one group selected from a halogen atom, an hydroxyl group, a (C₁-C₁₀)alkyl group, a (C₁-C₁₀)alkoxy group and aryl group;
R₂ represents an hydrogen atom, a halogen atom, a (C₁-C₁₀)alkyl group, —C(O)R, —C(O)₂R, —C(O)NRR′, —CONHOR, —CONHSO₂R, —NRR′, —N(R)C(O)R′, —N(R)NR′R″, —N(R)C(O)₂R′, —N(R)C(O)NR′R″, —N(R)S(O)₂R′, —OR, —SR, —S(O)R, —S(O₂)R, —S(O)NRR′, or —S(O)₂NRR′, R, R′, and R″ being independently H, (C₁-C₁₀)alkyl, carbocycle, aryl, heterocycle, heteroaryl, (C₁-C₁₀)alkylcarbocycle, (C₁-C₁₀)alkylaryl, (C₁-C₁₀)alkylheterocycle), (C₁-C₁₀)alkylheteroaryl, (C₁-C₁₀)alkoxycarbocycle, (C₁-C₁₀)alkoxyaryl, (C₁-C₁₀)alkoxyheterocycle), or (C₁-C₁₀)alkoxyheteroaryl group, or R and R′ or R′ and R″ may form a 5-10 membered ring, said 5-10 membered ring is optionally substituted by at least one —OH, halogen, (C₁-C₁₀)alkyl, or (C₁-C₁₀)alkyloxy; said R₂ group can be further substituted by at least one group selected from a halogen atom, an hydroxyl group, a (C₁-C₁₀)alkyl group, a (C₁-C₁₀)alkoxy group.

Compounds of formula (III) are preferably with one or more of the following features:
R₂ is H, a halogen atom, a (C₁-C₁₀)alkyl group (preferably (C₁-C₄)alkyl), or —OR, R is as defined above, and more preferably R is H or (C₁-C₁₀)alkyl; and/or
R₁ represents a halogen atom, a (C₁-C₁₀)alkyl group (preferably (C₁-C₄)alkyl), carbocycle (such as cyclopropyl, cyclopentyl), aryl (such as phenyl), or —OR, with R is as defined above, and more preferably R is H, (C₁-C₁₀)alkyl (such as methyl, ethyl, iso-propyl, or —CH₃(C₃H₅)), (C₁-C₁₀)alkylheterocycle (such as 1-piperidinylethyl) or carbocyclyl (such as cyclopropyl, cyclopentyl) as defined above; and/or
R₃ is a (C₁-C₄)alkyl (such as methyl or ethyl), NRR′, with R is H and R′ is H, (C₁-C₁₀)alkyl (more particularly methyl, n-butyl, ethyl, isopropyl), optionally substituted by an aryl (such as phenyl), by an alkoxy (such as methoxy), or by an heterocycle (such as piperidine), R′ can also be an heterocycle (such as piperidine), or alternatively R and R′ can form together an heterocycle with the nitrogen to which they are attached, such as piperidine; and/or
R₄ and R₅ are independently an hydrogen atom, an halogen atom, an (C₁-C₁₀)alkyl group, or an (C₁-C₁₀)alkoxy group, preferably R₄ and R₅ are both hydrogen atoms.

According to other particular aspects of the invention, the invention also relates to compounds of formula (III), compounds of embodiments A or B, as defined above, and uses thereof, more particularly for a use in the therapeutic field and more specifically in the treatment of pain.

The present invention also relates to a pharmaceutical composition comprising at least one compound of formula (III), of embodiment A or of embodiment B, in a pharmaceutically acceptable vehicle or support.

Compounds of formulas (I), (II) or (III) as defined above are illustrated in the following examples.
3-(2-chlorophenyl)pyridine-2,6-diamine (trifluoroacetate), 1b,
3-(o-tolyl)pyridine-2,6-diamine (trifluoroacetate), 1c,
3-(2-ethylphenyl)pyridine-2,6-diamine (hydrochloride), 1d,
3-(2-isopropylphenyl) pyridine-2,6-diamine (hydrochloride), 1e,
3-(2-(trifluoromethyl) phenyl) pyridine-2,6-diamine (trifluoroacetate), 1f,
3-(2-(methoxymethyl) phenyl) pyridine-2,6-diamine (hydrochloride), 1g,
3-(3-chlorophenyl)pyridine-2,6-diamine (trifluoroacetate), 1h,
3-(2,3-dichlorophenyl)pyridine-2,6-diamine, 1j,
3-(2,4-dichlorophenyl) pyridine-2,6-diamine (trifluoroacetate), 1k,
3-(2-chloro-3-(trifluoromethyl) phenyl) pyridine-2,6-diamine (trifluoroacetate), 1p,
3-([1,1′-biphenyl]-2-yl)pyridine-2,6-diamine (hydrochloride), 1r,
2-(2,6-diaminopyridin-3-yl)phenol, 2a,
3-(2-methoxyphenyl) pyridine-2,6-diamine (trifluoroacetate), 2b,
3-(2-(trifluoromethoxy) phenyl) pyridine-2,6-diamine (hydrochloride), 2c,
3-(2-ethoxyphenyl)pyridine-2,6-diamine (hydrochloride), 2d,
3-(2-butoxyphenyl)pyridine-2,6-diamine, 2e,
3-(2-isopropoxyphenyl)pyridine-2,6-diamine 2f,
3-(2-isobutoxyphenyl) pyridine-2,6-diamine (hydrochloride), 2g,
3-(2-methoxyethoxyphenyl)pyridine-2,6-diamine, 2h,
3-(2-(cyclopentyloxy)phenyl)pyridine-2,6-diamine 2i,
3-(2-(piperidin-1-yl)ethoxy)pyridine-2,6-diamine 2j,
3-(4-fluoro-2-methoxyphenyl) pyridine-2,6-diamine(hydrochloride), 2k,
3-(2,3-dimethoxyphenyl) pyridine-2,6-diamine (hydrochloride), 2l,
3-(2,4-dimethoxyphenyl pyridine-2,6-diamine (hydrochloride), 2m,
N-(6-amino-5-(2,3-dichlorophenyl)pyridin-2-yl)acetamide, 4a,
3-(2,3-dichlorophenyl)-N2-methylpyridine-2,6-diamine, 5a,
3-(2,3-dichlorophenyl)-N2-ethylpyridine-2,6-diamine, 5b,
N2-benzyl-3-(2,3-dichlorophenyl)pyridine-2,6-diamine, 5d,
5-(2,3-dichlorophenyl)-6-methylpyridin-2-amine, 6b,
5-(2,3-dichlorophenyl)-6-ethylpyridin-2-amine, 6c,
6-ethyl-5-(2-methoxyphenyl)pyridin-2-amine, 6d,
5-(2-methoxyphenyl)-6-propylpyridin-2-amine, 6g,
6-isopropyl-5-(2-methoxyphenyl)pyridin-2-amine, 6h,
6-cyclopropyl-5-(2-methoxyphenyl)pyridin-2-amine, 6i,
3-(2-chlorobenzyl)pyridine-2,6-diamine, 10e,
3-(2,4-dichlorobenzyl)pyridine-2,6-diamine (hydrochloride), 10f,
3-(4-chlorophenyl)pyridine-2,6-diamine (trifluoroacetate), 1i
3-(2,5-dichlorophenyl) pyridine-2,6-diamine (trifluoroacetate), 1l,
3-(2,6-dichlorophenyl) pyridine-2,6-diamine, 1m,
3-(3,4-dichlorophenyl) pyridine-2,6-diamine (trifluoroacetate), 1n,
3-(3,5-dichlorophenyl) pyridine-2,6-diamine (trifluoroacetate), 1o,
3-(3-chloro-2-methylphenyl) pyridine-2,6-diamine (trifluoroacetate), 1q,
3-(furan-2-yl)pyridine-2,6-diamine (trifluoroacetate), 3a
3-(furan-2-yl)pyridine-2,6-diamine (trifluoroacetate), 3b
3-(benzofuran-2-yl) pyridine-2,6-diamine (hydrochloride), 3c
[3,4′-bipyridine]-2,6-diamine, 3d
[3,3′-bipyridine]-2,6-diamine, 3e,
N2-butyl-3-(2,3-dichlorophenyl)pyridine-2,6-diamine, 5c
3-(2,3-dichlorophenyl)-N2-isopropylpyridine-2,6-diamine, 5e,
3-(2,3-dichlorophenyl)-N2-phenethylpyridine-2,6-diamine, 5f,
3-(2,3-dichlorophenyl)-N2-(2-methoxyethyl)pyridine-2,6-diamine, 5g,
3-(2,3-dichlorophenyl)-N2-(2-(piperidin-1-yl)ethyl)pyridine-2,6-diamine, 5h,
5-(2,3-dichlorophenyl)-6-(piperidin-1-yl)pyridin-2-amine, 5i,
5-(2,3-dichlorophenyl)pyridin-2-amine, 6a,
6-(methoxymethyl)-5-(2-methoxyphenyl)pyridin-2-amine, 6e
5-(2-methoxyphenyl)-6-(trifluoromethoxy)pyridin-2-amine, 6f,
5-(2,3-dichlorophenyl)-6-methoxypyridin-2-amine, 7a
4-methyl-3-(o-tolyl)pyridine-2,6-diamine, 8b
3-(2,3-dichlorophenyl)-4-methoxypyridine-2,6-diamine, 8a
3-(2,3-dichlorophenyl)-5-fluoropyridine-2,6-diamine, 9a
3-(2,3-dichlorophenyl)-5-ethylpyridine-2,6-diamine, 9b
3-benzylpyridine-2,6-diamine hydrochloride, 10a,
3-(4-fluorobenzyl)pyridine-2,6-diamine, 10b,
3-(4-chlorobenzyl)pyridine-2,6-diamine, 10c,
3-(3-chlorobenzyl)pyridine-2,6-diamine, 10d,
3-phenethylpyridine-2,6-diamine, 11a, or
3-phenylpyridine-2,6-diamine, 1a.
5-(2-Methoxy-phenyl)-3-methyl-pyridin-2-ylamine, 13
5-(2-Methoxy-phenyl)-3-trifluoromethyl-pyridin-2-ylamine, 14
3-Fluoro-5-(2-methoxy-phenyl)-pyridin-2-ylamine, 15
5-(2,3-Dichloro-phenyl)-3-fluoro-pyridin-2-ylamine, 16
5-(2,3-Dichloro-phenyl)-4-fluoro-pyridin-2-ylamine, 17
4-Fluoro-5-(2-methoxy-phenyl)-pyridin-2-ylamine, 18
5-(2,3-Dichloro-phenyl)-4-methoxy-pyridin-2-ylamine (hydrochloride), 19
4-Methoxy-5-(2-methoxy-phenyl)-pyridin-2-ylamine, 20
5-(2-Methoxy-phenyl)-4-methyl-pyridin-2-ylamine, 21
5-(2,3-Dichloro-phenyl)-4-methyl-pyridin-2-ylamine, 22
5-(2-Methoxy-phenyl)-4-(2,2,2-trifluoro-ethoxy)-pyridin-2-ylamine (hydrochloride), 23
5-(2,3-Dichloro-phenyl)-4-(2,2,2-trifluoro-ethoxy)-pyridin-2-ylamine (hydrochloride), 24
4-(2-aminoethoxy)-5-(2-methoxyphenyl)pyridin-2-amine (dihydrochloride), 25
5-(2-Methoxy-phenyl)-6-methyl-pyridin-2-ylamine, 26
5-(2-Methoxy-phenyl)-4,6-dimethyl-pyridin-2-ylamine, 27
5-(2,3-Dichloro-phenyl)-4,6-dimethyl-pyridin-2-ylamine, 28
5-(3-chloro-2-methyl-phenyl)-6-ethyl-pyridin-2-ylamine (hydrochloride), 29
5-(2-cyclopropylphenyl)-6-ethyl-pyridin-2-amine (hydrochloride), 30
5-[2-(cyclopropoxy)phenyl]-6-ethyl-pyridin-2-amine (hydrochloride), 31
6-Fluoro-5-(2-methoxy-phenyl)-pyridin-2-ylamine, 32
5-(2,3-Dichloro-phenyl)-6-fluoro-pyridin-2-ylamine, 33
5-(2,3-Dichloro-phenyl)-6-trifluoromethyl-pyridin-2-ylamine, 34
6-Methoxy-5-(2-methoxy-phenyl)-pyridin-2-ylamine, 35
5-(2-Methoxy-phenyl)-6-(2,2,2-trifluoro-ethoxy)-pyridin-2-ylamine, 36
6-(2-Amino-ethoxy)-5-(2-methoxy-phenyl)-pyridin-2-ylamine, 37
6-(2-Amino-ethoxy)-5-(2,3-dichloro-phenyl)-pyridin-2-ylamine (dihydrochloride), 38
3-(2-Isopropoxy-6-methoxy-phenyl)-pyridine-2,6-diamine, 39
3-(4-Methoxy-2-methyl-phenyl)-pyridine-2,6-diamine, 40
3-(4-Chloro-2-fluoro-phenyl)-pyridine-2,6-diamine, 41
3-(2-Cyclopropyl-phenyl)-pyridine-2,6-diamine (hydrochloride), 42
3-(2-Phenoxy-phenyl)-pyridine-2,6-diamine (hydrochloride), 43
3-(2-Benzyl-phenyl)-pyridine-2,6-diamine, 44
3-(2-Chloro-4-fluoro-phenyl)-pyridine-2,6-diamine, 45
3-(2-Isopropoxy-4-methyl-phenyl)-pyridine-2,6-diamine, 46
3-(4-Chloro-2-cyclopentyloxy-phenyl)-pyridine-2,6-diamine, 47
3-(2-Cyclopropoxy-phenyl)-pyridine-2,6-diamine, 48
3-(2-Isopropoxy-5-methyl-phenyl)-pyridine-2,6-diamine, 49
3-(5-Fluoro-2-isopropoxy-phenyl)-pyridine-2,6-diamine, 50
3-(2,6-Dimethyl-phenyl)-pyridine-2,6-diamine, 51
3-(2-Isopropoxy-5-trifluoromethyl-phenyl)-pyridine-2,6-diamine, 52
3-(4-Fluoro-2-isopropoxy-phenyl)-pyridine-2,6-diamine, 53
3-(4-Chloro-2-methyl-phenyl)-pyridine-2,6-diamine, 54
3-(5-Chloro-2-cyclopropyl-phenyl)-pyridine-2,6-diamine, 55
3-(5-Chloro-2-methyl-phenyl)-pyridine-2,6-diamine, 56
3-(2-Methyl-4-trifluoromethyl-phenyl)-pyridine-2,6-diamine, 57
3-(2-chloro-3-methyl-phenyl)-pyridine-2,6-diamine, 58
3-(2-Methylsulfanyl-phenyl)-pyridine-2,6-diamine (hydrochloride), 59
2-(2,6-Diamino-pyridin-3-yl)-N,N-diethyl-benzamide (hydrochloride), 60
3-(2-Dimethylamino-phenyl)-pyridine-2,6-diamine (hydrochloride), 61
N-[2-(2,6-Diamino-pyridin-3-yl)-phenyl]-acetamide, 62
3-(2-methylsulfonylphenyl)pyridine-2,6-diamine (hydrochloride), 63
3-(2-benzyloxyphenyl)pyridine-2,6-diamine (hydrochloride), 64
3-[2-(cyclopropylmethoxy)phenyl]pyridine-2,6-diamine (hydrochloride), 65
3-(3-Chloro-2-methyl-phenyl)-5-fluoro-pyridine-2,6-diamine, 66
6-ethyl-5-(1-naphthyl)pyridin-2-amine (hydrochloride), 67
3-(1-naphthyl)pyridine-2,6-diamine, 68
3-(2-methoxy-1-naphthyl)pyridine-2,6-diamine, 69
3-(2-isopropoxy-1-naphthyl)pyridine-2,6-diamine, 70
3-(4-methyl-1-naphthyl)pyridine-2,6-diamine (hydrochloride), 71
3-(4-fluoro-1-naphthyl)pyridine-2,6-diamine (hydrochloride), 72
3-(4-chloro-1-naphthyl)pyridine-2,6-diamine (hydrochloride), 73
4-(2,6-diamino-3-pyridyl)naphthalen-1-ol (hydrochloride), 74
3-[4-(dimethylamino)-1-naphthyl]pyridine-2,6-diamine (hydrochloride), 75
5-[2-(cyclopentoxy)phenyl]-6-ethyl-pyridin-2-amine (hydrochloride), 76
3-(4-bromophenyl)pyridine-2,6-diamine, 77
3-(6-morpholino-3-pyridyl)pyridine-2,6-diamine, 78
3-[6-(1-piperidyl)-3-pyridyl]pyridine-2,6-diamine, 79
3-[6-(methylamino)-3-pyridyl]pyridine-2,6-diamine, 80
3-(6-pyrrolidin-1-yl-3-pyridyl)pyridine-2,6-diamine, 81
3-(6-amino-3-pyridyl)pyridine-2,6-diamine, 82
3-[6-amino-5-(trifluoromethyl)-3-pyridyl]pyridine-2,6-diamine, 83
3-(2-methyl-3-pyridyl)pyridine-2,6-diamine, 84
3-(6-fluoro-2-methyl-3-pyridyl)pyridine-2,6-diamine, 85
3-(6-fluoro-3-pyridyl)pyridine-2,6-diamine, 86
3-(2-fluoro-3-pyridyl)pyridine-2,6-diamine, 87
3-(4-methoxy-3-pyridyl)pyridine-2,6-diamine, 88
3-(2-methoxy-3-pyridyl)pyridine-2,6-diamine, 89
3-(3,5-dimethyl-1H-pyrazol-4-yl)pyridine-2,6-diamine, 90
3-(5-methyl-1H-pyrazol-4-yl)pyridine-2,6-diamine, 91
2-(2,6-diamino-3-pyridyl)benzonitrile (hydrochloride), 96

According to a preferred embodiment, the compounds of formula (I), (II) or (III) are selected in the group consisting of:
3-(2-chlorophenyl)pyridine-2,6-diamine (trifluoroacetate), 1b,
3-(o-tolyl)pyridine-2,6-diamine (trifluoroacetate), 1c,
3-(2-ethylphenyl)pyridine-2,6-diamine (hydrochloride), 1d,
3-(2-isopropylphenyl) pyridine-2,6-diamine (hydrochloride), 1e,
3-(2-(trifluoromethyl) phenyl) pyridine-2,6-diamine (trifluoroacetate), 1f,
3-(2-(methoxymethyl) phenyl) pyridine-2,6-diamine (hydrochloride), 1g,
3-(3-chlorophenyl)pyridine-2,6-diamine (trifluoroacetate), 1h,
3-(2,3-dichlorophenyl)pyridine-2,6-diamine, 1j,
3-(2,4-dichlorophenyl) pyridine-2,6-diamine (trifluoroacetate), 1k,
3-(2-chloro-3-(trifluoromethyl) phenyl) pyridine-2,6-diamine (trifluoroacetate), 1p,
3-([1,1′-biphenyl]-2-yl)pyridine-2,6-diamine (hydrochloride), 1r,
2-(2,6-diaminopyridin-3-yl)phenol, 2a,
3-(2-methoxyphenyl) pyridine-2,6-diamine (trifluoroacetate), 2b,
3-(2-(trifluoromethoxy) phenyl) pyridine-2,6-diamine (hydrochloride), 2c,
3-(2-ethoxyphenyl)pyridine-2,6-diamine (hydrochloride), 2d,
3-(2-butoxyphenyl)pyridine-2,6-diamine, 2e,
3-(2-isopropoxyphenyl)pyridine-2,6-diamine 2f,
3-(2-isobutoxyphenyl) pyridine-2,6-diamine (hydrochloride), 2g,
3-(2-methoxyethoxyphenyl)pyridine-2,6-diamine, 2h,
3-(2-(cyclopentyloxy)phenyl)pyridine-2,6-diamine 2i,
3-(2-(piperidin-1-yl)ethoxy)pyridine-2,6-diamine 2j,
3-(4-fluoro-2-methoxyphenyl) pyridine-2,6-diamine(hydrochloride), 2k,
3-(2,3-dimethoxyphenyl) pyridine-2,6-diamine (hydrochloride), 2l,
3-(2,4-dimethoxyphenyl pyridine-2,6-diamine (hydrochloride), 2m,
N-(6-amino-5-(2,3-dichlorophenyl)pyridin-2-yl)acetamide, 4a,
3-(2,3-dichlorophenyl)-N2-methylpyridine-2,6-diamine, 5a,
3-(2,3-dichlorophenyl)-N2-ethylpyridine-2,6-diamine, 5b,
N2-benzyl-3-(2,3-dichlorophenyl)pyridine-2,6-diamine, 5d,
5-(2,3-dichlorophenyl)-6-methylpyridin-2-amine, 6b,
5-(2,3-dichlorophenyl)-6-ethylpyridin-2-amine, 6c,
6-ethyl-5-(2-methoxyphenyl)pyridin-2-amine, 6d,
5-(2-methoxyphenyl)-6-propylpyridin-2-amine, 6g,
6-isopropyl-5-(2-methoxyphenyl)pyridin-2-amine, 6h,
6-cyclopropyl-5-(2-methoxyphenyl)pyridin-2-amine, 6i,
3-(2-chlorobenzyl)pyridine-2,6-diamine, 10e,
3-(2,4-dichlorobenzyl)pyridine-2,6-diamine (hydrochloride), 10f,
   including their salts (such as hydrochloride or trifluoroacetate).

According to a preferred embodiment, the compounds of formula (III) are selected in the group consisting of:
3-(2-chlorophenyl)pyridine-2,6-diamine (trifluoroacetate), 1b,
3-(2-isopropylphenyl) pyridine-2,6-diamine (hydrochloride), 1e,
3-(2-(trifluoromethyl) phenyl) pyridine-2,6-diamine (trifluoroacetate), 1f,
3-(2-(methoxymethyl) phenyl) pyridine-2,6-diamine (hydrochloride), 1g,
3-(2,3-dichlorophenyl)pyridine-2,6-diamine, 1j,
3-(2,4-dichlorophenyl) pyridine-2,6-diamine (trifluoroacetate), 1k,
3-(2,5-dichlorophenyl) pyridine-2,6-diamine (trifluoroacetate), 1l,
3-(2,6-dichlorophenyl) pyridine-2,6-diamine, 1m,
3-(2-chloro-3-(trifluoromethyl) phenyl) pyridine-2,6-diamine (trifluoroacetate), 1p,
3-(3-chloro-2-methylphenyl) pyridine-2,6-diamine (trifluoroacetate), 1q,
3-([1,1′-biphenyl]-2-yl)pyridine-2,6-diamine (hydrochloride), 1r,
3-(2-methoxyphenyl) pyridine-2,6-diamine (trifluoroacetate), 2b,
3-(2-(trifluoromethoxy) phenyl) pyridine-2,6-diamine (hydrochloride), 2c,
3-(2-ethoxyphenyl)pyridine-2,6-diamine (hydrochloride), 2d,
3-(2-butoxyphenyl)pyridine-2,6-diamine, 2e,
3-(2-isopropoxyphenyl)pyridine-2,6-diamine 2f,
3-(2-isobutoxyphenyl) pyridine-2,6-diamine (hydrochloride), 2g,
3-(2-methoxyethoxyphenyl)pyridine-2,6-diamine, 2h,
3-(2-(cyclopentyloxy)phenyl)pyridine-2,6-diamine 2i,
3-(4-fluoro-2-methoxyphenyl) pyridine-2,6-diamine (hydrochloride), 2k,
3-(2,3-dimethoxyphenyl) pyridine-2,6-diamine (hydrochloride), 2l,
3-(2,4-dimethoxyphenyl) pyridine-2,6-diamine (hydrochloride), 2m,
3-(2,3-dichlorophenyl)-N2-methylpyridine-2,6-diamine, 5a,
3-(2,3-dichlorophenyl)-N2-ethylpyridine-2,6-diamine, 5b,
N2-butyl-3-(2,3-dichlorophenyl)pyridine-2,6-diamine, 5c,
N2-benzyl-3-(2,3-dichlorophenyl)pyridine-2,6-diamine, 5d,
3-(2,3-dichlorophenyl)-N2-isopropylpyridine-2,6-diamine, 5e,
3-(2,3-dichlorophenyl)-N2-phenethylpyridine-2,6-diamine, 5f,
3-(2,3-dichlorophenyl)-N2-(2-methoxyethyl)pyridine-2,6-diamine, 5g,
3-(2,3-dichlorophenyl)-N2-(2-(piperidin-1-yl)ethyl)pyridine-2,6-diamine, 5h,
5-(2,3-dichlorophenyl)-6-(piperidin-1-yl)pyridin-2-amine, 5i,
5-(2,3-dichlorophenyl)pyridin-2-amine, 6a,
5-(2,3-dichlorophenyl)-6-methylpyridin-2-amine, 6b,
5-(2,3-dichlorophenyl)-6-ethylpyridin-2-amine, 6c,
6-ethyl-5-(2-methoxyphenyl)pyridin-2-amine, 6d,
6-(methoxymethyl)-5-(2-methoxyphenyl)pyridin-2-amine, 6e,
5-(2-methoxyphenyl)-6-(trifluoromethoxy)pyridin-2-amine, 6f,
5-(2-methoxyphenyl)-6-propylpyridin-2-amine, 6g,
6-isopropyl-5-(2-methoxyphenyl)pyridin-2-amine, 6h,
6-cyclopropyl-5-(2-methoxyphenyl)pyridin-2-amine, 6i,
5-(2,3-dichlorophenyl)-6-methoxypyridin-2-amine, 7a,
3-(2,3-dichlorophenyl)-4-methoxypyridine-2,6-diamine, 8a,
3-(2,3-dichlorophenyl)-5-fluoropyridine-2,6-diamine 9a,
3-(2,3-dichlorophenyl)-5-ethylpyridine-2,6-diamine, 9b,
   and one of a salt thereof (such as hydrochloride or trifluoroacetate).

More particularly, compounds of formula (III) are selected in the group consisting of:
3-(2-chlorophenyl)pyridine-2,6-diamine, 1b,
3-(2-isopropylphenyl) pyridine-2,6-diamine, 1e,
3-(2-(trifluoromethyl) phenyl) pyridine-2,6-diamine, 1f,
3-(2-(methoxymethyl) phenyl) pyridine-2,6-diamine, 1g,
3-(2,3-dichlorophenyl)pyridine-2,6-diamine, 1j,
5-(2-Methoxy-phenyl)-3-methyl-pyridin-2-ylamine, 13
5-(2-Methoxy-phenyl)-3-trifluoromethyl-pyridin-2-ylamine, 14
3-Fluoro-5-(2-methoxy-phenyl)-pyridin-2-ylamine, 15
5-(2,3-Dichloro-phenyl)-3-fluoro-pyridin-2-ylamine, 16
5-(2,3-Dichloro-phenyl)-4-fluoro-pyridin-2-ylamine, 17
4-Fluoro-5-(2-methoxy-phenyl)-pyridin-2-ylamine, 18
5-(2,3-Dichloro-phenyl)-4-methoxy-pyridin-2-ylamine (hydrochloride), 19
4-Methoxy-5-(2-methoxy-phenyl)-pyridin-2-ylamine, 20
5-(2-Methoxy-phenyl)-4-methyl-pyridin-2-ylamine, 21
5-(2,3-Dichloro-phenyl)-4-methyl-pyridin-2-ylamine, 22
5-(2-Methoxy-phenyl)-4-(2,2,2-trifluoro-ethoxy)-pyridin-2-ylamine (hydrochloride), 23
5-(2,3-Dichloro-phenyl)-4-(2,2,2-trifluoro-ethoxy)-pyridin-2-ylamine (hydrochloride), 24
4-(2-aminoethoxy)-5-(2-methoxyphenyl)pyridin-2-amine (dihydrochloride), 25
5-(2-Methoxy-phenyl)-6-methyl-pyridin-2-ylamine, 26
5-(2-Methoxy-phenyl)-4,6-dimethyl-pyridin-2-ylamine, 27
5-(2,3-Dichloro-phenyl)-4,6-dimethyl-pyridin-2-ylamine, 28
5-(3-chloro-2-methyl-phenyl)-6-ethyl-pyridin-2-ylamine (hydrochloride), 29
5-(2-cyclopropylphenyl)-6-ethyl-pyridin-2-amine (hydrochloride), 30
5-[2-(cyclopropoxy)phenyl]-6-ethyl-pyridin-2-amine (hydrochloride), 31
6-Fluoro-5-(2-methoxy-phenyl)-pyridin-2-ylamine, 32
5-(2,3-Dichloro-phenyl)-6-fluoro-pyridin-2-ylamine, 33
5-(2,3-Dichloro-phenyl)-6-trifluoromethyl-pyridin-2-ylamine, 34
6-Methoxy-5-(2-methoxy-phenyl)-pyridin-2-ylamine, 35
5-(2-Methoxy-phenyl)-6-(2,2,2-trifluoro-ethoxy)-pyridin-2-ylamine, 36
6-(2-Amino-ethoxy)-5-(2-methoxy-phenyl)-pyridin-2-ylamine, 37
6-(2-Amino-ethoxy)-5-(2,3-dichloro-phenyl)-pyridin-2-ylamine (dihydrochloride), 38
3-(2-Isopropoxy-6-methoxy-phenyl)-pyridine-2,6-diamine, 39
3-(4-Methoxy-2-methyl-phenyl)-pyridine-2,6-diamine, 40
3-(4-Chloro-2-fluoro-phenyl)-pyridine-2,6-diamine, 41
3-(2-Cyclopropyl-phenyl)-pyridine-2,6-diamine (hydrochloride), 42
3-(2-Phenoxy-phenyl)-pyridine-2,6-diamine (hydrochloride), 43
3-(2-Benzyl-phenyl)-pyridine-2,6-diamine, 44
3-(2-Chloro-4-fluoro-phenyl)-pyridine-2,6-diamine, 45
3-(2-Isopropoxy-4-methyl-phenyl)-pyridine-2,6-diamine, 46
3-(4-Chloro-2-cyclopentyloxy-phenyl)-pyridine-2,6-diamine, 47
3-(2-Cyclopropoxy-phenyl)-pyridine-2,6-diamine, 48
3-(2-Isopropoxy-5-methyl-phenyl)-pyridine-2,6-diamine, 49
3-(5-Fluoro-2-isopropoxy-phenyl)-pyridine-2,6-diamine, 50
3-(2,6-Dimethyl-phenyl)-pyridine-2,6-diamine, 51
3-(2-Isopropoxy-5-trifluoromethyl-phenyl)-pyridine-2,6-diamine, 52
3-(4-Fluoro-2-isopropoxy-phenyl)-pyridine-2,6-diamine, 53
3-(4-Chloro-2-methyl-phenyl)-pyridine-2,6-diamine, 54
3-(5-Chloro-2-cyclopropyl-phenyl)-pyridine-2,6-diamine, 55
3-(5-Chloro-2-methyl-phenyl)-pyridine-2,6-diamine, 56
3-(2-Methyl-4-trifluoromethyl-phenyl)-pyridine-2,6-diamine, 57
3-(2-chloro-3-methyl-phenyl)-pyridine-2,6-diamine, 58
3-(2-Methylsulfanyl-phenyl)-pyridine-2,6-diamine (hydrochloride), 59
2-(2,6-Diamino-pyridin-3-yl)-N,N-diethyl-benzamide (hydrochloride), 60
3-(2-Dimethylamino-phenyl)-pyridine-2,6-diamine (hydrochloride), 61
N-[2-(2,6-Diamino-pyridin-3-yl)-phenyl]-acetamide, 62
3-(2-methylsulfonylphenyl)pyridine-2,6-diamine (hydrochloride), 63
3-(2-benzyloxyphenyl)pyridine-2,6-diamine (hydrochloride), 64
3-[2-(cyclopropylmethoxy)phenyl]pyridine-2,6-diamine (hydrochloride), 65
3-(3-Chloro-2-methyl-phenyl)-5-fluoro-pyridine-2,6-diamine, 66
5-[2-(cyclopentoxy)phenyl]-6-ethyl-pyridin-2-amine (hydrochloride), 76
   and one of a salt thereof (such as hydrochloride or trifluoroacetate).

According to a particular embodiment, the compounds of embodiment A are selected in the group consisting of:
6-ethyl-5-(1-naphthyl)pyridin-2-amine (hydrochloride), 67
3-(1-naphthyl)pyridine-2,6-diamine, 68
3-(2-methoxy-1-naphthyl)pyridine-2,6-diamine, 69
3-(2-isopropoxy-1-naphthyl)pyridine-2,6-diamine, 70
3-(4-methyl-1-naphthyl)pyridine-2,6-diamine (hydrochloride), 71
3-(4-fluoro-1-naphthyl)pyridine-2,6-diamine (hydrochloride), 72
3-(4-chloro-1-naphthyl)pyridine-2,6-diamine (hydrochloride), 73
4-(2,6-diamino-3-pyridyl)naphthalen-1-ol (hydrochloride), 74
3-[4-(dimethylamino)-1-naphthyl]pyridine-2,6-diamine (hydrochloride), 75

According to a particular embodiment, the compounds of embodiment B are selected in the group consisting of:
3-(benzofuran-2-yl) pyridine-2,6-diamine (hydrochloride), 3c
[3,4′-bipyridine]-2,6-diamine, 3d,
[3,3′-bipyridine]-2,6-diamine, 3e,
3-(6-morpholino-3-pyridyl)pyridine-2,6-diamine, 78
3-[6-(1-piperidyl)-3-pyridyl]pyridine-2,6-diamine, 79
3-[6-(methylamino)-3-pyridyl]pyridine-2,6-diamine, 80
3-(6-pyrrolidin-1-yl-3-pyridyl)pyridine-2,6-diamine, 81
3-(6-amino-3-pyridyl)pyridine-2,6-diamine, 82
3-[6-amino-5-(trifluoromethyl)-3-pyridyl]pyridine-2,6-diamine, 83
3-(2-methyl-3-pyridyl)pyridine-2,6-diamine, 84
3-(6-fluoro-2-methyl-3-pyridyl)pyridine-2,6-diamine, 85
3-(6-fluoro-3-pyridyl)pyridine-2,6-diamine, 86
3-(2-fluoro-3-pyridyl)pyridine-2,6-diamine, 87
3-(4-methoxy-3-pyridyl)pyridine-2,6-diamine, 88
3-(2-methoxy-3-pyridyl)pyridine-2,6-diamine, 89
3-(3,5-dimethyl-1H-pyrazol-4-yl)pyridine-2,6-diamine, 90
3-(5-methyl-1H-pyrazol-4-yl)pyridine-2,6-diamine, 91

The compounds of the invention as defined above, including compounds of formula (I), (II) or (III) or of embodiment A or B, are for use in the treatment of pain, and preferably chronic pain.

According to another specific embodiment, the compounds of the invention are for use to decrease or block hyperalgesia and/or tolerance effects linked to the use of an analgesic compound, in particular an opiate analgesic compound.

The compounds according to the invention also include enantiomers of same (pure or in mixtures, in particular racemic mixtures), geometric isomers of same, salts, hydrates and solvates of same, solid forms of same, as well as mixtures of said forms. When the compounds according to the invention are in the forms of salts, they are preferably pharmaceutically acceptable salts. Such salts include pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Further examples of pharmaceutically acceptable inorganic or organic acid addition salts include the pharmaceutically acceptable salts listed in J. Pharm. Sci. 1977, 66, 2.

The compounds of formulas (I) (including any of the particular embodiments as detailed above), (II) or (III) may be prepared according to techniques known to the person skilled in the art. The present invention describes in this respect various routes of synthesis, which are illustrated in the examples below and may be implemented by the person skilled in the art. The starting compounds may be obtained commercially or may be synthesized according to standard methods. It is understood that the present invention is not limited to a particular route of synthesis, and extends to other methods that enable the production of the indicated compounds. The compounds of the invention can be produced by any chemical or genetic technique commonly known in the art. More specifically, compounds of the invention may be prepared by one of the methods described by the following schemes.

The compounds according to the invention are powerful NPFF1 and/or NPFF2 receptor ligands (table 1). Ligands are compounds that bind to one or more binding sites of NPFF1 and/or NPFF2 receptors. They can be antagonists or agonists, partially or totally, of NPFF1 or NPFF2 receptors or both.

Certain compounds of the invention have Kᵢ<100 nM. Certain compounds show a certain selectivity for NPFF1 or NPFF2. In addition to these pharmacological properties, the compounds according to the invention can have highly satisfactory in vivo activities; they can decrease, even block, hyperalgesia induced by administration of opiate analgesics, as well as the development of analgesic tolerance.

One object of the invention thus relates to the compounds of general formula (III), the compounds of embodiment A, or the compounds of embodiment B, according to the invention, including variants, combinations of variants and the specific compounds specified above, as drugs, and to methods for preparing same.

The invention also relates to pharmaceutical compositions comprising the compounds of general formula (III), the compounds of embodiment A, or the compounds of embodiment B, according to the invention and a pharmaceutically acceptable carrier. “Pharmaceutically acceptable carrier, support or vehicle” refers to any carrier that is physiologically acceptable to the subject, in particular a human or animal subject, wherein said carrier depends on the type of administration.

The compounds and the pharmaceutical compositions according to the invention are particularly useful for a therapeutic method and in particular to the treatment of pain. In particular, the compounds and compositions according to the invention decrease or block hyperalgesia and/or tolerance effects related to the use of analgesic compounds, in particular opiate analgesic compounds. Thus, the compounds and the pharmaceutical compositions according to the invention may be used in the treatment of postoperative pain or of severe chronic pain caused by inflammation, neuropathy, cancer, diabetes or drugs.

The invention also relates to a method for treating pain in a subject, comprising the administration to said subject of an effective quantity of the compound or the pharmaceutical composition according to the invention.

The invention also relates to the use of at least one compound according to the invention for preparing a pharmaceutical composition intended to treat pain or to decrease or block hyperalgesia and/or tolerance effects related to the use of analgesic compounds, in particular opiate analgesic compounds.

In case where the compound or the pharmaceutical composition according to the invention is intended to decrease or block hyperalgesia and/or tolerance effects induced by the use of an analgesic compound, in particular an opiate analgesic compound, the compound or the pharmaceutical composition containing said compound may be administered simultaneously with, separately from or sequentially to the analgesic compound.

According to a particular variant, one object of the invention relates to a pharmaceutical composition comprising at least one compound according to the invention, at least one analgesic compound, in particular an opiate, and a pharmaceutically acceptable carrier.

The Analgesic Compounds

The analgesic compounds used in the context of the present invention are generally opiate compounds, i.e., compounds that act on opioid receptors. They are generally used to treat severe and long-lasting pain. Preferably, these are morphine compounds, notably morphine or morphinomimetic compounds, i.e., compounds that are derived from morphine and/or that act on morphine receptors and/or that recruit one or more metabolic pathways common to morphine. As particular examples, mention may be made of the following compounds in particular: morphine, fentanyl, sufentanil, alfentanyl, heroin, oxycodone, hydromorphone, levorphanol, methadone, buprenorphine, butorphanol, meperidine, etc.

The invention is quite particularly suited to the inhibition of hyperalgesia induced by morphine, fentanyl or heroin.

The term “treatment” comprises a curative treatment as well as a prophylactic treatment of pain. A curative treatment is defined as a treatment that eases, improves and/or eliminates, reduces and/or stabilizes suffering or pain. A prophylactic treatment comprises a treatment that prevents pain as well as a treatment that reduces and/or delays pain or the risk of the occurrence of pain.

By decreasing or blocking hyperalgesia and/or tolerance effects related to the use of opiates, the compounds according to the invention prolong the duration of action of opiates and/or increase the intensity of their analgesic effect, without causing hypersensitivity to pain. The growing need to increase the doses of opiates in order to maintain the same analgesic effect is thus decreased, even absent.

Generally, it appears that the administration of opiate analgesics to mammalian subjects is always accompanied by hyperalgesia, and thus the compound according to the invention may be used each time an opiate analgesic is administered to a subject.

Furthermore, as specified above, the administration of high doses of opiates leads to a certain number of side effects such as nausea, constipation, sedation and respiratory deficiencies (e.g.: delayed respiratory depression). The compounds according to the invention allows to use lower doses of opiates and should therefore limit adverse side effects of opiates, such as nausea, constipation, sedation or respiratory deficiencies, including delayed respiratory depression.

Furthermore, the effect of the compounds according to the invention on hypersensitivity to pain induced by opiates makes it possible to also envisage the administration of said compounds alone in the context of the prophylactic treatment of pain.

Hyperalgesia induced by stress or by opioids may be prolonged or brief, significant or moderate. The detection, measurement and characterization of the presence of hyperalgesia may be carried out by standard clinical tests (observation, etc.).

In the context of the present invention, the term “inhibit” means to decrease or block (or reduce or suppress) in a partial or total, transitory or prolonged manner. Thus, such terms in the present description are used interchangeably. The capacity to inhibit hyperalgesia and the degree of such inhibition may be determined according to various tests known to the person skilled in the art. Furthermore, the term “inhibit” refers to inhibition of the appearance of hyperalgesia (for a preventive treatment, for example) as well as to inhibition of the development or duration of hyperalgesia (for a curative treatment).

The compounds or compositions according to the invention may be administered in various ways and in various forms. Thus, they may be injected by oral or more generally by a systemic route, such as, for example, by intravenous, intramuscular, subcutaneous, transdermal, intra-arterial route, etc. Preferably, the compounds or compositions according to the invention are administered by oral route. For injections, the compounds are generally packaged in the form of liquid suspensions, which may be injected via syringes or perfusions, for example. In this respect, the compounds are generally dissolved in saline, physiological, isotonic or buffered solutions, etc., compatible with pharmaceutical use and known to the person skilled in the art. Thus, the compositions may contain one or more agents or excipients selected from dispersants, solubilizers, stabilizers, preservatives, etc. Agents or excipients that can be used in liquid and/or injectable formulations are notably methylcellulose, hydroxymethylcellulose, carboxymethylcellulose, polysorbate 80, mannitol, gelatin, lactose, vegetable oils, acacia, etc.

According to a particular variant, the compound according to the invention is administered by the same route as the analgesic compound, for example by oral route.

The compounds may also be administered in the form of gels, oils, tablets, suppositories, powders, gelatin capsules, capsules, etc., optionally by means of dosage forms or devices that ensure prolonged and/or delayed release. For this type of formulation, an agent such as cellulose, carbonate or starch is advantageously used.

It is understood that the flow rate and/or dose administered may be adjusted by the person skilled in the art according to the patient, the pain observed, the analgesic concerned, the mode of administration, etc. Typically, the compounds are administered at doses that may vary between 0.1 μg and 10 mg/kg of body weight, more generally from 1 μg to 1000 μg/kg. Furthermore, administration by oral route or by injection may comprise several (2, 3 or 4) administrations per day, if need be.

In addition, for chronic treatments, delayed or prolonged systems may be advantageous, ensuring the subject effective and long-lasting pain treatment.

The present invention may be used for the preventive or curative treatment of hyperalgesia in multiple situations, such as that occurring or associated with acute or chronic pain in response to surgery, trauma or pathology of a mammal.

It may be applied to any mammal, in particular humans, but also to animals, in particular domestic or breeding animals, in particular horses, dogs, etc.

It is particularly suited for preventing or treating sensitization processes induced by the limited administration of opiate analgesics, such as powerful morphinomimetics (morphine or fentanyl or derivatives thereof, for example), during surgical or trauma procedures.

It may also be used to prevent or treat chronic pain in mammals (particularly patients) suffering from pathologies such as cancer, burns, etc., for which generally analgesics (such as morphine) may be administered for a long period, optionally in delayed form.

The compounds according to the invention may also be used to prevent or reduce, in a highly significant manner, tolerance processes, thus making it possible to reduce daily doses of morphine and thus to improve the clinical picture of patients (side effects of morphinomimetics, such as intestinal disorders, for example).

Reversal of opiate-induced hyperalgesia makes it possible to maintain opiate effectiveness over time and thus to use lower doses of analgesics, which in turn causes fewer side effects.

The compounds of formula (I) (including any of the particular embodiments as detailed above), (II) or (III) according to the invention may also be used for the preventive or curative treatment of pain.

The compounds of formula (I) (including any of the particular embodiments as detailed above), (II) or (III) according to the invention may also be used for the treatment of opiate dependence (drug addiction).

According to a specific embodiment, the invention also relates to a kit that is suitable for the treatment by the methods described above. These kits comprise a composition containing the compound (I) (including any of the particular embodiments as detailed above), (II) or (III) of the invention in the dosages indicated above and a second composition containing an analgesic compound, preferably an opiate compound, in the dosages indicated above, for a simultaneous, separate or sequential administration, in effective amounts according to the invention.

Other aspects and advantages of the present invention will become apparent upon consideration of the following examples, which must be regarded as illustrative and nonrestrictive.

### EXAMPLES—SUMMARY OF THE EXAMPLES

1—General Synthetic Method for the Preparation of 3-Aryl (Heteroaryl)-2,6 diaminopyridine derivatives Starting from 2,6-diamino pyridine Methods 1-2
Example 1: Preparation of 3-(2,3-dichlorophenyl)pyridine-2,6-diamine, 1j, (Method 1)
Example 2: Preparation of 3-(furan-2-yl)pyridine-2,6-diamine, 3b (Method 2)
   2—Preparation of 3-Heteroaryl-2,6 diaminopyridine derivatives Starting from 3-Iodo-2,6-dichloropyridine (Method 3)
Example 3: Preparation of [3,4′-bipyridine]-2,6-diamine 3d
   3—Preparation of 3 (2-alkoxy phenyl)-2,6 diaminopyridine derivatives Starting from 2b: Methods 4 and 5
Example 4: Preparation of 3-(2-butoxyphenyl)pyridine-2,6-diamine, 2e
Example 5: Preparation of 3-(2-isopropoxyphenyl)pyridine-2,6-diamine, 2f
   4—Preparation of 3 (2-alkoxy phenyl)-2,6 diaminopyridine derivatives Starting from 3-Iodo-2,6-dichloropyridine: Method 6
Example 6: Preparation of 3-(2-(cyclopentyloxy)phenyl)pyridine-2,6-diamine, 2i
   5—Preparation of 3-Aryl-N2-alkylpyridine-2,6 diamine Starting from 1j by Reductive Amination of 4a (Method 7)
Example 7: Preparation of 3-(2,3-dichlorophenyl)-N2-methylpyridine-2,6-diamine, 5a
   6—Preparation of 3-Aryl-N2-alkylpyridine-2,6 diamine Starting from 6-fluoropyridin-2-amine (Method 8)
Example 8: Preparation of 3-(2,3-dichlorophenyl)-N2-phenethylpyridine-2,6-diamine, 5f
   7—Preparation of N2-alkyl-5-arylpyridine-2-amine: Method 9
Example 9: Preparation of 5-(2-methoxyphenyl)-6-propylpyridin-2-amine, 6g
Example 10: 6-cyclopropyl-5-(2-methoxyphenyl)pyridin-2-amine, 6i
Example 11: Preparation of 6-(methoxymethyl)-5-(2-methoxyphenyl)pyridin-2-amine, 6e
   8—Preparation of N2-alkoxy-5-arylpyridine-2-amine: Method 10
Example 12: Preparation of 5-(2,3-dichlorophenyl)-6-methoxypyridin-2-amine, 7a

### 9—Additional Substitution at Positions 4 (R4) or 5 (R5), Methods 11-13

Example 13: 4-methyl-3-(o-tolyl)pyridine-2,6-diamine, 8b,
Example 14: 3-(2,3-dichlorophenyl)-4-methoxypyridine-2,6-diamine, 8a
Example 15: Preparation of 3-(2,3-dichlorophenyl)-5-fluoropyridine-2,6-diamine, 9a
Example 16: 3-(2,3-dichlorophenyl)-5-ethylpyridine-2,6-diamine, 9b,
   10—General Synthetic Method for the Preparation of 3-benzyl pyridine-2,6-diamine derivatives 10, Methods 14-15
Example 17: 3-(2-chlorobenzyl)pyridine-2,6-diamine, 10e, Example 18: 3-(2,4-dichlorobenzyl)pyridine-2,6-diamine hydrochloride, 10f, (method 15)
   11—General Synthetic Method for the Preparation of 3-phenethyl pyridine-2,6-diamine derivatives 11, Method 16
Example 19: 3-phenethylpyridine-2,6-diamine, 11a

### 12—Methods for the Preparation of Compounds No 13-96, Methods 17-23

Example 20: Compounds 13-96
   Methods for preparation of compounds No 13-66 in substituted phenyl series
   Extension of methods 17 and 20 for preparation of compounds No 68-No 75 in substituted napthyl series
   Method 21 for preparation of compound No 76 in substituted phenyl series
   Method 22 for preparation of compound No 77 in substituted phenyl series
   Method 23 for the preparation of additional compounds No 78-No 91 in HetAr series
   Method A for the synthesis of non-commercially available aryl-bromides
   Method 20 for the preparation of compound No 96

### 13—Pharmacological Data:

Examples 21
   Binding assays with both receptors NPFFR1 and 2
   Glo Sensor cAMP assay in HEK-293 stably expressing hNPFFR1 and hNPFFR2

### Example 22: Human NPFFR1 Evaluation Using BRET Biosensors (IC50)

### 14—Activity of NPFF1R Receptor Antagonist Compound 1j in Models of Opioid-Induced Hyperalgesia and Analgesic Tolerance, Surgical Pain and Neuropathic Pain

### Example 23

### Examples

### General Synthetic Methods

The following synthetic methods and schemes illustrate the general methods by which the compounds of the present invention can be prepared. Starting materials can be obtained from commercially sources or prepared using methods well known to those of ordinary skill in the art.

1—General Synthetic Method for the Preparation of 3-Aryl (Heteroaryl)-2,6 diaminopyridine derivatives Starting from 2,6-diamino pyridine. Methods 1-2

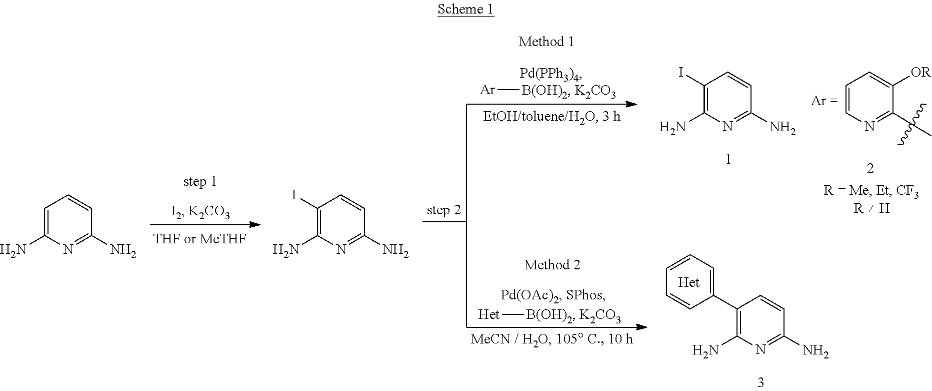

According to the above reaction scheme 1, the easily available 3-Iodo-2,6 diamino pyridine is the key intermediate for the preparation of the 3-aryl-2,6-diaminopyridine derivatives of the general formula 1-3. This approach involved a Suzuki-Miyaura cross coupling reaction using a tetrakis(triphenylphosphine palladium (0), K₂CO₃ catalyst system with toluene/ethanol/water as the solvents heated at reflux (Method 1). Introduction of heterocycles in position 3 needs the use of Pd(OAc)₂ in presence of SPhos in a mixture of acetonitrile and water to afford 3-heteroaryl 2,6-diaminopyridine derivatives of the general formula 3. (Method 2)

### Example 1: Preparation of 3-(2,3-dichlorophenyl)pyridine-2,6-diamine, 1j (Method 1)

### Step 1: 3-iodopyridine-2,6-diamine

To a solution of 2,6-diaminopyridine (3.0 g, 27.5 mmol, 1 eq.) in 2-methyl-tetrahydrofuran (60 mL) was added potassium carbonate (3.8 g, 27.5 mmol, 1 eq.). To this suspension was added a solution of iodine (6.98 g, 27.5 mmol, 1 eq.) in 2-methyl-tetrahydrofuran (50 mL) dropwise over 1 hour. The reaction was stirred for 2 hours at room temperature. The reaction was filtered through a pad of celite, and the filtrate collected and washed with water (50 ml) and saturated aqueous sodium thiosulphate solution (50 ml). The organic layer was dried over sodium sulphate and concentrated in vacuo, azeotroping with dichloromethane to afford a light brown solid. The solid was stirred in methanol (100 ml) for 15 minutes. The suspension was filtered and the filtrate collected and concentrated in vacuo. The residue was purified by flash column chromatography using a gradient of 33% to 50% ethyl acetate in heptane to give the 3 iodopyridine-2,6-diamine (4.85 g, 75%) as a light brown solid.

¹H NMR (400 MHz, CDCl₃): δ 7.49 (d, J=7.9 Hz, 1H), 5.97 (d, J=7.9 Hz, 1H), 4.62 (s, 2H), 4.20 (s, 2H).

¹³C-NMR (101 MHz, CDCl₃): δ 157.9, 156.7, 147.6, 100.8, 61.3.

### Step 2: 3-(2,3-dichlorophenyl)pyridine-2,6-diamine, 1j

A 5 ml microwave vial containing a Teflon® stirred bar was charged with 3-iodopyridine-2,6-diamine (1.0 g, 4.25 mmol, 1 eq.), 2,3-dichlorophenyl boronic acid (852 mg, 4.47 mmol, 1.05 eq.), Na₂CO₃ (1.36 g, 12.76 mmol, 3 eq.) followed by the addition of a mixture of Toluene/EtOH/H₂O: 6/1/1 (0.1 mmol/mL). The vessel was evacuated and backfilled with nitrogen (this process was repeated a total of 3 times) and Pd(PPh₃)₄ (248 mg, 0.21 mmol, 0.05 eq.) was introduced. The reaction mixture was then capped properly and placed in a preheated oil bath at 120° C. until complete conversion of the starting material was detected. The reaction mixture was monitored by HPLC analysis and was usually complete within 2-4 hours. The reaction mixture was then concentrated under vacuum and the crude product was purified by chromatography on silica gel using EtOAc/heptane: 1/1 to afford the expected product 1j as a white solid (1.0 g, 92%).

¹H-NMR (400 MHz, CDCl₃): δ 7.44 (t, J=4.7 Hz, 1H), 7.22 (d, J=4.7 Hz, 2H), 7.09 (d, J=7.9 Hz, 1H), 5.97 (d, J=7.9 Hz, 1H), 4.28 (s, 2H), 4.12 (s, 2H).

¹³C-NMR (101 MHz, CDCl₃): δ 157.9, 154.6, 140.6, 139.5, 134.0, 133.1, 130.6, 129.9, 127.8, 109.0, 98.1.

All Compounds 1, 2 reported in table 1 are prepared following general method of preparation of 1j.

TABLE 1

| | | Yield | |
|---|---|---|---|
| N° | Name | % | RMN |
| 1a | 3-phenylpyridine-2,6- | 80 | ¹H-NMR (400 MHz, CDCl₃): δ 7.38 (m, 4H), 7.28 (m, 1H), 7.18 |
| | diamine | | (d, J = 7.9 Hz, 1H), 5.97 (d, J = 7.9 Hz, 1H), 4.45 (bs, 2H), 4.32 |
| | | | (bs, 2H). |
| | | | ¹³C-NMR (101 MHz, CDCl₃): δ 157.0, 154.7, 140.5, 138.9, |
| | | | 129.1, 129.0, 127.0, 111.7, 98.6. |
| 1b^{a} | 3-(2-chlorophenyl)pyridine- | 86 | ¹H-NMR (400 MHz, MeOD-d4): δ 7.57-7.54 (m, 1H), 7.44-7.39 |
| | 2,6-diamine trifluoroacetate | | (m, 3H), 7.37-7.34 (m, 1H), 6.01 (d, J = 8.5 Hz, 1H). |
| | | | ¹³C-NMR (101 MHz, MeOD-d4): δ 153.9, 148.0, 135.9, |
| | | | 134.8, 133.6, 131.6, 131.4, 129.0, 108.2, 97.2. |
| 1c^{a} | 3-(o-tolyl)pyridine-2,6- | 50 | ¹H-NMR (400 MHz, DMSO-d6): δ 12.49 (bs, 1H), 7.36 (d, J = 8.4 |
| | diamine trifluoroacetate | | Hz, 1H), 7.32 (m, 2H), 7.29-7.25 (m, 3H), 7.13 (d, J = 7.4 Hz, |
| | | | 1H), 6.60 (bs, 2H), 6.02 (d, J = 8.4 Hz, 1H), 2.12 (s, 3H). |
| | | | ¹³C-NMR (101 MHz, DMSO-d6): δ 151.8, 149.0, 145.8, |
| | | | 137.0, 134.2, 130.5, 130.4, 128.2, 126.3, 107.3, 95.6, |
| | | | 19.2. |
| 1d^{b} | 3-(2-ethylphenyl)pyridine- | 47 | ¹H-NMR (400 MHz, DMSO-d6): δ 12.95 (bs, 1H), 7.37- |
| | 2,6-diamine hydrochloride | | 7.35 (m, 5H), 7.29- 7.25 (m, 1H), 7.11 (d, J = 7.5 Hz, |
| | | | 1H), 6.68 (bs, 2H), 6.03 (d, J = 8.4 Hz, 1H), 2.47-2.35 |
| | | | (m, 2H), 1.04 (t, J = 7.5 Hz, 3H). |
| | | | ¹³C-NMR (101 MHz, DMSO-d6): δ 151.4, 149.2, 145.8, |
| | | | 143.1, 133.5, 130.8, 128.8, 128.6, 126.4, 101.2, 95.7, |
| | | | 25.6, 15.2. |
| 1e^{b} | 3-(2-isopropylphenyl) | 35 | ¹H-NMR (400 MHz, DMSO-d6): δ 12.82 (bs, 1H), 7.46-7.40 (m, |
| | pyridine-2,6-diamine | | 2H), 7.36 (d, J = 8.3 Hz, 1H), 7.35 (s, 2H), 7.28 (td, J = 7.2 Hz, 1.3 |
| | hydrochloride | | Hz, 1H), 7.10 (d, J = 7.5 Hz, 1H), 6.67 (bs, 2H), 6.05 (d, J = 8.3 Hz, |
| | | | 1H), 2.73 (hept, J =6.8 Hz, 1H), 1.19 (d, J = 6.8 Hz, 3H), 1.07 (d, |
| | | | J = 6.8 Hz, 3H). |
| | | | ¹³C-NMR (101 MHz, DMSO-d6) δ: 150.8, 149.2, 148.5, |
| | | | 145.5, 131.4, 130.6, 129.8, 126.8, 126.6, 109.7, 97.2, |
| | | | 30.1, 24.6, 23.6. |
| 1f^{a} | 3-(2-(trifluoromethyl) | 35 | ¹H-NMR (400 MHz, DMSO-d6): δ 12.96 (bs, 1H), 7.85 (d, J = 8.0 |
| | phenyl) pyridine-2,6-diamine | | Hz, 1H), 7.74 (t, J = 7.6 Hz, 1H), 7.65 (t, J = 7.7 Hz, 1H), 7.40 (d, |
| | trifluoroacetate | | J = 7.5 Hz, 1H), 7.36 (bs, 2H), 7.32 (d, J = 8.5 Hz, 1H), 6.77 (bs, |
| | | | 2H), 6.01 (d, J = 8.5 Hz, 1H). |
| | | | ¹³C-NMR (101 MHz, DMSO-d6): δ 152.3, 149.7, 145.4, |
| | | | 133.5, 133.3 (d, J = 2.3 Hz), 133.0, 129.2 (q, J = 29 Hz), |
| | | | 129.0, 126.5 (q, J = 5.3 Hz), 124.0 (q, J = 275 Hz), |
| | | | 104.7, 95.1. |
| 1g^{a} | 3-(2-(methoxymethyl) | 31 | ¹H-NMR (400 MHz, DMSO-d6): δ 13.15 (bs, 1H), 7.50 (d, |
| | phenyl) pyridine-2,6-diamine | | J = 7.1 Hz, 1H), 7.44-7.37 (m, 4H), 7.35 (d, J = 8.5 Hz, |
| | hydrochloride | | 1H), 7.18 (d, J = 6.9 Hz, 1H), 6.71 (bs, 2H), 6.03 (d, J = |
| | | | 8.4 Hz, 1H), 4.25 (d, J = 12.0 Hz, 1H), 4.19 (d, J = 12.0 |
| | | | Hz, 1H), 3.21 (s, 3H). |
| | | | ¹³C-NMR (101 MHz, DMSO-d6): δ 151.6, 149.2, 145.6, |
| | | | 137.5, 133.5, 130.7, 128.6, 128.2, 128.0, 106.1, 95.7, |
| | | | 71.3, 57.7. |
| 2b^{a} | 3-(2-methoxyphenyl) | 57 | ¹H-NMR (400 MHz, DMSO-d6): δ 12.45 (bs, 1H), 7.40 (m, 2H), |
| | pyridine-2,6-diamine | | 7.24 (bs, 2H), 7.16 (d, J = 7.4 Hz, 1H), 7.10 (d, J = 7.4 Hz, 1H), |
| | trifluoroacetate | | 7.02 (t, J = 7.4 Hz, 1H), 6.63 (bs, 2H), 6.00 (d, J = 8.4 Hz, 1H), |
| | | | 3.76 (s, 3H). |
| | | | ¹³C-NMR (101 MHz, DMSO-d6): δ 156.7, 151.6, 149.2, |
| | | | 146.4, 131.2, 129.6, 123.2, 120.7, 111.7, 105.1, 95.6, |
| | | | 55.3. |
| 2c^{a} | 3-(2-(trifluoromethoxy) | 49 | ¹H-NMR (400 MHz, DMSO-d6): δ 12.85 (bs, 1H), 7.58- |
| | phenyl) pyridine-2,6-diamine | | 7.55 (m, 1H), 7.51-7.41 (m, 6H), 6.95 (bs, 2H), 6.04 (d, |
| | hydrochloride | | J = 8.4 Hz, 1H). |
| | | | ¹³C-NMR (101 MHz, DMSO-d6): δ 151.7, 148.4, 146.5, |
| | | | 145.2, 131.7, 129.5, 127.5, 127.2, 121.1, 102.3, 96.3. |
| 2d^{b} | 3-(2-ethoxyphenyl)pyridine- | 45 | ¹H-NMR (400 MHz, DMSO-d6): δ 12.86 (s, 1H), 7.40 (d, J = |
| | 2,6-diamine hydrochloride | | 7.5 Hz, 1H), 7.36 (td, J = 7.8 Hz, 1.7 Hz, 1H), 7.32 (bs, |
| | | | 2H), 7.16 (dd, J = 7.5 Hz, 1.8 Hz, 1H), 7.08 (d, J = 8.1 |
| | | | Hz, 1H), 7.00 (t, J = 7.2 Hz, 1H), 6.72 (bs, 2H), 6.01 (d, |
| | | | J = 8.5 Hz, 1H), 4.05 (q, J = 7.0 Hz, 2H), 1.25 (d, J = 7.0 |
| | | | Hz, 3H). |
| | | | ¹³C-NMR (101 MHz, DMSO-d6): δ 155.0, 151.2, 149.2, |
| | | | 146.3, 131.2, 129.5, 123.4, 120.7, 112.6, 105.2, 95.6, |
| | | | 63.3, 14.6. |
| 2g^{b} | 3-(2-isobutoxyphenyl) | 72 | ¹H-NMR (400 MHz, DMSO-d6): δ 13.33 (bs, 1H), 7.39-7.3 |
| | pyridine-2,6-diamine | | (m, 3H), 7.34 (d, J = 7.2 Hz, 1H), 7.16 (d, J = 7.2 Hz, 1H), |
| | hydrochloride | | 7.06 (d, J = 8.4 Hz, 1H), 6.99 (t, J = 7.2 Hz, 1H), 6.76 |
| | | | (bs, 2H), 6.02 (d, J = 8.4 Hz, 1H), 3.74 (d, J = 6.3 Hz, |
| | | | 2H), 1.92 (hept, J = 6.3 Hz, 1H), 0.88 (d, J = 6.3 Hz, 6H). |
| | | | ¹³C-NMR (101 MHz, DMSO-d6): δ 156.2, 151.2, 149.3, |
| | | | 146.2, 131.1, 129.5, 123.4, 120.6, 112.6, 105.0, 95.4, 73.8, |
| | | | 27.8, 19.0. |
| 2k^{b} | 3-(4-fluoro-2- | 59 | ¹H-NMR (400 MHz, DMSO-d6): δ 13.33 (bs, 1H), 7.39 (bs, |
| | methoxyphenyl) pyridine- | | 2H), 7.34 (d, J = 8.6 Hz, 1H), 7.17 (dd, J = 8.2 Hz, 7.1 |
| | 2,6-diamine hydrochloride | | Hz, 1H), 7.17 (dd, J = 11.5 Hz, 2.5 Hz, 1H), 6.84 (bs, |
| | | | 2H), 6.83 (td, J = 8.4 Hz, 2.5 Hz, 1H ), 6.00 (d, J = 8.4 |
| | | | Hz, 1H ), 3.76 (s, 3H). |
| | | | ¹³C-NMR (101 MHz, DMSO-d6): δ 163.1 (d, J = 244 Hz), |
| | | | 158.3 (d, J = 11.1 Hz), 151.4, 149.5, 146.2, 132.4 (d, J = 9.9 Hz), |
| | | | 119.5 (d, J = 2.9 Hz), 107.0 (d, J = 22 Hz), 104.1, 100.0 (d, J = 26 |
| | | | Hz), 95.5, 55.8. |
| 1h^{a} | 3-(3-chlorophenyl)pyridine- | 77 | ¹H-NMR (400 MHz, CDCl₃): δ 7.37-730 (m, 3H), 7.26 (t, J = 1.7 |
| | 2,6-diamine trifluoroacetate | | Hz, 1H), 7.17 (dt, J = 7.2 Hz, 1.7 Hz, 1H), 6.20 (bs, 2H), 6.12 (bs, |
| | | | 2H), 5.93 (d, J = 8.4 Hz, 1H). |
| | | | ¹³C-NMR (101 MHz, CDCl₃): δ 152.2, 150.0, 145.4, 136.5, |
| | | | 135.9, 131.1, 129.0, 128.9, 126.8, 109.0, 97.4. |
| 1i^{a} | 3-(4-chlorophenyl)pyridine- | 27 | ¹H-NMR (400 MHz, DMSO-d6): δ 12.78 (bs, 1H), 7.42-7.48 (m, |
| | 2,6-diamine trifluoroacetate | | 3H), 7.40-7.36 (m, 4H), 6.92 (bs, 2H), 6.06 (d, J = 8.5 Hz, 1H). |
| | | | ¹³C-NMR (101 MHz, DMSO-d6): δ 151.2, 149.3, 145.4, |
| | | | 134.2, 132.0, 130.6, 128.9, 106.5, 96.6. |
| 1k^{a} | 3-(2,4-dichlorophenyl) | 67 | ¹H-NMR (400 MHz, MeOD-d4): δ 7.62 (d, J = 2.2 Hz, 2H), 7.44 |
| | pyridine-2,6-diamine | | (dd, J = 8.2 Hz, , 2.2Hz, 1H), 7.42 (d, J = 8.5 Hz, 1H), 7.35 (d, J = |
| | trifluoroacetate | | 8.2 Hz, 1H), 6.94 (bs, 1H), 6.02 (d, J = 8.5 Hz, 1H). |
| | | | ¹³C-NMR (101 MHz, MeOD-d4): δ 154.1, 151.1, 147.9, |
| | | | 137.0, 136.6, 134.7, 133.7, 131.1, 129.3, 106.9, 97.4. |
| 1l^{a} | 3-(2,5-dichlorophenyl) | 63 | ¹H-NMR (400 MHz, DMSO-d6): δ 12.99 (bs, 1H), 7.60 (d, J = 8.5 |
| | pyridine-2,6-diamine | | Hz, 1H), 7.50 (dd, J = 8.5 Hz, 2.3 Hz, 1H), 745 (d, J = 2.3 Hz, 1H), |
| | trifluoroacetate | | 7.42 (bs, 2H), 7.41 (d, J = 8.5 Hz, 1H), 7.03 (bs, 2H), 6.05 (d, J = |
| | | | 8.5 Hz, 1H). |
| | | | ¹³C-NMR (101 MHz, DMSO-d6): δ 152.6, 149.4, 145.8, 135.5, |
| | | | 132.7, 132.2, 132.0, 131.4, 129.8, 104.2, 95.7. |
| 1m | 3-(2,6-dichlorophenyl) | 13 | ¹H-NMR (400 MHz, CDCl3): δ 7.47 (d, J = 8.3 Hz, 2H), 7.35 (t, J = |
| | pyridine-2,6-diamine | | 8.3 Hz, 1H), 7.32 (d, J = 8.5 Hz, 1H), 6.34 (bs, 2H), 6.02 (d, J = 8.5 |
| | | | Hz, 1H), 5.84 (bs, 2H). |
| | | | ¹³C-NMR (125 MHz, MeOD-d4): δ 154.2, 148.3, 141.9, 141.7, |
| | | | 138.2, 132.4, 130.0, 107.8, 97.4. |
| In^{a} | 3-(3,4-dichlorophenyl) | 61 | ¹H-NMR (400 MHz, DMSO-d6): δ 13.04 (bs, 1H), 7.69 (d, J = 8.3 |
| | pyridine-2,6-diamine | | Hz, 1H), 7.61 (d, J = 1.3 Hz, 1H), 7.51 (d, J = 8.5 Hz, 1H), 7.40 (bs, |
| | trifluoroacetate | | 2H), 7.34 (dd, J = 8.3 Hz, 1.3 Hz, 1H), 7.03 (bs, 2H), 6.05 (d, J = |
| | | | 8.5 Hz, 1H). |
| | | | ¹³C-NMR (101 MHz, DMSO-d6): δ 152.4, 149.5, 145.3, 136.1, |
| | | | 131.5, 131.0, 130.8, 129.9, 129.2, 105.4, 96.6. |
| 1o^{a} | 3-(3,5-dichlorophenyl) | 63 | ¹H-NMR (400 MHz, DMSO-d6): δ 12.94 (bs, 1H), 7.58 (s, 1H), |
| | pyridine-2,6-diamine | | 7.52 (d, J = 8.5 Hz, 1H), 7.41 (bs, 2H), 7.40 (s, 2H), 7.04 (bs, 2H), |
| | trifluoroacetate | | 6.05 (d, J = 8.5 Hz, 1H). |
| | | | ¹³C-NMR (101 MHz, DMSO-d6): δ 1526, 149.6, 145.3, 139.1, |
| | | | 134.4, 127.6, 126.8, 105.1, 96.6. |
| 2l^{b} | 3-(2,3-dimethoxyphenyl) | 50 | ¹H-NMR (400 MHz, DMSO-d6): δ 12.90 (bs, 1H), 7.39 (d, J = 8.3 |
| | pyridine-2,6-diamine | | Hz, 1H), 7.36 (bs, 2H), 7.15-7.08 (m, 2H), 7.76 (dd, J = 7.2 Hz, |
| | hydrochloride | | 1.9 Hz, 1H), 6.73 (bs, 2H), 6.02(d, J = 8.3 Hz, 1H), 3.83 (s, 3H), |
| | | | 3.58 (s, 3H). |
| | | | ¹³C-NMR (101 MHz, DMSO-d6): δ 152.8, 151.4, 149.3, |
| | | | 146.8, 145.9, 128.6, 124.4, 122.8, 113.0, 104.5, 95.7, |
| | | | 60.2, 55.7. |
| 2m^{b} | 3-(2,4-dimethoxyphenyl | 31 | ¹H-NMR (400 MHz, DMSO-d6): δ 12.91 (s, 1H), 7.33 (d, J = |
| | pyridine-2,6-diamine | | 8.4 Hz, 1H), 7.27 (bs, 2H), 7.05 (d, J = 8.3 Hz, 1H), 6.70 |
| | hydrochloride | | (bs, 2H), 6.64 (d, J = 2.4 Hz, 1H), 6.60 (dd, J = 8.3 Hz, |
| | | | 2.4 Hz, 1H), 5.98 (d, J = 8.4 Hz, 1H), 3.80 (s, 3H), 3.74 |
| | | | (s, 3H). |
| | | | ¹³C-NMR (101 MHz, DMSO-d6): δ 160.7, 157.8, 151.0, |
| | | | 149.5, 146.3, 131.8, 115.5, 105.4, 105.1, 98.8, 95.4, 55.4, 55.3. |
| 1p^{a} | 3-(2-chloro-3- | 30 | ¹H-NMR (400 MHz, DMSO-d6): δ 13.03 (bs, 1H), 7.90 (d, |
| | (trifluoromethyl) phenyl) | | J = 7.8 Hz, 1H), 7.66 (d, J = 6.9 Hz, 1H), 7.62 (t, J = 7.7 |
| | pyridine-2,6-diamine | | Hz, 1H), 7.43 (bs, 2H), 7.42 (d, J = 8.5 Hz, 1H), 6.94 |
| | trifluoroacetate | | (bs, 2H), 6.04 (d, J = 8.5 Hz, 1H). |
| | | | ¹³C-NMR (101 MHz, DMSO-d6): δ 152.6, 149.5, 145.8, |
| | | | 136.8, 136.5, 131.8, 128.1, 127.8 (q, J = 5.5 Hz), 127.6, |
| | | | 123.0 (q, J = 273 Hz), 104.3, 95.8. |
| 1q^{a} | 3-(3-chloro-2-methylphenyl) | 70 | ¹H-NMR (400 MHz, DMSO-d6): δ 12.73 (bs, 1H), 7.48 (d, |
| | pyridine-2,6-diamine | | J = 8.0 Hz, 1H), 7.37 (d, J = 8.4 Hz, 1H), 7.33 (bs, 2H), |
| | trifluoroacetate | | 7.29 (t, J = 7.9 Hz, 1H), 7.12 (d, J = 7.5 Hz, 1H), 6.74 |
| | | | (bs, 2H), 6.03 (d, J = 8.4 Hz, 1H), 2.14 (s, 3H). |
| | | | ¹³C-NMR (101 MHz, DMSO-d6): δ 152.7, 149.5, 146.0, |
| | | | 137.2, 135.6, 134.9, 130.0, 129.2, 128.0, 107.3, 96.3, 17.6. |
| 1r^{b} | 3-([1,1′-biphenyl]-2- | 28 | ¹H-NMR (400 MHz, DMSO-d6): δ 12.64 (bs, 1H), 7.52-7.42 (m, |
| | yl)pyridine-2,6-diamine | | 3H), 7.34-7.30 (m, 3H), 7.27-7.20 (m, 5H), 7.18 (d, J = 8.4 Hz, |
| | hydrochloride | | 1H), 6.70 (bs, 2H), 5.86 (d, J = 8.4 Hz, 1H). |
| | | | ¹³C-NMR (101 MHz, DMSO-d6) δ: 151.1, 149.0, 146.3, |
| | | | 141.7, 140.6, 133.0, 131.2, 130.5, 128.7, 128.5, 128.0, 127.9, |
| | | | 126.9, 107.6, 95.6. |

a: Purification by reversed-phase flash chromatography (MeOH, H2O + TFA 0.05%)
b: as a hydrochloride salt

### Example 2: Preparation of 3-(furan-3-yl)pyridine-2,6-diamine, 3b (Method 2)

A 5 ml microwave vial containing a Teflon® stirred bar was charged with: 3-iodopyridine-2,6-diamine (100 mg, 0.43 mmol, 1 eq.), 3-furanboronic acid (57 mg, 0.51 mmol, 1.2 eq.), K₂CO₃ (117.6 mg, 0.85 mmol, 2 eq.) followed by the addition of Pd(OAc)₂ (3.8 mg, 0.017 mmol, 0.04 eq.) and S-Phos (15.72 mg, 0.038 mmol, 0.09 eq.). A mixture of MeCN/H₂O: 3/1 (0.2 mmol/mL) was then introduced, the vessel was evacuated and backfilled with nitrogen (this process was repeated a total of 3 times). The reaction mixture was then capped properly and placed in a preheated oil bath at 100° C. until complete conversion of the starting material was detected (approximatively 4 hours). The reaction mixture was then concentrated under vacuum and the crude product was purified by reversed-phase flash chromatography (MeOH, H₂O+ TFA 0.05%) to afford 3b as a yellow solid after trituration in ether (50 mg, 67%).

¹H-NMR (400 MHz, DMSO-d6): δ 13.05 (bs, 1H), 7.90 (s, 1H), 7.79 (s, 1H), 7.64 (d, J=8.6 Hz, 1H), 7.36 (bs, 2H), 6.91 (bs, 2H), 6.72 (s, 1H), 6.06 (d, J=8.6 Hz, 1H).

¹³C-NMR (101 MHz, DMSO-d6) δ: 151.6, 148.9, 144.5, 143.7, 139.7, 119.3, 110.2, 99.1, 96.6.

Compounds 3a and 3c reported in table 2 are prepared following general method of preparation of 3b.

TABLE 2

| | | Yield | |
|---|---|---|---|
| N° | Name | % | RMN |
| 3a^{a} | 3-(furan-2- | 67 | ¹H-NMR (400 MHz, DMSO-d6): δ 12.83 (bs, 1H), 7.83 (d, |
| | yl)pyridine-2,6- | | J = 8.7 Hz, 1H), 7.71 (dd, J = 1.8 Hz, 0.6 Hz, 1H), 7.48 (bs, |
| | diamine | | 2H), 7.04 (bs, 2H), 6.66 (dd, J = 3.4 Hz, 0.6 Hz, 1H), 6.60 |
| | trifluoroacetate | | (dd, J = 3.4 Hz, 1.8 Hz, 1H), 6.09 (d, J = 8.7 Hz, 1H). |
| | | | ¹³C-NMR (101 MHz, DMSO-d6) δ: 152.2, 148.6, 147.8, |
| | | | 142.0, 141.8, 111.6, 105.6, 97.9, 97.2. |
| 3c^{b} | 3-(benzofuran-2-y1) | 51 | ¹H-NMR (400 MHz, DMSO-d6): δ 13.17 (bs, 1H), 8.01 (d, |
| | pyridine-2,6-diamine | | J = 8.6 Hz, 1H), 7.80 (bs, 2H), 7.60 (m, 2H), 7.46 (bs, 2H), |
| | hydrochloride | | 7.27(m, 2H), 7.10 (s, 1H), 6.16 (d, J = 8.6 Hz, 1H). |
| | | | ¹³C-NMR (101 MHz, CDCl3): δ 157.9, 155.3, 154.8, 154.2, |
| | | | 138.7, 129.3, 123.6, 123.2, 120.4, 110.9, 100.5, 100.5, 99.0. |

a: Purification by reversed-phase flash chromatography (MeOH, H2O + TFA 0.05%)
b: as a hydrochloride salt

2—Preparation of 3-Heteroaryl-2,6-diaminopyridine derivatives Starting from 3-Iodo-2,6-dichloropyridine (Method 3)

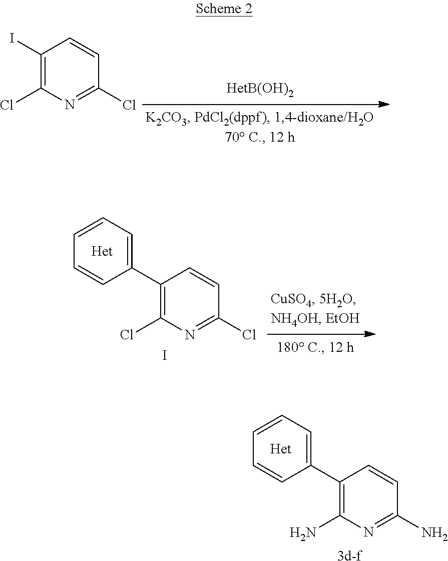

TABLE 2

| | | | Yield % | Yield % |
|---|---|---|---|---|
| entry | N° | Het = Ar | I | 3 |
| 1 | 3d | 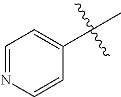 [SMILES: *c1ccncc1] | 95 | 64 |
| 2 | 3e | 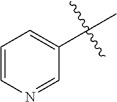 [SMILES: *c1cccnc1] | 81 | 76 |
| 3 | 3f | 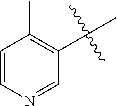 [SMILES: *c1cnccc1C] | 68 | 54 |

Introduction of a pyridine on position 3 failed under previous conditions (Pd(OAc)₂, S-Phos). However, according to the above reaction scheme, [3,4′-bipyridine]-2,6-diamine 3d-f could be prepared in a two-step sequence from 3-Iodo-2,6 dichloro pyridine. A convenient method for the formation of the C—C bond on position 3 involves the use of PdCl₂(dppf)/K₂CO₃ as the catalytic system (Tetrahedron Lett, 2009, 50, 3081-83). Starting from I, an Ullmann type reaction with the help of NH₄OH and CuSO₄, 5H₂O led to the formation of the bipyridine-2,6 diamine of general formula 3d-f (J. Org. Chem, 1983,48 (7), 1084-1091).

### Example 3: Preparation of [3,4′-bipyridine]-2,6-diamine 3d (Method 3)

### Step 1: 2,6-dichloro-3,4′-bipyridine

A 5 mL microwave vial containing a Teflon® stirred bar was charged with the commercial 2,6-dichloro-3-iodopyridine (200 mg, 0.71 mmol, 1 eq.), the pyridine-4-boronic acid (96.7 mg, 0.78 mmol, 1.1 eq.), K₂CO₃ (296 mg, 2.15 mmol, 3 eq.) followed by the addition of PdCl₂(dppf) (58.4 mg, 0.071 mmol, 0.1 eq). A mixture of 1,4-dioxane/H₂O: 4/1 (50 mL) was then introduced, the vessel was evacuated and backfilled with nitrogen (this process was repeated a total of 3 times). The reaction mixture was then capped properly and placed in a preheated oil bath at 70° C. until complete conversion of the starting material was detected (approximatively 16 hours). After evaporation of the volatiles the residue was diluted with EtOAc, successively washed with water and brine. The organic layer was dried over Na₂SO₄, filtered and concentrated under reduced pressure. The crude product was purified by chromatography on silica gel using EtOAc/heptane: 1/1 to afford the title compound as a white solid (150 mg, 93%).

¹H-NMR (400 MHz, CDCl₃): δ 8.69 (d, J=5.5 Hz, 2H), 7.61 (d, J=8.0 Hz, 1H), 7.36 (d, J=8.0 Hz, H), 7.34 (d, J=5.5 Hz, 2H).

¹³C-NMR (101 MHz, CDCl₃): δ 150.5, 150.2, 148.4, 144.2, 141.5, 133.2, 124, 123.6.

### Step 2: Preparation of [3,4′-bipyridine]-2,6-diamine, 3d

A 10 ml microwave vial containing a Teflon® stirred bar was charged with the 2,6-dichloro-3,4′-bipyridine (75 mg, 0.33 mmol, 1 eq.), copper sulfate hydrate (112.3 mg, 0.44 mmol, 1.33 eq.), aqueous NH₃ (28%, 4.5 mL, 100 eq.) and ethanol (2.23 mL). The reaction mixture was then capped properly and placed in a preheated oil bath at 180° C. during 12 hours. After cooling to room temperature the mixture was poured into distilled water (25 mL). After extraction with ethyl acetate (3×30 mL) the combined extracts were washed with distilled water (3×20 mL), dried over sodium sulfate and evaporated under reduced pressure. The crude product was purified on silica gel using EtOAc to afford the title compound 3d as a pale yellow solid (40 mg, 64%).

¹H-NMR (400 MHz, DMSO-d6): δ 8.48 (dd, J=4.6 Hz, 1.4 Hz, 2H), 7.38 (dd, J=4.6 Hz, 1.4 Hz, 2H), 7.18 (d, J=8.2 Hz, 1H), 5.85 (d, J=8.0 Hz, 1H), 5.77 (bs, 2H), 5.39 (bs, 2H).

¹³C-NMR (101 MHz, DMSO-d6): δ 159.0, 155.3, 149.7, 147.0, 139.3, 122.4, 104.6, 97.7.

[3,3′-bipyridine]-2,6-diamine, 3e. Following general procedure of preparation of 3d, 3e was obtained as a pale yellow solid (47.4 mg, 76%).

¹H-NMR (400 MHz, DMSO-d6): δ 8.54 (d, J=1.9 Hz, 1H), 8.41 (dd, J=4.6 Hz, 1.9 Hz, 1H), 8.41 (dt, J=7.9 Hz, 1.9 Hz, 1H), 7.37 (dd, J=7.9 Hz, 4.6 Hz, 1H), 7.08 (d, J=8.1 Hz, 1H), 5.84 (d, J=8.1 Hz, 1H), 5.64 (bs, 2H), 5.23 (bs, 2H).

¹³C-NMR (101 MHz, DMSO-d6): δ 158.7, 155.4, 149.0, 146.6, 139.6, 135.4, 135.3, 123.6, 104.3, 93.4.

4′-methyl-[3,4′-bipyridine]-2,6-diamine, 3f. Following general procedure of preparation of 3d was obtained as a yellow solid (15 mg, 45%).

¹H-NMR (400 MHz, CDCl₃): δ 8.41 (d, J=5.0 Hz, 1H), 8.35 (s, 1H), 7.17 (d, J=5.0 Hz, 1H), 7.03 (d, J=7.8 Hz, 1H), 5.95 (d, J=7.8 Hz, 1H), 4.55 (bs, 2H), 4.29 (bs, 2H), 2.19 (s, 3H).

¹³C-NMR (125 MHz, CDCl₃): δ 157.6, 154.9, 151.3, 149.0, 147.2, 140.9, 133.9, 125.5, 106.8, 98.2, 19.5.

3—Preparation of 3 (2-alkoxy phenyl)-2,6 diaminopyridine derivatives Starting from 2b: Methods 4 and 5

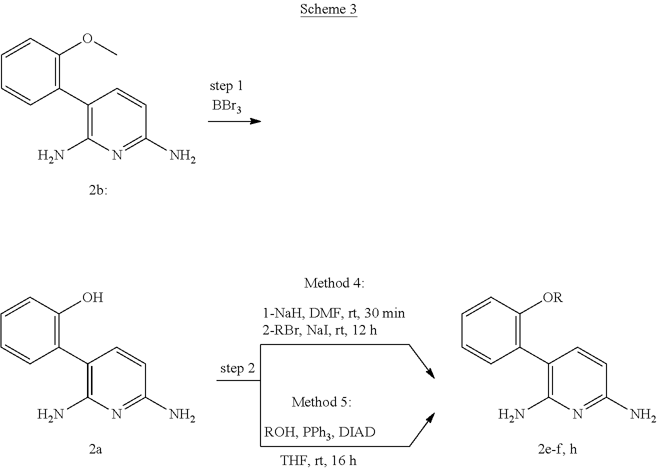

According to the above scheme 3,3-(2-methoxyphenyl)-2,6 diaminopyridine 2b is deprotected with BBr₃ following classical literature procedure. The resulting phenol 2a could react with alkyl halides in presence of NaH (Method 4) or with an appropriate alcohol under standard Mitsunobu conditions (Method 5) leading to 3 (2-alkoxy phenyl)-2,6 diaminopyridine derivatives of general formula 2e-f and 2 h.

### Example 4: Preparation of 3-(2-butoxyphenyl)pyridine-2,6-diamine 2e

### Step 1: Preparation of 2-(2,6-diaminopyridin-3-yl)phenol 2a

3-(2-methoxyphenyl)pyridine-2,6-diamine 2b (720 mg, 3.34 mmol, 1 eq.) was dissolved in DCM (34 mL), and cooled to −78° C. under a nitrogen atmosphere. Boron tribromide (1.0 M in DCM) (11.7 mL, 11.7 mmol, 3.5 eq.) was added dropwise over 20 min and the reaction mixture was warmed to ambient temperature and stirred for 3 h. The resultant mixture was basified to pH=8 by the dropwise addition of aqueous saturated sodium hydrogen carbonate solution. The organic layer was removed and the aqueous residue was reextracted with EtOAc (15 ml×3). The organic layers were combined, dried over Na₂SO₄ and concentrated under reduced pressure. The crude residue was purified by chromatography on silica gel using EtOAc/heptane: 4/1 then pure EtOAc to afford 2a as a white solid (627 mg, 93%).

¹H-NMR (400 MHz, DMSO-d6): δ 9.46 (s, 1H), 7.1 (td, J=7.7 Hz, 1.5 Hz, 1H), 7.06 (dd, J=1.4 Hz, 1.5 Hz, 1H), 6.99 (d, J=7.9 Hz, 1H), 6.89 (d, J=7.7 Hz, 1H), 6.83 (t, J=7.4 Hz, 1H), 5.82 (d, J=7.9 Hz, 1H), 5.47 (s, 2H), 4.84 (s, 2H).

¹³C-NMR (101 MHz, DMSO-d6): δ 156.7, 154.3, 154.1, 141.3, 131.3, 127.9, 125.2, 119.4, 115.8, 106.2, 96.7.

### Step 2: Preparation of 3-(2-butoxyphenyl)pyridine-2,6-diamine 2e (Method 4)

To a solution of 2-(2,6-diaminopyridin-3-yl)phenol 2a (40 mg, 0.2 mmol, 1 eq.)) in anhydrous DMF (1 mL) was added NaH (7.6 mg, 0.3 mmol, 1.5 eq) and the resulting mixture was stirred at RT under argon. After 30 min the corresponding 1-bromobutane (40.9 mg, 32 μL, 0.3 mmol, 1.5 eq.) was added and the solution was stirred an additional 12 hours. After evaporation of the volatiles the residue was diluted with EtOAc, successively washed with water and brine. The organic layer was dried over Na₂SO₄, filtered and concentrated under reduced pressure. The crude product was purified by chromatography on silica gel using EtOAc/heptane: 2/1 to afford 2e (44.1 mg, 86% yield) after preparation of the corresponding hydrochloride salt.

¹H-NMR (400 MHz, DMSO-d6): δ 13.28 (s, 1H), 7.39-7.33 (m, 4H), 7.16 (d, J=7.4 Hz, 1H), 7.08 (d, J=8.3 Hz, 1H), 6.99 (t, J=7.4 Hz, 1H), 6.76 (bs, 2H), 6.01 (d, J=8.3 Hz, 1H), 3.97 (t, J=6.4 Hz, 2H), 1.61 (qt, J=6.8 Hz, 2H), 1.34 (sext, J=7.3 Hz, 2H), 0.87 (t, J=7.3 Hz, 3H).

¹³C-NMR (101 MHz, DMSO-d6): δ 156.2, 151.2, 149.4, 146.2, 131.2, 129.5, 123.5, 120.7, 112.6, 105.1, 95.5, 67.4, 30.7, 18.7, 13.6.

### Example 5: Preparation of 3-(2-isopropoxyphenyl)pyridine-2,6-diamine 2f (Method 5)

To a solution of 2-(2,6-diaminopyridin-3-yl)phenol 2a (80 mg, 0.4 mmol, 1 eq.) in anhydrous THF (3.77 mL) and at RT were added triphenylphosphine (156.4 mg, 0.6 mmol, 1.5 eq.) and isopropanol (45 μL, 0.6 mmol, 1.5 eq.) followed by addition of azodicarboxylic acid diisopropyl ether (120 μL, 0.6 mmol, 1.5 eq.). The resulting mixture was stirred at room temperature overnight, concentrated under reduced pressure and purified by chromatography on silica gel using EtOAc/heptane: 4/1 to afford 2f as white solid (50 mg, 45%) after preparation of the corresponding hydrochloride salt.

¹H-NMR (400 MHz, DMSO-d6): δ 12.73 (s, 1H), 7.40 (d, J=8.4 Hz, 1H), 7.35 (td, J=7.8 Hz, 1.6 Hz, 1H), 7.30 (bs, 2H), 7.16 (dd, J=1.4 Hz, 1.6 Hz, 1H), 7.10 (d, J=8.4 Hz, 1H), 6.99 (t, J=7.4 Hz, 1H), 6.67 (bs, 2H), 6.01 (d, J=8.3 Hz, 1H), 4.56 (hept, J=6.0 Hz, 1H), 1.20 (d, J=6.0 Hz, 6H).

¹³C-NMR (101 MHz, DMSO-d6): δ 155.1, 151.2, 149.2, 146.3, 131.5, 129.4, 124.3, 120.7, 114.3, 105.3, 95.6, 70.1, 21.9.

3-(2-methoxyethoxyphenyl)pyridine-2,6-diamine, 2h: Following general method 5 and starting from 2a and 2-methoxyethan-1-ol, 2 h was obtained as an white solid (76 mg, 52%) after preparation of the corresponding hydrochloride salt.

¹H-NMR (400 MHz, DMSO-d6): δ 12.71 (s, 1H), 7.42 (d, J=8.4 Hz, 1H), 7.36 (td, J=7.8 Hz, 1.4 Hz, 1H), 7.33 (bs, 2H), 7.18 (td, J=7.4 Hz, 1.4 Hz, 1H), 7.12 (d, J=8.4 Hz, 1H), 7.02 (t, J=7.4 Hz, 1H), 6.71 (bs, 2H), 6.02 (d, J=8.4 Hz, 1H), 4.13 (t, J=4.7 Hz, 2H), 3.60 (t, J=4.7 Hz, 2H), 3.24 (s, 3H).

¹³C-NMR (101 MHz, DMSO-d6): δ 155.9, 151.3, 149.3, 146.4, 131.3, 129.5, 123.6, 121.0, 112.9, 105.1, 95.7, 70.2, 67.3, 58.2.

4—Preparation of 3 (2-alkoxy phenyl)-2,6 diaminopyridine derivatives Starting from 3-Iodo-2,6-dichloropyridine: Method 6

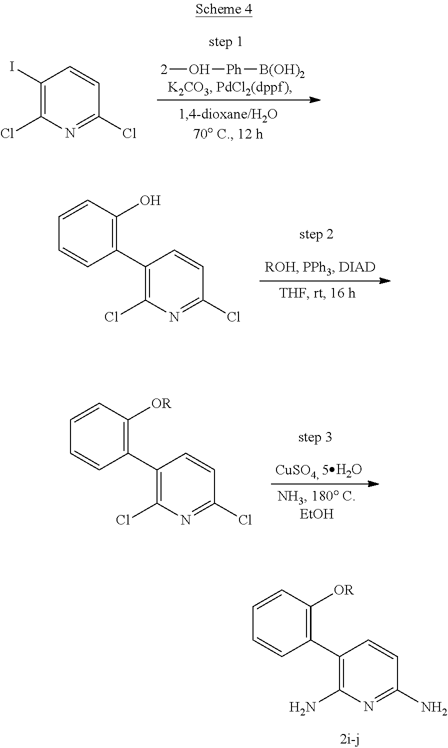

An alternative method for the preparation of 2-alkoxy phenyl derivatives of the general formula 2 is described in scheme 4. According to the above reaction scheme, 3 (2-alkoxy phenyl)-2,6 diaminopyridine derivatives 2i-j could be prepared in a three-step sequence from commercially available 3-Iodo-2,6 dichloropyridine. A Suzuki-Miyaura reaction in presence of 2-Hydroxy phenyl boronic acid and with the help of PdCl₂(dppf)/K₂CO₃ led to the corresponding phenol derivative. Alkylation of the phenol with appropriate alcohols under Mitsunobu conditions followed by a Ullmann type reaction (CuSO₄, 5H₂O+NH₄OH) as described in scheme 4, led to the alkoxy derivatives of the general formula 2.

### Example 6: Preparation of 3-(2-(cyclopentyloxy)phenyl)pyridine-2,6-diamine, 2i, Method 6

### Step 1: 2-(2,6-dichloropyridin-3-yl)phenol

A 5 ml microwave vial containing a Teflon® stirred bar was charged with the commercial 2,6-dichloro-3-iodopyridine (2.0 g, 7.16 mmol, 1 eq.), the 2-hydroxyphenyl boronic acid (1.08 g, 7.87 mmol, 1.1 eq.), K₂CO₃ (2.97 g, 21.47 mmol, 3 eq.) followed by the addition of PdCl₂(dppf) (262 mg, 0.36 mmol, 0.05 eq). A mixture of 1,4-dioxane/H₂O: 4/1 (50 mL) was then introduced, the vessel was evacuated and backfilled with nitrogen (this process was repeated a total of 3 times). The reaction mixture was then capped properly and placed in a preheated oil bath at 70° C. until complete conversion of the starting material was detected (approximatively 16 hours). After evaporation of the volatiles the residue was diluted with EtOAc, successively washed with brine and water. The organic layer was dried over Na₂SO₄, filtered and concentrated under reduced pressure. The crude product was purified by chromatography on silica gel using EtOAc/heptane:1/2 to afford the expected 2-(2,6-dichloropyridin-3-yl)phenol as a white solid (1.52 g, 88%).

¹H-NMR (400 MHz, DMSO-d6): δ 9.73 (s, 1H), 7.85 (d, J=7.7 Hz, 1H), 7.60 (d, J=8.2 Hz, 1H), 7.26 (t, J=7.7 Hz, 1H), 7.16 (d, J=7.4 Hz, 1H), 6.95 (d, J=8.2 Hz, 1H), 6.89 (t, J=7.4 Hz, 1H).

¹³C-NMR (101 MHz, DMSO-d6): δ 154.4, 148.7, 147.3, 143.9, 133.4, 130.5, 130.1, 123.2, 123.0, 118.9, 115.7.

### Step 2

### 2,6-dichloro-3-(2-(cyclopentyloxy)phenyl)pyridine

Following method of preparation for 2f (method 5, Mitsunobu conditions) and starting from 2-(2,6-dichloropyridin-3-yl)phenol (56.6 mg, 0.23 mmol, 1 eq.) and cyclopentanol (40.6 mg, 0.48 mmol, 2 eq.), 2,6-dichloro-3-(2-(cyclopentyloxy)phenyl)pyridine was obtained as a clear oil after a purification by chromatography on silica gel (Eluant: AcOEt/heptane:1/9; 63.3 mg, 87%).

¹H-NMR (400 MHz, DMSO-d6): δ 7.83 (d, J=8.0 Hz, 1H), 7.61 (d, J=8.0 Hz, 1H), 7.41 (td, J=7.8 Hz, 1.5 Hz, 1H), 7.23 (dd, J=7.5 Hz, 1.5 Hz, 1H), 7.11 (d, J=8.3 Hz, 1H), 7.02 (t, J=7.5 Hz, 1H), 4.84 (m, 1H), 1.86-1.77 (m, 2H), 1.60-1.57 (m, 2H), 1.52-1.48 (m, 4H).

¹³C-NMR (101 MHz, CDCl₃): δ 155.2, 150.1, 148.7, 142.7, 133.5, 130.9, 130.3, 126.2, 122.5, 120.2, 113.4, 79.9, 32.9, 24.2.

### Step 3

### 3-(2-(cyclopentyloxy)phenyl)pyridine-2,6-diamine 2i

A 10 ml microwave vial containing a Teflon® stirred bar was charged with the 2,6-dichloro-3-(2-(cyclopentyloxy)phenyl)pyridine (60 mg, 0.19 mmol, 1 eq.), copper sulfate hydrate (65.6 mg, 0.26 mmol, 1.33 eq.), aqueous NH₃ (28%, 2.7 mL, 100 eq.) and ethanol (1.33 mL). The reaction mixture was then capped properly and placed in a preheated oil bath at 180° C. during 24 hours. After cooling to room temperature the mixture was poured into distilled water (25 mL). After extraction with EtOAC (3×30 mL) the combined extracts were washed with distilled water (3×20 mL), dried over sodium sulfate and evaporated under reduced pressure. The crude product was purified on silica gel using EtOAc-Heptane: 3/1 to afford the expected: 3-(2-(cyclopentyloxy)phenyl)pyridine-2,6-diamine 2i as a white solid (36.5 mg, 61%) after preparation of the corresponding hydrochloride salt.

¹H-NMR (400 MHz, DMSO-d6): δ 13.08 (bs, 1H), 7.37-733 (m, 4H), 7.16 (d, J=7.2 Hz, 1H), 7.07 (d, J=8.3 Hz, 1H), 6.99 (t, J=7.2 Hz, 1H), 6.69 (bs, 2H), 6.00 (d, J=8.3 Hz, 1H), 4.81 (m, 1H), 1.83-1.79 (m, 2H), 1.68-1.64 (m, 2H), 1.60-1.51 (m, 4H).

¹³C-NMR (101 MHz, DMSO-d6): δ 155.1, 151.2, 149.3, 146.2, 131.3, 129.4, 124.2, 120.6, 114.0, 105.3, 95.5, 79.2, 32.2, 23.5.

3-(2-(piperidin-1-yl)ethoxy)pyridine-2,6-diamine 2i: Following general method of preparation of 2i and using 2-(2,6-dichloropyridin-3-yl)phenol and 2-(piperidin-1-yl)ethan-1-ol, 2j was obtained as white solid after treatment with aqueous NH₃ in presence of copper sulfate hydrate (92% and 26% respectively for steps 2 and 3) and after preparation of the corresponding dihydrochloride salt.

¹H-NMR (400 MHz, DMSO-d6): δ 13.17 (s, 1H), 10.63 (bs, 1H), 7.43-7.40 (m, 4H), 7.20 (d, J=7.3 Hz, 1H), 7.14 (d, J=8.4 Hz, 1H), 7.08 (t, J=7.3 Hz, 1H), 6.85 (bs, 2H), 6.01 (d, J=8.4 Hz, 1H), 4.42 (t, J=4.5 Hz, 2H), 3.60 (m, 2H), 3.37 (m, 2H), 2.89 (m, 2H), 1.74-1.65 (m, 6H).

¹³C-NMR (101 MHz, DMSO-d6): δ 155.3, 151.4, 149.3, 146.3, 131.6, 129.7, 123.4, 121.6, 112.5, 104.8, 95.6, 62.9, 54.8, 52.6, 22.3, 21.1.

5—Preparation of 3-Aryl-N2-alkylpyridine-2,6-diamine Starting from 1j, by Reductive Amination of 4a (Method 7).

According to the above reaction scheme 5, 1j is acylated under standard literature procedures leading to 4a. A convenient method is the use of acetic anhydride in presence of pyridine. A reductive amination of 4a with a suitable aldehyde followed by deprotection of the acetyl moiety under acidic condition produce 3-Aryl N2-alkyl pyridine 2,6 diamine derivatives of the general formula 5. A convenient method for the reductive amination involves the use of NaBH₃CN in Methanol.

Scheme 5

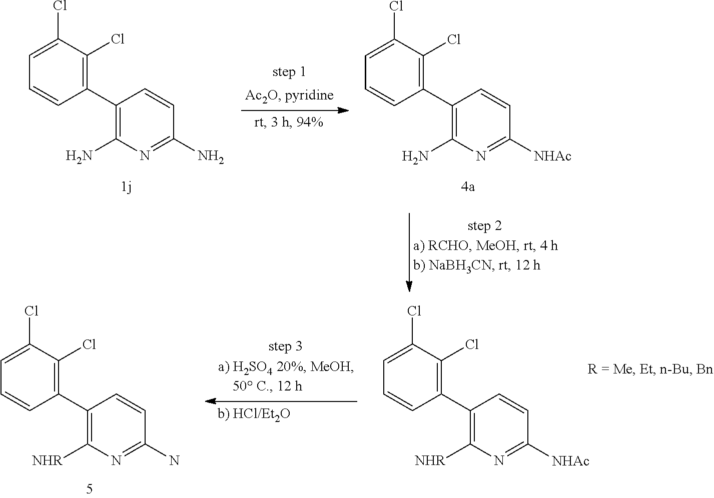

| | | | Step 2, | Step 3, |
|---|---|---|---|---|
| Entry | No | R | Yield % | Yield % |
| 1 | 5a | Me | 68 | 76 |
| 2 | 5b | Et | 75 | 70 |
| 3 | 5c | n-Bu | 72 | 71 |
| 4 | 5d | Bn | 65 | 74 |

### Example 7: Preparation of 3-(2,3-dichlorophenyl)-N2-methylpyridine-2,6-diamine 5a, Method 7

Step 1: Preparation of N-(6-amino-5-(2,3-dichlorophenyl)pyridin-2-yl)acetamide 4a A round bottomed flask containing a stirrer bar was charged with 3-(2,3-dichlorophenyl)-pyridine-2,6-diamine 1j (500 mg, 1.97 mmol, 1 eq.) and pyridine (2.1 mL). Acetic anhydride (332 μL, 3.54 mmol, 1.8 eq.) was then added and the mixture was stirred at RT until complete conversion of the starting material was detected. The reaction mixture was monitored by HPLC analysis and was complete within 2 h30. After evaporation of the volatiles, the residue was diluted with EtOAc and successively washed with brine and water. The organic layer was dried over Na₂SO₄, filtered and concentrated in vacuo. The crude product was purified by chromatography on silica gel using a gradient of 50% to 75% EtOAc in heptane to afford 4a as a white solid (547 mg, 94%)

¹H-NMR (400 MHz, CDCl₃): δ 8.17 (bs, 1H), 7.60 (d, J=7.5 Hz, 1H), 7.46 (dd, J=7.9 Hz, 1.8 Hz, 1H), 7.30 (d, J=7.9 Hz, 1H), 7.24 (t, J=7.7 Hz, 1H), 7.19 (dd, J=7.7 Hz, 1.8 Hz, 1H), 4.28 (s, 2H), 2.14 (s, 3H).

¹³C-NMR (101 MHz, CDCl₃): δ 168.9, 154.3, 150.2, 141.0, 138.5, 134.2, 132.8, 130.5, 130.2, 128.0, 115.3, 103.7, 24.9.

### Step 2: N-(5-(2,3-dichlorophenyl)-6-(methylamino)pyridin-2-yl)acetamide

A round bottomed flask containing a stirrer bar was charged with N-(6-amino-5-(2,3-dichloro-phenyl)pyridin-2-yl)acetamide 4a (100 mg, 0.34 mmol, 1 eq.) in MeOH (10 mL) followed by the addition of formaldehyde (50.6 μL, 0.67 mmol, 2 eq.) and acetic acid (58 μL, 1 mmol, 3 eq.). The resulting mixture was stirred at room temperature for 4 hours. NaBH₃CN (44.7 mg, 0.67 mmol, 2 eq.) was then added and the solution was stirred an additional 12 hours until TLC showed complete disappearance of the starting aminopyridine derivative. The reaction mixture was concentrated under reduced pressure.

The residue was dissolved in EtOAc, and successively washed with brine and water. The organic layer was dried over Na₂SO₄, filtered and concentrated in vacuo. The crude product was purified by chromatography on silica gel using a gradient of 25% to 30% ethyl acetate in heptane to afford the expected product as a clear oil (71 mg, 68%).

¹H-NMR (400 MHz, DMSO-d6): δ 9.96 (s, 1H), 7.64 (dd, J=8.1 Hz, 1.6 Hz, 1H), 7.40 (t, J=7.9 Hz, 1H), 7.29 (m, 1H), 7.26 (dd, J=7.7 Hz, 1.4 Hz, 1H), 7.16 (d, J=7.9 Hz, 1H), 5.57 (q, J=4.6 Hz, 1H), 2.76 (d, J=4.6 Hz, 3H), 2.09 (s, 3H).

¹³C-NMR (101 MHz, CDCl₃): δ 168.6, 154.9, 150.0, 139.8, 138.6, 134.2, 133.1, 130.5, 130.4, 128.1, 115.7, 101.1, 28.7, 25.0.

### Step 3: 3-(2,3-dichlorophenyl)-N2-methylpyridine-2,6-diamine 5a

A 5 mL microwave vial containing a Teflon® stirred bar was charged with N-(5-(2,3-dichlorophenyl)-6-(methylamino)pyridin-2-yl)acetamide (65 mg, 0.21 mmol, 1 eq.) in MeOH (1.5 mL) followed by the addition of H₂SO₄ 20% (1.3 mL). The resulting mixture was than stirred at 50° C. for 16 hours, reaction was cooled to rt and basified with NH₄OH. The reaction mixture was extracted twice with EtOAc and successively washed with brine and water. The organic layer was dried over Na₂SO₄, filtered and concentrated in vacuo. The crude product was purified by chromatography on silica gel using EtOAc/Hept: 1/1 to afford a light white solid (48.3 mg, 76%) after preparation of the corresponding hydrochloride salt.

¹H-NMR (400 MHz, DMSO-d6): δ 12.50 (bs, 1H), 7.71 (s, 2H), 7.70 (dd, J=8.0 Hz, 1.4 Hz, 1H), 7.44 (t, J=1.1 Hz, 1H), 7.36 (d, J=8.5 Hz, 1H), 7.31 (dd, J=1.1 Hz, 1.4 Hz, 1H), 7.14 (bs, 1H), 5.98 (d, J=8.5 Hz, 1H), 2.93 (d, J=4.8 Hz, 3H).

¹³C-NMR (101 MHz, CDCl₃): δ 157.9, 155.4, 139.6, 139.5, 133.9, 133.4, 130.9, 129.8, 127.8, 109.4, 95.4, 28.7.

3-(2,3-dichlorophenyl)-N2-ethylpyridine-2,6-diamine 5b. Following general method 7 and starting from 4a and acetaldehyde, 5b was obtained as a solid.

¹H-NMR (400 MHz, CDCl₃): δ 7.48 (d, J=7.9 Hz, 1H), 7.28 (d, J=7.4 Hz, 1H), 7.25-7.22 (m, 1H), 7.04 (d, J=7.9 Hz, 1H), 5.89 (d, J=7.9 Hz, 1H), 4.31 (bs, 2H), 3.86 (bs, 1H), 3.42 (m, 2H), 1.15 (t, J=7.2 Hz, 3H).

¹³C-NMR (101 MHz, CDCl₃): δ 157.8, 154.8, 139.8, 139.7, 134.0, 133.4, 131.0, 129.8, 127.9, 109.3, 95.4, 36.4, 15.5.

N2-butyl-3-(2,3-dichlorophenyl)pyridine-2,6-diamine 5c. Following general method 7 and starting from 4a and butyraldehyde, 5c was obtained as a white solid after preparation of the corresponding hydrochloride salt.

¹H-NMR (400 MHz, DMSO-d6): δ 12.57 (bs, 1H), 7.76 (bs, 2H), 7.69 (d, J=7.7 Hz, 1H), 7.44 (t, J=7.7 Hz, 1H), 7.33 (d, J=7.7 Hz, 1H), 7.30 (d, J=7.7 Hz, 1H), 7.09 (bs, 1H), 5.95 (d, J=8.1 Hz, 1H), 3.34 (m, 2H), 1.48 (m, 2H), 1.31 (m, 2H), 0.87 (t, J=7.2 Hz, 3H).

¹³C-NMR (101 MHz, DMSO-d6) δ: 152.8, 148.0, 144.8, 136.0, 132.4, 132.3, 131.5, 130.5, 128.7, 105.9, 93.9, 41.5, 30.8, 19.2, 13.7.

N2-benzyl-3-(2,3-dichlorophenyl)pyridine-2,6-diamine 5d Following general method 7 and starting from 4a and benzaldehyde, 5d was obtained as a white solid after preparation of the corresponding hydrochloride salt.

¹H-NMR (400 MHz, DMSO-d6): δ 13.01 (bs, 1H), 7.82 (bs, 2H), 7.71 (d, J=8.2 Hz, 1H), 7.59 (bs, 1H), 7.45 (t, J=7.8 Hz, 1H), 7.38-7.31 (m, 6H), 7.25 (m, 1H), 6.01 (d, J=8.2 Hz, 1H), 4.74 (d, J=5.4 Hz, 2H).

¹³C-NMR (101 MHz, DMSO): δ 152.9, 147.9, 145.0, 137.6, 135.9, 132.6, 132.3, 131.5, 130.7, 128.8, 128.3, 127.2, 127.1, 106.2, 95.0, 44.5.

6—Preparation of 3-Aryl-N2-alkylpyridine-2,6-diamine Starting from 6-fluoropyridin-2-amine (Method 8)

An alternative method for the preparation of 3-bromo-N2-alkylpyridine-2,6 diamine of the general formula 5 involves the use of the easily available 5-bromo-6-fluoropyridin-2-amine. According to procedure well known in the art, the nucleophilic aromatic substitution of the fluoride with appropriate amines can be performed in DMSO under microwave irradiations (160° C., 30 min) or at 100° C. for 24 h. Resulting diaminopyridine derivatives react further with suitably boronic acids to produce 3-Aryl-N2-alkylpyridine-2,6 diamine derivatives of the general formula 5. A convenient method involves the use of Pd(PPh₃)₄ in presence of K₂CO₃ in a mixture of toluene/EtOH/H₂O.

Scheme 6

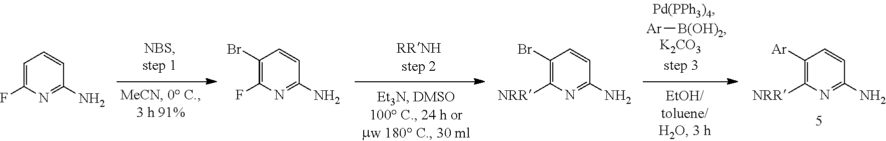

| | | | | Yield %, | Yield %, |
|---|---|---|---|---|---|
| Entry | N° | R | R′ | step 2 | step 3 |
| 1 | 5e | i-Pr | H | 81 | 37 |
| 2 | 5f | (CH₂)₂—Ph | H | 83 | 53 |
| 3 | 5g | (CH₂)₂—OMe | H | 72 | 58 |
| 4 | 5h | 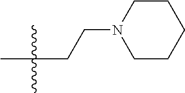 [SMILES: *CCN1CCCCC1] | H | 86 | 54 |
| 5 | 5i | 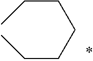 | — | 58 | 11 |

*NRR′ where R and R′ form a carbocycle with N, i.e. piperidine

### Example 8: Preparation of 3-(2,3-dichlorophenyl)-N2-phenethylpyridine-2,6-diamine 5f

### Step 1

### 5-bromo-6-fluoropyridin-2-amine

A solution of commercially available 6-fluoro-pyridin-2-ylamine (1.0 g, 8.74 mmol, 1 eq.) in acetonitrile (44 mL), protected from light and under nitrogen atmosphere, was set stirring at 0° C. before adding a solution of N-bromosuccinimide (0.79 g, 8.74 mmol, 1 eq.) in acetonitrile (19 mL) over 30 min. After complete addition, the resulting solution was stirred for an additional 2 h30. The reaction mixture was then concentrated under reduced pressure and the residue was dissolved in EtOAc, successively washed with brine and water. The organic layer was dried over Na₂SO₄, filtered and concentrated in vacuo. The crude material was purified by flash column chromatography using a gradient of 25% to 50% EtOAc in heptane to give 5-bromo-6-fluoro-pyridin-2-ylamine (1.45 g, 91%) as a white solid.

¹H-NMR (400 MHz, CDCl₃): δ 7.55 (t, J=8.6 Hz, 1H), 6.22 (dd, J=8.3 Hz, 1.5 Hz, 1H), 4.70 (bs, 2H).

¹³C-NMR (101 MHz, CDCl₃): δ 158.8 (d, J=235 Hz), 156.7 (d, J=16 Hz), 144.9 (d, J=2.9 Hz), 106.7 (d, J=5.0 Hz), 89.4 (d, J=38 Hz).

### Step 2

### 3-bromo-N2-phenethylpyridine-2,6-diamine

A 2 mL microwave vial containing a Teflon® stirred bar was charged with 5-bromo-6-fluoropyridin-2-amine (50 mg, 0.26 mmol, 1 eq.), phenethylamine (127 mg, 1.0 mmol, 4 eq.), triethylamine (72 μL, 0.52 mmol, 2 eq.) and anhydrous DMSO (0.2 mL). The reaction mixture was then capped properly and placed in a preheated oil bath at 100° C. until complete conversion of the starting material was detected (approximatively 24 hours). The resulting solution was then diluted with water (10 mL) and extracted with EtOAc (2×15 mL). The combined organic extracts were washed with water (15 mL) and brine (15 mL), dried over Na₂SO₄, and filtered. The filtrate was evaporated in vacuo and the residue was purified by flash column chromatography on silica gel using EtOAc/heptane: 1/3 to give the title product as a yellow oil (63.6 mg, 83%).

¹H-NMR (400 MHz, CDCl₃): δ 7.28-7.25 (m, 2H), 7.21-7.15 (m, 4H), 5.66 (d, J=8.2 Hz, 1H), 4.8 (t, J=4.8 Hz, 1H), 4.14 (bs, 2H), 3.59 (q, J=6.7 Hz, 2H), 3.59 (t, J=7.0 Hz, 2H).

¹³C-NMR (101 MHz, CDCl₃): δ 156.9, 153.6, 141.1, 139.9, 129.1, 128.7, 126.5, 97.4, 92.6, 43.1, 36.2.

### Step 3: 3-(2,3-dichlorophenyl)-N2-phenethylpyridine-2,6-diamine, 5f

A 5 mL microwave vial containing a Teflon® stirred bar was charged 3-bromo-N2-phenethylpyridine-2,6-diamine (60 mg, 0.20 mmol, 1 eq.), 2,3-dichloro phenylboronic acid (47 mg, 0.24 mmol, 1.2 eq.), Na₂CO₃ (65.6 mg, 0.60 mmol, 3 eq.) followed by the addition of a mixture of Toluene/EtOH/H₂O: 6/1/1 (0.1 mmol/mL). The vessel was evacuated and backfilled with nitrogen (this process was repeated a total of 3 times) and Pd(PPh₃)₄ (12.0 mg, 0.0103 mmole, 0.05 eq.) was introduced. The reaction mixture was then capped properly and placed in a preheated oil bath at 100° C. until complete conversion of the starting material was detected. The reaction mixture was monitored by HPLC analysis and was usually complete within 4 h30 hours. The reaction mixture was then concentrated under vacuum and the crude product was purified by chromatography on silica gel using a gradient of 25% to 70% ethyl acetate in hexane to afford the expected product 5f as a light yellow solid (42.8 mg, 53%) after preparation of the corresponding hydrochloride salt.

¹H-NMR (400 MHz, DMSO-d6): δ 12.60 (bs, 1H), 7.70 (bs, 2H), 7.69 (d, J=7.9 Hz, 1H), 7.42 (t, J=7.7 Hz, 1H), 7.35 (m, 1H), 7.29-7.28 (m, 4H), 7.23-7.20 (m, 2H), 7.02 (bs, 1H), 5.97 (d, J=8.3 Hz, 1H), 3.52 (m, 2H), 2.82 (m, 2H).

¹³C-NMR (101 MHz, CDCl3): δ 157.8, 154.3, 139.8, 139.7, 139.2, 133.9, 133.3, 130.7, 129.8, 129.0, 128.6, 127.7, 126.3, 109.3, 95.6, 42.8, 36.0.

3-(2,3-dichlorophenyl)-N2-isopropylpyridine-2,6-diamine, 5e was obtained following general method 8, starting from 5-bromo-6-fluoropyridin-2-amine and isopropylamine for step 2, and 2,3-Cl₂Ph boronic acid for step 3 as a white solid (46 mg, 37%), after preparation of the corresponding hydrochloride salt.

¹H-NMR (400 MHz, DMSO-d6): δ 12.71 (bs, 1H), 7.84 (bs, 2H), 7.68 (d, J=7.8 Hz, 1H), 7.43 (t, J=7.8 Hz, 1H), 7.34-7.30 (m, 2H), 6.74 (bs, 1H), 5.96 (d, J=8.3 Hz, 1H), 4.33 (m, 1H), 1.14 (m, 6H).

¹³C-NMR (101 MHz, CDCl₃): δ 157.8, 154.2, 139.8, 139.7, 133.9, 133.2, 130.9, 129.7, 127.8, 109.3, 95.3, 42.5, 23.4, 23.3.

3-(2,3-dichlorophenyl)-N2-(2-methoxyethyl)pyridine-2,6-diamine, 5g was obtained following general method 8, starting from 5-bromo-6-fluoropyridin-2-amine and 2-methoxyethan-1-amine for step 2, and 2,3-Cl₂Ph boronic acid for step 3 as a solid (46 mg, 58%), after preparation of the corresponding hydrochloride salt.

¹H-NMR (400 MHz, DMSO-d6): δ 12.48 (bs, 1H), 7.70 (d, J=7.9 Hz, 1H), 7.69 (bs, 2H), 7.44 (t, J=7.8 Hz, 1H), 7.36 (d, J=8.0 Hz, 1H), 7.30 (d, J=7.8 Hz, 1H), 7.07 (bs, 1H), 5.99 (d, J=8.4 Hz, 1H), 3.57 (m, 2H), 3.44 (m, 2H), 3.25 (s, 3H).

¹³C-NMR (101 MHz, CDCl₃): δ 157.7, 154.6, 139.8, 139.5, 133.9, 133.3, 130.8, 129.8, 127.8, 109.5, 95.8, 71.9, 58.8, 41.1.

3-(2,3-dichlorophenyl)-N2-(2-(piperidin-1-yl)ethyl)pyridine-2,6-diamine, 5 h was obtained following general method 8, starting from 5-bromo-6-fluoropyridin-2-amine and 2-(piperidin-1-yl)ethan-1-amine for step 2, and 2,3-Cl₂Ph boronic acid for step 3 as a solid (59 mg, 54%), after preparation of the corresponding hydrochloride salt.

¹H-NMR (400 MHz, DMSO-d6): δ 13.24 (bs, 1H), 10.14 (bs, 1H), 7.85 (bs, 2H), 7.69 (d, J=7.8 Hz, 1H), 7.43 (t, J=7.8 Hz, 1H), 7.34 (bs, 1H), 7.33 (d, J=7.5 Hz, 1H), 6.04 (d, J=7.4 Hz, 1H), 3.80 (m, 2H), 3.66 (m, 4H), 3.21 (m, 2H), 2.98 (m, 2H), 1.75 (m, 4H).

¹³C-NMR (101 MHz, CDCl₃): δ 157.9, 155.0, 139.8, 139.3, 133.7, 133.3, 130.9, 129.5, 127.7, 109.6, 95.2, 57.3, 54.2, 38.2, 26.2, 24.6.

5-(2,3-dichlorophenyl)-6-(piperidin-1-yl)pyridin-2-amine, 5i was obtained following general method 8, starting from 5-bromo-6-fluoropyridin-2-amine and piperidine for step 2, and 2,3-Cl₂Ph boronic acid for step 3 as a solid (17 mg, 11%), after preparation of the corresponding hydrochloride salt.

¹H-NMR (400 MHz, DMSO-d6): δ 13.03 (bs, 1H), 7.93 (bs, 1H), 7.66 (d, J=7.6 Hz, 1H), 7.52 (d, J=8.5 Hz, 1H), 7.46 (t, J=7.8 Hz, 1H), 7.42 (bs, 1H), 7.33 (d, J=7.5 Hz, 1H), 6.33 (d, J=8.3 Hz, 1H), 3.05 (m, 4H), 1.45-1.35 (m, 6H).

¹³C-NMR (101 MHz, CDCl₃): δ 159.4, 157.0, 142.4, 142.2, 133.6, 131.8, 130.1, 128.6, 127.2, 113.8, 99.2, 49.9, 26.0, 24.9.

7—Preparation of N2-alkyl-5-arylpyridine-2-amine 6: Method 9

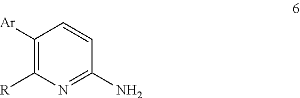

Compounds of the general formula 6 could be prepared by a Suzuki-Miyaura reaction using palladium tetrakis in presence of K₂CO₃ according to conventional procedures between a boronic acid and the appropriate heteroaryl halide. The later could either be commercially available or obtained by halogenation of the corresponding 2-amino-6-alkyl-pyridine derivatives. Furthermore, those 2-amino-6-alkyl-pyridine derivatives could be synthesized using well described procedures. In particular preparation of 2-amino 6-alkyl-pyridine derivatives is outlined in scheme 7.

### Preparation of 2-amino-6-alkyl pyridine derivatives, Scheme 7

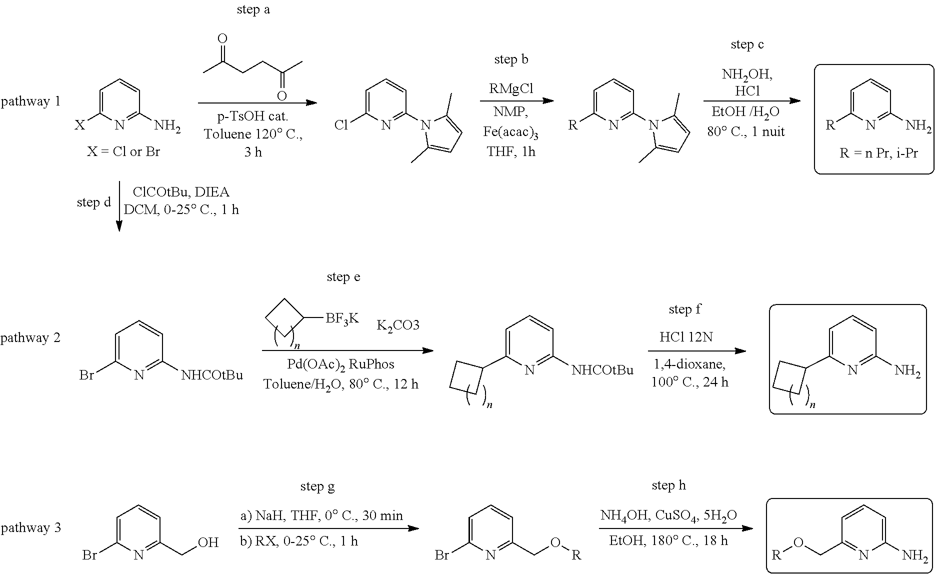

A first pathway involved the cyclocondensation of the commercially available 6-chloro-2 aminopyridine with 2,5-butanedione in presence of a catalytic amount of p-toluene sulfonic acid (Synthesis, 2007, 17, 2711-2719). The resulting 6-chloro 2-(2,5-dimethyl-pyrrol-1-yl-pyridine was then treated with a Grignard reagent (RMgX) in dry THF in presence of iron (III) acetylacetonate and 1-methyl-2 pyrrolidinone (NMP) (J. Am. Chem. Soc., 2002, 124, 13856-1313863). The resulting compound can be directly converted to 2-amino-6-alkyl-pyridine by treatment with hydroxyl amine hydrochloride. The 6-cycloalkyl-2-amino-pyridine derivatives were prepared as presented in pathway 2, scheme 7 using a Suzuki cross coupling reaction between the N-(6-Bromopyridin-2-yl)pivalamide and potassium cycloalkyl-trifluoroborate in presence of palladium acetate and RuPhos. Deprotection of the pivaloyl moiety was performed under acidic conditions. Finally the O-alkylation reaction of the commercially available 6-bromo-pyridin-2-yl-methanol with appropriate alkyl halides led to 6-alkoxymethyl aminopyridines after an Ullmann cross coupling reaction, as described in scheme 7 (pathway 3).

### Preparation of Non-Commercially Available 6-alkyl-2amino-pyridine derivatives

### Preparation of 6-propylpyridin-2-amine

### Step a

### 2-chloro-6-(2,5-dimethyl-1H-pyrrol-1-yl)pyridine

A solution of commercially available 6-chloropyridin-2 amine (1.0 g, 7.78 mmol, 1 eq.), 2,5-butanedione (1.06 g, 9.33 mmol, 1.2 eq.) and p-toluenesulfonic acid monohydrate (9 mg, 0.093 mmol, 0.01 eq.) in toluene (10 mL) was heated at 120° C. with azeotropic removal of water for about 3 hours. After cooling to room temperature, the resulting mixture was diluted with EtOAc (10 mL), washed successively with a saturated solution of NaHCO₃, water and brine. The organic fraction was than dried with Na₂SO₄. The reaction mixture was then concentrated under vacuum and the crude product was purified by chromatography on silica gel using a EtOAc/heptane: 1/8 as eluent to afford the expected product 2-chloro-6-(2,5-dimethyl-1H-pyrrol-1-yl)pyridine (1,542 g, 96%).

¹H-NMR (400 MHz, CDCl₃): δ 7.76 (t, J=7.8 Hz, 1H), 7.31 (d, J=7.8 Hz, 1H), 7.13 (d, J=7.8 Hz, 1H), 5.88 (s, 3H), 2.14 (s, 6H).

¹³C-NMR (101 MHz, CDCl₃): δ 151.9, 150.6, 140.3, 128.9, 122.8, 120.2, 107.7, 13.5.

### Step b

### 2-(2,5-dimethyl-1H-pyrrol-1-yl)-6-propylpyridine

A round bottomed flask (oven-dried and under Argon) containing a stirrer bar was charged with 2-chloro-6-(2,5-dimethyl-1H-pyrrol-1-yl)pyridine (300 mg, 1.45 mmol, 1 eq.), 1-methyl-2-pyrrolidone (1.26 mL, 13 mmol, 9 eq.), Fe(acac)₃ (25.63 mg, 0.07 mmol, 0.05 eq.) and dry THF (7.5 mL). The resulting mixture was cooled to 0° C. and a 2M solution of n-propylmagnesiumchloride in diethyl ether (1.09 mL, 2.18 mmol, 1.5 eq.) was added dropwise. After the end of the addition the mixture was stirred at RT for 1 h. The reaction was quenched with saturated solution of NH₄Cl (5 mL) extracted twice with diethyl ether. The organic layers were combined and washed with brine and water. The organic layer was dried over Na₂SO₄, filtered and evaporated in vacuo. The crude product was purified by chromatography on silica gel using EtOAc/heptane (98:2) as eluant. 2-(2,5-dimethyl-1H-pyrrol-1-yl)-6-propylpyridine was obtained as a yellow oil (235 mg, 76%).

¹H-NMR (400 MHz, CDCl₃): δ 7.70 (t, J=7.7 Hz, 1H), 7.12 (d, J=7.7 Hz, 1H), 7.01 (d, J=7.7 Hz, 1H), 5.88 (s, 2H), 2.79 (t, J=7.5 Hz, 2H), 2.12 (s, 6H), 1.78 (sext, J=7.5 Hz, 2H), 0.95 (t, J=7.5 Hz, 3H).

¹³C-NMR (101 MHz, CDCl₃): δ 162.6, 151.6, 138.1, 128.7, 121.5, 119.1, 106.9, 40.2, 23.1, 14.0, 13.4.

### Step c

### 6-propylpyridin-2-amine

A solution of 2-(2,5-dimethyl-1H-pyrrol-1-yl)-6-propylpyridine (235 mg, 1.1 mmol, 1 eq.) in a mixture of ethanol/water: 3/1 (4.2 mL) and hydroxylamine hydrochloride (385.2 mg, 5.5 mmol, 5 eq.) was heated a 100° C. for about 16 hours, cooled to room temperature and extracted with ethyle acetate. The organic layer was dried over Na₂SO₄, filtered and evaporated in vacuo. The crude product was purified by chromatography on silica gel using a gradient of 50% to 75% ethyl acetate in heptane to afford 6-propylpyridine-2 amine as a solid.

¹H-NMR (400 MHz, CDCl₃): δ 7.33 (t, J=7.7 Hz, 1H), 6.49 (d, J=7.4 Hz, 1H), 6.31 (d, J=7.7 Hz, 1H), 4.50 (bs, 2H), 2.57 (t, J=7.5 Hz, 2H), 1.69 (sext, J=7.5 Hz, 2H), 0.95 (t, J=7.5 Hz, 3H).

¹³C-NMR (101 MHz, CDCl₃): δ 160.9, 158.3, 138.3, 112.6, 106.0, 40.2, 23.1, 14.1

### Preparation of 6-cyclopropylpyridin-2-amine

### Step d: N-(6-Bromopyridin-2-yl)pivalamide

To a mixture of 6-bromo-pyridin-2-ylamine (1.0 g, 6 mmol, 1 eq.) and DIEA (2.07 mL, 11.85 mmol, 2.05 eq.) in dichloromethane (8.6 mL) was added dropwise pivaloyl chloride (0.75 mL, 6.07 mmol, 1.05 eq.) at 0° C. and the reaction mixture was stirred at room temperature for 4 h. The reaction mixture was washed with water, dried over sodium sulfate, and evaporated. The crude product was purified by chromatography on silica gel using EtOAc/heptane: 1/1 to afford the expected product N-(6-bromopyridin-2-yl)pivalamide as white solid (1.49 g, 99%).

¹H-NMR (400 MHz, CDCl₃): δ 8.18 (d, J=8.2 Hz, 1H), 7.94 (bs, 1H), 7.51 (t, J=7.9 Hz, 1H), 7.16 (d, J=7.7 Hz, 1H), 1.27 (s, 9H).

¹³C-NMR (101 MHz, CDCl₃) δ: 177.3, 151.9, 140.8, 139.3, 123.6, 112.5, 40.1, 27.6.

### Step e

### N-(6-Cyclopropyl-pyridin-2-yl)-2,2-dimethyl-propionamide

A 20 mL microwave vial (oven-dried and under Argon) containing a Teflon® stirred bar was charged with of N-(6-Bromopyridin-2-yl)pivalamide (200 mg, 0.78 mmol, 1 eq.), potassium cyclopropyltrifluoroborate (172.7 mg, 1.16 mmol, 1.5 eq.), K₂CO₃ (322.5 mg, 2.33 mmol, 3 eq.), Ru-Phos (29.04 mg, 0.062 mmol, 0.08 eq.), toluene (4.5 mL and H₂O (0.45 mL). The vessel was evacuated and backfilled with nitrogen (this process was repeated a total of 3 times) than Pd(OAc)₂ (7.13 mg, 0.031 mmol, 0.04 eq.) was introduced. The reaction mixture was then capped properly and placed in a preheated oil bath at 80° C. until complete conversion of the starting material was detected (approximatively 18 h). The resulting solution was then diluted with water (10 mL) and extracted with EtOAc (2×15 mL). The combined organic extracts were washed with water (15 mL) and brine (15 mL), dried over Na2SO4, and filtered. The filtrate was evaporated in vacuo and the residue was purified by flash column chromatography on silica gel (EtOAc/heptane: 1/4) to give the title product as a clear oil (100 mg, 59%).

¹H-NMR (400 MHz, CDCl₃): δ 7.95 (d, J=8.2 Hz, 1H), 7.81 (bs, 1H), 7.50 (t, J=7.9 Hz, 1H), 6.81 (d, J=7.6 Hz, 1H), 1.92 (qt, J=6.5 Hz, 1H), 1.29 (s, 9H), 0.92 (m, 2H), 0.91 (m, 2H).

¹³C-NMR (101 MHz, CDCl₃) δ: 177.1, 161.5, 151.2, 138.3, 117.0, 110.4, 39.9, 27.7, 17.0, 9.6.

### Step f

6-cyclopropylpyridin-2-amine: To a solution of N-(6-Cyclopropyl-pyridin-2-yl)-2,2-dimethyl-propionamide (92 mg, 0.42 mmol, 1 eq.) in 1,4-dioxane (1 mL) was added HCl (12 N, 0.5 mL). The mixture was stirred for 24 hours at 100° C. After cooling to 25° C., the pH of the reaction mixture was adjusted with NaOH to achieve pH=9. The solution was diluted with ethyl acetate (120 mL) and washed with saturated aqueous sodium bicarbonate (2×30 mL). Next, the organic layer was azeotroped with toluene (10 mL) to afford 6-cyclopropylpyridin-2-amine as clear oil (56 mg, 100%).

¹H-NMR (400 MHz, CDCl₃): δ 7.25 (t, J=7.8 Hz, 1H), 6.43 (d, J=7.5 Hz, 1H), 6.22 (d, J=8.0 Hz, 1H), 4.35 (bs, 2H), 1.86 (m, 1H), 0.91-0.83 (m, 4H).

¹³C-NMR (101 MHz, CDCl₃) δ: 161.5, 158.2, 137.8, 111.0, 105.3, 17.0, 9.1.

### Preparation of 6-(methoxymethyl)pyridin-2-amine

### Step g

### 2-bromo-6-methoxymethyl-pyridine

Under nitrogen, a solution of (6-bromo-pyridin-2-yl)-methanol (300 mg, 1.53 mmol, 1 eq.) in anhydrous tetrahydrofuran (1 mL) was added dropwise to a stirring suspension of sodium hydride (60% dispersion in oil, 44.1 mg, 1.84 mmol, 1.2 eq.) in anhydrous tetrahydrofuran (2 mL) cooled at 0° C. After gas evolution ceased, methyl iodide (143 μL, 2.23 mmol, 1.5 eq.) was added dropwise. The mixture was allowed to raise room temperature over 1 hour. The reaction was quenched by addition of cold H₂O (5 mL) and diluted with brine (5 mL) and extracted with EtOAc (20 mL) the organic layer was washed with brine (10 mL), dried over sodium sulfate and filtered and concentrated in vacuo. The residue was purified by flash column chromatography on silica gel (EtOAc/heptane 1/5) to give the title product as a clear oil (286 mg, 92%).

¹H-NMR (400 MHz, CDCl₃): δ 7.52 (t, J=7.7 Hz, 1H), 7.36 (d, J=8.4 Hz, 1H), 7.34 (d, J=8.2 Hz, 1H), 4.52 (s, 2H), 3.43 (s, 3H).

¹³C-NMR (101 MHz, CDCl₃): δ 160.4, 141.5, 139.2, 126.8, 120.0, 74.8, 59.1.

### Step h

### 6-(methoxymethyl)pyridin-2-amine

A 50 ml microwave vial containing a Teflon® stirred bar was charged with 2-bromo-6-methoxymethyl-pyridine (211 mg, 1.04 mmol, 1 eq.), copper sulfate hydrate (352 mg, 1.39 mmol, 1.33 eq.), aqueous NH₃ (28%, 8 mL, 55 eq.) and ethanol (2 mL). The reaction mixture was then capped properly and placed in a preheated oil bath at 180° C. during 18 hours. After cooling to room temperature the mixture was poured into distilled water (25 mL). After extraction with ethyl acetate (3×30 mL) the combined extracts were washed with distilled water (3×20 mL), dried over sodium sulfate and evaporated under reduced pressure. The crude product was purified on silica gel using a gradient of 60% to 75% EtOAc in heptane to afford the expected product 6-(methoxymethyl)pyridin-2-amine as a yellow oil (84 mg, 58%)

¹H-NMR (400 MHz, CDCl₃): δ 7.39 (t, J=7.5 Hz, 1H), 6.70 (bs, 1H), 6.38 (bs, 1H), 4.49 (bs, 2H), 4.35 (s, 2H), 3.42 (s, 3H).

¹³C-NMR (101 MHz, CDCl₃) δ: 158.2, 156.6, 138.4, 111.8, 107.7, 75.7, 58.9.

The non commercially available compounds in hand, the examples 6 have been prepared followed method 9 described in scheme 8

### Preparation of N2-alkyl-5-arylpyridine-2-amine (6): method 9

Scheme 8

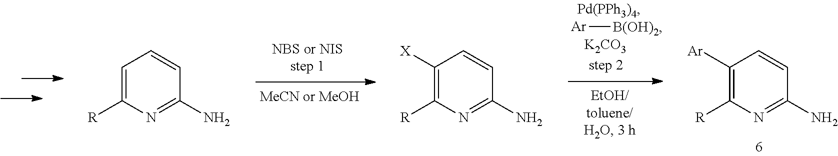

R = Me, Et, CH₂OMe, CF₃, n-Pr, i-Pr, c-Pr . . . etc

| | | | | | Step 1, | Step 2, |
|---|---|---|---|---|---|---|
| Entry | N° | X | R | Ar | yield % | yield % |
| 1 | 6a | Br | H | 2,3-Cl₂—Ph | 85 | 49 |
| 6 | 6b | I | Me | 2,3-Cl₂—Ph | 58 | 69 |
| 3 | 6c | Br | Et | 2,3-Cl₂—Ph | 84 | 34 |
| 4 | 6d | Br | Et | 2-OMe—Ph | 84 | 72 |
| 6 | 6e | Br | CH₂OMe | 2-OMe—Ph | 100 | 50 |
| 6 | 6f | Br | CF₃ | 2-OMe—Ph | 100 | 76 |
| 7 | 6g | Br | n-Pr | 2-OMe—Ph | 47 | 68 |
| 8 | 6h | Br | i-Pr | 2-OMe—Ph | 71 | 73 |
| 9 | 6i | Br | c-Pr | 2-OMe—Ph | 46 | 64 |

### Example 9: Preparation of 5-(2-methoxyphenyl)-6-propylpyridin-2-amine, 6g

### Step 1: Preparation of 5-bromo 6-propyl 2-amine

To a solution of 6 propylpyridine-2 amine (100 mg, 0.73 mmol, 1 eq.) in methanol (2.5 mL) and cooled by an ice bath was added NBS (137 mg, 1.05 eq.). The resulting mixture was stirred for 1 hour and then concentrated. Purification by flash chromatography (EtOAc/heptane: 1/4) afforded the title compound (74 mg, 47%)

¹H-NMR (400 MHz, CDCl₃): δ 7.45 (d, J=8.5 Hz, 1H), 6.20 (d, J=8.5 Hz, 1H), 4.41 (bs, 2H), 2.71 (t, J=7.8 Hz, 2H), 1.67 (sext, J=7.7 Hz, 2H), 0.96 (t, J=7.4 Hz, 3H).

¹³C-NMR (101 MHz, CDCl₃): δ 158.8, 157.0, 141.7, 108.7, 107.8, 39.5, 22.2, 14.2.

### Step 2: 5-(2-methoxyphenyl)-6-propylpyridin-2-amine, 6g

A 5 ml microwave vial containing a Teflon® stirred bar was charged with 3-bromo 6-propyl 2-amine (74 mg, 0.34 mmol, 1 eq.), 2-methoxyphenylboronic acid (62.74 mg, 0.41 mmol, 1.2 eq.), Na₂CO₃ (110 mg, 1 mmol, 3 eq.) followed by the addition of a mixture of Toluene/EtOH/H₂O: 2.5/0.5/0.5 (0.1 mmol/mL). The vessel was evacuated and backfilled with nitrogen (this process was repeated a total of 3 times) and Pd(PPh₃)₄ (20.1 mg, 0.017 mmol, 0.05 eq.) was introduced. The reaction mixture was then capped properly and placed in a preheated oil bath at 120° C. until complete conversion of the starting material was detected. The reaction mixture was monitored by HPLC analysis and was usually complete within 4 hours. The reaction mixture was then concentrated under vacuum and the crude product was purified by chromatography on silica gel using EtOAc/heptane: 1/3 to afford the expected product 6g as a white solid (65.2 mg, 68%) after preparation of the corresponding hydrochloride salt.

¹H-NMR (400 MHz, DMSO-d6): δ 14.10 (s, 1H), 7.90 (bs, 2H), 7.69 (d, J=8.9 Hz, 1H), 7.43 (t, J=7.9 Hz, 1H), 7.17 (d, J=7.3 Hz, 1H), 7.14 (d, J=8.2 Hz, 1H), 7.04 (t, J=7.4 Hz, 1H), 6.88 (d, J=8.9 Hz, 1H), 3.74 (s, 3H), 2.48 (m, 2H), 1.55 (sext, J=7.3 Hz, 2H), 0.73 (t, J=7.3 Hz, 3H).

¹³C-NMR (101 MHz, CDCl₃): δ 158.8, 157.3, 157.1, 140.2, 131.7, 129.5, 128.7, 123.6, 120.6, 110.9, 105.7, 55.5, 37.6, 22.9, 14.3.

5-(2,3-dichlorophen yl)pyridin-2-amine, 6a. Following general method 9 and starting from 5-bromopyridin-2-amine and 2,3-Cl₂Ph boronic acid, 6a was obtained as a white solid (82 mg, 49%).

¹H-NMR (400 MHz, CDCl₃): δ 8.14 (d, J=2.2 Hz, 1H), 7.57 (dd, J=8.5 Hz, 2.2 Hz, 1H), 7.47 (dd, J=7.5 Hz, 2.2 Hz, 1H), 7.27 (d, J=8.5 Hz, 1H), 7.23 (m, 1H), 6.59 (d, J=8.5 Hz, 1H), 4.60 (bs, 2H).

¹³C-NMR (101 MHz, CDCl₃): δ 157.8, 148.0, 139.8, 138.9, 133.8, 131.5, 129.5, 129.3, 127.3, 125.5, 107.7.

5-(2,3-dichlorophenyl)-6-methylpyridin-2-amine, 6b. Following general method 9 and starting from 5-iodo-6-methylpyridin-2-amine and 2,3-Cl₂Ph boronic acid, 6b was obtained as a solid (60 mg, 69%)

¹H-NMR (400 MHz, DMSO-d6): δ 7.61 (d, J=1.1 Hz, 1H), 7.38 (t, J=1.1 Hz, 1H), 7.26 (d, J=7.3 Hz, 1H), 7.12 (d, J=8.2 Hz, 1H), 6.34 (d, J=8.2 Hz, 1H), 6.04 (bs, 2H), 2.01 (s, 3H).

¹³C-NMR (101 MHz, CDCl₃): δ 157.6, 154.3, 141.2, 140.2, 139.4, 133.1, 129.8, 129.5, 126.9, 124.3, 105.6, 22.0.

5-(2,3-dichlorophenyl)-6-ethylpyridin-2-amine, 6c. Following general method 9 and starting from 5-bromo-6-ethylpyridin-2-amine and 2,3-Cl₂Ph boronic acid, 6c was obtained as a white solid (43.5 mg, 34%) after preparation of the corresponding hydrochloride salt.

¹H-NMR (400 MHz, DMSO-d6): δ 14.36 (s, 1H), 8.07 (bs, 2H), 7.76 (m, 2H), 7.49 (t, J=7.8 Hz, 1H), 7.41 (dd, J=7.7 Hz, 1.4 Hz, 1H), 6.93 (d, J=8.9 Hz, 1H), 2.54 (m, 1H), 2.42 (m, 1H), 1.11 (t, J=7.6 Hz, 3H).

¹³C-NMR (101 MHz, CDCl₃): δ 159.4, 158.0, 141.5, 139.5, 133.5, 132.9, 130.1, 129.7, 127.2, 124.1, 105.7, 28.8, 13.8.

6-ethyl-5-(2-methoxyphenyl)pyridin-2-amine, 6d. Following general method 9 and starting from 5-bromo-6-ethylpyridin-2-amine and 2-OMe-Phenyl boronic acid, 6d was obtained a white solid (80.9 mg, 72%) after preparation of the corresponding hydrochloride salt.

¹H-NMR (400 MHz, DMSO-d6): δ 14.16 (s, 1H), 7.94 (bs, 2H), 7.69 (d, J=9.0 Hz, 1H), 7.43 (td, J=7.8 Hz, 1.8 Hz, 1H), 7.18 (dd, J=7.4 Hz, 1.7 Hz, 1H), 7.14 (d, J=8.2 Hz, 1H), 7.05 (t, J=7.4 Hz, 1H), 6.88 (d, J=9.0 Hz, 1H), 3.74 (s, 3H), 2.50 (m, 2H), 1.12 (t, J=7.6 Hz, 3H).

¹³C-NMR (101 MHz, CDCl₃): δ 160.0, 157.4, 157.1, 140.2, 131.7, 129.5, 128.7, 123.2, 120.6, 110.9, 105.7, 55.5, 28.9, 13.9.

5-(2-methoxyphenyl)-6-(trifluoromethoxy)pyridin-2-amine, 6f. Following general method 9 and starting 5-bromo-6-(trifluoromethoxy)pyridin-2-amine and 2-OMe-Phenyl boronic acid, 6f was obtained as a white solid (96.6 mg, 76%) after preparation of the corresponding hydrochloride salt.

¹H-NMR (400 MHz, DMSO-d6): δ 7.75 (bs, 3H), 7.35 (td, J=7.9 Hz, 1.5 Hz, 1H), 7.31 (d, J=8.6 Hz, 1H), 7.08 (dd, J=7.3 Hz, 1.5 Hz, 1H), 7.04 (d, J=8.4 Hz, 1H), 6.97 (t, J=7.4 Hz, 1H), 6.71 (d, J=8.6 Hz, 1H), 3.69 (s, 3H).

¹³C-NMR (101 MHz, CDCl₃): δ 157.1, 144.0, 143.7, 142.7, 131.0, 129.5, 127.0, 123.1, 122.1 (q, J=276 Hz), 120.3, 111.2, 110.8, 55.6.

6-isopropyl-5-(2-methoxyphenyl)pyridin-2-amine, 6 h. Following general method 9 and starting from 5-bromo-6-isopropylpyridin-2-amine and 2-OMe-Phenyl boronic acid, 6h was obtained as a white solid (100 mg, 73%) after preparation of the corresponding hydrochloride salt.

¹H-NMR (400 MHz, DMSO-d6): δ 13.91 (bs, 1H), 8.24 (bs, 2H), 7.68 (d, J=9.0 Hz, 1H), 7.43 (t, J=7.7 Hz, 1H), 7.17 (d, J=7.3 Hz, 1H), 7.13 (d, J=8.3 Hz, 1H), 7.04 (t, J=7.3 Hz, 1H), 6.88 (d, J=9.0 Hz, 1H), 3.74 (s, 3H), 2.77 (hept, J=7.0 Hz, 1H), 1.29 (d, J=7.0 Hz, 3H), 1.22 (d, J=7.0 Hz, 3H).

¹³C-NMR (101 MHz, CDCl₃): δ 163.6, 157.6, 157.2, 140.0, 131.7, 129.8, 128.6, 122.6, 120.6, 110.8, 105.6, 55.5, 32.0, 22.9, 21.8.

### Example 10: 6-cyclopropyl-5-(2-methoxyphenyl)pyridin-2-amine 6i

### Step 1: Preparation of 5-bromo-6-cyclopropylpyridin-2-amine

To a solution of 6-cyclopropylpyridin-2-amine (85 mg, 0.63 mmol, 1 eq.) in methanol (2.3 mL) and cooled by an ice bath was added NBS (118 mg, 1.05 eq.). The resulting mixture was stirred for 1 hour and then concentrated. Purification by flash chromatography (AcOEt/heptane: 1/4) afforded the title compound (63 mg, 46%)

¹H-NMR (400 MHz, CDCl₃): δ 7.42 (d, J=8.6 Hz, 1H), 6.12 (d, J=8.6 Hz, 1H), 4.27 (bs, 2H), 2.35 (m, 1H), 0.97 (m, 2H), 0.89 (m, 2H).

¹³C-NMR (101 MHz, CDCl₃) δ: 158.5, 157.0, 141.1, 108.9, 106.8, 15.6, 9.7.

### Step 2: Preparation of 6-cyclopropyl-5-(2-methoxyphenyl)pyridin-2-amine, 6i

A 5 mL microwave vial containing a Teflon® stirred bar was charged with 5-bromo 6-cyclopropyl-2-amine (63 mg, 0.3 mmol, 1 eq.), 2-methoxy phenylboronic acid (53.74 mg, 0.36 mmol, 1.2 eq.), Na₂CO₃ (94.2 mg, 0.9 mmol, 3 eq.) followed by the addition of a mixture of Toluene/EtOH/H₂O: 2.1/0.35/0.35 (0.1 mmol/mL). The vessel was evacuated and backfilled with nitrogen (this process was repeated a total of 3 times) and Pd(PPh₃)₄ (17.2 mg, 0.015 mmol, 0.05 eq.) was introduced. The reaction mixture was then capped properly and placed in a preheated oil bath at 120° C. until complete conversion (usually 4 h). The reaction mixture was then concentrated under vacuum and the crude product was purified by chromatography on silica gel using EtOAc/heptane: 1/1 to afford the expected product 6i as a white solid (52.6 mg, 64%) after preparation of the corresponding hydrochloride salt.

¹H-NMR (400 MHz, DMSO-d6): δ 13.00 (bs, 1H), 8.14 (bs, 2H), 7.66 (d, J=8.3 Hz, 1H), 7.42 (t, J=7.7 Hz, 1H), 7.24 (d, J=7.4 Hz, 1H), 7.14 (d, J=8.3 Hz, 1H), 7.05 (t, J=7.4 Hz, 1H), 6.80 (d, J=8.3 Hz, 1H), 3.76 (s, 3H), 1.80 (m, 1H), 1.14 (m, 2H), 0.97 (m, 2H).

¹³C-NMR (101 MHz, CDCl₃): δ 158.7, 157.4, 157.3, 139.6, 132.1, 129.4, 128.6, 123.4, 120.6, 111.1, 104.7, 55.7, 14.4, 9.4.

### Example 11: Preparation of 6-(methoxymethyl)-5-(2-methoxyphenyl)pyridin-2-amine, 6e

### Step 1: Preparation of 5-bromo-6-(methoxymethyl)pyridin-2-amine

To a solution of 6-(methoxymethyl)pyridin-2-amine (61.4 mg, 0.44 mmol, 1 eq.) in methanol (1.5 mL) and cooled by an ice bath was added NBS (80.7 mg, 0.45 mmol, 1.02 eq.). The resulting mixture was stirred for 1 hour and then concentrated. Purification by flash chromatography (EtOAc/heptane: 1/1) afforded the title compound as a light brown solid in (96 mg, 99%)

¹H-NMR (400 MHz, CDCl₃): δ 7.47 (d, J=8.7 Hz, 1H), 6.29 (d, J=8.7 Hz, 1H), 4.79 (bs, 2H), 4.52 (s, 2H), 3.44 (s, 3H).

¹³C-NMR (101 MHz, CDCl₃) δ: 157.6, 153.1, 141.9, 109.7, 107.7, 74.0, 58.9.

### Step 2: Preparation of 6-(methoxymethyl)-5-(2-methoxyphenyl)pyridin-2-amine, 6e

A 10 mL microwave vial containing a Teflon® stirred bar was charged with 5-bromo-6-(methoxymethyl)pyridin-2-amine (70 mg, 0.32 mmol, 1 eq.), 2-methoxyphenylboronic acid (58.8 mg, 0.39 mmol, 1.2 eq.), Na₂CO₃ (103 mg, 0.96 mmol, 3 eq.) followed by the addition of a mixture of Toluene/EtOH/H₂O: 2.3/0.4/0.4 (0.1 mmol/mL). The vessel was evacuated and backfilled with nitrogen (this process was repeated a total of 3 times) and Pd(PPh₃)₄ (18.8 mg, 0.016 mmol, 0.05 eq.) was introduced. The reaction mixture was then capped properly and placed in a preheated oil bath at 120° C. until complete conversion of the starting material (usually 4 h). The reaction mixture was then concentrated under vacuum and the crude product was purified by chromatography on silica gel using EtOAc/heptane: 3/1 to afford the expected product 6e as a white solid (45.2 mg, 50%) after preparation of the corresponding hydrochloride salt.

¹H-NMR (400 MHz, DMSO-d6): δ 13.28 (bs, 1H), 7.97 (bs, 2H), 7.60 (d, J=9.0 Hz, 1H), 7.26 (td, J=7.9 Hz, 1.6 Hz, 1H), 7.02 (dd, J=7.9 Hz, 1.6 Hz, 1H), 6.96 (d, J=8.3 Hz, 1H), 6.87 (t, J=7.4 Hz, 1H), 6.83 (d, J=9.0 Hz, 1H), 4.10 (s, 2H), 3.57 (s, 3H), 3.09 (s, 3H).

¹³C-NMR (101 MHz, CDCl₃): δ 157.6, 157.0, 153.4, 140.6, 131.6, 129.1, 128.2, 123.7, 120.7, 110.9, 107.7, 73.2, 58.7, 55.6.

8—Preparation of N2-alkoxy-5-arylpyridine-2-amine (Method 10)

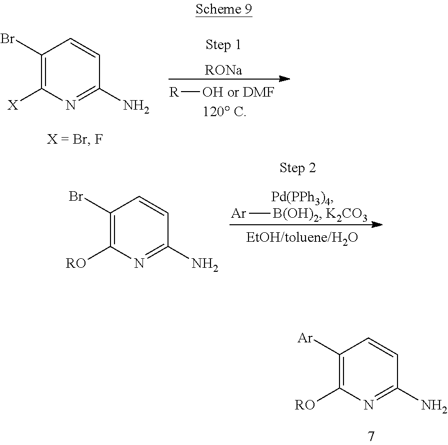

According to the above scheme 9, compounds of the general formula 7 are prepared by aromatic nucleophilic substitution of the well-known 5,6-dihalogeno-2-aminopyridines with the appropriate alkoxyde. This reaction is preferably carried out at 120° C. for 48 h in an alcoholic solvent or in DMF. The second step of the reaction is a Suzuki-Miyaura reaction according to conventional conditions.

### Example 12: Preparation of 5-(2,3-dichlorophenyl)-6-methoxypyridin-2-amine 7a

### Step 1

### Preparation of 5-bromo-6-methoxypyridin-2-amine

A 20 mL microwave vial containing a Teflon® stirred bar was charged with 5,6 dibromo pyridine-2 amine (300 mg, 1.12 mmol, 1 eq.), sodium methoxide (167.3 mg, 3.1 mmol, 2.6 eq.), and MeOH (4 mL). The vial was then capped properly and placed in a preheated oil bath at 120° C. until complete conversion of the starting material was detected (usually 24 hours). The reaction mixture was then concentrated under vacuum and the crude product was purified by chromatography on silica gel using EtOAc/heptane: 1/1 to afford the expected product as an yellow oil (186 mg, 77%).

¹H-NMR (400 MHz, CDCl₃): δ 7.49 (d, J=8.0 Hz, 1H), 5.99 (d, J=8.0 Hz, 1H), 4.31 (bs, 2H), 3.91 (s, 3H).

### Step 2

### Preparation of 5-(2,3-dichlorophenyl)-6-methoxypyridin-2-amine, 7a

A 5 mL microwave vial containing a Teflon® stirred bar was charged with 5-bromo-6-methoxypyridin-2-amine (90 mg, 0.44 mmol, 1 eq.), 2,3-dichloro phenylboronic acid (97.2 mg, 0.51 mmol, 1.15 eq.), Na₂CO₃ (140 mg, 1.32 mmol, 3 eq.) followed by the addition of a mixture of Toluene/EtOH/H₂O: 3.2/0.5/0.5 (0.1 mmol/mL). The vessel was evacuated and backfilled with nitrogen (this process was repeated a total of 3 times) and Pd(PPh₃)₄ (25.6 mg, 0.022 mmol, 0.05 eq.) was introduced. The reaction mixture was then capped properly and placed in a preheated oil bath at 120° C. until complete conversion of the starting material (usually 3 h). The reaction mixture was then concentrated under vacuum and the crude product was purified by chromatography on silica gel using EtOAc/heptane: 1/1 to afford the expected product 7a as a solid (70 mg, 59%)

¹H-NMR (400 MHz, CDCl₃): δ 7.39 (dd, J=6.9 Hz, 2.8 Hz, 1H), 7.25 (d, J=7.8 Hz, 1H), 7.18 (m, 2H), 6.13 (d, J=7.8 Hz, 1H), 4.28 (bs, 2H), 3.83 (s, 3H).

¹³C-NMR (101 MHz, CDCl₃) δ: 159.9, 157.0, 141.4, 138.8, 133.2, 132.8, 130.2, 129.2, 126.8, 111.2, 99.4, 53.6.

### 9—Additional Substitution at Positions 4 (R4) or 5 (R5), Methods 11-13

Compounds of formula 8 and 9 can be prepared following the synthetic sequences described in the above scheme. Iodination reaction of 2-amino-6-chloro pyridine derivatives or the well-known 2,6-diaminopyridines derivatives followed by a Suzuki cross coupling reaction led to compounds of formula 8 and 9 (Methods 11 and 12). A third possible pathway (Method 13), involving a reductive amination of compounds of the general formula 1-3, in presence of an appropriate aldehyde, NaBH₃CN and acetic acid surprisingly lead to derivatives 9 (scheme 10).

Scheme 10

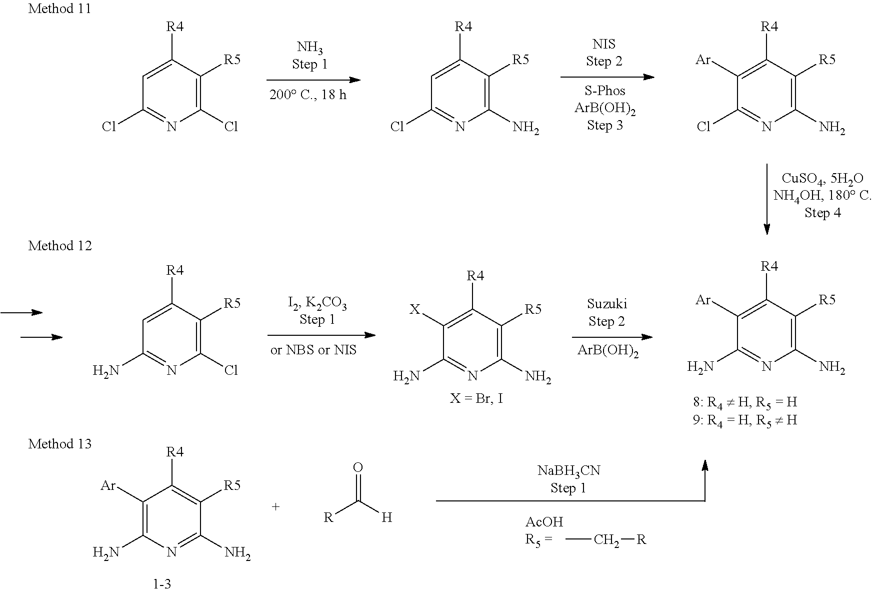

| Entry | N° | R⁴ | R⁵ | Ar | Method |
|---|---|---|---|---|---|
| 1 | 8a | OMe | H | 2,3-Cl₂—Ph | 12 |
| 2 | 8b | Me | H | 2-Me—Ph | 11 |
| 3 | 9a | H | F | 2,3-Cl₂—Ph | 12 |
| 4 | 9b | H | Et | 2,3-Cl₂—Ph | 13 |

### Example 13: 4-methyl-3-(o-tolyl)pyridine-2,6-diamine, 8b, Method 11

### Step 1

6-Chloro-4-methylpyridin-2-amine: A solution of 2,6-Dichloro-4-methylpyridine (0.5 g, 3.09 mmol, 1 eq.) in ammonium hydroxide (2.5 mL, 28% solution in water) was heated at 200° C. in a pressure vessel for 12 h. The reaction mixture was then concentrated under reduced pressure. The resulting residue was dissolved in EtOAc (3×30 mL) the organic layer was washed with distilled water (3×20 mL), dried over sodium sulfate and evaporated under reduced pressure. The crude product was purified on silica gel using EtOAc/heptane: 1/1 to afford the expected product as a white solid (308 mg, 70%).

¹H-NMR (400 MHz, CDCl₃): δ 6.45 (s, 1H), 6.15 (s, 1H), 4.63 (bs, 2H), 2.16 (s, 3H).

¹³C-NMR (101 MHz, CDCl₃) δ: 158.7, 151.8, 149.5, 114.4, 107.1, 20.9.

### Step 2

### 6-Chloro-5-iodo-4-methylpyridin-2-amine

To a N,N-dimethyl formamide (4.2 mL) solution of 2-amino-4-methyl-6-chloropyridine (100 mg, 0.70 mmol, 1 eq.) was added N-iodo succinimide (189.3 mg, 0.84 mmol, 1.2 eq.) and the mixture was heated at 80° C. for 2 hours. The reaction mixture was then concentrated under reduced pressure. The resulting residue was dissolved in EtOAc (3×30 mL) the organic layer was washed with distilled water (3×20 mL), dried over sodium sulfate and evaporated under reduced pressure. The crude product was purified on silica gel using EtOAc/heptane: 1/3 to afford the expected product as a yellow solid (152 mg, 81%).

¹H-NMR (400 MHz, CDCl₃): δ 6.29 (s, 1H), 4.55 (bs, 2H), 2.34 (s, 3H).

¹³C-NMR (101 MHz, CDCl₃): δ 157.7, 155.5, 153.4, 108.1, 85.7, 29.7.

### Step 3

### 6-chloro-4-methyl-5-(o-tolyl)pyridin-2-amine

A 5 mL microwave vial containing a Teflon® stirred bar was charged with: 6-Chloro-5-iodo-4-methylpyridin-2-amine (30 mg, 0.11 mmol, 1 eq.), 3-tolylboronic acid (19.2 mg, 0.13 mmol, 1.2 eq.), K₂CO₃ (30.9 mg, 0.22 mmol, 2 eq.) followed by the addition of Pd(OAc)₂ (1.28 mg, 5.6 μmol, 0.05 eq.) and S-Phos (4.6 mg, 0.011 mmol, 0.10 eq.). A mixture of MeCN/H₂O: 0.8/1 mL was then introduced, the vessel was evacuated and backfilled with nitrogen (this process was repeated a total of 3 times). The reaction mixture was then capped properly and placed in a preheated oil bath at 105° C. until complete conversion of the starting material was detected (approximatively 15 hours). The resulting residue was dissolved in ethyl acetate and the organic layer was washed with distilled water and brine, dried over sodium sulfate and evaporated under reduced pressure. The crude product was purified by chromatography on silica gel using EtOAc/heptane: 1/4 to afford 3b as a yellow solid (17.7 mg, 68%).

¹H-NMR (400 MHz, CDCl₃): δ 7.27-7.21 (m, 3H), 7.02 (d, J=7.5 Hz, 1H), 6.35 (s, 1H), 4.46 (bs, 2H), 2.05 (s, 3H), 1.89 (s, 3H).

¹³C-NMR (101 MHz, CDCl₃) δ: 157.3, 150.6, 148.6, 137.0, 136.9, 130.2, 130.0, 128.1, 126.2, 125.9, 108.0, 20.7, 19.7.

### Step 4

### 4-methyl-3-(o-tolyl)pyridine-2,6-diamine, 8b

A 5 ml microwave vial containing a Teflon® stirred bar was charged with the -chloro-4-methyl-5-(o-tolyl)pyridin-2-amine (43 mg, 0.18 mmol, 1 eq.), copper sulfate hydrate (62.3 mg, 0.25 mmol, 1.33 eq.), aqueous NH₃ (28%, 2.5 mL, 100 eq.) and ethanol (3 mL). The reaction mixture was then capped properly and placed in a preheated oil bath at 180° C. during 12 hours. After cooling to room temperature the mixture was poured into distilled water (25 mL). After extraction with ethyl acetate (3×30 mL) the combined extracts were washed with distilled water (3×20 mL), dried over sodium sulfate and evaporated under reduced pressure. The crude product was purified on silica gel using EtOAc/heptane: 1/1 to afford the title compound 8b as a pale yellow solid (27.1 mg, 59%) after preparation of the corresponding hydrochloride salt.

¹H-NMR (400 MHz, DMSO-d6): δ 13.07, (bs, 1H), 7.37-7.29 (m, 3H), 7.26 (bs, 2H), 7.06 (d, J=7.2 Hz, 1H), 6.43 (bs, 2H), 5.95 (s, 1H), 2.04 (s, 3H), 1.76 (s, 3H).

¹³C-NMR (101 MHz, DMSO-d6) δ: 154.7, 150.4, 148.6, 137.5, 132.4, 130.9, 130.5, 128.5, 126.7, 107.4, 96.8, 20.4, 18.8.

### Example 14: 3-(2,3-dichlorophenyl)-4-methoxypyridine-2,6-diamine, 8a, Method 12

For example 14, the starting 2,6-diamino-4-methoxy-pyridine was not commercially available and then subsequently prepared according to the following 3 steps procedure.

Preparation of 4-methoxypyridine-2,6-diamine (adapted from Chem. Eur. J. 2001, 1889-1898)

### Step a

Dimethyl 4-Methoxypyridine-2,6-dicarboxylate: (Inorganic Chemistry, 50(9), 4125-4141; 2011) and 4-Hydroxypyridine-2,6-dicarboxylic acid (1.5 g, 8.2 mmol, 1 eq.) were dissolved in methanol (40 mL), and 2.5 mL of concentrated H₂SO₄ was added. The mixture was refluxed for 24 h and then allowed to cool to room temperature. A saturated NaHCO₃ solution was added (25 mL), and the mixture was extracted with CH₂Cl₂ (3×20 mL). The combined organic extracts were dried over Na₂SO₄ and evaporated to dryness. The crude product was purified by column chromatography on SiO₂ with a CH₂Cl₂/MeOH 10% mixture as the eluent to give Dimethyl 4-Methoxypyridine-2,6-dicarboxylate as a white solid (1.45 g, 78%).

¹H-NMR (400 MHz, CDCl₃): δ 7.80 (s, 2H), 3.99 (s, 6H), 3.99 (s, 3H).

¹³C NMR (400 MHz, CDCl₃): δ 167.6, 165.1, 149.8, 114.1, 56.0, 53.2

### Step b

4-methoxypyridine-2,6-dicarboxamide: (adapted from Chem. Eur. J. 2001, 1889-1898) To a solution of dimethyl 4-methoxypyridine-2,6-dicarboxylate (200 mg, 0.89 mmol, 1 eq.) in methanol (4 mL) was added dropwise a solution of NH₄OH 30% (4 mL). The resulting mixture was refluxed for 1 h. The solvent was removed under vacuum to afford the title diamide as a white powder (161 mg, 93%).

¹H-NMR (400 MHz, DMSO-d6): δ 8.83 (bs, 2H), 7.70 (bs, 2H), 7.66 (s, 2H), 3.95 (s, 3H).

¹³C-NMR (101 MHz, DMSO-d6) δ: 167.7, 165.1, 151.2, 109.7, 55.9.

### Step c

### 4-methoxypyridine-2,6-diamine

To a solution of bromine (65.8 μL, 1.28 mmol, 2.5 eq.) in potassium hydroxide solution (0.6 g in 1 ml of H₂O), was added a solution of the diamide prepared on step b (100 mg, 0.51 mmol, 1 eq.) in 1,4-dioxane (2.25 mL). The resulting mixture was first stirred at RT for 1 h, then was heated to 100° C. for 2 h. After extraction with EtOAc (3×20 mL), the combined organic layers were dried over Na₂SO₄ filtered and concentrated in vacuo. The crude product was purified by chromatography on silica gel using pure EtOAc to afford 4-methoxypyridine-2,6-diamine as a solid (54 mg, 75%).

¹H-NMR (400 MHz, CDCl₃): δ 5.46 (s, 2H), 4.15 (bs, 4H), 3.71 (s, 3H).

¹³C-NMR (101 MHz, CDCl₃) δ: 169.4, 159.3, 84.4, 55.9.

### Step 1

### 3-Iodo-4-methoxypyridine-2,6-diamine

To a solution of 4-MeO-2,6-diaminopyridine (46 mg, 0.33 mmol, 1 eq.) in 2-methyl-tetrahydrofuran (1 mL) was added potassium carbonate (45.7 mg, 0.33 mmol, 1 eq.). To this suspension was added a solution of iodine (84.1 mg, 0.33 mmol, 1 eq.) in 2-methyl-tetrahydrofuran (1 mL) dropwise over 1 hour. The reaction was stirred for 2 hours at room temperature. The reaction was filtered through a pad of celite and washed with ethyl acetate, and the filtrate collected and washed with water (10 mL), saturated aqueous sodium thiosulphate solution and brine. The organic layer was dried over sodium sulphate and concentrated in vacuo. The residue was purified by flash column chromatography using a gradient of 50% to 100% ethyl acetate in heptane to give the 3-iodo 4-MeO-pyridine-2,6-diamine as a light brown solid (70 mg, 70%).

¹H-NMR (400 MHz, DMSO-d6): δ 5.63 (bs, 2H), 5.43 (s, 1H), 5.35 (bs, 2H), 3.71 (s, 3H).

¹³C-NMR (101 MHz, DMSO-d6) δ: 165.6, 160.6, 158.7, 81.9, 56.1, 50.7.

### Step 2

### 3-(2,3-dichlorophenyl)-4-methoxypyridine-2,6-diamine, 8a

A 10 ml microwave vial containing a Teflon® stirred bar was charged with 3-iodo-4-methoxypyridine-2,6-diamine (60 mg, 0.23 mmol, 1 eq.), 3,4-dichloro phenylboronic acid (45.4 mg, 0.39 mmol, 1.05 eq.), Na₂CO₃ (72.3 mg, 0.69 mmol, 3 eq.) followed by the addition of a mixture of Toluene/EtOH/H₂O: 2.3/0.4/0.4 (0.1 mmol/mL). The vessel was evacuated and backfilled with nitrogen (this process was repeated a total of 3 times) and Pd(PPh₃)₄ (13.2 mg, 0.012 mmol, 0.05 eq.) was introduced. The reaction mixture was then capped properly and placed in a preheated oil bath at 120° C. until complete conversion of the starting material was detected. The reaction mixture was monitored by HPLC analysis and was usually complete within 4 hours. The reaction mixture was then concentrated under vacuum and the crude product was purified by chromatography on silica gel using a gradient of 75% to 100% EtOAc in hexane to afford the expected product 8a as a white solid (23 mg, 32%) after preparation of the corresponding hydrochloride salt.

¹H-NMR (400 MHz, DMSO-d6): δ 12.33, (bs, 1H), 7.69 (dd, J=8.0 Hz, 1.5 Hz, 1H), 7.42 (bs, 2H), 7.40 (t, J=7.9 Hz, 1H), 7.24 (dd, J=7.7 Hz, 1.5 Hz, 1H), 6.62 (bs, 2H), 5.76 (s, 1H), 3.72 (s, 3H).

¹³C-NMR (101 MHz, DMSO-d6) δ: 168.0, 153.5, 149.4, 133.3, 132.3, 132.2, 132.1, 130.5, 128.6, 94.3, 79.7, 56.4.

### Example 15: Preparation of 3-(2,3-dichlorophenyl)-5-fluoropyridine-2,6-diamine, 9a

For example 15, the starting 2,6-diamino-5-fluoro-pyridine was not commercially available and then subsequently prepared according to the following 1 step procedure.

3-Fluoropyridine-2,6-diamine (Tetrahedron Lett, 2007, 48 (46) 8199-8191).

A 20 mL microwave vial containing a Teflon® stirred bar was charged with 2,6-dichloro-5-fluoronicotinic acid (1.0 g, 4.58 mmol, 1 eq.) followed by the addition of NH₄OH (8.2 mL). The vial was then capped properly and placed in a preheated oil bath at 150° C. until complete conversion of the starting material was detected (24 h). The reaction mixture was then concentrated under vacuum and the crude product was purified by chromatography on silica gel using EtOAct/heptane: 1/1 to afford the title compound (110 mg, 19%).

¹H-NMR (400 MHz, CDCl₃): δ 7.00 (dd, J=10.2 Hz, 8.4 Hz, 1H), 5.76 (dd, J=8.4 Hz, 2.1 Hz, 1H), 4.31 (bs, 2H), 4.03 (bs, 2H).

¹³C-NMR (101 MHz, CDCl₃): δ 153.2, 146.0, 140.8 (d, J=246 Hz), 124.4 (d, J=17 Hz), 96.9 (d, J=1.7 Hz).

### Step 1

### 3-fluoro-5-iodopyridine-2,6-diamine

To a solution of 3-Fluoropyridine-2,6-diamine (100 mg, 0.79 mmol, 1 eq.) in 2-methyl-tetrahydrofuran (2 mL) was added potassium carbonate (108.7 mg, 0.79 mmol, 1 eq.). To this suspension was added a solution of iodine (200.1 mg, 0.79 mmol, 1 eq.) in 2-methyl-tetrahydrofuran (2 mL) dropwise over 1 hour. The reaction was stirred for 2 hours at room temperature. The reaction was filtered through a pad of celite and washed with EtOAc, and the filtrate collected and washed with water (10 ml), saturated aqueous sodium thiosulphate solution and brine. The organic layer was dried over sodium sulphate and concentrated in vacuo. The residue was purified by flash column chromatography AcOEt/Heptane: 1/2 to give the title compound (180 mg, 90%).

¹H-NMR (400 MHz, CDCl₃): δ 7.35 (d, J=9.3 Hz, 1H), 4.50 (bs, 2H), 4.38 (bs, 2H).

¹³C-NMR (101 MHz, CDCl₃): δ 152.7, 146.6 (d, J=14 Hz), 140.2 (d, J=243 Hz), 132.3 (d, J=19 Hz), 57.7.

### Step 2

### 3-(2,3-dichlorophenyl)-5-fluoropyridine-2,6-diamine, 9a

A 10 ml microwave vial containing a Teflon® stirred bar was charged with 3-fluoro-5-iodopyridine-2,6-diamine (107.6 mg, 0.42 mmol, 1 eq.), 3,4-dichloro phenylboronic acid (85.2 mg, 0.45 mmol, 1.05 eq.), Na₂CO₃ (135.9 mg, 1.26 mmol, 3 eq.) followed by the addition of a mixture of Toluene/EtOH/H₂O: 3/0.5/0.5 (0.1 mmol/mL). The vessel was evacuated and backfilled with nitrogen (this process was repeated a total of 3 times) and Pd(PPh₃)₄ (13.2 mg, 0.021 mmol, 0.05 eq.) was introduced. The reaction mixture was then capped properly and placed in a preheated oil bath at 120° C. until complete conversion of the starting material (usually 4 h). The reaction mixture was then concentrated under vacuum and the crude product was purified by chromatography on silica gel using a gradient of 75% to 100% ethyl acetate in heptane to afford the expected product 9a as a white solid (72 mg, 55%) after preparation of the corresponding hydrochloride salt.

¹H-NMR (400 MHz, DMSO-d6): δ 7.68 (dd, J=8.0 Hz, 1.6 Hz, 1H), 7.59 (d, J=11.1 Hz, 1H), 7.50 (bs, 1H) 7.43 (t, J=7.9 Hz, 1H), 7.34 (dd, J=7.7 Hz, 1.5 Hz, 1H), 6.61 (bs, 2H), 4.10 (bs, 2H).

¹³C-NMR (101 MHz, DMSO-d6) δ: 151.0 (d, J=1.9 Hz), 147.1 (d, J=13.4 Hz), 138.8, 138.6 (d, J=235 Hz), 132.1, 131.8, 131.1, 129.3, 128.3, 124.1 (d, J=20 Hz), 104.0.

### Example 16: 3-(2,3-dichlorophenyl)-5-ethylpyridine-2,6-diamine, 9b, Method 13

A round bottomed flask containing a stirrer bar was charged with 1j (50 mg, 0.19 mmol, 1 eq.) in MeOH (4.5 mL) followed by the addition of acetaldehyde (49.6 μL, 0.88 mmol, 4.5 eq.) and acetic acid (30 μL, 0.57 mmol, 3 eq.). The resulting mixture was stirred at room temperature for 4 h. NaBH₃CN (44.7 mg, 0.48 mmol, 2.6 eq.) was then added and the solution was stirred an additional 16 hours. The reaction mixture was concentrated under reduced pressure. The residue was dissolved in EtOAc, and successively washed with brine and water. The organic layer was dried over Na₂SO₄, filtered and concentrated in vacuo. The crude product was purified by chromatography on silica gel using a gradient of 25% to 30% EtOAc in heptane to afford the expected product (10 mg, 18%).

¹H-NMR (400 MHz, CDCl₃): δ 7.39 (dd, J=8.1 Hz, J=3.3 Hz, 1H), 7.23-7.26 (m, 2H), 7.02 (s, 1H), 4.78 (bs, 2H); 4.36 (bs, 2H), 2.38 (q, J=7.6 Hz, 2H), 1.18 (t, J=7.6 Hz, 3H).

¹³C-NMR (101 MHz, CDCl₃): δ 149.6, 146.9, 144.3, 137.8, 134.6, 133.1, 131.2, 130.1, 128.3, 110.9, 107.5, 21.5, 12.3

10—General Synthetic Method for the Preparation of 3-benzyl pyridine-2,6-diamine derivatives 10. (Methods 14-15)

Compounds of general formula 10 can be prepared following two synthetic procedures described in the above scheme 11. The first pathway (method 14) is a known process of the literature (Polish Journal of Chemistry, 55(4), 931-4, 1981; Ger. Offen., DE 1986-3637829, 11 May 1988) based on a solvent free condensation of the 2,6-diaminopyrine derivatives in presence of the corresponding benzyl chlorides (yields <42%). Starting from 3-iodopyridine-2,6-diamine we developed a new method of preparation of 10, more efficient in term of yield (yield >60%) via a Negishi cross-coupling reaction with the help of S-Phos (Method 15, scheme 11).

Scheme 11

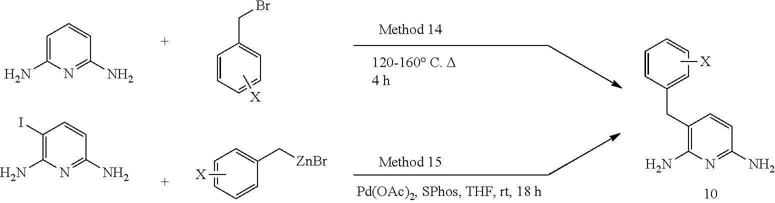

| Entry | N° | X | method | Yield % |
|---|---|---|---|---|
| 1 | 10a | H | 15 | 60 |
| 1 | 10b | 4-F | 14 | 20 |
| 2 | 10c | 4-Cl | 14 | 8 |
| 3 | 10d | 3-Cl | 14 | 29 |
| 4 | 10e | 2-Cl | 14 | 35 |
| 5 | 10f | 2,4-Cl₂ | 15 | 64 |

### Example 17

### 3-(2-chlorobenzyl)pyridine-2,6-diamine, 10e method 14

2,6-diaminopyridine (218.3 mg, 2 mmol, 1 eq.) was slowly heated to melting and 2-chlorobenzyl chloride (0.26 mL, 2 mmol, 1 eq.) was added dropwise. The resulting mixture was stirred at 160° C. for 4 hours. The residue was dissolved in DCM, and successively washed with NH₄OH and water and brine. The organic layer was dried over Na₂SO₄, filtered and concentrated in vacuo. The crude product was purified by chromatography on silica gel using a gradient of 50% to 100% EtOAc in heptane to afford the expected product as a solid (190 mg, 35%) after preparation of the corresponding hydrochloride salt.

¹H-NMR (400 MHz, DMSO-d6: δ 13.17 (bs, 1H), 7.47 (m, 1H), 7.33-7.26 (m, 6H), 7.17 (m, 1H), 7.06 (d, J=8.4 Hz, 1H), 5.90 (d, J=8.4 Hz, 1H), 3.76 (s, 2H).

¹³C-NMR (101 MHz, DMSO-d6): δ 150.8, 149.8, 144.8, 136.2, 133.4, 130.5, 129.4, 128.4, 127.4, 103.7, 95.7, 31.4.

### 3-(4-chlorobenzyl)pyridine-2,6-diamine, 10c

10c was analogously obtained following the method 14 in presence 4-chlorobenzyl chloride as a white solid (45 mg, 8%) after preparation of the corresponding hydrochloride salt.

¹H-NMR (400 MHz, DMSO-d6): δ 12.92 (bs, 1H), 7.37 (d, J=8.4 Hz, 1H), 7.35 (d, J=8.3 Hz, 2H), 7.24-7.21 (m, 4H), 7.18 (bs, 2H), 5.93 (d, J=8.4 Hz, 1H), 3.72 (s, 2H).

¹³C-NMR (101 MHz, DMSO-d6): δ 150.8, 149.6, 145.6, 138.4, 130.8, 130.2, 128.3, 105.9, 95.7, 32.4.

### 3-(3-chlorobenzyl)pyridine-2,6-diamine, 10d

10d was analogously obtained following the method 14 in presence 3-chlorobenzyl bromide as a white solid (134 mg, 29%) after preparation of the corresponding hydrochloride salt.

¹H-NMR (400 MHz, DMSO-d6): δ 13.05 (s, 1H), 7.42 (d, J=8.4 Hz, 1H), 7.32 (t, J=7.8 Hz, 1H), 7.29-7.25 (m, 4H), 7.22 (bs, 2H), 7.17 (d, J=7.3 Hz, 1H), 5.94 (d, J=8.4 Hz, 1H), 3.74 (s, 2H).

¹³C-NMR (101 MHz, DMSO-d6): δ 150.9, 149.6, 145.8, 142.0, 133.0, 130.2, 128.2, 127.0, 126.2, 105.3, 95.8, 32.7.

### 3-(4-fluorobenzyl)pyridine-2,6-diamine, 10b

10b was analogously obtained following the method 14 in presence 4-fluorobenzyl bromide, as a white solid (86 mg, 20%) after preparation of the corresponding hydrochloride salt.

¹H-NMR (400 MHz, DMSO-d6): δ 13.21 (bs, 1H), 7.33 (d, J=8.4 Hz, 1H), 7.24 (dd, J=8.0 Hz, 5.7 Hz, 2H), 7.16 (bs, 4H), 7.10 (t, J=8.7 Hz, 2H), 5.92 (d, J=8.4 Hz, 1H), 3.71 (s, 2H).

¹³C-NMR (101 MHz, DMSO-d6): δ 162.0, 159.6, 150.5 (d, J=115 Hz), 145.2, 135.5 (d, J=3.2 Hz), 130.2 (d, J=8.2 Hz), 115.0 (d, J=21 Hz), 106.0, 95.7, 32.3.

### Example 18: 3-(2,4-dichlorobenzyl)pyridine-2,6-diamine hydrochloride, 10f, (Method 15)

A 10 ml microwave vial containing a Teflon® stirred bar was charged with 3-iodopyridine-2,6-diamine (100 mg, 0.42 mmol, 1 eq.), Pd(OAc)₂ (4.84 mg, 0.021 mmol, 0.05 eq.), S-Phos (17.5 mg, 0.042 mmol, 0.1 eq), followed by the addition of anhydrous THF (5 mL). The vessel was evacuated and backfilled with nitrogen (this process was repeated a total of 3 times) and (2,4-dichlorobenzyl)zinc(II) chloride (4.5 ml, 0.28 mol/L in THF, 1.26 mmol, 3 eq.) was introduced drop by drop. The reaction mixture was then capped properly and stirred at room temperature until complete conversion of the starting material was detected. The reaction mixture was monitored by HPLC analysis and was usually complete within 3 hours. The reaction was quenched with saturated solution of NH₄Cl (5 mL), extracted twice with EtOAc. The organic layers were combined and washed with brine and water. The organic layer was dried over Na₂SO₄, filtered and evaporated in vacuo. The crude product was purified by chromatography on silica gel using a gradient of 50% to 80% ethyl acetate in hexane to give the expected 10f as a light brown solid (64 mg, 64%) after preparation of the corresponding hydrochloride salt.

¹H-NMR (400 MHz, DMSO-d6): δ 12.93 (bs, 1H), 7.64 (d, J=1.7 Hz, 1H), 7.39 (dd, J=8.2 Hz, J=1.7 Hz, 1H), 7.24 (bs, 2H), 7.22 (bs, 2H), 7.17 (d, J=8.2 Hz, 1H), 7.11 (d, J=8.4 Hz, 1H), 5.91 (d, J=8.4 Hz, 1H), 3.75 (s, 2H).

¹³C-NMR (101 MHz, DMSO-d6): δ 151.0, 149.8, 145.0, 135.4, 134.4, 131.9, 131.6, 128.8, 127.4, 103.2, 95.8, 31.0.

3-benzylpyridine-2,6-diamine hydrochloride, 10a was analogously obtained following the method 15 using benzyl zinc(II) chloride, as a brown light solid (78 mg, 60%) after preparation of the corresponding hydrochloride salt.

¹H-NMR (400 MHz, DMSO-d6): δ 13.12 (bs, 1H), 7.34 (d, J=8.4 Hz, 1H), 7.31-7.27 (m, 2H), 7.22-7.18 (m, 7H), 5.93 (d, J=8.4 Hz, 1H), 3.72 (s, 2H).

¹³C-NMR (101 MHz, DMSO-d6): δ 150.7, 149.6, 145.5, 139.3, 128.4, 126.2, 106.1, 95.6, 33.1.

11—General Synthetic Method for the Preparation of 3-phenethyl pyridine-2,6-diamine derivatives 11 (Method 16).

According to the above scheme 12, the preparation of compounds of the general formula 11 involved a Suzuki Miyaura cross-coupling reaction between 3-iodopyridine-2,6-diamine and corresponding stryryl boronic acid followed by a catalytic hydrogenation.

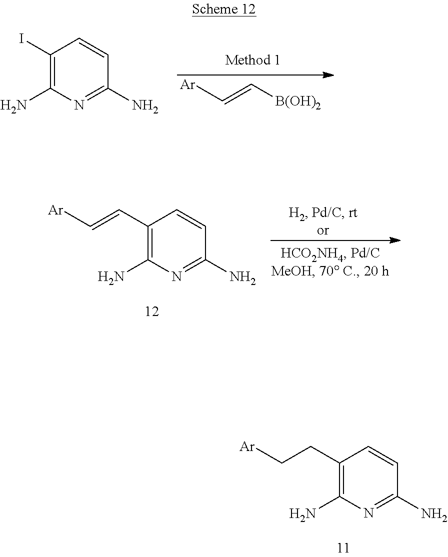

### Example 19: Preparation of 3-phenethylpyridine-2,6-diamine 11a

Step 1: A 20 mL microwave vial containing a Teflon® stirred bar was charged with 3 iodopyridine-2,6-diamine (100 mg, 0.425 mmol, 1 eq.), E-styrylboronic acid (94.4 mg, 0.64 mmol, 1.5 eq.), Na₂CO₃ (135.3 mg, 1.28 mmol, 3 eq.) followed by the addition of a mixture of Toluene/EtOH/H₂O: 6/1/1 (0.1 mmol/mL). The vessel was evacuated and backfilled with nitrogen (this process was repeated a total of 3 times) and Pd(PPh₃)₄ (24.8 mg, 0.021 mmol, 0.05 eq.) was introduced. The reaction mixture was then capped properly and placed in a preheated oil bath at 120° C. until complete conversion of the starting material was detected. The reaction mixture was monitored by HPLC analysis and was usually complete within 16 hours. The reaction mixture was then concentrated under vacuum and the crude product was purified by chromatography on silica gel using a gradient of 75% to 100% EtOAc in hexane to afford the expected product 12 as a yellow solid. (56%)

¹H-NMR (400 MHz, CDCl3): δ 7.45 (d, J=8.2 Hz, 1H), 7.43 (d, J=7.6 Hz, 2H), 7.31 (t, J=7.7 Hz, 2H), 7.20 (t, J=7.4 Hz, 1H), 6.92 (d, J=16.1 Hz, 1H), 6.78 (d, J=16.1 Hz, 1H), 5.96 (d, J=8.2 Hz, 1H), 4.45 (bs, 2H), 4.45 (bs, 2H).

Step 2: A 20 mL microwave vial containing a Teflon® stirred bar was charged with (E)-3-styrylpyridine-2,6-diamine (37 mg, 0.17 mmol, 1 eq.), HCO₂NH₄ (66 mg, 1.02 mmol, 6 eq.), Pd/C 10% (7 mg) followed by the addition of MeOH (5.2 mL). The reaction mixture was then capped properly and the vessel was evacuated and backfilled with nitrogen (this process was repeated a total of 3 times). The resulting mixture was heated at 70° C. for 20 h. After evaporation of the volatile the crude product was purified by reverse phase chromatography (H₂O/MeOH) to yield the desired product 11a (10 mg, 27%).

¹H-NMR (400 MHz, CDCl3): δ 7.27 (m, 2H), 7.20 (m, 1H), 7.13 (m, 2H), 7.05 (d, J=8.0 Hz, 1H), 5.86 (d, J=8.0 Hz, 1H), 4.50 (bs, 4H), 2.84 (t, J=7.6 Hz, 2H), 2.62 (t, J=7.6 Hz, 2H).

¹³C-NMR (101 MHz, CDCl3): δ 154.4, 153.9, 141.3, 139.8, 128.8, 128.6, 126.5, 109.1, 98.2, 35.2, 32.1.

### 12—Methods for the Preparation of Compounds No 13-91, Methods 17-23

### Example 20: Compounds 13-91

### Methods for Preparation of Compounds No 13-66 in Substituted Phenyl Series

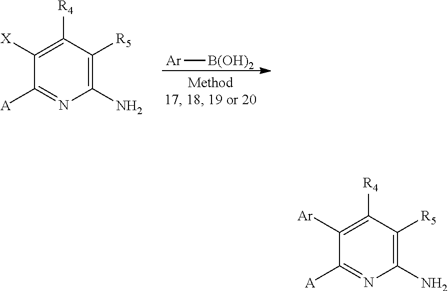

### Compounds of Formula (I) where n=0

| | | | | | | Yield | Compound | HCl |
|---|---|---|---|---|---|---|---|---|
| A═R₃ | R₄ | R₅ | X | Ar | Method | (%) | N° | salt ? |
| H | H | Me | Br | 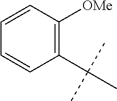 | 17 | 89 | 13 | — |
| | | CF₃ | Br | 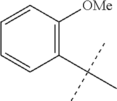 | 18 | 91 | 14 | — |
| | | F | Br | 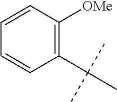 | 18 | 82 | 15 | — |
| | | F | Br | 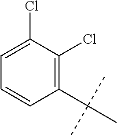 | 18 | 51 | 16 | — |
| | F | H | Br | 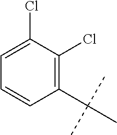 | 17 | 33 | 17 | — |
| | | | | 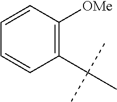 | 17 | 42 | 18 | — |
| | OMe | H | Br | 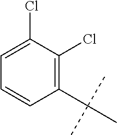 | 17 | 7 | 19 | HCl |
| | | | | 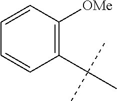 | 17 | 30 | 20 | — |
| | Me | H | Br | 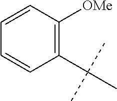 | 17 | 52 | 21 | — |
| | | | | 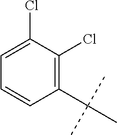 | 18 | 46 | 22 | — |
| | O—CH₂—CF₃ | H | Br | 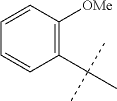 | 17 | 26 | 23 | HCl |
| | | | | 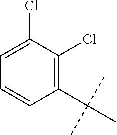 | 17 | 27 | 24 | di HCl |
| | O—(CH₂)₂—NH₂ | H | Br | 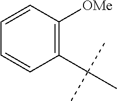 | 17 | 13 | 25 | HCl |
| Me | H | H | Br | 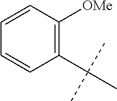 | 18 | 44 | 26 | — |
| | Me | H | Br | 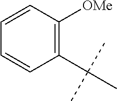 | 18^{a} | 28 | 27^{a} | — |
| Me | Me | H | Br | 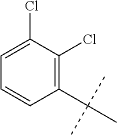 | 18 | 13 | 28 | — |
| Et | H | H | Br | 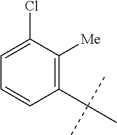 | 19 | 8 | 29 | HCl |
| | | | | 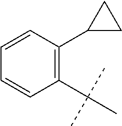 | 19 | 38 | 30 | HCl |
| | | | | 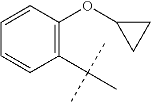 | 19^{e} | 3 | 31^{e} | HCl |
| F | H | H | Br | 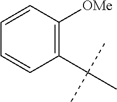 | 17 | 69 | 32 | — |
| | | | | 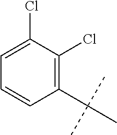 | 17 | 19 | 33 | — |
| CF₃ | H | H | Br | 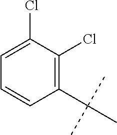 | 18 | 63 | 34 | — |
| OMe | H | H | Br | 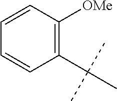 | 17 | 57 | 35 | — |
| O—CH₂—CF₃ | H | H | Br | 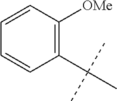 | 17 | 65 | 36 | — |
| O—(CH₂)₂—NH₂ | H | H | Br | 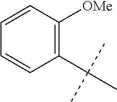 | 18 | 18 | 37 | — |
| | | | | 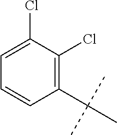 | 18 | 17 | 38 | diHCl |
| NH₂ | H | H | I | 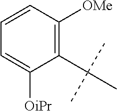 | 20 | 69 | 39 | — |
| | | | | 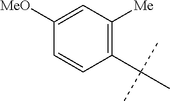 | 20^{c} | 39 | 40^{c} | — |
| | | | | 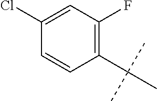 | 20^{c} | 37 | 41^{c} | — |
| | | | | 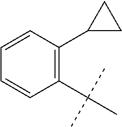 | 20^{c} | 25 | 42^{c} | HCl |
| | | | | 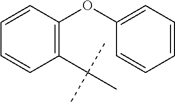 | 20^{c} | 20 | 43^{c} | HCl |
| | | | | 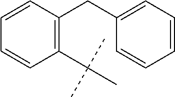 | 20^{c} | 22 | 44^{c} | — |
| | | | | 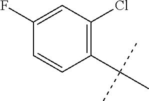 | 20 | 58 | 45 | — |
| NH₂ | H | H | I | 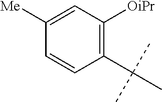 | 20 | 42 | 46 | — |
| | | | | 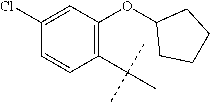 | 20 | 30 | 47 | — |
| | | | | 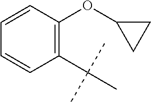 | 20 | 44 | 48 | — |
| | | | | 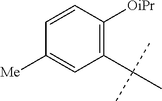 | 20 | 58 | 49 | — |
| | | | | 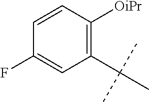 | 20 | 79 | 50 | — |
| | | | | 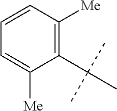 | 20^{d} | 15 | 51^{d} | — |
| | | | | 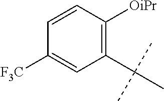 | 20 | 64 | 52 | — |
| | | | | 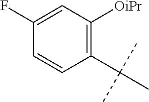 | 20 | 59 | 53 | — |
| | | | | 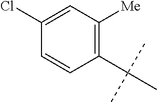 | 20 | 30 | 54 | — |
| | | | | 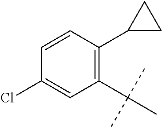 | 20^{e} | 45 | 55^{e} | — |
| | | | | 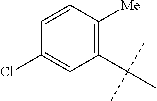 | 20 | 61 | 56 | — |
| | | | | 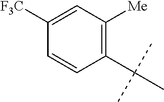 | 20 | 35 | 57 | — |
| | | | | 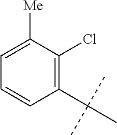 | 20 | 83 | 58 | — |
| | | | | 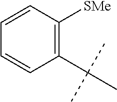 | 20 | 63 | 59 | HCl |
| | | | | 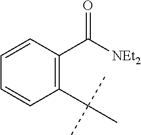 | 20 | 8 | 60 | HCl |
| | | | | 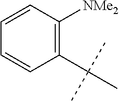 | 20^{e} | 6 | 61^{e} | HCl |
| | | | | 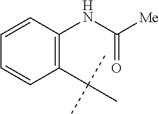 | 20^{e} | 38 | 62^{e} | — |
| | | | | 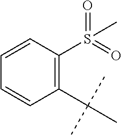 | 20 | 31 | 63 | HCl |
| NH₂ | H | H | I | 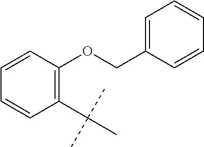 | 20 | 67 | 64 | HCl |
| | | | | 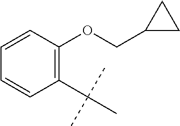 | 20 | 48 | 65 | HCl |
| NH₂ | H | F | I | 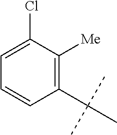 | 20 | 62 | 66 | — |

^{a}7.5 mol % of Pd-116 precatalyst were used instead of 5 mol %.
^{b}Obtained as an overeaction side product using 3-chloro-2-methylPhenylboronic acid.
^{c}Reaction performed at 60° C. instead of 80° C.
^{d}Reaction performed at 110° C. for 48 h.
^{e}Pinacol ester was used instead of the respective boronic acid.
Method 17: PdCl₂(dppf)CH₂Cl₂ (5 mol %), K₂CO₃ aq (1.2M), dioxane, 90° C.
Method 18: PdP(tBu)₃PdG2 (5 mol %), K₂CO₃ aq (1.2M), dioxane, 60° C.
Method 19: SPhosPdG₂ (5 mol %), K₂CO_{3 aq} (1.2M), dioxane, 80° C.
Method 20: PdP(tBu)₃PdG2 (7 mol %), K₂CO_{3 aq} (1.2M), dioxane, 80° C.

Method 17:

In a microwave vial, to a suspension of aryl bromide (1.0 eq.) and aryl boronic acid (1.0-1.2 eq.) in dioxane (C=0.2 M) was added dropwise an aqueous solution of K₂CO₃ (1.2 M, 2.0 eq.). The resulting suspension was degassed with argon bubbling for 15 min and PdCl₂(dppf)CH₂C₁₋₂ (5 mol %) was then added in one portion. The vial was sealed and the mixture was stirred at 90° C. until no more evolution was noticed by UPLC-MS (overnight, unless mentioned otherwise). The reaction mixture was cooled to rt and subsequently hydrolysed. The aqueous layer was extracted twice with EtOAc and the combined organic layers were washed with brine, dried over MgSO₄ or hydrophobic filter, filtered and concentrated in vacuo. The residue was purified by flash chromatography. The obtained solid was further purified when necessary. For specific examples, the corresponding hydrochloride salt has been prepared.

Method 18:

In a microwave vial, to a suspension of aryl bromide (1.0 eq.) and aryl boronic acid (1.0-1.2 eq.) in dioxane (C=0.2 M) was added dropwise an aqueous solution of K₂CO₃ (1.2 M, 2.0 eq.). The resulting suspension was degassed with argon bubbling for 15 min and PdP(tBu)₃PdG2 (5 mol %) was then added in one portion. The vial was sealed and the mixture was stirred at 60° C. until no more evolution was noticed by UPLC-MS (overnight unless mentioned otherwise). The reaction mixture was cooled to rt and subsequently hydrolysed. The aqueous layer was extracted twice with EtOAc and the combined organic layers were washed with brine, dried over MgSO₄ or hydrophobic filter, filtered and concentrated in vacuo. The residue was purified by flash chromatography. The obtained solid was further purified when necessary. For specific examples, the corresponding hydrochloride salt has been prepared.

Method 19:

In a microwave vial, a suspension of aryl bromide (1.0 eq.) and aryl boronic acid (1.5 eq.) in a mixture of dioxane/(1.2 M) aqueous K₂CO₃ (3/1 v/v, final concentration: C=0.15-0.20 M) was degassed with argon bubbling for 15 min. SPhosPdG2 (5 mol %) was then added in one portion. The vial was sealed and the mixture was stirred at 80° C. for 17 h. The reaction mixture was cooled to rt and subsequently hydrolysed. The aqueous layer was extracted twice with EtOAc and the combined organic layers were washed with brine, dried over MgSO₄ or hydrophobic filter, filtered and concentrated in vacuo. The residue was purified by chromatography. The obtained solid was further purified when necessary. For specific examples, the corresponding hydrochloride salt has been prepared.

Method 20:

In a microwave vial, to a suspension of aryl iodide (1.0 eq.) and aryl boronic acid (1.0-2.5 eq.) in dioxane (C=0.2 M) was added dropwise an aqueous solution of K₂CO₃ (1.2 M, 2.0 eq.). The resulting suspension was degassed with argon bubbling for 15 min and PdP(tBu)₃PdG2 (7 mol %) was then added in one portion. The vial was sealed and the mixture was stirred at 80° C. until no more evolution was noticed by UPLC-MS (overnight, unless mentioned otherwise). The reaction mixture was cooled to rt, filtered on a Celite® pad and the cake was washed with MeOH. The filtrate was concentrated in vacuo and the residue was purified. The obtained solid was further purified when necessary. For specific examples, the corresponding hydrochloride salt has been prepared.

### 5-(2-Methoxy-phenyl)-3-methyl-pyridin-2-ylamine Compound No 13

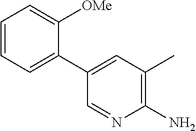 [SMILES: COc1ccccc1-c1cnc(N)c(C)c1]

Compound No 13 was prepared according to method 17 starting from 2-amino-5-bromo-3-methylpyridine (300 mg, 1.60 mmol, 1.0 eq.) and 2-methoxyphenylboronic acid (267 mg, 1.76 mmol, 1.1 eq.). The crude was purified by flash chromatography (SiO₂, CycloHex/EtOAc, 100/0 to 50/50). The obtained foam was taken up in a mixture of ACN/water and the resulting solution was lyophilised to afford compound No 13 as a beige powder (305 mg, 89%).

Mp: 92-95° C.; ¹H NMR (400 MHz, DMSO-d₆) δ: 2.07 (s, 3H, CH₃); 3.75 (s, 3H, O—CH₃); 5.70 (bs, 2H, NH₂); 6.98 (td, J 7.4, 1.0 Hz, 1H, Ar); 7.05 (dd, J 8.2, 0.8 Hz, 1H, Ar); 7.22 (dd, J 7.4, 1.7 Hz, 1H, Ar); 7.27 (ddd, J 8.2, 7.4, 1.7 Hz, 1H, Ar); 7.37 (dd, J 2.2, 0.8 Hz, 1H, Ar); 7.89 (d, J 2.2 Hz, 1H, Ar); M/Z (M+H)⁺: 215.6.

### 5-(2-Methoxy-phenyl)-3-trifluoromethyl-pyridin-2-ylamine Compound No 14

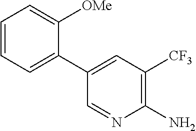 [SMILES: COc1ccccc1-c1cnc(N)c(C(F)(F)F)c1]

Compound No 14 was prepared according to method 18 starting from 2-amino-5-bromo-3-trifluoromethylpyridine (330 mg, 1.37 mmol, 1.0 eq.) and 2-methoxyphenylboronic acid (225 mg, 1.55 mmol, 1.1 eq.). The crude was purified by flash chromatography (SiO₂, CycloHex/EtOAc, 100/0 to 50/50). The obtained foam was taken up in a mixture of ACN/water and the resulting solution was lyophilised to afford compound No 14 as an off-white powder (335 mg, 91%).

Mp: 63-67° C.; ¹H NMR (400 MHz, DMSO-d₆) δ: 3.78 (s, 3H, O—CH₃); 6.48 (bs, 2H, NH₂); 7.02 (td, J 7.4, 1.0 Hz, 1H, Ar); 7.08-7.12 (m, 1H, Ar); 7.30-7.37 (m, 2H, Ar); 7.83 (d, J 2.0 Hz, 1H, Ar); 7.31 (d, J 2.0 Hz, 1H, Ar); M/Z (M+H)⁺: 269.6.

### 3-Fluoro-5-(2-methoxy-phenyl)-pyridin-2-ylamine Compound No 15

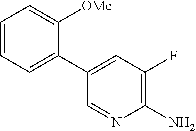 [SMILES: COc1ccccc1-c1cnc(N)c(F)c1]

Compound No 15 was prepared according to method 18 starting from 2-amino-5-bromo-3-fluoropyridine (330 mg, 1.37 mmol, 1.0 eq.) and 2-methoxyphenylboronic acid (261 mg, 1.72 mmol, 1.1 eq.). The crude was purified by flash chromatography (SiO₂, CycloHex/EtOAc, 100/0 to 50/50). The obtained foam was taken up in a mixture of ACN/water and the resulting solution was lyophilised to afford compound No 15 as an off-white powder (335 mg, 82%).

Mp: 75-79° C.; ¹H NMR (400 MHz, DMSO-d₆) δ: 3.77 (s, 3H, O—CH₃); 6.21 (bs, 2H, NH₂); 6.99 (td, J 7.5, 1.0 Hz, 1H, Ar); 7.06-7.09 (m, 1H, Ar); 7.27-7.34 (m, 2H, Ar); 7.50 (dd, J 12.7, 1.9 Hz, 1H, Ar); 7.89 (t, J 1.6 Hz, 1H, Ar); M/Z (M+H)⁺: 219.6.

### 5-(2,3-Dichloro-phenyl)-3-fluoro-pyridin-2-ylamine Compound No 16

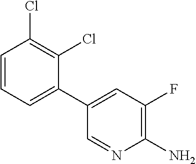 [SMILES: Nc1ncc(-c2cccc(Cl)c2Cl)cc1F]

Compound No 16 was prepared according to method 18 starting from 2-amino-5-bromo-3-fluoropyridine (100 mg, 0.52 mmol, 1.0 eq.) and 2,3-dichlorophenylboronic acid (109 mg, 0.57 mmol, 1.1 eq.). The crude was purified by flash chromatography (SiO₂, CycloHex/EtOAc, 100/0 to 50/50). The obtained foam was taken up in a mixture of ACN/water and the resulting solution was lyophilised to afford compound No 16 as a beige solid (69 mg, 51%).

Mp: 130-136° C.; ¹H NMR (400 MHz, DMSO-d₆) δ: 6.46 (bs, 2H, NH₂); 7.37-7.44 (m, 2H, Ar); 7.53 (dd, J 12.2, 1.9 Hz, 1H, Ar); 7.63 (dd, J 7.0, 2.7 Hz, 1H, Ar); 7.82 (dd, J 1.9, 1.2 Hz, 1H, Ar); M/Z (M[³⁵Cl]₂+H)⁺: 257.5.

### 5-(2,3-Dichloro-phenyl)-4-fluoro-pyridin-2-ylamine Compound No 17

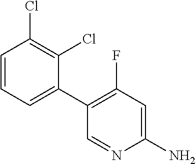 [SMILES: Nc1cc(F)c(-c2cccc(Cl)c2Cl)cn1]

Compound No 17 was prepared according to method 17 starting from 2-amino-5-bromo-4-fluoropyridine (100 mg, 0.52 mmol, 1.0 eq.) and 2,3-dichlorophenylboronic acid (109 mg, 0.57 mmol, 1.1 eq.). The reaction mixture was stirred for 1.5 h instead of 17 h. The crude was purified by flash chromatography (SiO₂, CycloHex/EtOAc, 100/0 to 40/60). The resulting foam was triturated thrice in pentane to afford compound No 17 as a beige solid which was dried at 70° C. under high vacuum for 48 h (45 mg, 33%).

Mp: 107-112° C.; ¹H NMR (400 MHz, DMSO-d₆) δ: 6.30 (d, J 12.4 Hz, 1H, Ar); 6.46 (s, 2H, NH₂); 7.37 (dd, J 8.0, 1.6 Hz, 1H, Ar); 7.43 (t, J 8.0 Hz, 1H, Ar); 7.67 (dd, J 8.0, 1.6 Hz, 1H, Ar); 7.87 (d, J 11.2 Hz, 1H, Ar); M/Z (M[³⁵Cl]₂+H)⁺: 257.5.

### 4-Fluoro-5-(2-methoxy-phenyl)-pyridin-2-ylamine Compound No 18

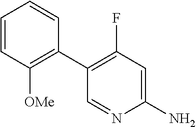 [SMILES: COc1ccccc1-c1cnc(N)cc1F]

Compound No 18 was prepared according to method 17 starting from 2-amino-5-bromo-4-fluoropyridine (100 mg, 0.52 mmol, 1.0 eq.) and 2-methoxyphenylboronic acid (87 mg, 0.57 mmol, 1.1 eq.). The crude was purified by flash chromatography (SiO₂, CycloHex/EtOAc, 100/0 to 30/70). The resulting foam was triturated thrice in pentane to afford compound No 18 as an off-white solid which was dried at 70° C. under high vacuum for 48 h (48 mg, 42%).

Mp: 65-70° C.; ¹H NMR (400 MHz, DMSO-d₆) δ: 3.73 (s, 3H, O—CH₃); 6.22 (s, 2H, NH₂); 6.24 (d, J 11.2 Hz, 1H, Ar); 6.99 (td, J 7.5, 1.0 Hz, 1H, Ar); 7.08 (dd, J 8.2, 1.0 Hz, 1H, Ar); 7.18 (dd, J 7.5, 1.8 Hz, 1H, Ar); 7.35 (ddd, J 8.2, 7.5, 1.8 Hz, 1H, Ar); 7.82 (d, J 11.2 Hz, 1H, Ar); M/Z (M+H)⁺: 219.6.

### 5-(2,3-Dichloro-phenyl)-4-methoxy-pyridin-2-ylamine hydrochloride Compound No 19

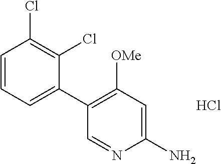 [SMILES: COc1cc(N)ncc1-c1cccc(Cl)c1Cl ; Cl]

Compound No 19 was prepared according to method 17 starting from 2-amino-5-bromo-4-methoxypyridine (100 mg, 0.49 mmol, 1.0 eq.) and 2,3-dichlorophenylboronic acid (103 mg, 0.53 mmol, 1.1 eq.). The crude was purified by flash chromatography (SiO₂, DCM/MeOH, 100/0 to 90/10). The resulting foam was triturated thrice in pentane to afford a yellow solid which was dried at 70° C. under high vacuum for 48 h (23 mg, 17%). The obtained solid was taken up in Et₂O and a solution of HCl (1 M in Et₂O) was added dropwise. The resulting precipitate was collected by filtration, triturated in Et₂O, taken up in a mixture ACN/H₂O and lyophilised to afford compound No 19 as a white powder (11 mg, 7%).

Mp>250° C.; ¹H NMR (400 MHz, DMSO-d₆) δ: 3.86 (s, 3H, O—CH₃); 6.49 (s, 1H, Ar); 7.37 (dd, J 7.8, 1.6 Hz, 1H, Ar); 7.44 (t, J 7.8 Hz, 1H, Ar); 7.71 (dd, J 7.8, 1.6 Hz, 1H, Ar); 7.88 (s, 1H, Ar); 7.92 (bs, 2H, NH₂); 13.4 (bs, 1H, HCl salt); M/Z (M[³⁵Cl]₂+H)⁺: 269.5.

### 4-Methoxy-5-(2-methoxy-phenyl)-pyridin-2-ylamine Compound No 20

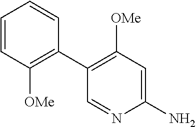 [SMILES: COc1ccccc1-c1cnc(N)cc1OC]

Compound No 20 was prepared according to method 17 starting from 2-amino-5-bromo-4-methoxypyridine (100 mg, 0.49 mmol, 1.0 eq.) and 2-methoxyphenylboronic acid (82 mg, 0.53 mmol, 1.1 eq.). The crude was purified by flash chromatography (SiO₂, DCM/MeOH, 100/0 to 90/10). The resulting foam was triturated thrice in pentane. The collected solid was dried at 70° C. under high vacuum for 48 h to afford compound No 20 as an off-white solid (36 mg, 30%).

Mp: 140-144° C.; ¹H NMR (400 MHz, DMSO-d₆) δ: 3.66 (s, 3H, O—CH₃); 3.69 (s, 3H, O—CH₃); 5.82 (bs, 2H, NH₂); 6.08 (s, 1H, Ar); 6.93 (td, J 7.6, 1.2 Hz, 1H, Ar); 7.01 (dd, J 8.4, 1.2 Hz, 1H, Ar); 7.08 (dd, J 7.6, 1.6 Hz, 1H, Ar); 7.23-7.32 (ddd, J 8.4, 7.6, 1.6 Hz 1H, Ar); 7.53 (s, 1H, Ar); M/Z (M+H)⁺: 231.7.

### 5-(2-Methoxy-phenyl)-4-methyl-pyridin-2-ylamine compound No 21

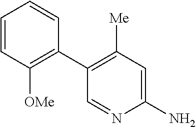 [SMILES: COc1ccccc1-c1cnc(N)cc1C]

Compound No 21 was prepared according to the method 17 starting from 2-amino-5-bromo-4-methylpyridine (100 mg, 0.53 mmol, 1.0 eq.) and 2-methoxyphenylboronic acid (88 mg, 0.58 mmol, 1.1 eq.). The crude was purified by flash chromatography (SiO₂, CycloHex/EtOAc, 100/0 to 50/50). The resulting foam was triturated thrice in Et₂O and the collected precipitate was dried at 70° C. under high vacuum for 48 h to afford compound No 21 as a beige solid (59 mg, 52%).

Mp: 85-88° C.; ¹H NMR (400 MHz, DMSO-d₆) δ: 1.91 (s, 3H, CH₃); 3.70 (s, 3H, O—CH₃); 5.74 (bs, 2H, NH₂); 6.32 (bs, 1H, Ar); 6.97 (td, J 7.3, 1.0 Hz, 1H, Ar); 7.03-7.09 (m, 2H, Ar); 7.33 (ddd, J 8.2, 7.3, 2.0 Hz, 1H, Ar); 7.58 (s, 1H, Ar); M/Z (M+H)⁺: 215.6.

### 5-(2,3-Dichloro-phenyl)-4-methyl-pyridin-2-ylamine Compound No 22

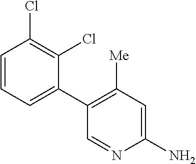 [SMILES: Cc1cc(N)ncc1-c1cccc(Cl)c1Cl]

Compound No 22 was prepared according to method 18 starting from 2-amino-5-bromo-4-methylpyridine (100 mg, 0.53 mmol, 1.0 eq.) and 2,3-dichlorophenylboronic acid (111 mg, 0.58 mmol, 1.1 eq.). The crude was purified by flash chromatography (SiO₂, CycloHex/EtOAc, 100/0 to 50/50). The resulting foam was triturated thrice in Et₂O and the collected precipitate was dried at 70° C. under high vacuum for 48 h to afford compound No 22 as a beige solid (62 mg, 46%).

Mp: 94-97° C.; ¹H NMR (400 MHz, DMSO-d₆) δ: 1.91 (s, 3H, CH₃); 5.96 (bs, 2H, NH₂); 6.37 (bs, 1H, Ar); 7.27 (dd, J 8.0, 1.5 Hz, 1H, Ar); 7.44 (t, J 8.0 Hz, 1H, Ar); 7.62 (bs, 1H, Ar); 7.64 (dd, J 8.0, 1.5 Hz, 1H, Ar); M/Z (M[³⁵Cl]₂+H)⁺: 253.5.

### 5-(2-Methoxy-phenyl)-4-(2,2,2-trifluoro-ethoxy)-pyridin-2-ylamine hydrochloride Compound No 23

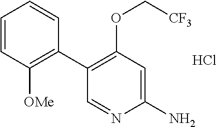 [SMILES: COc1ccccc1-c1cnc(N)cc1OCC(F)(F)F ; Cl]

Compound No 23 was prepared according to method 17 starting from 2-amino-5-bromo-4-(2,2,2-trifluoroethoxy)-pyridine (100 mg, 0.37 mmol, 1.0 eq.) and 2-methoxyphenylboronic acid (61 mg, 0.41 mmol, 1.1 eq.). The crude was purified by flash chromatography (SiO₂, CycloHex/EtOAc, 100/0 to 0/100). The resulting solid was further purified by preparative HPLC. After lyophilisation, the resulting foam (35 mg) was suspended in H₂O and an aqueous solution of HCl (1 M, 300 μL) was added dropwise. The obtained solution was lyophilised to afford compound No 23 as a white foam (29 mg, 26%).

Mp: 238-240° C.; ¹H NMR (400 MHz, DMSO-d₆) δ: 3.71 (s, 3H, O—CH₃); 4.95 (q, J 8.4 Hz, 2H, O—CH₂—CF₃); 6.49 (s, 1H, Ar); 7.00 (td, J 7.6, 1.6 Hz, 1H, Ar); 7.09 (dd, J 7.6, 0.4 Hz, 1H, Ar); 7.20 (dd, J 7.6, 1.6 Hz, 1H, Ar); 7.39-7.41 (m, 1H, Ar); 7.84 (s, 1H, Ar); 7.88 (bs, 2H, NH₂); 13.30 (bs, 1H, HCl salt); M/Z (M+H)⁺: 299.5.

### 5-(2,3-Dichloro-phenyl)-4-(2,2,2-trifluoro-ethoxy)-pyridin-2-ylamine hydrochloride Compound No 24

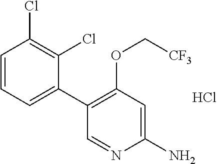 [SMILES: Nc1cc(OCC(F)(F)F)c(-c2cccc(Cl)c2Cl)cn1 ; Cl]

Compound No 24 was prepared according to method 17 starting from 2-amino-5-bromo-4-(2,2,2-trifluoroethoxy)-pyridine (100 mg, 0.37 mmol, 1.0 eq.) and 2,3-dichlorophenylboronic acid (85 mg, 0.44 mmol, 1.2 eq.). The crude was purified by flash chromatography (SiO₂, CycloHex/EtOAc, 100/0 to 0/100). The resulting solid was further purified by preparative HPLC. After lyophilisation, the resulting foam (42 mg) was suspended in H₂O and an aqueous solution of HCl (1M, 300 μL) was added dropwise. The obtained solution was lyophilised to afford compound No 24 as a white foam (33 mg, 27%).

Mp>250° C.; ¹H NMR (400 MHz, DMSO-d₆) δ: 4.99 (q, J 8.4 Hz, 2H, O—CH₂—CF₃); 6.54 (s, 1H, Ar); 7.37 (dd, J 7.6, 1.6 Hz, 1H, Ar); 7.46 (t, J 7.6 Hz, 1H, Ar); 7.73 (dd, J 7.6, 1.6 Hz, 1H, Ar); 7.99 (s, 1H, Ar); 8.04 (bs, 2H, NH₂); 13.59 (bs, 1H, HCl salt); M/Z (M[³⁵Cl]₂+H)⁺: 337.5.

### 4-(2-aminoethoxy)-5-(2-methoxyphenyl)pyridin-2-amine dihydrochloride Compound No 25

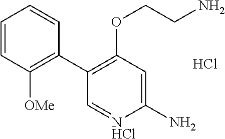

Compound No 25 was prepared according to method 17 starting from 2-amino-5-bromo-4-(2-aminoethoxy)-pyridine (114 mg, 0.49 mmol, 1.0 eq.) and 2-methoxyphenylboronic acid (82 mg, 0.54 mmol, 1.1 eq.). The crude was purified by flash chromatography (Biotage® KP-NH—SiO₂, DCM/MeOH, 100/0 to 90/10). The resulting solid was triturated in Et₂O and the precipitate was dried under high vacuum at 80° C. for 24 h. The resulting powder was solubilised in DCM and a solution of HCl (2 M in Et₂O, 1 mL) was added dropwise. The resulting precipitate was collected, triturated in Et₂O and dried under high vacuum at 80° C. to afford compound No 25 as white solid (16 mg, 13%).

Mp: 218-224° C.; ¹H NMR (400 MHz, DMSO-d₆) δ: 3.15 (t, J 6.0 Hz, 2H, O—CH₂—CH₂—NH₂); 3.74 (s, 3H, O—CH₃); 4.32 (t, J 6.0 Hz, 2H, O—CH₂—CH₂—NH₂); 6.56 (s, 1H, Ar); 7.01 (td, J 7.2, 0.8 Hz, 1H, Ar); 7.10 (dd, J 8.0, 0.4 Hz, 1H, Ar); 7.30 (dd, J 7.2, 1.6 Hz 1H, Ar); 7.35-7.44 (m, 1H, Ar); 7.79 (s, 1H, Ar); 7.94 (bs, 2H, NH₂); 8.21 (bs, 3H, NH₃⁺); 13.38 (bs, 1H, HCl salt); M/Z (M+H)⁺: 260.1.

### 5-(2-Methoxy-phenyl)-6-methyl-pyridin-2-ylamine Compound No 26

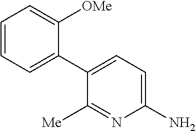 [SMILES: COc1ccccc1-c1ccc(N)nc1C]

Compound No 26 was prepared according to method 18 starting from 2-amino-5-bromo-6-methylpyridine (100 mg, 0.53 mmol, 1.0 eq.) and 2-methoxyphenylboronic acid (88 mg, 0.58 mmol, 1.1 eq.). The crude was purified by flash chromatography (SiO₂, CycloHex/EtOAc, 100/0 to 0/100). The resulting foam was triturated thrice in Et₂O and the precipitate was dried at 70° C. under high vacuum overnight to afford compound No 26 as a beige solid (50 mg, 44%).

Mp: 132-140° C.; ¹H NMR (400 MHz, DMSO-d₆) δ: 2.02 (s, 3H, CH₃); 3.71 (s, 3H, O—CH₃); 5.78 (bs, 2H, NH₂); 6.30 (dd, J 8.2, 0.4 Hz, 1H, Ar); 6.96 (td, J 7.2, 0.8 Hz, 1H, Ar); 7.02-7.10 (m, 3H, Ar); 7.30 (ddd, J 8.2, 7.2, 1.6 Hz, 1H, Ar). M/Z (M+H)⁺: 215.6.

### 5-(2-Methoxy-phenyl)-4,6-dimethyl-pyridin-2-ylamine Compound No 27

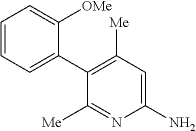 [SMILES: COc1ccccc1-c1c(C)cc(N)nc1C]

Compound No 27 was prepared according to method 18 using PdP(t-Bu)₃PdG2 (10 mg, 0.015 mmol, 7.5 mol %), starting from 2-amino-5-bromo-4,6-dimethylpyridine (50 mg, 0.25 mmol, 1.0 eq.) and 2-methoxyphenylboronic acid (43 mg, 0.28 mmol, 1.1 eq.). The crude was purified by flash chromatography (SiO₂, DCM/MeOH, 100/0 to 90/10). The resulting foam was triturated thrice in Et₂O and the precipitate was dried at 70° C. under high vacuum overnight to afford compound No 27 as a beige solid (32 mg, 28%).

Mp: 178-181° C.; ¹H NMR (400 MHz, DMSO-d₆) δ: 1.76 (s, 3H, CH₃); 1.90 (s, 3H, CH₃); 3.69 (s, 3H, O—CH₃); 5.61 (bs, 2H, NH₂); 6.18 (bs, 1H, Ar); 6.96-7.00 (m, 2H, Ar); 7.06 (d, J 8.0 Hz, 1H, Ar); 7.28-7.35 (m, 1H, Ar); M/Z (M+H)⁺: 229.7.

### 5-(2,3-Dichloro-phenyl)-4,6-dimethyl-pyridin-2-ylamine Compound No 28

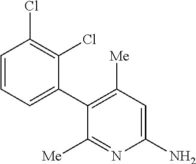 [SMILES: Cc1cc(N)nc(C)c1-c1cccc(Cl)c1Cl]

Compound No 28 was prepared according to method 18 starting from 2-amino-5-bromo-4,6-dimethylpyridine (150 mg, 0.75 mmol, 1.0 eq.) and 2,3-dichlorophenylboronic acid (158 mg, 0.83 mmol, 1.1 eq.). The crude was purified by flash chromatography (SiO₂, CycloHex/EtOAc, 100/0 to 50/50) to afford compound No 28 as a beige solid (26 mg, 13%).

Mp: 140-145° C.; ¹H NMR (400 MHz, DMSO-d₆) δ: 1.77 (s, 3H, CH₃); 1.89 (s, 3H, CH₃); 5.84 (bs, 2H, NH₂); 6.22 (s, 1H, Ar); 7.22 (dd, J 7.8, 1.6 Hz, 1H, Ar); 7.42 (t, J 7.8 Hz, 1H, Ar); 7.64 (dd, J 7.8, 1.6 Hz, 1H, Ar); M/Z (M[³⁵Cl]₂+H)⁺: 267.5.

### 5-(3-chloro-2-methyl-phenyl)-6-ethyl-pyridin-2-ylamine hydrochloride Compound No 29

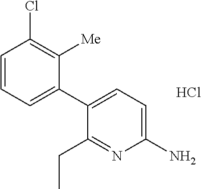 [SMILES: CCc1nc(N)ccc1-c1cccc(Cl)c1C ; Cl]

Compound No 29 was prepared according to method 19 starting from 2-amino-5-bromo-6-ethylpyridine (100 mg, 0.50 mmol, 1.0 eq.) and 3-chloro-2-methylphenylboronic acid (128 mg, 0.75 mmol, 1.5 eq.). The crude was purified by flash chromatography (SiO₂, DCM/MeOH, 100/0 to 95/5). The resulting foam was further purified by preparative HPLC. The obtained solid was taken up in a mixture of 1 M aqueous HCl/ACN and the resulting solution was lyophilised to afford compound No 29 as a white solid (9 mg, 8%).

Mp: 200-210° C.; ¹H NMR (400 MHz, DMSO-d₆) δ: 1.08 (t, J 7.6 Hz, 3H, CHₐH_{b}—CH₃); 2.11 (s, 3H, CH₃); 2.32-2.41 (m, 1H, CHₐH_{b}—CH₃); 2.52-2.57 (m, 1H, CHₐH_{b}—CH₃); 6.90 (d, J 9.0 Hz, 1H, Ar); 7.17 (dd, J 8.0, 1.2 Hz, 1H, Ar); 7.32 (d, J 8.0 Hz, 1H, Ar); 7.53 (dd, J 8.0, 1.2 Hz, 1H, Ar); 7.69 (d, J 9.0 Hz, 1H, Ar); 7.94 (bs, 2H, NH₂); 14.15 (bs, 1H, HCl salt); M/Z (M[³⁵Cl]₂+H)⁺: 247.7.

### 5-(2-cyclopropylphenyl)-6-ethyl-pyridin-2-amine hydrochloride Compound No 30

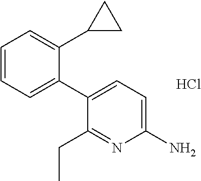 [SMILES: CCc1nc(N)ccc1-c1ccccc1C1CC1 ; Cl]

Compound No 30 was prepared according to method 19 starting from 2-amino-5-bromo-6-ethylpyridine (100 mg, 0.50 mmol, 1.0 eq.) and 2-cyclopropylbenzeneboronic acid (122 mg, 0.75 mmol, 1.5 eq.). The crude was purified by flash chromatography (SiO₂, DCM/MeOH, 100/0 to 97/3). The resulting foam was further purified by preparative HPLC. The obtained solid was taken up in a mixture of 1 M aqueous HCl/ACN and the resulting solution was lyophilised to afford compound No 30 as a white solid (53 mg, 38%).

Mp: 180-190° C.; ¹H NMR (400 MHz, DMSO-d₆) δ: 0.64-0.69 (m, 2H, CyPr); 0.78-0.86 (m, 2H, CyPr); 1.11 (t, J 7.6 Hz, 3H, CHₐH_{b}—CH₃); 1.51-1.58 (m, 1H, CyPr); 2.40-2.48 (m, 1H, CHₐH_{b}—CH₃); 2.52-2.60 (m, 1H, 3H, CHₐH_{b}—CH₃); 6.91 (d, J 9.0 Hz, 1H, Ar); 6.99 (d, J 7.6 Hz, 1H, Ar); 7.14 (dd, J 7.6, 1.0 Hz, 1H, Ar); 7.24 (td, J 7.6, 1.0 Hz, 1H, Ar); 7.34 (td, J 7.6, 1.0 Hz, 1H, Ar); 7.74 (d, J 9.0 Hz, 1H, Ar); 7.91 (bs, 2H, NH₂); 14.12 (bs, 1H, HCl salt); M/Z (M+H)⁺: 239.8.

### 5-[2-(cyclopropoxy)phenyl]-6-ethyl-pyridin-2-amine hydrochloride Compound No 31

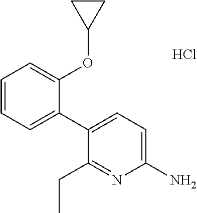 [SMILES: CCc1nc(N)ccc1-c1ccccc1OC1CC1 ; Cl]

Compound No 31 was prepared according to method 19 starting from 2-amino-5-bromo-6-ethylpyridine (100 mg, 0.50 mmol, 1.0 eq.) and 2-(2-cyclopropoxyphenyl)-4,4,5,5-tetramethyl-1,3,2-dioxaborolane (195 mg, 0.75 mmol, 1.5 eq.). The crude was purified by flash chromatography (SiO₂, DCM/MeOH, 100/0 to 97/3). The resulting foam was further purified by preparative HPLC. The obtained solid was taken up in a mixture of 1 M aqueous HCl/ACN and the resulting solution was lyophilised to afford compound No 31 as a white solid (4 mg, 3%).

Mp: 60-80° C.; ¹H NMR (400 MHz, DMSO-d₆) δ: 0.53-0.59 (m, 2H, O-CyPr); 0.74-0.79 (m, 2H, O-CyPr); 1.10 (t, J 7.6 Hz, 3H, CH₂—CH₃); 2.39-2.48 (m, 2H, CH₂—CH₃); 3.84 (qt, 73.0 Hz, 1H, CH); 6.86 (d, J 9.0 Hz, 1H, Ar); 7.05-7.09 (m, 1H, Ar); 7.17 (dd, J 7.6, 1.5 Hz, 1H, Ar); 7.40-7.47 (m, 2H, Ar); 7.65 (d, J 9.0 Hz, 1H, Ar); 7.92 (bs, 2H, NH₂); 14.08 (bs, 1H, HCl salt); M/Z (M+H)⁺: 255.8.

### 6-Fluoro-5-(2-methoxy-phenyl)-pyridin-2-ylamine Compound No 32

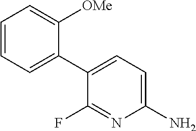 [SMILES: COc1ccccc1-c1ccc(N)nc1F]

Compound No 32 was prepared according to method 17 starting from 2-amino-5-bromo-6-fluoropyridine (100 mg, 0.52 mmol, 1.0 eq.) and 2-methoxyphenylboronic acid (87 mg, 0.57 mmol, 1.1 eq.). The crude was purified by flash chromatography (SiO₂, CycloHex/EtOAc, 100/0 to 50/50). The resulting beige foam was triturated thrice in Et₂O and the precipitate was dried at 80° C. under high vacuum overnight to afford compound No 32 as a white powder (78 mg, 69%).

Mp: 175-178° C.; ¹H NMR (400 MHz, DMSO-d₆) δ: 3.73 (s, 3H, O—CH₃); 6.30 (s, 2H, NH₂); 6.35 (dd, J 8.0, 2.0 Hz, 1H, Ar); 6.97 (td, J 7.2, 1.2 Hz, 1H, Ar); 7.06 (dd, J 8.4, 0.8 Hz, 1H, Ar); 7.17 (ddd, J 7.2, 1.8, 0.8 Hz, 1H, Ar); 7.31 (ddd, J 8.4, 7.2, 1.8 Hz, 1H, Ar); 7.43 (dd, J 10.2, 8.0 Hz, 1H, Ar); M/Z (M+H)⁺: 219.6.

### 5-(2,3-Dichloro-phenyl)-6-fluoro-pyridin-2-ylamine Compound No 33

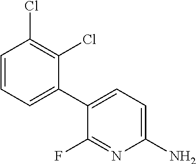 [SMILES: Nc1ccc(-c2cccc(Cl)c2Cl)c(F)n1]

Compound No 33 was prepared according to method 17 starting from 2-amino-5-bromo-6-fluoropyridine (100 mg, 0.52 mmol, 1.0 eq.) 2,3-dichlorophenylboronic acid (109 mg, 0.57 mmol, 1.1 eq.). The crude was purified by flash chromatography (SiO₂, CycloHex/EtOAc, 100/0 to 50/50). The resulting beige foam was triturated thrice in Et₂O and the precipitate was dried at 80° C. under high vacuum overnight to afford compound No 33 as a white powder (25 mg, 19%).

Mp: 139-144° C.; ¹H NMR (400 MHz, DMSO-d₆) δ: 6.40 (dd, J 8.1, 2.0 Hz, 1H, Ar); 6.56 (s, 2H, NH₂); 7.35 (dd, J 7.8, 1.6 Hz, 1H, Ar); 7.40 (t, 77.8 Hz, 1H, Ar); 7.46 (dd, J 10.4, 8.1 Hz, 1H, Ar); 7.64 (dd, J 7.8, 1.6 Hz, 1H, Ar); M/Z (M[³⁵Cl]₂+H)⁺: 257.5.

### 5-(2,3-Dichloro-phenyl)-6-trifluoromethyl-pyridin-2-ylamine Compound No 34

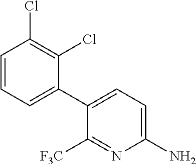 [SMILES: Nc1ccc(-c2cccc(Cl)c2Cl)c(C(F)(F)F)n1]

Compound No 34 was prepared according to method 18 starting from 2-amino-5-bromo-6-trifluoromethylpyridine (100 mg, 0.41 mmol, 1.0 eq.) and 2,3-dichlorophenylboronic acid (94 mg, 0.49 mmol, 1.1 eq.). The crude was purified by flash chromatography (SiO₂, CycloHex/EtOAc, 100/0 to 70/30). The resulting beige foam was triturated thrice in Et₂O and the precipitate was dried at 80° C. under high vacuum overnight to afford compound No 34 as a white powder (79 mg, 63%).

Mp: 165-170° C.; ¹H NMR (400 MHz, DMSO-d₆) δ: 6.70 (bs, 2H, NH₂); 6.74 (d, J 8.4 Hz, 1H, Ar); 7.28 (dd, J 7.6, 1.5 Hz, 1H, Ar); 7.37 (d, J 8.4 Hz, 1H, Ar); 7.40 (dd, J 8.0, 7.6 Hz, 1H, Ar); 7.67 (dd, J 8.0, 1.5 Hz, 1H, Ar); M/Z (M[³⁵Cl]₂+H)⁺: 307.4.

### 6-Methoxy-5-(2-methoxy-phenyl)-pyridin-2-ylamine Compound No 35

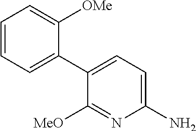 [SMILES: COc1ccccc1-c1ccc(N)nc1OC]

Compound No 35 was prepared according to method 17 starting from 2-amino-5-bromo-6-methoxypyridine (Wang, Y. et al., PCT Int. Appl., 2013029338, 2013) (100 mg, 0.49 mmol, 1.0 eq.) and 2-methoxyphenylboronic acid (82 mg, 0.54 mmol, 1.1 eq.). The crude was purified by flash chromatography (SiO₂, CycloHex/EtOAc, 100/0 to 50/50). The resulting beige foam was triturated thrice in Et₂O and the precipitate was dried at 80° C. under high vacuum overnight to afford compound No 35 as a white powder (64 mg, 57%).

Mp: 85-87° C.; ¹H NMR (400 MHz, DMSO-d₆) δ: 3.69 (s, 6H, 2 O—CH₃); 5.84 (bs, 2H, NH₂); 6.05 (d, J 8.0 Hz, 1H, Ar); 6.91 (td, J 7.4, 1.0 Hz, 1H, Ar); 7.00 (dd, J 8.3, 1.0 Hz, 1H, Ar); 7.11 (dd, J 7.4, 1.8 Hz, 1H, Ar); 7.16 (d, J 8.0 Hz, 1H, Ar); 7.23 (ddd, J 8.3, 7.4, 1.8 Hz, 1H, Ar); M/Z (M+H)⁺: 231.7.

### 5-(2-Methoxy-phenyl)-6-(2,2,2-trifluoro-ethoxy)-pyridin-2-ylamine Compound No 36

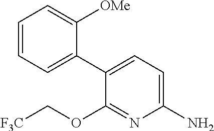 [SMILES: COc1ccccc1-c1ccc(N)nc1OCC(F)(F)F]

Compound No 36 was prepared according to method 17 starting from 2-amino-5-bromo-6-(2,2,2-trifluoro-ethoxy)-pyridine (100 mg, 0.37 mmol, 1.0 eq.) and 2-methoxyphenylboronic acid (67 mg, 0.41 mmol, 1.1 eq.). The crude was purified by flash chromatography (SiO₂, CycloHex/EtOAc, 100/0 to 50/50). The resulting beige gum was triturated thrice in Et₂O. The supernatant was removed and the remaining gum was dried at 80° C. under high vacuum overnight to afford compound No 36 as a beige gum (72 mg, 65%).

¹H NMR (400 MHz, DMSO-d₆) δ: 3.68 (s, 3H, O—CH₃); 4.85 (q, J 9.4 Hz, 2H, O—CH₂—CF₃); 6.04 (bs, 2H, NH₂); 6.16 (d, J 7.4 Hz, 1H, Ar); 6.92 (td, J 7.4, 0.9 Hz, 1H, Ar); 7.00 (dd, J 7.4, 0.9 Hz, 1H, Ar); 7.12 (dd, J 7.4, 1.7 Hz, 1H, Ar); 7.23-7.29 (m, 2H, Ar); M/Z (M+H)⁺: 299.6.

### 6-(2-Amino-ethoxy)-5-(2-methoxy-phenyl)-pyridin-2-ylamine Compound No 37

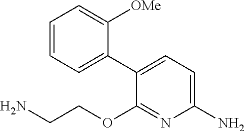 [SMILES: COc1ccccc1-c1ccc(N)nc1OCCN]

Compound No 37 was prepared according to method 18 starting from 2-amino-5-bromo-6-(2-amino-ethoxy)-pyridine (100 mg, 0.49 mmol, 1.0 eq.) and 2-methoxyphenylboronic acid (82 mg, 0.54 mmol, 1.1 eq.). The crude was purified by flash chromatography (SiO₂, DCM/MeOH, 100/0 to 90/10). The resulting beige foam was triturated thrice in Et₂O and the precipitate was dried at 80° C. under high vacuum overnight to afford compound No 37 as a white solid (23 mg, 18%).

Mp: 197-203° C.; ¹H NMR (400 MHz, DMSO-d₆) δ: 3.03 (t, J 5.6 Hz, 2H, O—CH₂—CH₂—NH₂); 3.71 (s, 3H, O—CH₃); 4.29 (t, 75.6 Hz, 2H, O—CH₂—CH₂—NH₂); 5.97 (bs, 2H, NH₂); 6.11 (d, J 8.0 Hz, 1H, Ar); 6.92 (td, J 7.4, 0.9 Hz, 1H, Ar); 7.01 (dd, J 8.2, 0.9 Hz, 1H, Ar); 7.10-7.62 (m, 5H, 3H Ar+NH₂); M/Z (M+H)⁺: 260.6.

### 6-(2-Amino-ethoxy)-5-(2,3-dichloro-phenyl)-pyridin-2-ylamine dihydrochloride Compound No 38

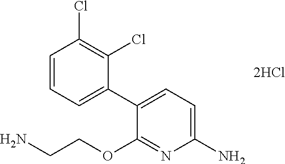 [SMILES: NCCOc1nc(N)ccc1-c1cccc(Cl)c1Cl]

Compound No 38 was prepared according to method 18 starting from 2-amino-5-bromo-6-(2-amino-ethoxy)-pyridine (100 mg, 0.43 mmol, 1.0 eq.) and 2,3-dichlorophenylboronic acid (89 mg, 0.52 mmol, 1.2 eq.). The crude was purified by flash chromatography (SiO₂, DCM/MeOH, 100/0 to 90/10). The resulting foam was suspended in aqueous HCl (1 M, 5 mL) and lyophilised. The resulting powder was triturated in Et₂O to afford compound No 38 as a very hygroscopic white powder (21 mg, 17%).

Mp: 183-190° C.; ¹H NMR (400 MHz, DMSO-d₆) δ: 3.10 (sex, J 5.5 Hz, 2H, O—CH₂—CH₂—NH₂); 4.36 (t, J 5.5 Hz, 2H, O—CH₂—CH₂—NH₂); 6.17 (d, J 8.1 Hz, 1H, Ar); 7.27 (d, J 8.1 Hz, 1H, Ar); 7.31-7.37 (m, 2H, Ar); 7.52-7.58 (m, 1H, Ar); 8.00 (bs, 3H, NH₃⁺), signal of NH₂ was not observed; M/Z (M[³⁵Cl]₂+H)⁺: 298.5.

### 3-(2-Isopropoxy-6-methoxy-phenyl)-pyridine-2,6-diamine Compound No 39

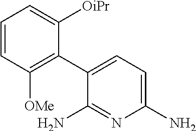 [SMILES: COc1cccc(OC(C)C)c1-c1ccc(N)nc1N]

Compound No 39 was prepared according to method 20 starting from 2,6-diamino-5-iodopyridine (100 mg, 0.43 mmol, 1.0 eq.) and 2-isopropoxy-6-methoxyphenylboronic acid (137 mg, 0.65 mmol, 1.5 eq.). The reaction mixture was stirred for 48 h. The crude was purified by flash chromatography (SiO₂, CycloHex/EtOAc, 100/0 to 0/100) to afford compound No 39 as a beige solid (81 mg, 69%).

¹H NMR (400 MHz, DMSO-d₆) δ: 1.10 (d, J 5.6 Hz, 3H, O—CH—(CH₃)₂); 1.11 (d, J 5.6 Hz, 3H, O—CH—(CH₃)₂); 3.66 (s, 3H, O—CH₃); 4.39 (sep, J 5.6 Hz, 1H, O—CH—(CH₃)₂); 4.49 (bs, 2H, NH₂); 5.34 (bs, 2H, NH₂); 5.75 (d, J 8.0 Hz, 1H, Ar); 6.66 (d, J 8.4 Hz, 1H, Ar); 6.68 (d, J 8.4 Hz, 1H, Ar); 6.81 (d, J 8.0 Hz, 1H, Ar); 7.21 (t, J 8.4 Hz, 1H, Ar); M/Z (M+H)⁺: 274.9.

### 3-(4-Methoxy-2-methyl-phenyl)-pyridine-2,6-diamine Compound No 40

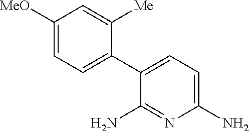 [SMILES: COc1ccc(-c2ccc(N)nc2N)c(C)c1]

compound No 40 was prepared according to method 20 at 60° C., starting from 2,6-diamino-5-iodopyridine (100 mg, 0.43 mmol, 1.0 eq.) and 4-methoxy-2-methylphenylboronic acid (78 mg, 0.47 mmol, 1.1 eq.). The reaction mixture was stirred for 48 h. The crude was purified by flash chromatography (SiO₂, CycloHex/EtOAc, 100/0 to 50/50). The resulting foam was triturated in Et₂O and the resulting solid was lyophilised to afford compound No 40 as a white powder (39 mg, 39%).

Mp: 150-158° C.; ¹H NMR (400 MHz, DMSO-d₆) δ: 2.08 (s, 3H, CH₃); 3.75 (s, 3H, O—CH₃); 4.64 (s, 2H, NH₂); 5.39 (s, 2H, NH₂); 7.76 (d, J 8.0 Hz, 1H, Ar); 6.77 (dd, J 8.4, 2.8 Hz, 1H, Ar); 6.83 (d, J 8.0 Hz, 1H, Ar); 6.83 (s, 1H, Ar); 6.98 (d, J 8.4 Hz, 1H, Ar); M/Z (M+H)⁺: 230.8.

### 3-(4-Chloro-2-fluoro-phenyl)-pyridine-2,6-diamine Compound No 41

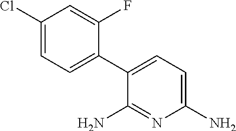 [SMILES: Nc1ccc(-c2ccc(Cl)cc2F)c(N)n1]

Compound No 41 was prepared according to method 20 at 60° C., starting from 2,6-diamino-5-iodopyridine (100 mg, 0.43 mmol, 1.0 eq.) and 4-chloro-2-fluorophenylboronic acid (82 mg, 0.47 mmol, 1.1 eq.). The reaction mixture was stirred for 48 h. The crude was purified by flash chromatography (SiO₂, CycloHex/EtOAc, 100/0 to 0/100). The resulting foam was triturated in Et₂O and the resulting solid was lyophilised to afford compound No 41 as a white powder (38 mg, 37%).

Mp: 134-139° C.; ¹H NMR (400 MHz, DMSO-d₆) δ: 5.09 (s, 2H, NH₂); 5.61 (s, 2H, NH₂); 5.79 (d, J 8.0 Hz, 1H, Ar); 6.96 (dd, J 8.0, 0.6 Hz, 1H, Ar); 7.27 (dd, J 8.0, 2.0 Hz, 1H, Ar); 7.32 (t, J 8.0 Hz, 1H, Ar); 7.40 (dd, J 10.2, 2.0 Hz, 1H, Ar); M/Z (M[³⁵C1]₂+H)⁺: 238.7.

### 3-(2-Cyclopropyl-phenyl)-pyridine-2,6-diamine hydrochloride Compound No 42

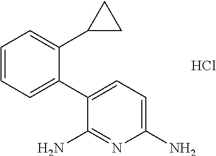 [SMILES: Nc1ccc(-c2ccccc2C2CC2)c(N)n1 ; Cl]

Compound No 42 was prepared according to method 20 at 60° C., starting from 2,6-diamino-5-iodopyridine (100 mg, 0.43 mmol, 1.0 eq.) and 2-cyclopropylphenylboronic acid (76 mg, 0.47 mmol, 1.1 eq.). The reaction mixture was stirred for 48 h. The crude was purified by flash chromatography (SiO₂, CycloHex/EtOAc, 100/0 to 0/100). The resulting foam was triturated in pentane. The collected solid was taken up in a mixture of 1 M aqueous HCl/ACN (1/1 v/v) and the resulting solution was lyophilised to afford compound No 42 as a pale yellow powder (28 mg, 25%).

¹H NMR (400 MHz, DMSO-d₆) δ: 0.51-0.60 (m, 1H, CyPr), 0.68-0.88 (m, 3H, CyPr), 1.63-1.70 (m, 1H, CyPr), 6.04 (d, J 8.4 Hz, 1H, Ar); 6.69 (bs, 2H, NH₂); 7.00 (dd, J 7.6, 1.2 Hz, 1H, Ar), 7.12 (dd, J 7.6, 1.2 Hz, 1H, Ar); 7.23 (td, J 7.6, 1.2 Hz, 1H, Ar); 7.31 (td, J 7.6, 1.2 Hz, 1H, Ar); 7.35 (bs, 2H, NH₂); 7.41 (d, J 8.4 Hz, 1H, Ar); 12.99 (s, 1H, HCl salt); M/Z (M+H)⁺: 226.8.

### 3-(2-Phenoxy-phenyl)-pyridine-2,6-diamine hydrochloride compound No 43

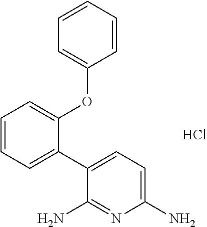 [SMILES: Nc1ccc(-c2ccccc2Oc2ccccc2)c(N)n1 ; Cl]

Compound No 43 was prepared according to method 20 at 60° C., starting from 2,6-diamino-5-iodopyridine (100 mg, 0.43 mmol, 1.0 eq.) and 2-phenoxyphenylboronic acid (101 mg, 0.47 mmol, 1.1 eq.). The crude was purified by flash chromatography (SiO₂, CycloHex/EtOAc, 100/0 to 0/100). The obtained solid was taken up in a mixture of 1 M aqueous HCl/ACN (1/1 v/v) and the resulting solution was lyophilised. The resulted solid was further triturated in Et₂O and was dried overnight in vacuo at 70° C. over P₂O₅ to afford compound No 43 hydrochloride as a yellow powder (27 mg, 20%).

¹H NMR (400 MHz, DMSO-d₆) δ: 5.96 (d, J 8.4 Hz, 1H, Ar); 6.91 (s, 2H, NH₂); 6.94-6.99 (m, 3H, Ar); 7.08 (tt, J 7.2, 1.6 Hz, 1H, Ar); 7.25 (td, J 7.2, 1.6 Hz, 1H, Ar); 7.29-7.45 (m, 7H, 5H Ar+NH₂) 12.90 (s, 1H, HCl salt); M/Z (M+H)⁺: 278.8.

### 3-(2-Benzyl-phenyl)-pyridine-2,6-diamine Compound No 44

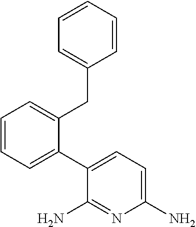 [SMILES: Nc1ccc(-c2ccccc2Cc2ccccc2)c(N)n1]

Compound No 44 was prepared according to method 20 at 60° C., starting from 2,6-diamino-5-iodopyridine (100 mg, 0.43 mmol, 1.0 eq.) and 2-benzylphenylboronic acid (100 mg, 0.47 mmol, 1.1 eq.). The crude was purified by flash chromatography (SiO₂, CycloHex/EtOAc, 100/0 to 0/100). The resulted solid was further triturated in Et₂O and was dried in vacuo over P₂O₅ to afford compound No 44 as a beige solid (27 mg, 22%).

¹H NMR (400 MHz, DMSO-d₆) δ: 3.75 (d, J 15.2 Hz, 1H, CHₐH_{b}-Ph); 3.85 (d, J 15.2 Hz, 1H, CHₐH_{b}-Ph); 5.88 (d, J 8.4 Hz, 1H, Ar); 5.98 (bs, 2H, NH₂); 6.68 (bs, 2H, NH₂); 6.96-7.02 (m, 2H, Ar); 7.07 (d, J 8.4 Hz, 1H, Ar); 7.10 (m, 7H, Ar); M/Z (M+H)⁺: 276.9.

### 3-(2-Chloro-4-fluoro-phenyl)-pyridine-2,6-diamine Compound No 45

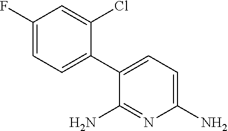 [SMILES: Nc1ccc(-c2ccc(F)cc2Cl)c(N)n1]

Compound No 45 was prepared according to the method 20 starting from 2,6-diamino-5-iodopyridine (100 mg, 0.43 mmol, 1.0 eq.) and 2-chloro-4-fluorophenylboronic acid (82 mg, 0.47 mmol, 1.1 eq.). The crude was purified by flash chromatography (SiO₂, CycloHex/EtOAc, 100/0 to 0/100). The resulting foam was triturated in pentane to afford compound No 45 as a white powder (59 mg, 58%).

Mp: 150-155° C.; ¹H NMR (400 MHz, DMSO-d₆) δ: 4.92 (s, 2H, NH₂); 5.54 (s, 2H, NH₂); 5.77 (d, J 8.0 Hz, 1H, Ar); 6.88 (d, J 8.0 Hz, 1H, Ar); 7.21 (td, J 8.4, 2.6 Hz, 1H, Ar); 7.31 (dd, J 8.4, 6.8 Hz, 1H, Ar); 7.46 (dd, J 9.0, 2.6 Hz, 1H, Ar); M/Z (M[³⁵Cl]₂+H)⁺: 238.7.

### 3-(2-Isopropoxy-4-methyl-phenyl)-pyridine-2,6-diamine Compound No 46

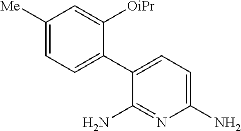 [SMILES: Cc1ccc(-c2ccc(N)nc2N)c(OC(C)C)c1]

Compound No 46 was prepared according to method 20 starting from 2,6-diamino-5-iodopyridine (100 mg, 0.43 mmol, 1.0 eq.) and 2-isopropoxy-4-methylphenylboronic acid (126 mg, 0.65 mmol, 1.5 eq.). The crude was purified by flash chromatography (SiO₂, CycloHex/EtOAc, 100/0 to 0/100). The resulting foam was triturated in pentane to afford compound No 46 as a white powder (46 mg, 42%).

Mp: 75-80° C.; ¹H NMR (400 MHz, DMSO-d₆) δ: 1.16 (d, J 6.0 Hz, 6H, O—CH—(CH₃)₂); 2.30 (s, 3H, CH₃); 4.46 (sep, J 6.0 Hz, 1H, O—CH—(CH₃)₂); 4.74 (s, 2H, NH₂); 5.41 (s, 2H, NH₂); 5.78 (d, J 8.0 Hz, 1H, Ar); 6.77 (ddd, J 7.6, 1.6, 0.8 Hz, 1H, Ar); 6.85-6.86 (m, 1H, Ar); 6.93 (d, J 8.0 Hz, 1H, Ar); 6.98 (d, J 7.6 Hz, 1H, Ar); M/Z (M+H)⁺: 258.9.

### 3-(4-Chloro-2-cyclopentyloxy-phenyl)-pyridine-2,6-diamine Compound No 47

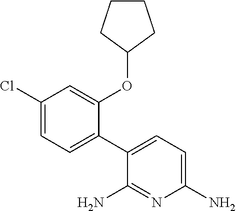 [SMILES: Nc1ccc(-c2ccc(Cl)cc2OC2CCCC2)c(N)n1]

Compound No 47 was prepared according to method 20 starting from 2,6-diamino-5-iodopyridine (100 mg, 0.43 mmol, 1.0 eq.) and 4-chloro-2-(cyclopentyloxy)-phenylboronic acid (156 mg, 0.65 mmol, 1.5 eq.). The crude was purified by flash chromatography (SiO₂, CycloHex/EtOAc, 100/0 to 0/100). The resulting foam was triturated in pentane to afford compound No 47 as a white powder (39 mg, 30%).

¹H NMR (400 MHz, DMSO-d₆) δ: 1.43-1.73 (m, 6H, CyPent); 1.77-1.88 (m, 2H, CyPent); 4.79-4.91 (m, 3H, O—CH, NH₂); 5.48 (bs, 2H, NH₂); 5.76 (d, J 8.0 Hz, 1H, Ar); 6.91 (d, J 8.0 Hz, 1H, Ar); 6.98 (dd, J 8.0, 2.0 Hz, 1H, Ar); 7.05 (d, J 2.0 Hz, 1H, Ar); 7.12 (d, J 8.0 Hz, 1H, Ar); M/Z (M[³⁵Cl]₂+H)⁺: 304.9.

### 3-(2-Cyclopropoxy-phenyl)-pyridine-2,6-diamine Compound No 48

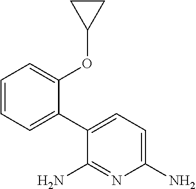 [SMILES: Nc1ccc(-c2ccccc2OC2CC2)c(N)n1]

Compound No 48 was prepared according to method 20 starting from 2,6-diamino-5-iodopyridine (60 mg, 0.25 mmol, 1.0 eq.) and 2-cyclopropyloxy-phenylboronic acid (100 mg, 0.38 mmol, 1.5 eq.). The crude was purified by flash chromatography (SiO₂, CycloHex/EtOAc, 100/0 to 0/100). The resulting foam was triturated in pentane to afford compound No 48 as a white powder (26 mg, 44%).

¹H NMR (400 MHz, DMSO-d₆) δ: 0.58-0.79 (m, 4H, O-CyPr); 3.81 (qt, J 3.0 Hz, 1H, CH); 4.69 (s, 2H, NH₂); 5.43 (s, 2H, NH₂); 5.78 (d, J 8.0 Hz, 1H, Ar); 6.89 (d, J 8.0 Hz, 1H, Ar); 6.98 (td, J 7.4, 1.6 Hz, 1H, Ar); 7.10 (dd, J 7.4, 1.6 Hz, 1H, Ar); 7.26-7.30 (m, 1H, Ar); 7.34 (dd, J 8.4, 1.2 Hz, 1H, Ar); M/Z (M+H)⁺: 242.9.

### 3-(2-Isopropoxy-5-methyl-phenyl)-pyridine-2,6-diamine Compound No 49

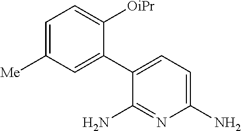 [SMILES: Cc1ccc(OC(C)C)c(-c2ccc(N)nc2N)c1]

Compound No 49 was prepared according to method 20 starting from 2,6-diamino-5-iodopyridine (100 mg, 0.43 mmol, 1.0 eq.) and 2-(isopropyloxy)-5-methylphenylboronic acid (126 mg, 0.65 mmol, 1.5 eq.). The crude was purified by flash chromatography (SiO₂, CycloHex/EtOAc, 100/0 to 0/100). The resulting foam was triturated in pentane to afford compound No 49 as a white powder (64 mg, 58%).

Mp: 78-81° C.; ¹H NMR (400 MHz, DMSO-d₆) δ: 1.14 (d, J 6.0 Hz, 6H, O—CH—(CH₃)₂); 2.24 (s, 3H, CH₃); 4.37 (sep, J 6.0 Hz, 1H, O—CH—(CH₃)₂); 4.77 (s, 2H, NH₂); 5.43 (s, 2H, NH₂); 5.77 (d, J 8.0 Hz, 1H, Ar); 6.91 (d, J 6.0 Hz, 1H, Ar); 6.92 (bs, 1H, Ar); 6.96 (d, J 8.0 Hz, 1H, Ar); 7.01-7.06 (m, 1H, Ar); M/Z (M+H)⁺: 258.9.

### 3-(5-Fluoro-2-isopropoxy-phenyl)-pyridine-2,6-diamine Compound No 50

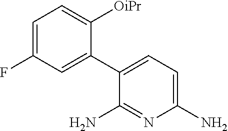 [SMILES: CC(C)Oc1ccc(F)cc1-c1ccc(N)nc1N]

Compound No 50 was prepared according to method 20 starting from 2,6-diamino-5-iodopyridine (100 mg, 0.43 mmol, 1.0 eq.) and 2-(isopropyloxy)-5-fluorophenylboronic acid (125 mg, 0.65 mmol, 1.5 eq.). The crude was purified by flash chromatography (SiO₂, CycloHex/EtOAc, 100/0 to 0/100). The resulting foam was triturated in pentane to afford compound No 50 as a white powder (88 mg, 79%).

Mp: 93-97° C.; ¹H NMR (400 MHz, DMSO-d₆) δ: 1.14 (d, J 6.0 Hz, 6H, O—CH—(CH₃)₂); 4.36 (sep, J 6.0 Hz, 1H, O—CH—(CH₃)₂); 4.89 (s, 2H, NH₂); 5.51 (s, 2H, NH₂); 5.79 (d, J 8.0 Hz, 1H, Ar); 6.90-6.96 (m, 1H, Ar); 6.99 (d, J 8.0 Hz, 1H, Ar); 7.02-7.06 (m, 2H, Ar); M/Z (M+H)⁺: 262.9.

### 3-(2,6-Dimethyl-phenyl)-pyridine-2,6-diamine Compound No 51

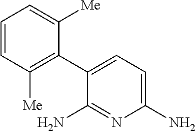 [SMILES: Cc1cccc(C)c1-c1ccc(N)nc1N]

Compound No 51 was prepared according to method 20 starting from 2,6-diamino-5-iodopyridine (100 mg, 0.43 mmol, 1.0 eq.) and 2,6-dimethyphenylboronic acid (97 mg, 0.65 mmol, 1.5 eq.). The reaction mixture was further stirred 48 h at 110° C. The crude was purified by flash chromatography (SiO₂, DCM/MeOH, 100/0 to 95/5). The resulting foam was triturated in Et₂O and then in pentane to afford compound No 51 as a white powder (14 mg, 15%).

Mp: 104-108° C.; ¹H NMR (400 MHz, DMSO-d₆) δ: 2.05 (s, 6H, 2×CH₃); 4.51 (bs, 2H, NH₂); 5.40 (bs, 2H, NH₂); 5.81 (d, J 8.0 Hz, 1H, Ar); 6.77 (d, J 8.0 Hz, 1H, Ar); 7.07-7.13 (m, 3H, Ar); M/Z (M+H)⁺: 214.8.

### 3-(2-Isopropoxy-5-trifluoromethyl-phenyl)-pyridine-2,6-diamine Compound No 52

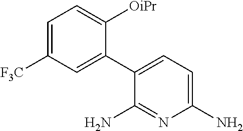 [SMILES: CC(C)Oc1ccc(C(F)(F)F)cc1-c1ccc(N)nc1N]

Compound No 52 was prepared according to method 20 starting from 2,6-diamino-5-iodopyridine (100 mg, 0.43 mmol, 1.0 eq.) and 2-(isopropyloxy)-5-trifluoromethylphenylboronic acid (161 mg, 0.65 mmol, 1.5 eq.). The crude was purified by flash chromatography (SiO₂, CycloHex/EtOAc, 100/0 to 0/100). The resulting foam was triturated in pentane to afford compound No 52 as a white powder (86 mg, 64%).

Mp: 100-103° C.; ¹H NMR (400 MHz, DMSO-d₆) δ: 1.23 (d, J 6.0 Hz, 6H, O—CH—(CH₃)₂); 4.69 (sep, J 6.0 Hz, 1H, O—CH—(CH₃)₂); 4.87 (s, 2H, NH₂); 5.52 (s, 2H, NH₂); 5.79 (d, J 8.0 Hz, 1H, Ar); 6.97 (d, J 8.0 Hz, 1H, Ar); 7.21 (d, J 8.8 Hz, 1H, Ar); 7.38 (d, J 2.4 Hz, 1H, Ar); 7.57-7.59 (m, 1H, Ar); M/Z (M+H)⁺: 312.9.

### 3-(4-Fluoro-2-isopropoxy-phenyl)-pyridine-2,6-diamine Compound No 53

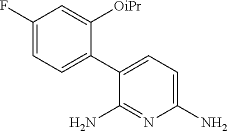 [SMILES: CC(C)Oc1cc(F)ccc1-c1ccc(N)nc1N]

Compound No 53 was prepared according to method 20 starting from 2,6-diamino-5-iodopyridine (100 mg, 0.43 mmol, 1.0 eq.) and 2-(isopropyloxy)-4-fluorophenylboronic acid (129 mg, 0.65 mmol, 1.5 eq.). The crude was purified by flash chromatography (SiO₂, CycloHex/EtOAc, 100/0 to 0/100). The resulting foam was triturated in pentane to afford compound No 53 as a white powder (67 mg, 59%).

Mp: 59-63° C.; ¹H NMR (400 MHz, DMSO-d₆) δ: 1.20 (d, J 6.0 Hz, 6H, O—CH—(CH₃)₂); 4.56 (sep, J 6.0 Hz, 1H, O—CH—(CH₃)₂); 4.76 (bs, 2H, NH₂); 5.42 (bs, 2H, NH₂); 5.77 (d, J 8.0 Hz, 1H, Ar); 6.76 (td, J 8.4, 2.8 Hz, 1H, Ar); 6.89-6.95 (m, 2H, Ar); 7.11 (d, J 8.4, 7.2 Hz, 1H, Ar); M/Z (M+H)⁺: 262.9.

### 3-(4-Chloro-2-methyl-phenyl)-pyridine-2,6-diamine Compound No 54

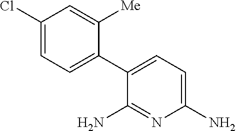 [SMILES: Cc1cc(Cl)ccc1-c1ccc(N)nc1N]

Compound No 54 was prepared according to method 20 starting from 2,6-diamino-5-iodopyridine (100 mg, 0.43 mmol, 1.0 eq.) and 4-chloro-2-methyphenylboronic acid (111 mg, 0.65 mmol, 1.5 eq.). The crude was purified by flash chromatography (SiO₂, CycloHex/EtOAc, 100/0 to 0/100). The resulting foam was triturated in pentane to afford compound No 54 as a white powder (30 mg, 30%).

Mp: 80-85° C.; ¹H NMR (400 MHz, DMSO-d₆) δ: 2.12 (s, 3H, CH₃); 4.81 (bs, 2H, NH₂); 5.49 (bs, 2H, NH₂); 5.79 (d, J 8.0 Hz, 1H, Ar); 6.85 (d, J 8.0 Hz, 1H, Ar); 7.08 (d, J 8.0 Hz, 1H, Ar), 7.23 (dd, J 8.0, 2.0 Hz, 1H, Ar); 7.32 (d, J 2.0 Hz, 1H, Ar); M/Z (M[³⁵Cl]₂+H)⁺:234.8.

### 3-(5-Chloro-2-cyclopropyl-phenyl)-pyridine-2,6-diamine Compound No 55

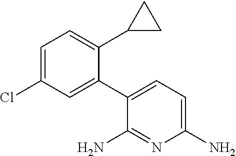 [SMILES: Nc1ccc(-c2cc(Cl)ccc2C2CC2)c(N)n1]

Compound No 55 was prepared according to method 20 starting from 2,6-diamino-5-iodopyridine (100 mg, 0.43 mmol, 1.0 eq.) and 5-chloro-2-cyclopropylphenylboronic acid pinacol ester (111 mg, 0.65 mmol, 1.5 eq.). The crude was purified by flash chromatography (SiO₂, DCM/MeOH, 100/0 to 95/5). The resulting foam was triturated in Et₂O and then in pentane to afford compound No 55 as a yellow solid (50 mg, 45%).

Mp: 39-44° C.; ¹H NMR (400 MHz, DMSO-d₆) δ: 0.50-0.59 (m, 1H, CyPr); 0.67-0.75 (m, 1H, CyPr); 0.78-0.87 (m, 2H, CyPr); 1.68-1.75 (m, 1H, CH); 4.84 (bs, 2H, NH₂); 5.51 (bs, 2H, NH₂); 5.80 (d, J 8.0 Hz, 1H, Ar); 6.89 (d, J 8.4 Hz, 1H, Ar); 6.95 (d, J 8.0 Hz, 1H, Ar); 7.07 (d, J 2.4 Hz, 1H, Ar); 7.23 (dd, J 8.4, 2.4 Hz, 1H, Ar); M/Z (M[³⁵C1]₂+H)⁺: 260.7.

### 3-(5-Chloro-2-methyl-phenyl)-pyridine-2,6-diamine Compound No 56

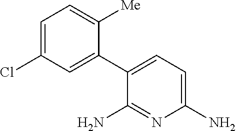 [SMILES: Cc1ccc(Cl)cc1-c1ccc(N)nc1N]

Compound No 56 was prepared according to method 20 starting from 2,6-diamino-5-iodopyridine (100 mg, 0.43 mmol, 1.0 eq.) and 5-chloro-2-methylphenylboronic acid (111 mg, 0.65 mmol, 1.5 eq.). The crude was purified by flash chromatography (SiO₂, CycloHex/EtOAc, 100/0 to 0/100). The resulting foam was triturated in pentane to afford compound No 56 as a beige solid (61 mg, 61%).

Mp: 95-97° C.; ¹H NMR (400 MHz, DMSO-d₆) δ: 2.10 (s, 3H, CH₃); 4.83 (bs, 2H, NH₂); 5.50 (bs, 2H, NH₂); 5.78 (d, J 8.0 Hz, 1H, Ar); 6.87 (d, J 8.0 Hz, 1H, Ar); 7.09 (d, J 2.4 Hz, 1H, Ar); 7.24 (dd, J 8.0, 2.4 Hz, 1H, Ar); 7.28 (d, J 8.0 Hz, 1H, Ar); M/Z (M+H)⁺: 234.7/236.7.

### 3-(2-Methyl-4-trifluoromethyl-phenyl)-pyridine-2,6-diamine Compound No 57

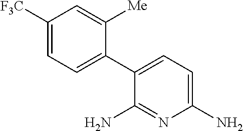 [SMILES: Cc1cc(C(F)(F)F)ccc1-c1ccc(N)nc1N]

Compound No 57 was prepared according to method 20 starting from 2,6-diamino-5-iodopyridine (100 mg, 0.43 mmol, 1.0 eq.) and 2-methyl-4-(trifluoromethyl)-phenylboronic acid (133 mg, 0.65 mmol, 1.5 eq.). The crude was purified by flash chromatography (SiO₂, CycloHex/EtOAc, 100/0 to 0/100). The resulting foam was triturated in pentane to afford compound No 57 as a beige solid (41 mg, 35%).

Mp: 60-65° C.; ¹H NMR (400 MHz, DMSO-d₆) δ: 2.21 (s, 3H, CH₃); 4.88 (bs, 2H, NH₂); 5.53 (bs, 2H, NH₂); 5.79 (d, J 8.0 Hz, 1H, Ar); 6.89 (d, J 8.0 Hz, 1H, Ar); 7.29 (d, J 8.0 Hz, 1H, Ar); 7.52 (dd, J 8.0, 1.6 Hz, 1H, Ar); 7.72 (bs, 1H, Ar); M/Z (M+H)⁺: 268.7.

### 3-(2-chloro-3-methyl-phenyl)-pyridine-2,6-diamine Compound No 58

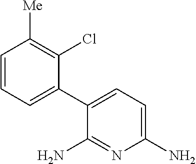 [SMILES: Cc1cccc(-c2ccc(N)nc2N)c1Cl]

Compound No 58 was prepared according to method 20 starting from 2,6-diamino-5-iodopyridine (100 mg, 0.43 mmol, 1.0 eq.) and 2-chloro-3-methylphenylboronic acid (109 mg, 0.65 mmol, 1.5 eq.). The crude was purified by flash chromatography (SiO₂, CycloHex/EtOAc, 80/20 to 0/100). The resulting foam was triturated in pentane to afford compound No 58 as a beige solid (82 mg, 83%).

Mp: 109-114° C.; ¹H NMR (400 MHz, DMSO-d₆) δ: 2.49 (s, 3H, CH₃); 4.78 (bs, 2H, NH₂); 5.51 (s, 2H, NH₂); 5.78 (d, J 8.0 Hz, 1H, Ar); 6.89 (d, J 8.0 Hz, 1H, Ar); 7.09-7.12 (m, 1H, Ar); 7.24 (t, 77.5 Hz, 1H, Ar), 7.28-7.30 (m, 1H, Ar); M/Z (M[³⁵C1]₂+H)⁺: 234.8.

### 3-(2-Methylsulfanyl-phenyl)-pyridine-2,6-diamine hydrochloride Compound No 59

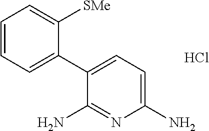 [SMILES: CSc1ccccc1-c1ccc(N)nc1N ; Cl]

Compound No 59 was prepared according to method 20 starting from 2,6-diamino-5-iodopyridine (100 mg, 0.43 mmol, 1.0 eq.) and 2-(methylthio)-phenylboronic acid (109 mg, 0.65 mmol, 1.5 eq.). The crude was purified by flash chromatography (SiO₂, CycloHex/EtOAc, 100/0 to 0/100). The resulting foam was triturated in pentane. The obtained solid was taken up in a mixture of 1 M aqueous HCl/ACN (1/1 v/v) and the resulting solution was lyophilised to afford compound No 59 a beige solid (72 mg, 63%).

Mp: 73-78° C.; ¹H NMR (400 MHz, DMSO-d₆) δ: 2.39 (s, 3H, S—CH₃); 6.01 (d, J 8.4 Hz, 1H, Ar); 7.73 (bs, 2H, NH₂); 7.15 (dd, J 7.6, 1.2 Hz, 1H, Ar); 7.23 (td, J 7.6, 1.2 Hz, 1H, Ar); 7.33 (d, J 8.4 Hz, 1H, Ar); 7.33-7.38 (m, 1H, Ar); 7.39-7.47 (m, 1H, Ar); 7.41 (bs, 2H, NH₂); 12.91 (s, 1H, HCl salt); M/Z (M+H)⁺: 232.8.

### 2-(2,6-Diamino-pyridin-3-yl)-N,N-diethyl-benzamide hydrochloride Compound No 60

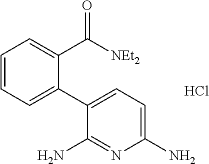

Compound No 60 was prepared according to method 20 starting from 2,6-diamino-5-iodopyridine (100 mg, 0.43 mmol, 1.0 eq.) and 2-(N,N-diethylaminocarbonyl)-phenylboronic acid (243 mg, 1.08 mmol, 2.5 eq.). The crude was purified by preparative HPLC. The obtained solid was taken up in a mixture of 1 M aqueous HCl/ACN (1/1 v/v) and the resulting solution was lyophilised to afford compound No 60 as a yellow solid (11 mg, 8%).

¹H NMR (400 MHz, DMSO-d₆) δ: 0.87 (t, J 7.2 Hz, 3H, N—CH₂—CH₃); 0.92 (t, J 7.2 Hz, 3H, N—CH₂—CH₃); 2.50-2.54 (m, 2H, CH₂); 3.01-3.04 (m, 2H, CH₂); 5.98 (d, J 8.4 Hz, 1H, Ar); 6.87 (bs, 2H, NH₂); 7.31 (d, J 8.4 Hz, 1H, Ar); 7.32-7.54 (m, 6H, Ar); 12.97 (s, 1H, HCl salt); M/Z (M+H)⁺: 285.8.

### 3-(2-Dimethylamino-phenyl)-pyridine-2,6-diamine hydrochloride Compound No 61

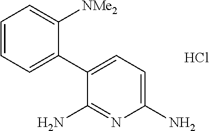 [SMILES: CN(C)c1ccccc1-c1ccc(N)nc1N ; Cl]

Compound No 61 was prepared according to method 20 starting from 2,6-diamino-5-iodopyridine (100 mg, 0.43 mmol, 1.0 eq.) and 2-(dimethylamino)-phenylboronic acid (161 mg, 0.65 mmol, 1.5 eq.). The crude was purified by flash chromatography (SiO₂, CycloHex/EtOAc, 100/00 to 0/100). The resulting foam was further purified by flash chromatography (15 μm, SiO₂, CycloHex/EtOAc, 100/0 to 0/100). The obtained solid was taken up in a mixture of 1 M aqueous HCl/ACN (1/1 v/v) and the resulting solution was lyophilised to afford compound No 61 as a beige solid (7 mg, 6%).

¹H NMR (400 MHz, DMSO-d₆) 80° C. δ: 2.70 (s, 6H, N(CH₃)₂); 6.15 (d, J 8.4 Hz, 1H, Ar); 7.05-7.18 (m, 2H, Ar); 7.20-7.28 (m, 1H, Ar); 7.32-7.42 (m, 1H, Ar); 7.51 (d, J 8.4 Hz, 1H, Ar) NH₂ signals were not observed; M/Z (M+H)⁺: 229.9.

### N-[2-(2,6-Diamino-pyridin-3-yl)-phenyl]-acetamide Compound No 62

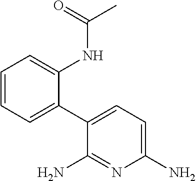 [SMILES: CC(=O)Nc1ccccc1-c1ccc(N)nc1N]

Compound No 62 was prepared according to method 20 starting from 2,6-diamino-5-iodopyridine (100 mg, 0.43 mmol, 1.0 eq.) and 2-acetamidophenylboronic acid (170 mg, 0.65 mmol, 1.5 eq.). The crude was purified by flash chromatography (SiO₂, DCM/MeOH, 100/0 to 95/5). The resulting foam was triturated in pentane, taken up in a 1/1 H₂O/ACN mixture and the resulting solution was lyophilised to afford compound No 62 as a beige solid (40 mg, 38%).

Mp: 60-70° C.; ¹H NMR (400 MHz, DMSO-d₆) δ: 1.84 (s, 3H, C(O)—CH₃); 4.78 (bs, 2H, NH₂); 5.56 (bs, 2H, NH₂); 5.77 (d, J 8.0 Hz, 1H, Ar); 6.88 (d, J 8.0 Hz, 1H, Ar); 7.08-7.14 (m, 2H, NH, Ar); 7.17-7.24 (m, 1H, Ar); 7.55 (d, J 8.0 Hz, 1H, Ar); 8.77 (s, 1H, Ar); M/Z (M+H)⁺: 243.8.

### 3-(2-methylsulfonylphenyl)pyridine-2,6-diamine hydrochloride Compound No 63

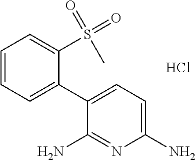 [SMILES: CS(=O)(=O)c1ccccc1-c1ccc(N)nc1N ; Cl]

Compound No 63 was prepared according to method 20 starting from 2,6-diamino-5-iodopyridine (100 mg, 0.43 mmol, 1.0 eq.) and 2-methylsulfonylphenylboronic acid (130 mg, 0.65 mmol, 1.5 eq.). The crude was purified by flash chromatography (SiO₂, DCM/MeOH, 100/0 to 95/5). The resulting foam was further purified by flash chromatography (15 μm, SiO₂, DCM/MeOH, 100/0 to 98/2). The obtained solid was triturated in pentane, then taken up in a mixture of 1 M aqueous HCl/ACN and the resulting solution was lyophilised to afford compound No 63 as a white solid (40 mg, 31%).

Mp: 55-65° C.; ¹H NMR (400 MHz, DMSO-d₆) δ: 3.02 (s, 3H, CH₃); 6.01 (d, J 8.5 Hz, 1H, Ar); 6.84 (bs, 2H, NH₂); 7.38-7.41 (m, 2H, Ar); 7.44 (bs, 2H, NH₂); 7.71 (td, J 7.8, 1.3 Hz, 1H, Ar); 7.79 (td, J 7.8, 1.3 Hz, 1H, Ar); 8.09 (dd, J 7.8, 1.3 Hz, 1H, Ar); 12.94 (s, 1H, HCl salt); M/Z (M+H)⁺: 264.7.

### 3-(2-benzyloxyphenyl)pyridine-2,6-diamine hydrochloride Compound No 64

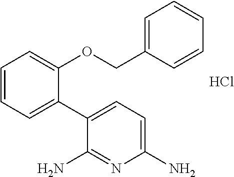 [SMILES: Nc1ccc(-c2ccccc2OCc2ccccc2)c(N)n1 ; Cl]

Compound No 64 was prepared according to method 20 starting from 2,6-diamino-5-iodopyridine (100 mg, 0.43 mmol, 1.0 eq.) and 2-(benzyloxy)phenylboronic acid (148 mg, 0.65 mmol, 1.5 eq.). The crude was purified by flash chromatography (SiO₂, CycloHex/EtOAc, 100/0 to 0/100). The obtained solid was triturated in pentane, then taken up in a mixture of 1 M aqueous HCl/ACN and the resulting solution was lyophilised to afford compound No 64 as a white solid (94 mg, 67%).

Mp: 50-60° C.; ¹H NMR (400 MHz, DMSO-d₆) δ: 5.12 (s, 2H, O—CH₂); 6.00 (d, J 8.5 Hz, 1H, Ar); 6.81 (bs, 2H, NH₂); 7.05 (td, J 7.5, 1.0 Hz, 1H, Ar); 7.17-7.23 (m, 2H, Ar); 7.27-7.41 (m, 8H, Ar and NH₂); 7.44 (d, J 8.5 Hz, 1H, Ar); 12.85 (bs, 1H, HCl salt). M/Z (M+H)⁺: 292.7.

### 3-[2-(cyclopropylmethoxy)phenyl]pyridine-2,6-diamine hydrochloride Compound No 65

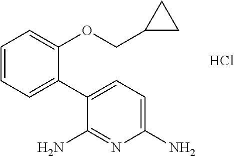 [SMILES: Nc1ccc(-c2ccccc2OCC2CC2)c(N)n1 ; Cl]

Compound No 65 was prepared according to method 20 starting from 2,6-diamino-5-iodopyridine (100 mg, 0.43 mmol, 1.0 eq.) and [2-(cyclopropylmethoxy)phenyl]boronic acid (125 mg, 0.65 mmol, 1.5 eq.). The crude was purified by flash chromatography (SiO₂, CycloHex/EtOAc, 100/0 to 0/100). The obtained solid was triturated in pentane, then taken up in a mixture of 1 M aqueous HCl/ACN and the resulting solution was lyophilised to afford compound No 65 as a beige solid (61 mg, 48%).

Mp: 63-68° C.; ¹H NMR (400 MHz, DMSO-d₆) δ: 0.24-0.32 (m, 2H, CyPr); 0.45-0.54 (m, 2H, CyPr); 1.06-1.18 (m, 1H, CH); 3.85 (d, J 6.7 Hz, 2H, O—CH₂—CyPr); 6.02 (d, J 8.5 Hz, 1H, Ar); 6.72 (bs, 2H, NH₂); 7.00 (td, J 7.5, 0.9 Hz, 1H, Ar); 7.06 (td, J 8.4, 0.9 Hz, 1H, Ar); 7.16 (dd, J 7.5, 1.8 Hz, 1H, Ar); 7.28-7.38 (m, 3H, NH₂+Ar); 7.42 (d, J 8.5 Hz, 1H, Ar); 12.80 (bs, 1H, HCl salt); M/Z (M+H)⁺: 256.9.

### 3-(3-Chloro-2-methyl-phenyl)-5-fluoro-pyridine-2,6-diamine Compound No 66

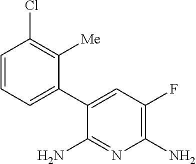 [SMILES: Cc1c(Cl)cccc1-c1cc(F)c(N)nc1N]

Compound No 66 was prepared according to method 20 starting from 2,6-diamino-3-fluoro-5-iodopyridine (100 mg, 0.39 mmol, 1.0 eq.) and 3-chloro-2-methylphenylboronic acid (100 mg, 0.59 mmol, 1.5 eq.). The crude was purified by flash chromatography (SiO₂, CycloHex/EtOAc, 100/0 to 50/50). The resulting foam was further purified by preparative HPLC to afford compound No 66 as a green solid (61 mg, 62%).

Mp: 128-135° C.; ¹H NMR (400 MHz, DMSO-d₆) δ: 2.16 (s, 3H, CH₃); 4.77 (bs, 2H, NH₂); 5.75 (bs, 2H, NH₂); 6.92 (d, J 11.2 Hz, 1H, Ar); 7.10 (dd, J 7.6, 1.2 Hz, 1H, Ar); 7.23 (t, J 7.6 Hz, 1H, Ar); 7.39 (dd, J 7.6, 1.2 Hz, 1H, Ar); M/Z (M[³⁵Cl]₂+H)⁺: 252.8.

### Extension of Methods 17 and 20 for Preparation of Compounds No 67-No 75 in Substituted Napthyl Series

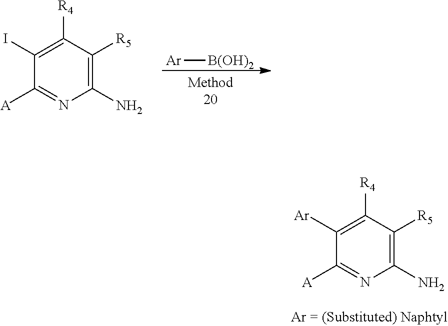

### Compounds of Formula (I) with n is 0

| | | | | | | Com- | HCl |
|---|---|---|---|---|---|---|---|
| A | | | | Meth- | Yield | pound | salt |
| (═R₃) | R₄ | R₅ | Ar | od | (%) | N° | ? |
| Et | H | H | 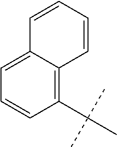 | 17^{a} | 12 | 67^{a} | HCl |
| | | | 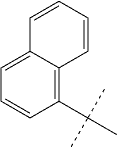 | 20 | 47 | 68 | — |
| | | | 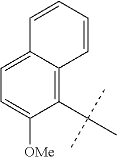 | 20 | 66 | 69 | — |
| | | | 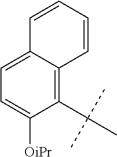 | 20 | 48 | 70 | — |
| NH₂ | H | H | 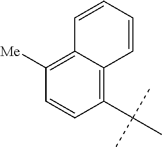 | 20 | 65 | 71 | HCl |
| | | | 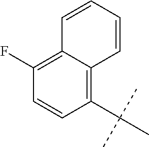 | 20 | 39 | 72 | HCl |
| | | | 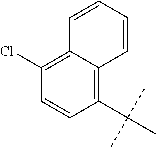 | 20 | 39 | 73 | HCl |
| | | | 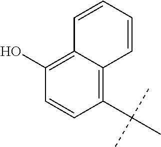 | 20^{b} | 41 | 74^{b} | HCl |
| | | | 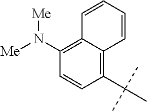 | 20^{b} | 37 | 75^{b} | HCl |

^{a}Reaction performed at 80° C. instead of 90° C.
^{b}Pinacol ester was used instead of the respective boronic acid.
Method 17: PdCl₂(dppf)CH₂Cl₂ (5 mol %), K₂CO₃ aq (1.2M), dioxane, 90° C.
Method 20: PdP(tBu)₃PdG2 (7 mol %), K₂CO₃ aq (1.2M), dioxane, 80° C.

### 6-ethyl-5-(1-naphthyl)pyridin-2-amine hydrochloride Compound No 67

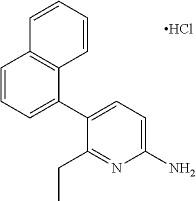 [SMILES: CCc1nc(N)ccc1-c1cccc2ccccc12 ; Cl]

Compound No 67 was prepared according to method 17 starting from 5-bromo-6-ethyl-pyridin-2-amine (100 mg, 0.50 mmol, 1.0 eq.) and 1-naphthylboronic acid (129 mg, 0.75 mmol, 1.5 eq.). The reaction mixture was stirred at 80° C. The crude was purified by flash chromatography (SiO₂, DCM/MeOH 100/0 to 97/3). The resulting foam was further purified by flash chromatography (15 μm, SiO₂, DCM/MeOH 100/0 to 97/3) and triturated in diethyl ether and pentane. The collected solid was taken up in a mixture of 1 M aqueous HCl/ACN and the resulting solution was lyophilised to afford compound No 67 as a brown solid (19 mg, 12%).

Mp: 90-120° C.; ¹H NMR (400 MHz, DMSO-d₆) δ: 1.04 (t, J 7.5 Hz, 3H, CHₐH_{b}—CH₃); 2.28-2.37 (m, 1H, CHₐH_{b}—CH₃); 2.43-2.47 (m, 1H, CHₐH_{b}—CH₃); 6.97 (d, J 8.9 Hz, 1H, Ar); 7.44 (d, J 7.0, 1.0 Hz, 1H, Ar); 7.52-7.53 (m, 2H, Ar); 7.57-7.63 (m, 2H, Ar); 7.77 (d, J 8.9 Hz, 1H, Ar); 7.96-8.05 (m, 4H, NH₂+Ar); 14.20 (bs, 1H, HCl salt); M/Z (M+H)⁺: 249.7.

### 3-(1-naphthyl)pyridine-2,6-diamine Compound No 68

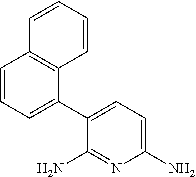 [SMILES: Nc1ccc(-c2cccc3ccccc23)c(N)n1]

Compound No 68 was prepared according to method 20 starting from 2,6-diamino-3-iodopyridine (100 mg, 0.43 mmol, 1.0 eq.) and 1-naphthylboronic acid (112 mg, 0.65 mmol, 1.5 eq.). The crude was purified by flash chromatography (SiO₂, CycloHex/EtOAc 100/0 to 0/100). The resulting foam was triturated twice in pentane to afford compound No 68 as a beige solid (48 mg, 47%).

Mp: 152-158° C.; ¹H NMR (400 MHz, DMSO-d₆) δ: 4.73 (bs, 2H, NH₂); 5.60 (bs, 2H, NH₂); 5.88 (d, J 7.8 Hz, 1H, Ar); 7.01 (d, J 7.8 Hz, 1H, Ar); 7.36 (dd, J 7.0, 1.1 Hz, 1H, Ar); 7.46 (ddd, J 8.2, 6.8, 1.4 Hz, 1H, Ar); 7.51 (ddd, J 8.2, 6.8, 1.4 Hz, 1H, Ar); 7.54 (dd, J 8.2, 7.0 Hz, 1H, Ar); 7.61-7.65 (m, 1H, Ar); 7.86-7.90 (m, 1H, Ar); 7.93-7.97 (m, 1H, Ar); M/Z (M+H)⁺: 236.8.

### 3-(2-methoxy-1-naphthyl)pyridine-2,6-diamine Compound No 69

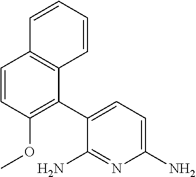 [SMILES: COc1ccc2ccccc2c1-c1ccc(N)nc1N]

Compound No 69 was prepared according to method 20 starting from 2,6-diamino-3-iodopyridine (100 mg, 0.43 mmol, 1.0 eq.) and (2-methoxy-1-naphthyl)boronic acid (150 mg, 0.65 mmol, 1.5 eq.). The crude was purified by flash chromatography (SiO₂, CycloHex/EtOAc 100/0 to 0/100). The resulting foam was triturated twice in pentane to afford compound No 69 as a beige solid (76 mg, 66%).

Mp: 63-67° C.; ¹H NMR (400 MHz, DMSO-d₆) δ: 3.90 (s, 3H, O—CHs); 4.74 (bs, 2H, NH₂); 5.58 (bs, 2H, NH₂); 5.86 (d, J 8.0 Hz, 1H, Ar); 6.96-7.01 (m, 2H, Ar); 7.25-7.32 (m, 2H, Ar); 7.45 (ddd, J 8.0, 6.7, 1.2 Hz, 1H, Ar); 7.52 (d, J 8.0 Hz, 1H, Ar); 7.85 (d, J 8.0 Hz, 1H, Ar); M/Z (M+H)⁺: 266.7.

### 3-(2-isopropoxy-1-naphthyl)pyridine-2,6-diamine Compound No 70

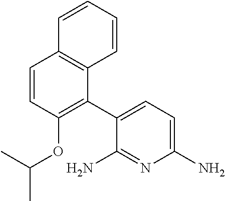 [SMILES: CC(C)Oc1ccc2ccccc2c1-c1ccc(N)nc1N]

Compound No 70 was prepared according to method 20 starting from 2,6-diamino-3-iodopyridine (100 mg, 0.43 mmol, 1.0 eq.) and (2-isopropoxy-1-naphthyl)boronic acid (150 mg, 0.65 mmol, 1.5 eq.). The crude was purified by flash chromatography (SiO₂, CycloHex/EtOAc 100/0 to 0/100). The resulting foam was triturated twice in pentane to afford compound No 70 as a beige solid (60 mg, 48%).

Mp: 40-43° C.; ¹H NMR (400 MHz, DMSO-d₆) δ: 1.16 (dd, J 11.9, 6.0 Hz, 6H, O—CH(CH₃)₂); 4.48-4.58 (m, 3H, O—CH(CH₃)₂ and NH₂); 5.47 (bs, 2H, NH₂); 5.86 (d, J 8.0 Hz, 1H, Ar); 6.90 (d, J 8.0 Hz, 1H, Ar); 7.31-7.46 (m, 4H, Ar); 7.82-7.91 (m, 2H, Ar); M/Z (M+H)⁺: 294.7.

### 3-(4-methyl-1-naphthyl)pyridine-2,6-diamine hydrochloride Compound No 71

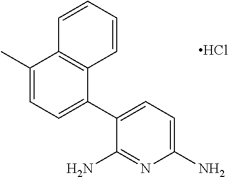 [SMILES: Cc1ccc(-c2ccc(N)nc2N)c2ccccc12 ; Cl]

Compound No 71 was prepared according to method 20 starting from 2,6-diamino-3-iodopyridine (100 mg, 0.43 mmol, 1.0 eq.) and (4-methyl-1-naphthyl)boronic acid (121 mg, 0.65 mmol, 1.5 eq.). The crude was purified by flash chromatography (SiO₂, CycloHex/EtOAc 100/0 to 0/100). The resulting foam was triturated in pentane. The collected solid was taken up in a mixture of 1 M aqueous HCl/ACN and the resulting solution was lyophilised to afford compound No 71 as a beige solid (80 mg, 65%).

Mp: 127-132° C.; ¹H NMR (400 MHz, DMSO-d₆) δ: 2.70 (s, 3H, CH₃); 6.10 (d, J 8.5 Hz, 1H, Ar); 7.68 (bs, 2H, NH₂); 7.30 (d, J 7.1 Hz, 1H, Ar); 7.38-7.47 (m, 4H, NH₂+Ar); 7.45 (ddd, J 8.2, 7.0, 1.2 Hz, 1H, Ar); 7.57-7.63 (m, 2H, Ar); 8.06-8.14 (m, 1H, Ar); 12.99 (bs, 1H, HCl salt); M/Z (M+H)⁺: 250.8.

### 3-(4-fluoro-1-naphthyl)pyridine-2,6-diamine hydrochloride Compound No 72

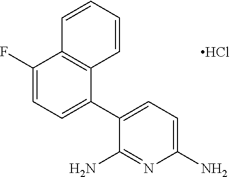 [SMILES: Nc1ccc(-c2ccc(F)c3ccccc23)c(N)n1 ; Cl]

Compound No 72 was prepared according to method 20 starting from 2,6-diamino-3-iodopyridine (100 mg, 0.43 mmol, 1.0 eq.) and (4-fluoro-1-naphthyl)boronic acid (123 mg, 0.65 mmol, 1.5 eq.). The crude was purified by flash chromatography (SiO₂, CycloHex/EtOAc 100/0 to 0/100). The resulting foam was triturated in pentane. The collected solid was taken up in a mixture of 1 M aqueous HCl/ACN and the resulting solution was lyophilised to afford compound No 72 as a beige solid (48 mg, 39%).

Mp: 119-126° C.; ¹H NMR (400 MHz, DMSO-d₆) δ: 6.11 (d, J 8.5 Hz, 1H, Ar); 7.77 (bs, 2H, NH₂); 7.38-7.50 (m, 5H, Ar); 7.60-7.72 (m, 3H, Ar); 8.14 (dt, J 8.5, 1.2 Hz, 1H, Ar); 13.07 (bs, 1H, HCl salt); M/Z (M+H)⁺: 254.8.

### 3-(4-chloro-1-naphthyl)pyridine-2,6-diamine hydrochloride Compound No 73

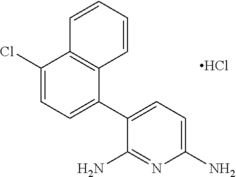 [SMILES: Nc1ccc(-c2ccc(Cl)c3ccccc23)c(N)n1 ; Cl]

Compound No 73 was prepared according to method 20 starting from 2,6-diamino-3-iodopyridine (100 mg, 0.43 mmol, 1.0 eq.) and (4-chloro-1-naphthyl)boronic acid (134 mg, 0.65 mmol, 1.5 eq.). The crude was purified by flash chromatography (SiO₂, CycloHex/EtOAc 100/0 to 0/100). The resulting foam was triturated in pentane. The collected solid was taken up in a mixture of 1 M aqueous HCl/ACN and the resulting solution was lyophilised to afford compound No 73 as a white solid (51 mg, 39%).

Mp: 135-140° C.; ¹H NMR (400 MHz, DMSO-d₆) δ: 6.10 (d, J 8.5 Hz, 1H, Ar); 6.80 (bs, 2H, NH₂); 7.41 (d, J 7.6 Hz, 1H, Ar); 7.47 (d, J 8.5 Hz, 1H, Ar); 7.49 (bs, 2H, NH₂); 7.59-7.69 (m, 2H, Ar); 7.74 (ddd, J 8.2, 6.6, 1.8 Hz, 1H, Ar); 7.77 (d, J 7.6 Hz, 1H, Ar); 8.28 (dt, J 8.5, 0.9 Hz, 1H, Ar); 13.07 (bs, 1H, HCl salt); M/Z (M+H)⁺: 270.7.

### 4-(2,6-diamino-3-pyridyl)naphthalen-1-ol hydrochloride Compound No 74

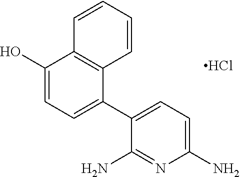 [SMILES: Nc1ccc(-c2ccc(O)c3ccccc23)c(N)n1 ; Cl]

Compound No 74 was prepared according to method 20 starting from 2,6-diamino-3-iodopyridine (100 mg, 0.43 mmol, 1.0 eq.) and 4-(4,4,5,5-tetramethyl-1,3,2-dioxaborolan-2-yl)naphthalen-1-ol (176 mg, 0.65 mmol, 1.5 eq.). The crude was purified by flash chromatography (SiO₂, CycloHex/EtOAc 100/0 to 0/100). The resulting foam was triturated in pentane. The collected solid was suspended in an aqueous solution of 1M HCl and the resulting suspension was lyophilised to afford compound No 74 as a beige solid (51 mg, 41%).

Mp: 160-166° C.; ¹H NMR (400 MHz, DMSO-d₆) δ: 6.07 (d, J 8.5 Hz, 1H, Ar); 6.61 (bs, 2H, NH₂); 7.94 (d, J 1.1 Hz, 1H, Ar); 7.19 (d, J 1.1 Hz, 1H, Ar); 7.36 (bs, 2H, NH₂); 7.42 (d, J 8.5 Hz, 1H, Ar); 7.44-7.50 (m, 3H, Ar); 8.19-8.25 (m, 1H, Ar); 10.37 (s, 1H, OH); 12.79 (bs, 1H, HCl salt); M/Z (M+H)⁺: 252.8.

### 3-[4-(dimethylamino)-1-naphthyl]pyridine-2,6-diamine hydrochloride Compound No 75

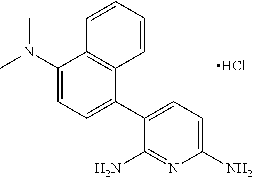 [SMILES: CN(C)c1ccc(-c2ccc(N)nc2N)c2ccccc12 ; Cl]

Compound No 75 was prepared according to method 20 starting from 2,6-diamino-3-iodopyridine (100 mg, 0.43 mmol, 1.0 eq.) and N,N-dimethyl-4-(4,4,5,5-tetramethyl-1,3,2-dioxaborolan-2-yl)naphthalen-1-amine (153 mg, 0.65 mmol, 1.5 eq.). The crude was purified by flash chromatography (SiO₂, CycloHex/EtOAc 100/0 to 0/100). The resulting foam was triturated in pentane. The collected solid was taken up in a mixture of 1 M aqueous HCl/ACN and the resulting solution was lyophilised to afford compound No 75 as a brown solid (50 mg, 37%).

Mp: 175-181° C.; ¹H NMR (400 MHz, DMSO-d₆) 80° C. δ: 2.95 (s, 6H, N—(CH₃)₂); 6.17 (d, J 8.5 Hz, 1H, Ar); 125-129 (m, 1H, Ar); 7.33-7.37 (m, 1H, Ar); 7.45 (d, J 8.5 Hz, 1H, Ar); 7.49-7.61 (m, 3H, Ar); 8.30-8.35 (m, 1H, Ar); NH₂ signals were not observed; HCl salt signal was not observed; M/Z (M+H)⁺: 279.8.

### Method 21 for Preparation of Compound No 76 in Substituted Phenyl Series

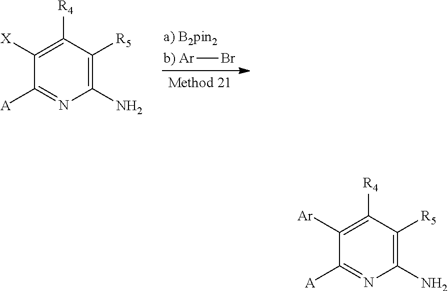

### Compounds of Formula (I) with n is 0

| | | | | | | | Com- | HCl |
|---|---|---|---|---|---|---|---|---|
| A | | | | | Meth- | Yield | pound | salt |
| (═R₃) | R₄ | R₅ | X | Ar | od | (%) | N° | ? |
| Et | H | H | Br | 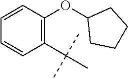 | 21 | 38 | 76 | HCl |

Method 21:
a) Pd₂(dba)₃ (3.6 mol %), PdCl₂dppf (14 mol %), B₂Pin₂ (1.7 eq.), KOAc (2.8 eq.), dioxane, 90° C.
b) SPhosPdG₂ (5 mol %), K₂CO_{3 aq} (1.2M), dioxane, 90° C.

Method 21:

Step a: Formation of the Aryl Boronate

In a microwave vial, to a solution of the aryl bromide (1.4 eq.) in dioxane (C=0.2 M) were successively added bis(pinacolato)diboron (1.7 eq.) and 1,1′-bis(diphenylphosphino)ferrocene (14 mol %) and KOAc (2.8 eq.). The resulting mixture was degassed with argon bubbling for 15 min. Tris(dibenzylideneacetone)dipalladium (3.6 mol %) was then added in one portion. The vial was sealed and the mixture was stirred at 90° C. for 40 h.

Step b. Suzuki Coupling

The reaction mixture of step a was cooled to rt and then at rt under Ar, were successfully added 1-bromo-2-(cyclopentyloxy)benzene (1.0 eq.) and aqueous K₂CO₃ (1.2 M, 2.0 eq.). The resulting mixture was further degassed with argon bubbling for 15 min and SPhosPdG2 (10 mol %) was then added in one portion. The vial was sealed and the mixture was stirred at 90° C. for 17 h. The reaction mixture was cooled to rt and subsequently hydrolysed. The aqueous layer was extracted with DCM, washed with brine, and the organic layer was dried over MgSO₄, filtered and concentrated in vacuo. The residue was purified by chromatography. The obtained solid was further purified when necessary. For specific examples, the corresponding hydrochloride salt has been prepared.

### 5-[2-(cyclopentoxy)phenyl]-6-ethyl-pyridin-2-amine hydrochloride Compound No 76

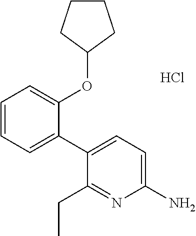 [SMILES: CCc1nc(N)ccc1-c1ccccc1OC1CCCC1 ; Cl]

Compound No 76 was prepared according to method 21 starting from 2-amino-5-bromo-6-ethylpyridine (100 mg, 0.50 mmol, 1.4 eq.). The crude was purified by flash chromatography (SiO₂, DCM/MeOH, 100/0 to 95/5). The resulting foam was further purified by flash chromatography (SiO₂, Biotage® SNAP KP-NH, CycloHex/EtOAc, 100/0 to 0/100). The obtained solid was taken up in a mixture of 1 M aqueous HCl/ACN and the resulting solution was lyophilised to afford compound No 76 as a white solid (43 mg, 38%).

Mp: 80-93 NMR (400 MHz, DMSO-d₆) δ: 1.10 (t, 77.6 Hz, 3H, CH₂—CH₃); 1.49-1.60 (m, 6H, CyPent); 1.79-1.87 (m, 2H, CH₂); 2.50-2.52 (m, 2H, CH₂—CH₃); 4.81-4.85 (m, 1H, O—CH); 6.87 (d, J 9.0 Hz, 1H, Ar); 7.02 (td, J 7.5, 0.9 Hz, 1H, Ar); 7.12 (dd, J 8.2, 0.9 Hz, 1H, Ar); 7.17 (dd, J 7.5, 1.8 Hz, 1H, Ar); 7.39 (ddd, J 8.2, 7.5, 1.8 Hz, 1H, Ar); 7.67 (d, J 9.0 Hz, 1H, Ar); 7.89 (bs, 2H, NH₂); 14.06 (bs, 1H, HCl salt); M/Z (M+H)⁺: 283.8.

### Method 22 for Preparation of Compound No 77 in Substituted Phenyl Series

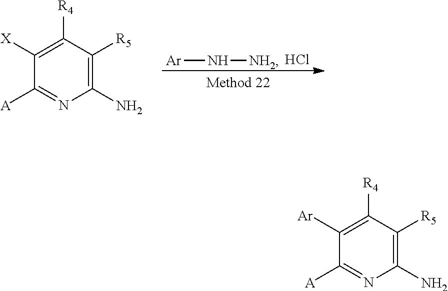

### Compounds of Formula (I) with n is 0

| | | | | | | | Com- | HCl |
|---|---|---|---|---|---|---|---|---|
| A | | | | | Meth- | Yield | pound | salt |
| (═R₃) | R₄ | R₅ | X | Ar | od | (%) | N° | ? |
| NH₂ | H | H | H | 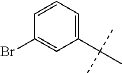 | 22 | 49 | 77 | — |

Method 22: K₂CO₃, DMSO, rt.

Method 22:

In a round bottom flask, solution of 2,6-diaminopyridine (10.0 eq.) and aryl hydrazine hydrochloride (1.0 eq.) in DMSO (C=0.1 M) was stirred overnight at rt. The reaction mixture was then hydrolysed and the aqueous layer was extracted once with EtOAc and washed with brine. The organic layer was dried over MgSO₄, filtered and concentrated in vacuo. The residue was purified by chromatography. The obtained solid was further purified when necessary.

### 3-(4-bromophenyl)pyridine-2,6-diamine Compound No 77

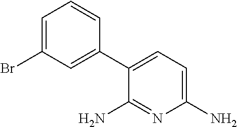 [SMILES: Nc1ccc(-c2cccc(Br)c2)c(N)n1]

Compound No 77 was prepared according to method 22 starting from 2,6-diaminopyridine (571 mg, 5.23 mmol, 10.0 eq.) and 3-bromophenylhydrazine hydrochloride (117 mg, 0.52 mmol, 1.0 eq.). The crude was purified by flash chromatography (SiO₂, DCM/MeOH, 100/0 to 80/20). The obtained solid was triturated in pentane to afford compound No 77 as a brown solid (68 mg, 49%).

Mp: 60-62° C.; ¹H NMR (400 MHz, DMSO-d₆) δ: 5.18 (bs, 2H, NH₂); 5.62 (s, 2H, NH₂); 5.83 (d, J 8.1 Hz, 1H, Ar); 7.07 (d, J 8.1 Hz, 1H, Ar); 7.29-7.44 (m, 3H, Ar); 7.51 (t, J 1.8 Hz, 1H, Ar); M/Z (M+H[⁷⁹Br])⁺: 264.5.

### Method 23 for the Preparation of Additional Compounds No 78-No 91 in HetAr Series

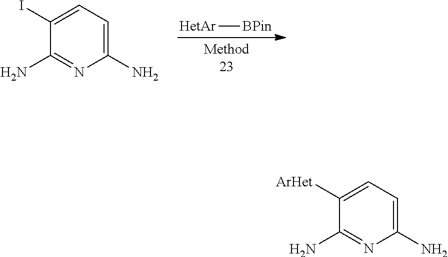

### Compounds of Formula (I) with n is 0

| | Yield | | Compound |
|---|---|---|---|
| HetAr (═Ar) | (%) | Method | N° |
| 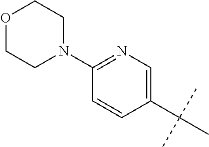 | 47 | 23 | 78 |
| 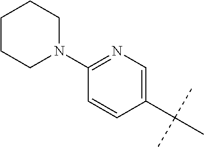 | 20 | 23 | 79 |
| 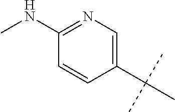 | 63 | 23 | 80 |
| 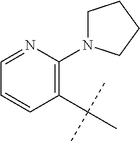 | 39^{a} | 23 | 81^{a} |
| 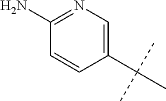 | 65 | 23 | 82 |
| 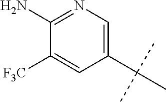 | 49 | 23 | 83 |
| 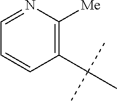 | 61 | 23 | 84 |
| 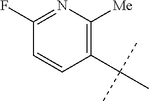 | 30^{a} | 23 | 85^{a} |
| 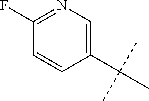 | 47^{a} | 23 | 86^{a} |
| 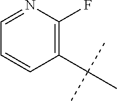 | 67^{a} | 23 | 87^{a} |
| 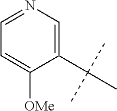 | 59^{a, b} | 23 | 88 |
| 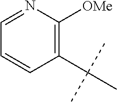 | 43^{a} | 23 | 89^{a} |
| 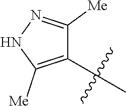 [SMILES: *c1c(C)n[nH]c1C] | 56^{a, b} | 23 | 90^{a, b} |
| 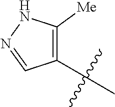 [SMILES: *c1cn[nH]c1C] | 88 | 23 | 91 |

Method 23: XPhosPdG₂ (5 mol %), Na₂CO₃ aq, EtOH, 90° C.
^{a}The heteroaryl pinacol boronic acid was used instead of the corresponding heteroaryl pinacol boronate,
^{b}Performed in EtOH/toluene 9/1 instead of EtOH.

Method 23:

In a microwave vial, to a suspension of 3-iodopyridine-2,6-diamine (1.0 eq.) in absolute EtOH (C=0.2 M) were added successively the heteroaryl pinacol boronate (1.2-1.5 eq.) and an aqueous solution of Na₂CO₃ (1.2 M, 1.5 eq.). The resulting suspension was degassed by 15 min of Ar bubbling and XPhosPdG2 (5 mol %) was then added in one portion. The vial was sealed and the mixture was stirred at 90° C. until no more evolution was noticed by UPLC-MS (overnight, unless mentioned otherwise). The reaction mixture was cooled to rt and subsequently filtered through a Celite® pad and rinsed with MeOH and/or DCM. The filtrate was concentrated to dryness and purified by flash chromatography (conditions summarised below). The product was further purified when necessary (conditions summarised below).

### 3-(6-morpholino-3-pyridyl)pyridine-2,6-diamine Compound No 78

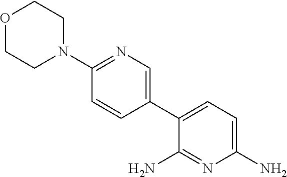 [SMILES: Nc1ccc(-c2ccc(N3CCOCC3)nc2)c(N)n1]

Compound No 78 was prepared according to method 23 starting from 2,6-diamino-3-iodopyridine (100 mg, 0.43 mmol, 1.0 eq.) and 6-(morpholin-4-yl)pyridine-3-boronic acid pinacol ester (151 mg, 0.52 mmol, 1.2 eq.). The crude was purified by flash chromatography (SiO₂, DCM/MeOH, 100/0 to 90/10) to afford compound No 78 as a grey solid (55 mg, 47%).

Mp: 201-205° C.; ¹H NMR (400 MHz, DMSO-d₆) δ: 3.41-3.45 (m, 4H, 2 N—CH₂—CH₂—O); 3.68-3.73 (m, 4H, 2 N—CH₂—CH₂—O); 5.02 (bs, 2H, NH₂); 5.48 (bs, 2H, NH₂); 5.80 (d, J 8.0 Hz, 1H, Ar); 6.85 (d, J 8.7 Hz, 1H, Ar); 6.99 (d, J 8.0 Hz, 1H, Ar); 7.54 (dd, J 8.7, 2.6 Hz, 1H, Ar); 8.09 (dd, J 2.6 Hz, 1H, Ar); M/Z (M+H)⁺: 272.6.

### 3-[6-(1-piperidyl)-3-pyridyl]pyridine-2,6-diamine Compound No 79

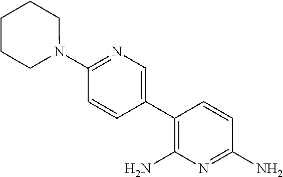 [SMILES: Nc1ccc(-c2ccc(N3CCCCC3)nc2)c(N)n1]

Compound No 79 was prepared according to method 23 starting from 2,6-diamino-3-iodopyridine (100 mg, 0.43 mmol, 1.0 eq.) and 6-(piperidin-1-yl)pyridine-3-boronic acid pinacol ester (187 mg, 0.65 mmol, 1.5 eq.). The crude was purified by flash chromatography (SiO₂, DCM/MeOH, 100/0 to 95/5) and then triturated in Et₂O and in MeOH to afford compound No 79 as a white solid (23 mg, 20%).

Mp: 155-159° C.; ¹H NMR (400 MHz, DMSO-d₆) δ: 1.55-1.61 (m, 6H, 3 CH₂); 3.47-3.54 (m, 4H, 2 N—CH₂); 4.99 (bs, 2H, NH₂); 5.46 (bs, 2H, NH₂); 5.79 (d, J 8.0 Hz, 1H, Ar); 6.81 (d, J 8.8 Hz, 1H, Ar); 6.98 (d, J 8.0 Hz, 1H, Ar); 7.47 (dd, J 8.8, 2.5 Hz, 1H, Ar); 8.04 (d, J 2.5 Hz, 1H, Ar); M/Z (M+H)⁺: 270.6.

### 3-[6-(methylamino)-3-pyridyl]pyridine-2,6-diamine Compound No 80

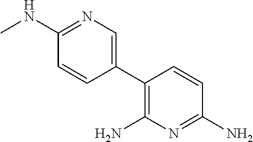 [SMILES: CNc1ccc(-c2ccc(N)nc2N)cn1]

Compound No 80 was prepared according to method 23 starting from 2,6-diamino-3-iodopyridine (33 mg, 0.14 mmol, 1.0 eq.) and 6-(Methylamino)-3-pyridinyl boronic acid pinacol ester (51 mg, 0.22 mmol, 1.5 eq.). The crude was purified by flash chromatography (SiO₂, DCM/MeOH, 95/5 to 90/10) and then triturated in Et₂O to afford compound No 80 as a grey solid (19 mg, 63%).

Mp: 178-182° C.; ¹H NMR (400 MHz, DMSO-d₆) δ: 2.78 (d, J 4.9 Hz, 3H, NH—CHs); 5.00 (bs, 2H, NH₂); 5.48 (bs, 2H, NH₂); 5.80 (d, J 8.0 Hz, 1H, Ar), 6.40 (q, 74.9 Hz, 1H, NH—CH₃); 6.46 (dd, J 8.6, 0.6 Hz, 1H, Ar); 6.96 (d, J 8.0 Hz, 1H, Ar); 7.35 (dd, J 8.6, 2.4 Hz, 1H, Ar); 7.92 (dd, J 2.4, 0.6 Hz, 1H, Ar); M/Z (M+H)⁺: 216.6.

### 3-(6-pyrrolidin-1-yl-3-pyridyl)pyridine-2,6-diamine Compound No 81

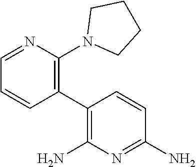 [SMILES: Nc1ccc(-c2cccnc2N2CCCC2)c(N)n1]

Compound No 81 was prepared according to method 23 starting from 2,6-diamino-3-iodopyridine (100 mg, 0.43 mmol, 1.0 eq.) and 2-(pyrrolidin-1-yl)pyridine-3-boronic acid (125 mg, 0.65 mmol, 1.5 eq.). The crude was purified by flash chromatography (SiO₂, DCM/MeOH, 100/0 to 90/10) and then triturated in Et₂O to afford compound No 81 as a pale grey solid (43 mg, 39%).

Mp: 186-192° C.; ¹H NMR (400 MHz, DMSO-d₆) δ: 1.71-1.74 (m, 4H, 2 CH₂); 3.12-3.15 (m, 4H, 2 N—CH₂); 4.84 (bs, 2H, NH₂); 5.47 (bs, 2H, NH₂); 5.78 (d, J 8.0 Hz, 1H, Ar); 6.67 (dd, J 1.2, 4.8 Hz, 1H, Ar); 6.92 (d, J 8.0 Hz, 1H, Ar); 7.24 (dd, J 7.2, 1.9 Hz, 1H, Ar); 8.03 (dd, J 4.8, 1.9 Hz, 1H, Ar); M/Z (M+H)⁺: 256.6.

### 3-(6-amino-3-pyridyl)pyridine-2,6-diamine Compound No 82

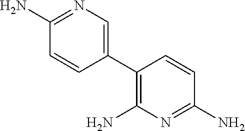 [SMILES: Nc1ccc(-c2ccc(N)nc2N)cn1]

Compound No 82 was prepared according to method 23 starting from 2,6-diamino-3-iodopyridine (100 mg, 0.43 mmol, 1.0 eq.) and 2-aminopyridine-5-boronic acid pinacol ester (125 mg, 0.52 mmol, 1.2 eq.). 2.2 mL of an aqueous solution of Na₂CO₃ (0.6 M, 1.29 mmol, 3.0 eq.). The crude was purified by flash chromatography (SiO₂, DCM/MeOH, 100/0 to 90/10), triturated in Et₂O and then further purified by flash chromatography (15 μm, SiO₂, DCM/MeOH, 95/5 to 90/10) to afford compound No 82 as a yellow solid (56 mg, 65%).

Mp: 220-240° C.; ¹H NMR (400 MHz, DMSO-d₆) δ: 5.83 (bs, 2H, NH₂); 5.89 (d, J 8.2 Hz, 1H, Ar); 6.20 (bs, 2H, NH₂); 6.38 (bs, 2H, NH₂); 6.60 (d, J 8.7 Hz, 1H, Ar); 7.17 (d, J 8.2 Hz, 1H, Ar); 7.45 (dd, J 8.7, 2.3 Hz, 1H, Ar); 7.85 (d, J 2.3 Hz, 1H, Ar); M/Z (M+H)⁺: 202.5.

### 3-[6-amino-5-(trifluoromethyl)-3-pyridyl]pyridine-2,6-diamine Compound No 83

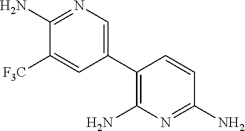 [SMILES: Nc1ccc(-c2cnc(N)c(C(F)(F)F)c2)c(N)n1]

Compound No 83 was prepared according to method 23 starting from 2,6-diamino-3-iodopyridine (75 mg, 0.32 mmol, 1.0 eq.) and 2-amino-3-(trifluoro)pyridine-5-boronic acid pinacol ester (138 mg, 0.48 mmol, 1.5 eq.). The crude was purified by flash chromatography (SiO₂, DCM/MeOH, 100/0 to 90/10) and then triturated in Et₂O, to afford compound No 83 as a light beige solid (42 mg, 49%).

Mp: 154-155° C.; ¹H NMR (400 MHz, DMSO-d₆) δ: 5.13 (bs, 2H, NH₂); 5.51 (bs, 2H, NH₂); 5.79 (d, J 8.0 Hz, 1H, Ar); 6.30 (s, 2H, NH₂); 7.00 (d, J 8.0 Hz, 1H, Ar); 7.62 (d, J 2.0 Hz, 1H, Ar); 8.14 (d, J 2.0 Hz, 1H, Ar); M/Z (M+H)⁺: 270.6.

### 3-(2-methyl-3-pyridyl)pyridine-2,6-diamine Compound No 84

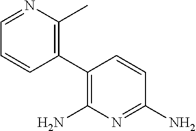

Compound No 84 was prepared according to method 23 starting from 2,6-diamino-3-iodopyridine (75 mg, 0.32 mmol, 1.0 eq.) and 2-methylpyridine-3-boronic acid pinacol ester (105 mg, 0.48 mmol, 1.5 eq.). The crude was purified by flash chromatography (SiO₂, DCM/MeOH, 97/13 to 90/10) and then triturated in Et₂O, to afford compound No 84 as a white solid (39 mg, 61%).

Mp: 192-198° C.; ¹H NMR (400 MHz, DMSO-d₆) δ: 2.31 (s, 3H, CH₃); 4.88 (bs, 2H, NH₂); 5.51 (bs, 2H, NH₂); 5.79 (d, J 8.0 Hz, 1H, Ar); 6.89 (d, J 8.0 Hz, 1H, Ar); 7.21 (dd, J 7.6, 4.7 Hz, 1H, Ar); 7.45 (dd, J 7.6, 1.7 Hz, 1H, Ar); 8.38 (dd, J 4.7, 1.7 Hz, 1H, Ar); M/Z (M+H)⁺: 201.5.

### 3-(6-fluoro-2-methyl-3-pyridyl)pyridine-2,6-diamine Compound No 85

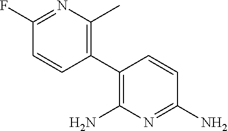 [SMILES: Cc1nc(F)ccc1-c1ccc(N)nc1N]

Compound No 85 was prepared according to method 23 starting from 2,6-diamino-3-iodopyridine (75 mg, 0.32 mmol, 1.0 eq.) and 2-fluoro-6-picoline-5-boronic acid (74 mg, 0.48 mmol, 1.5 eq.). The crude was purified by flash chromatography (SiO₂, DCM/MeOH, 100/0 to 90/10) and then triturated in Et₂O, to afford compound No 85 as a white solid (21 mg, 30%).

Mp: 186-192° C.; ¹H NMR (400 MHz, DMSO-d₆) δ: 2.24 (s, 3H, CH₃); 5.00 (bs, 2H, NH₂); 5.52 (bs, 2H, NH₂); 5.78 (d, J 8.0 Hz, 1H, Ar); 6.88 (d, J 8.0 Hz, 1H, Ar); 6.96 (dd, J 8.3, 3.0 Hz, 1H, Ar); 7.61 (t, J 8.3 Hz, 1H, Ar); M/Z (M+H)⁺: 219.6.

### 3-(6-fluoro-3-pyridyl)pyridine-2,6-diamine Compound No 86

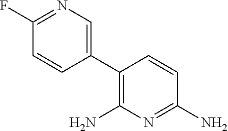 [SMILES: Nc1ccc(-c2ccc(F)nc2)c(N)n1]

Compound No 86 was prepared according to method 23 starting from 2,6-diamino-3-iodopyridine (75 mg, 0.32 mmol, 1.0 eq.) and 6-fluoro-3-pyridinylboronic acid (68 mg, 0.48 mmol, 1.5 eq.). The crude was purified by flash chromatography (SiO₂, DCM/MeOH, 100/0 to 95/5) and then triturated in Et₂O, to afford compound No 86 as a beige solid (31 mg, 47%).

Mp: 170-178° C.; ¹H NMR (400 MHz, DMSO-d₆) δ: 5.26 (bs, 2H, NH₂); 5.63 (bs, 2H, NH₂); 5.83 (d, J 8.0 Hz, 1H, Ar); 7.06 (d, J 8.0 Hz, 1H, Ar); 7.15 (dd, J 8.4, 2.7 Hz, 1H, Ar); 7.92 (td, J 8.4, 2.7 Hz, 1H, Ar); 8.14-8.15 (m, 1H, Ar); M/Z (M+H)⁺: 205.6.

### 3-(2-fluoro-3-pyridyl)pyridine-2,6-diamine Compound No 87

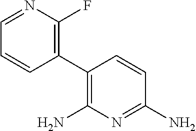 [SMILES: Nc1ccc(-c2cccnc2F)c(N)n1]

Compound No 87 was prepared according to method 23 starting from 2,6-diamino-3-iodopyridine (75 mg, 0.32 mmol, 1.0 eq.) and 2-fluoro-3-pyridineboronic acid (68 mg, 0.48 mmol, 1.5 eq.). The crude was purified by flash chromatography (SiO₂, DCM/MeOH, 100/0 to 95/5) and then triturated in Et₂O, to afford compound No 87 as a beige solid (44 mg, 67%).

Mp: 184-188° C.; ¹H NMR (400 MHz, DMSO-d₆) δ: 5.19 (bs, 2H, NH₂); 5.64 (bs, 2H, NH₂); 5.80 (d, J 8.0 Hz, 1H, Ar); 7.00 (dd, J 8.0, 0.8 Hz, 1H, Ar); 7.34 (ddd, J 7.3, 4.8, 2.0 Hz, 1H, Ar); 7.84 (ddd, J 10.1, 7.3, 2.0 Hz, 1H, Ar); 8.12 (ddd, J 4.8, 2.0, 0.8 Hz, 1H, Ar); M/Z (M+H)⁺: 205.6.

### 3-(4-methoxy-3-pyridyl)pyridine-2,6-diamine Compound No 88

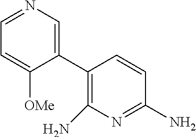 [SMILES: COc1ccncc1-c1ccc(N)nc1N]

Compound No 88 was prepared according to method 23 starting from 2,6-diamino-3-iodopyridine (100 mg, 0.43 mmol, 1.0 eq.) and 4-methoxy-3-piridinebornic acid pinacol ester (150 mg, 0.64 mmol, 1.5 eq.). The crude was purified by flash chromatography (SiO₂, DCM/MeOH, 100/0 to 90/10) and then triturated in Et₂O, to afford compound No 88 as a beige solid (55 mg, 59%).

Mp: 129-199° C.; ¹H NMR (400 MHz, DMSO-d₆) δ: 3.83 (s, 3H, O—CHs); 5.51 (bs, 2H, NH₂); 5.85 (d, J 8.0 Hz, 1H, Ar); 6.08 (bs, 2H, NH₂); 7.09 (d, J 8.0 Hz, 1H, Ar); 7.12 (d, 75.8 Hz, 1H, Ar); 8.19 (s, 1H, Ar); 8.42 (d, J 5.8 Hz, 1H, Ar); M/Z (M+H)⁺: 217.3.

### 3-(2-methoxy-3-pyridyl)pyridine-2,6-diamine Compound No 89

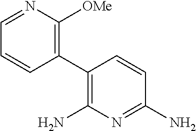 [SMILES: COc1ncccc1-c1ccc(N)nc1N]

Compound No 89 was prepared according to method 23 starting from 2,6-diamino-3-iodopyridine (100 mg, 0.43 mmol, 1.0 eq.) and 2-methoxy-3pyridinylboronic acid (98 mg, 0.64 mmol, 1.5 eq.). The crude was purified by flash chromatography (SiO₂, DCM/MeOH, 100/0 to 90/10) and then triturated in Et₂O, to afford compound No 89 as a white solid (40 mg, 43%).

Mp: 174-176° C.; ¹H NMR (400 MHz, DMSO-d₆) δ: 3.84 (s, 3H, O—CHs); 4.89 (bs, 2H, NH₂); 5.51 (bs, 2H, NH₂); 5.78 (d, J 8.0 Hz, 1H, Ar); 6.95 (d, J 8.0 Hz, 1H, Ar); 7.00 (dd, J 7.2, 4.9 Hz, 1H, Ar); 7.51 (dd, J 7.2, 1.9 Hz, 1H, Ar); 8.10 (dd, J 4.9, 1.9 Hz, 1H, Ar); M/Z (M+H)⁺: 217.3.

### 3-(3,5-dimethyl-1H-pyrazol-4-yl)pyridine-2,6-diamine Compound No 90

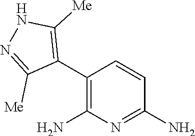 [SMILES: Cc1n[nH]c(C)c1-c1ccc(N)nc1N]

Compound No 90 was prepared according to method 23 starting from 2,6-diamino-3-iodopyridine (50 mg, 0.21 mmol, 1.0 eq.) and 3,5-dimethylpyrazole-4-boronic acid pinacol ester (56 mg, 0.25 mmol, 1.2 eq.) in EtOH/toluene 9/1. The crude was purified by flash chromatography (SiO₂, DCM/MeOH, 95/5 to 90/10) and then triturated in Et₂O and pentane, to afford compound No 90 as a grey solid (24 mg, 56%).

Mp>250° C.; ¹H NMR (400 MHz, DMSO-d₆) δ: 2.00 (s, 6H, 2 CH₃); 5.94 (d, J 8.3 Hz, 1H, Ar); 6.18 (bs, 2H, NH₂); 6.76 (bs, 2H, NH₂); 7.22 (d, J 8.3 Hz, 1H, Ar); 12.27 (bs, 1H, NH); M/Z (M+H)⁺: 204.5.

### 3-(5-methyl-1H-pyrazol-4-yl)pyridine-2,6-diamine Compound No 91

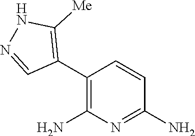 [SMILES: Cc1[nH]ncc1-c1ccc(N)nc1N]

Compound No 91 was prepared according to method 23 starting from 2,6-diamino-3-iodopyridine (75 mg, 0.32 mmol, 1.0 eq.) and 3-methyl-1H-pyrazole-4-boronic acid pinacol ester (100 mg, 0.48 mmol, 1.5 eq.), and using 3.0 eq. of Na₂CO₃ (1.2 M in water). The crude was purified by flash chromatography (SiO₂, DCM/MeOH, 95/5 to 90/10) and then triturated in Et₂O, to afford compound No 91 as a grey solid (53 mg, 88%).

Mp: 118-222° C.; ¹H NMR (400 MHz, DMSO-d₆) δ: 2.10 (s, 3H, CH₃); 5.77 (bs, 2H, NH₂); 5.88 (d, J 8.0 Hz, 1H, Ar); 6.30 (bs, 2H, NH₂); 7.15 (d, J 8.0 Hz, 1H, Ar); 7.42 (bs, 1H, Ar); 12.58 (bs, 1H, NH); M/Z (M+H)⁺: 190.5.

### Method A for the Synthesis of Non-Commercially Available Aryl-Bromides:

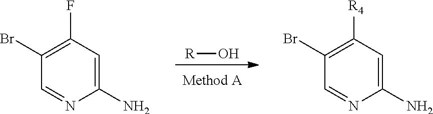

| | | Yield | |
|---|---|---|---|
| R | R₄ | (%) | Compound N° |
| —(CH₂)₂—NH₂ | —O—(CH₂)₂—NH₂ | 53 | 92 |
| —CH₂—CF₃ | —O—CH₂—CF₃ | 76 | 93 |

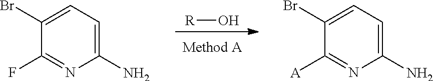

| | | Yield | |
|---|---|---|---|
| R | A | (%) | Compound N° |
| —(CH₂)₂—NH₂ | —O—(CH₂)₂—NH₂ | 81 | 94 |
| —CH₂—CF₃ | —O—CH₂—CF₃ | 80 | 95 |

Method A:

To a suspension of sodium hydride (1.5 eq.) in DMF (C=0.2 M) at 0° C. was added the alcohol (2.0 eq.) and the resulting mixture was stirred for 10 min at 0° C. Then the aryl fluoride (1.0 eq.) was added and the mixture was stirred for 3-16 h at rt. The reaction mixture was subsequently hydrolysed. The aqueous layer was extracted twice with EtOAc, washed with brine, and the organic layer was dried over a hydrophobic filter or over MgSO₄, filtered and concentrated in vacuo. The residue was purified by flash chromatography (conditions summarised below) to afford the desired aryl bromide.

### 6-(2-aminoethoxy)-5-bromo-pyridin-2-amine

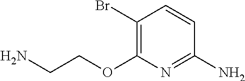 [SMILES: NCCOc1nc(N)ccc1Br]

6-(2-aminoethoxy)-5-bromo-pyridin-2-amine was prepared according to method A starting from 5-bromo-6-fluoro-pyridin-2-amine (400 mg, 2.09 mmol, 1.0 eq.) and ethanolamine (252 μL, 4.2 mmol, 2.0 mmol). The crude was purified by flash chromatography (SiO₂, Biotage® SNAP KP-NH, DCM/MeOH, 100/0 to 95/5) to afford 6-(2-aminoethoxy)-5-bromo-pyridin-2-amine as a yellow oil (444 mg, 81%).

¹H NMR (400 MHz, DMSO-d₆) δ: 2.82 (t, J 6.0 Hz, 2H, N—CH₂); 4.13 (t, J 6.0 Hz, 2H, O—CH₂); 5.97 (d, J 8.3 Hz, 1H, Ar); 6.06 (bs, 2H, NH₂); 7.45 (d, J 8.3 Hz, 1H, Ar); CH₂—NH₂ signal not observed; M/Z (M[⁷⁹Br]+H)⁺: 232.5.

### 5-bromo-6-(2,2,2-trifluoroethoxy)pyridin-2-amine

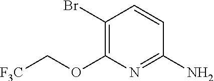 [SMILES: Nc1ccc(Br)c(OCC(F)(F)F)n1]

5-bromo-6-(2,2,2-trifluoroethoxy)pyridin-2-amine was prepared according to method A starting from 5-bromo-6-fluoro-pyridin-2-amine (400 mg, 2.09 mmol, 1.0 eq.) and 2.2.2-trifluoroethanol (305 μL, 4.2 mmol, 2.0 eq.). The crude was purified by flash chromatography (SiO₂, Biotage® SNAP KP-NH, CycloHex/EtOAc, 100/0 to 50/50) to afford 5-bromo-6-(2,2,2-trifluoroethoxy)pyridin-2-amine as a beige solid (510 mg, 80%).

¹H NMR (400 MHz, DMSO-d₆) δ: 4.92 (q, 79.1 Hz, 2H, O—CH₂—CF₃); 6.08 (d, J 8.3 Hz, 1H, Ar); 6.28 (bs, 2H, NH₂); 7.54 (d, J 8.3 Hz, 1H, Ar); M/Z (M[⁷⁹Br]+H)⁺: 271.5.

### 4-(2-aminoethoxy)-5-bromo-pyridin-2-amine

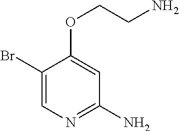 [SMILES: NCCOc1cc(N)ncc1Br]

4-(2-aminoethoxy)-5-bromo-pyridin-2-amine was prepared according to method A starting from 5-bromo-4-fluoro-pyridin-2-amine (430 mg, 2.25 mmol, 1.0 eq.) and ethanolamine (271 μL, 4.50 mmol, 2.0 eq.). After hydrolysis and before the general work-up, the pH of the reaction mixture was adjusted to 8. The crude was purified by flash chromatography (SiO₂, Biotage® SNAP KP-NH, DCM/MeOH, 100/0 to 95/5) to afford 4-(2-aminoethoxy)-5-bromo-pyridin-2-amine (276 mg, 53%).

¹H NMR (400 MHz, DMSO-d₆) δ: 1.51 (bs, 2H, CH₂—NH₂); 2.88 (t, J 5.8 Hz, 2H, N—CH₂—CH₂—O); 3.92 (t, 75.8 Hz, 2H, N—CH₂—CH₂—O); 6.01 (bs, 2H, NH₂); 6.10 (s, 1H, Ar); 7.83 (s, 1H, Ar); M/Z (M+H[⁷⁹Br])⁺: 232.4.

### 5-bromo-4-(2,2,2-trifluoroethoxy)pyridin-2-amine

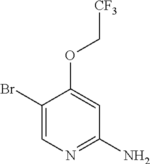 [SMILES: Nc1cc(OCC(F)(F)F)c(Br)cn1]

5-bromo-4-(2,2,2-trifluoroethoxy)pyridin-2-amine was prepared according to method A starting from 5-bromo-4-fluoro-pyridin-2-amine (360 mg, 1.88 mmol, 1.0 eq.) and 2.2.2-trifluoroethanol (275 μL, 3.76 mmol, 2.0 eq.). The crude was purified by flash chromatography (SiO₂, DCM/MeOH, 100/0 to 80/20) to afford 5-bromo-4-(2,2,2-trifluoroethoxy)pyridin-2-amine (398 mg, 76%). ¹H NMR (400 MHz, DMSO-d₆) δ: 4.82 (q, J 8.7 Hz, 2H, O—CH₂—CF₃); 6.16 (s, 1H, Ar); 6.17 (bs, 2H, NH₂); 7.90 (s, 1H, Ar); M/Z (M[⁷⁹Br]+H)⁺: 271.5.

### Method 20 for the Preparation of Compound No 96

### 2-(2,6-diamino-3-pyridyl)benzonitrile hydrochloride Compound No 96

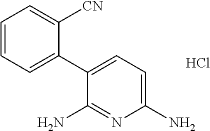 [SMILES: N#Cc1ccccc1-c1ccc(N)nc1N ; Cl]

Compound No 96 was prepared according to method 20 starting from 2,6-diamino-5-iodopyridine (100 mg, 0.43 mmol, 1.0 eq.) and 2-cyanophenylboronic acid (96 mg, 0.65 mmol, 1.5 eq.). The crude was purified by flash chromatography (SiO₂, DCM/MeOH, 100/0 to 90/10). The obtained solid was triturated in pentane, then taken up in a mixture of 1 M aqueous HCl/ACN and the resulting solution was lyophilised to afford Compound No 96 as a brown solid (21 mg, 20%).

Mp>250° C.; ¹H NMR (400 MHz, DMSO-d₆) 80° C. δ: 6.76 (d, J 9.0 Hz, 1H, Ar); 7.44 (bs, 2H, NH₂); 7.59-7.69 (m, 1H, Ar); 7.86-7.96 (m, 1H, Ar); 8.15-8.59 (m, 4H, Ar+NH₂); 8.69 (d, J 9.0 Hz, 1H, Ar); HCl salt signal was not observed; M/Z (M+H)⁺: 211.8.

### 13—Pharmacological Data

### Example 21

Radioligand Binding Assays

Competition experiments with membranes from CHO cells stably expressing hNPFFR1 and hNPFFR2 were performed essentially as described in Elhabazi, K., et al. Neuropharmacology, 2013. 75C, 164-171. [1], Briefly, hNPFFR1 or hNPFFR2 membranes (5 to 10 μg of proteins) were incubated (1 hr at 25° C.; 0.25 mL total volume) with 0.015 nM [D-Tyr1[125I], N-MePhe3]-NPFF (Hartmann Analytic GmbH) in 50 mM HEPES (pH 7.4), 1 mM CaCl2, 1 mM MgCl2 and 0.1% bovine serum albumin. Non-specific binding was determined in the presence of 10 μM RFRP-3. Incubation was terminated by rapid filtration through a 96-well GF/B unifilter apparatus (Perkin Elmer Life and Analytical Sciences, Courtaboeuf, France). Unifilters were washed five times with binding buffer, then dried for 1 h at 65° C. After addition of 40 μL scintillation cocktail (Microscint-O, Perkin Elmer) per well, bound radioactivity was determined on a TopCount scintillation counter (Perkin Elmer).

In Vitro Results

Tables 6-13: Binding assay with both receptors NPFFR1 & 2

TABLE 6
3-aryl 2,6 diaminopyridines

| N° | 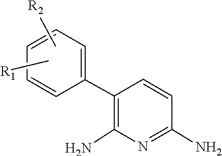 R₁ | R₂ | NPFF1-R Ki (nM) | NPFF2-R Ki (nM) |
|---|---|---|---|---|
| 1a | H | H | 188 | 7,300 |
| 1b | 2-Cl | H | 39 | 1,300 |
| 1c | 2-Me | H | 23 | 30,000 |
| 1d | 2-Et | H | 6,2 | 2,000 |
| 1e | 2-i-Pr | H | 8 | 980 |
| 1f | 2-CF₃ | H | 22.4 | 6,700 |
| 1g | 2-CH₂—OMe | H | 58 | 1,200 |
| 2a | 2-OH | H | 11.8 | 1,100 |
| 2b | 2-OMe | H | 13.8 | 3,000 |
| 2c | 2-OCF₃ | H | 34 | 10,000 |
| 2d | 2-OEt | H | 15 | 700 |
| 2e | 2-O-nBu | H | 10.5 | 1,800 |
| 2f | 2-OiPr | H | 11.3 | 2,100 |
| 2g | 2-O-iBu | H | 9 | 700 |
| 2h | 2-O(CH₂)₂OMe | | 26 | 35,700 |
| 2j | 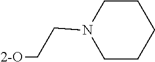 | H | 33 | 450 |
| 2i | 2-O-cPent | H | 4 | 280 |
| 2k | 2-OMe | 4-F | 27.6 | 3,200 |
| 1r | 2-Ph | H | 20.7 | 700 |
| 1h | 3-Cl | H | 90 | 300 |
| 1i | 4-Cl | H | 786 | 8,800 |
| 1j | 2-Cl | 3-Cl | 20 ± 2 | 2,000 ± |
| | | | | 300 |
| lk | 2-Cl | 4-Cl | 28 | 1,180 |
| 1l | 2-Cl | 5-Cl | 335 | 26,650 |
| lm | 2-Cl | 6-Cl | 94 | >10,000 |
| 1p | 2-Cl | 3-CF₃ | 65 | 4,600 |
| lq | 2-Me | 3-Cl | 112 | 7,300 |
| 2l | 2-MeO | 3-MeO | 26 | 900 |
| 2m | 2-MeO | 4-MeO | 45.7 | 1,870 |
| 1n | 3-Cl | 4-Cl | 313 | 2,560 |
| 1o | 3-Cl | 5-Cl | ~500 | ~5,000 |

TABLE 7
2,6-diamino 3-heteroaryl pyridines

| | 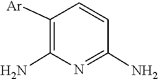 | NPFF1-R | NPFF2-R |
|---|---|---|---|
| N° | Ar | Ki (nM) | Ki (nM) |
| 1j | 2,3Cl₂—Ph | 20 ± 2 | 2,000 ± 300 |
| 3b | 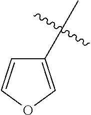 [SMILES: *c1ccoc1] | n.s. | n.s. |
| 3a | 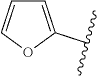 [SMILES: *c1ccco1] | 5,080 ^{a} | 106,000 |
| 3c | 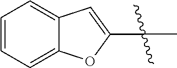 [SMILES: *c1cc2ccccc2o1] | 524 | 34,000 |
| 3d | 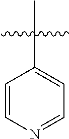 [SMILES: *c1ccncc1] | 5,390 | — |
| 3e | 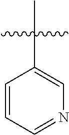 [SMILES: *c1cccnc1] | 1,930 | — |
| 3f | 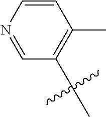 [SMILES: *c1cnccc1C] | 515 | ns |

ns: not significant,
—: not determined

TABLE 8
Substitutions of the nitrogen in position 2

| | 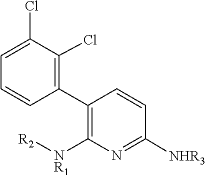 | | | NPFF1-R | NPFF2-R |
|---|---|---|---|---|---|
| N° | R₁ | R₂ | R₃ | Ki (nM) | Ki (nM) |
| lj | H | H | H | 20 ± 2 | 2,000 ± 300 |
| 5a | Me | H | H | 81.2 | 2,300 |
| 5b | Et | H | H | 79.2 | 2,200 |
| 5c | n-Bu | H | H | 416 | 6,900 |
| 5e | 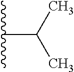 [SMILES: *C(C)C] | H | H | 250 | 1,900 |
| 5d | CH₂—Ph | H | H | 84 | 1,900 |
| 5f | (CH₂)₂—Ph | H | H | 424 | 800 |
| 5g | (CH₂)₂—OMe | H | H | 481 | ns |
| 5h | 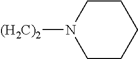 | H | H | 1,920 | 3,200 |
| 5i | 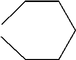 | — | H | 440 | 6,300 |

ns: not significant,
-: not determined

TABLE 9
Replacement of 2-amino group by alkyl or alkoxy groups

| | 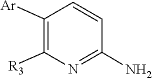 | | NPFF1-R | NPFF2-R |
|---|---|---|---|---|
| N° | Ar | R₃ | Ki nM | Ki nM |
| 6a | 2,3-Cl₂—Ph | H | 577 | 4,200 |
| 6b | 2,3-Cl₂—Ph | Me | 81.4 | 2,000 |
| 6c | 2,3-Cl₂—Ph | Et | 25 | 800 |
| 6d | 2-OMe—Ph | Et | 15.2 | 960 |
| 6e | 2-OMe—Ph | CH₂OMe | 461 | 18,600 |
| 6f | 2-OMe—Ph | CF₃ | >5,000 | >10,000 |
| 6g | 2-OMe—Ph | n-Pr | 6.3 | 470 |
| 6h | 2-OMe—Ph | i-Pr | 35.7 | 1,500 |
| 6i | 2-OMe—Ph | c-Pr | 31 | 1,400 |
| 7a | 2-OMe—Ph | OMe | ns | ns |
| 1j | 2,3-Cl₂—Ph | NH₂ | 20 ± 2 | 2,000 ± 300 |

TABLE 10
Additional substitutions at position 5 (R)

| | 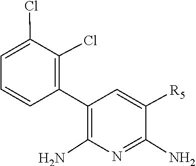 | NPFF1-R | NPFF2-R |
|---|---|---|---|
| N° | R5 | Ki nM | Ki nM |
| 9a | F | 3,235 | 1,800 |
| 9b | Et | 1,090 | 3,300 |

TABLE 11
Additional substitutions at position 4 (R4)

| | 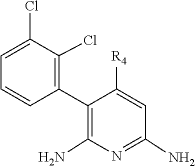 | NPFF1-R | NPFF2-R |
|---|---|---|---|
| N° | R4 | Ki nM | Ki (nM) |
| 8a | OMe | 4,080 | >50,000 |
| 8b | 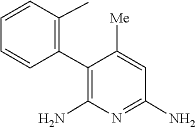 | 183 | 4,600 |

### Tables 12 and 13: Homologues

| | 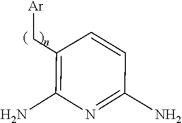 | NPFF1-R | NPFF2-R |
|---|---|---|---|
| N° | Ar(CH₂)n— | Ki nM | Ki (nM) |
| 1a | Ph | 188 | 7.3 |
| 10a | —CH₂—Ph | 76 ± 12 | 2,800 |
| 11a | —(CH₂)₂—Ph | 189 | 5,400 |

| | 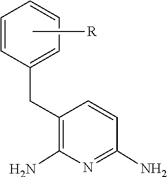 | NPFF1-R | NPFF2-R |
|---|---|---|---|
| N° | R | Ki nM | Ki (nM) |
| 10a | H | 76 ± 12 | 2,800 |
| 10b | 4-F | 122 | 4,200 |
| 10c | 4-Cl | 56 | 2,700 |
| 10d | 3-Cl | 19 | 900 |
| 10e | 2-Cl | 3.7 | 450 |
| 10f | 2, 4-Cl₂ | 3.7 | 500 |

Glo Sensor cAMP assay in HEK-293 stably expressing hNPFFR1 and hNPFFR2 Methods

The functional characterization of the selected compounds was performed by Glo Sensor cAMP kinetic assay, a validated approach for measuring Gαi/s activation by GPCRs (DiRaddo et al., 2014; Gilissen et al., 2015). HEK-Glo cells expressing either NPFF1 or NPFF2 receptors were suspended (10⁶ cells per mF) in physiological Hepes buffer (10 mM HEPES, 0.4 mM NaH₂PO₄, 137.5 mM NaCl, 1.25 mM MgCl₂, 1.25 mM CaCl₂, 6 mM KCl, 10 mM glucose and 1 mg/mF bovine serum albumin, pH 7.4) supplemented with 1 mM D-Fuciferine. After equilibration for 2 h at 25° C., luminescence levels were recorded in real time in 96-well plates using a Flexstation^{R} 3 (Molecular Devices, Sunnyvale Calif., USA). Non-specific effect of the different compounds was evaluated in HEK-Glo cells without any recombinant human RF-amide receptors. To test the agonist activity on NPFF1R and NPFF2R, compounds were injected 15 minutes before addition of forskolin (0.5 μM) and readings were pursued for 90 minutes. To evaluate their antagonist properties, compounds were pre-incubated with cells for 15 minutes before prototypical agonists for NPFF1R (RFRP-3) and NPFF2R (NPFF) receptors. According to their preferential coupling to G_{i/o} proteins, the stimulation of NPFF1R and NPFF2R by agonists was monitored as a dose-dependent reduction in steady-state luminescence levels, reflecting the inhibition of forskolin-induced cAMP accumulation. Experiments were performed at 25° C. in the presence of 0.5 mM IBMX to prevent the degradation of cAMP by phosphodiesterases.

Results

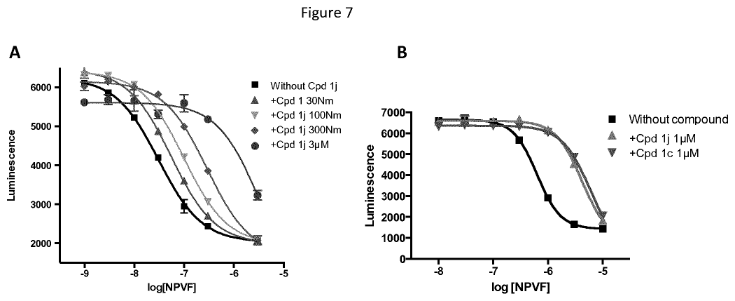

Please refer to FIG. 7.

When tested alone compound 1j had no effect on cells expressing hNPFF1R and hNPFF2R. (A) In hNPFF1R cells, compound 1j efficiently shifted to the right the dose response curve of NPVF, the endogenous agonist of NPFF1R, demonstrating that this compound displays efficient antagonist activity on this receptor. (B) compound 1c displayed antagonist activity similar as compound 1j when tested on HEK-293 cells expressing hNPFF1R.

### Example 22: Human NPFFR1 Evaluation Using BRET Biosensors (IC50)

Compounds of the present invention were tested successively for their agonist and antagonist activities on human NPFFR1 (hNPFFR1) receptor transiently over-expressed in HEK-293 T cells. Compounds exert agonist activity if, by themselves in absence of neuropeptide RFRP-3 (also named NPVF) they activate hNPFFR1; and they exert antagonist activity if they decrease the action of RFRP-3 on the receptor. The assay used to measure compound activity is based on BRET (Bioluminescence Resonance Energy Transfer) biosensors and is designed to monitor the plasma membrane translocation of protein that interacts with specific Ga subunit. The specific effector (luciferase tagged: BRET donor) recruited at the membrane will be in close proximity to a plasma membrane anchor (GFP tagged: BRET acceptor) to induce a BRET signal (Ramdan et al, 2006, Chapter 5, Current Protocols in Neuroscience).

Cell Culture and Transfection

HEK-293 T cells are maintained in Dulbecco's Modified Eagle's Medium supplemented with 10% Foetal Calf Serum, 1% Penicillin/Streptomycin at 37° C./5% CO₂. Cells are co-transfected using polyethylenimine (25 kDa linear) with four DNA plasmids encoding; hNPFFR1, GαoB, a Gi family specific intracellular effector fused to luciferase (BRET donor), a plasma membrane effector fused to GFP (BRET acceptor). After transfection, cells are cultured for 48 h at 37° C./5% CO₂.

BRET Assay

Receptor activity is detected by changes in BRET signal.

On the day of the assay, cells are detached using trypsin 0.05%, resuspended in assay buffer (1.8 mM CaCl2, 1 mM MgCl2, 2.7 mM KCl, 137 mM NaCl, 0.4 mM NaH2PO4, 5.5 mM D-Glucose, 11.9 mM NaHCO3, 25 mM Hepes) and seeded in 384 well plate at a density of 20,000 cells per well. Then, plates are equilibrated 3.5 hours at 37° C. before adding compounds.

Compounds and luciferase substrate are added to the cells using an automated device (Freedom Evo®, Tecan) and BRET readings are collected on EnVision (PerkinElmer) with specific filters (410 nm BW 80 nm, 515 nm BW 30 nm).

Agonist and antagonist activities of compounds are consecutively evaluated on the same cell plate. Agonist activity is first measured after 10 minutes incubation with compound alone on the cells. Then, cells are stimulated by an EC80 RFRP-3 concentration and luminescence is recorded for additional 10 minutes. EC80 RFRP-3 concentration is the concentration giving 80% of the maximal RFRP-3 response. Agonist or antagonist activities are evaluated in comparison to basal signals evoked by assay buffer or EC80 RFRP-3 alone, respectively.

IC50 Determination

For IC50 determination, a dose-response test is performed using 20 concentrations (ranging over 6 logs) of each compound. Dose-response curves are fitted using the sigmoidal dose-response (variable slope) analysis in GraphPad Prism software (GraphPad Software) and IC50 of antagonist activity is calculated. Dose-response experiments are performed in duplicate, in two independent experiments.

According to the biological test procedure, the following compounds showed IC₅₀ ranges as detailed below.

IC₅₀>1000 nM:

Compounds 1i, 86, 41, 89, 27, 84, 57, 6a, 2l, 1n, 85, 6e, 2m, 63, 52, 8b, 10a, 88, 10b, 22, 1o, 81, 46, 5d, 5a, 5e, 1a, 2j, 40, 39, 26, 54.

IC₅₀ between 1000-500 nM:

Compounds 10c, 1h, 1g, 6b, 70, 5b, 2a, 49, 10d, 50, 47, 74, 1b, 2k, 6h, 45, 2h, 6i, 2c, 1f, 6c.

IC₅₀ between 500-100 nM:

Compounds 1p, 1c, 2b, 55, 69, 61, 71, 29, 56, 2d, 6d, 53, 59, 44, 2e, 1d, 51, 43, 6g, 1l, 1k, 58, 1r, 10e, 1e, 2f, 2g, 1j, 10f, 48, 67, 30, 2i, 96.

IC₅₀<100 nM:

Compounds 73, 42, 65, 64, 76, 68, 72, 31.

### 14—Activity of NPFF1R Receptor Antagonist Compound 1j in Models of Opioid-Induced Hyperalgesia and Analgesic Tolerance, Surgical Pain and Neuropathic Pain Example 23

### 1. Purpose:

Evaluation of the activity of NPFF1R receptor antagonist compound 1j in models of opioid-induced hyperalgesia and analgesic tolerance, surgical pain and neuropathic pain

Material

Fentanyl citrate, Gabapentine, Nonidet P40, Tween 80 and Kolliphor EF were purchased from Sigma-Aldrich (Saint Quentin Fallavier, France). Morphine hydrochloride was from Francopia (Paris, France). Ketamine (Imalgene), Xylasine (Rompun) and Isoflurane (Vetflurane) were purchased from Centravet (Nancy, France). Chlorehexidine alcoolique (2%) was from Mediq (Fretin, France), compound 1j was dissolved either in Tween 80 (0.5%) or Kolliphor EF (10%). Fentanyl, morphine, gabapentine and RF9 were dissolved in physiological saline (0.9%). All compounds were administered at 10 mF/kg (vol/body weight). Isoflurane was dissolved in Nonidet P40 (Ethyl phenyl-polyethylene-glycol).

Nociception tests were performed on C57BF6/N male mice (25-30 g weight; Janvier Fabs). NPFFR1 KO and wild-type littermates on a 100% C57BF6/N genetic background were obtained from the Mouse Clinical Institute, (Illkirch). Animals were housed in groups of two to five per cage and kept under a 12 h/12 h light/dark cycle at 21±1° C. with ad libitum access to food and water and were habituated to the testing room and equipment and handled for one week before starting behavioral experiments. All experiments were carried out in strict accordance with the European guidelines for the care of laboratory animals (European Communities Council Directive 2010/63/EU) and approved by the local ethical committee (CREMEAS). All efforts were made to minimize animal discomfort and to reduce the number of animals used.

### 2. Methods

### 2.1. Opioid-Induced Hyperalgesia

### 2.1.1 Fentanyl-Induced Hyperalgesia

Experiments were designed in mice according to a protocol enabling the visualization of an early analgesic effect of fentanyl and of a delayed hyperalgesic state lasting for several days (Celerier et al., 2000; Elhabazi et al., 2012). After habituation and handling of tested animals, nociceptive baseline was assessed at d-4, d-3, d-2 and d-1 using tail immersion test. At d0, animals received four consecutive injections (4×60 μg/kg, s.c., 15 min interval) of fentanyl and nociceptive responses were measured every 1 h after the last fentanyl injection until return to baseline values to monitor its short lasting analgesic effect. The long lasting hyperalgesic effect of fentanyl was tested the days after fentanyl injections (days 1-4) using the tail immersion test. The anti-hyperalgesic preventive effect of compound 1j (5 mg/kg, p.o.) was evaluated by pretreatment of mice with this compound 35 min before fentanyl administration at d0. In a subsequent experiment, we tested the dose-effect of compound 1j on fentanyl-induced hyperalgesia at three doses, 0.2, 1 and 5 mg/kg (sc).

### 2.1.2. Morphine-Induced Hyperalgesia and Tolerance

In this model, hyperalgesia and tolerance were induced by daily administration of morphine (10 mg/kg, s.c.) for 10 days (day0 to day9). After habituation and handling of tested animals, nociceptive baseline was assessed at d-4, d-3, d-2 and d-1 using tail immersion test. To evaluate the short lasting morphine analgesia at day 0 and development of tolerance to morphine effect at day 9, nociceptive response was measured 30 min after morphine injection (5 mg/kg, s.c.) at the first and at the last day of treatments (d0 and d9, respectively). The long lasting morphine hyperalgesia was evaluated by once-daily measurement (d1 to d8) of the basal nociceptive response 60 min before morphine administration (10 mg/kg, s.c.).

The anti-hyperalgesic effect of compound 1j (5 mg/kg, p.o.) was evaluated by pretreatment of mice with this compound 35 min before each administration of morphine.

### 2.1.3 Measurement of the Heat Nociceptive Response

The nociceptive response of mice was determined by using tail immersion test as previously described (Elhabazi et al., 2014; Simonin et al., 1998). Briefly, mice were restrained in a grid pocket and their tail was immersed in a thermostated water bath. The latency (in sec) for tail withdrawal from hot water (48±0.5° C.) was taken as a measure of the nociceptive response. In the absence of any nociceptive reaction, a cut-off value of 25 sec was set to avoid tissue damage.

2.2 Pain Models Associated with Nociceptive Hypersensitivity

### 2.2.1 Incisional Pain Model

### 2.2.1.1 Surgery

The incisional pain procedure was conducted in mice as previously described by Pogatzki and Raja, (2003) with slight modifications. Mice were anesthetized with isoflurane (30%) delivered via a nose cone. After antiseptic preparation of the right hind paw with 2% chlorhexidine alcoolique solution, a 0.7 cm longitudinal incision was made with a number 11 blade through the skin and fascia of the plantar surface of the right hind paw, starting 0.3 cm from the proximal edge of the heel and extending toward the toes. The underlying plantaris muscle was then elevated with a curved forceps and incised longitudinally leaving the muscle origin and insertion intact. Finally, the skin was closed by two 5-0 nylon sutures and the wound was covered with 2% chlorhexidine alcoolique solution. After surgery, mice were allowed to recover in their cages under a heat source.

### 2.2.1.2 Experimental Protocol

In a first set of experiments before incision, mice were allowed to habituate to restrainer boxes for 30 min during three consecutive days. After habituation, nociceptive mechanical baseline was assessed at d-3, d-2 and d-1 before incision procedure using Von Frey filaments. After incision, all mice were treated daily from d1 to d6 with Compound 1j (5 mg/kg, po) in combination or not with morphine (2.5 mg/kg, sc.) Mechanical nociceptive thresholds were measured daily 30 min after the sc. injection of morphine using Von Frey filaments.

### 2.2.2 Neuropathic Pain Model

### 2.2.2.1 Surgery

To induce neuropathic pain in mice, it was performed a chronic constriction injury (CCI) of sciatic nerve following the method originally described in rats by Bennett et al., (1986) and adapted for mice by Mika et al., (2007). Surgical procedure was performed under aseptic conditions and deep anesthesia (ketamine+xylasine). After shaving and cleaning the hindpaw with chlorhexidine solution, an incision at the mid-thigh level was performed to expose the common left sciatic nerve. Three ligatures of 4-0 nylon spaced 1 min were tied loosely around the nerve until a slight twitch in the ipsilateral hind limb was observed. Finally, the skin was closed by two 5-0 nylon sutures and the wound was covered with chlorhexidine solution. After surgery, mice were allowed to recover in their cages under a heat source.

### 2.2.2.1 Experimental Protocol

In a first set of experiments before surgery, mice were allowed to habituate individually to restrainer boxes for 30 min during three consecutive days. After habituation, nociceptive mechanical baseline was assessed at d-3, d-2 and d-1 pre-CCI using Von Frey filaments. After surgery (d11 post-CCI), mice were subjected before any treatment to a pre-test using Von Frey filaments to verify the development of neuropathic pain. On the basis of the pre-test data, mice were randomly divided into four groups treated orally with compound 1j (5 mg/kg) or vehicle 35 min before subcutaneous injection of morphine (3 mg/kg) or saline.

Administration of all drugs started after surgery at d11 and continues until d 21. Mechanical nociceptive thresholds were assessed 30 min after the sc. injection of morphine or saline at the following days: d11, d13, d15, d17, d19 and d21. In order to test a reference compound in our experimental conditions, mice from group 1 were treated at d22 by gabapentin (5 mg/kg, sc.) and their mechanical threshold was measured in Von frey test at 30 and 60 min after treatment.

### 2.2.3 Von Frey Test

Mechanical allodynia was assessed by Von Frey test as previously described by (Celerier et al., 2006). This procedure consists in measuring nociceptive responses to Von Frey filaments stimulation, which are normally non-noxious punctuate mechanical stimuli. Mice were placed individually in transparent boxes with a wire grid bottom through which the filaments were applied under the hind paws (both ipsi and contralateral paw). Clear withdrawal, shaking or licking of the paw were considered as nociceptive responses. It was started with the filament 0.4 g, then the strength of the next filament was increased or decreased according to the up down procedure (Chaplan et al., 1994). In total, eight filaments were used (0.008 to 2 g), the upper limit value (2 g) was recorded even if there was no withdrawal response to this force. The threshold of response was calculated from the sequence of filament strength by using an Excel program that includes curve fitting of the data.

### 2.3 Data Analyses

Data for nociceptive tests are expressed as mean values±S.E.M. for 6 to 12 animals depending on the group and the experiment. Analgesia was quantified as the area under the curve (AUC) calculated by the trapezoidal method (Celerier et al., 2000). In the fentanyl and morphine induced hyperalgesia experiments, the overall hyperalgesia was quantified as hyperalgesia index, which is calculated by the trapezoidal method and in which the baseline value of each mouse determined before treatment was subtracted from each experimental value. In the incisional pain experiment, the overall allodynia during the whole course of the experiment was quantified as the allodynia index. Allodynia index was calculated by the trapezoidal method in which the baseline value determined before paw incision for each animal was subtracted from each experimental value. In the CCI experiment, we subtracted from each experimental value the mechanical threshold value obtained with the pre-test at d11. According to the experiment (see results section and legends), data were analyzed using one-way, two-way or repeated measures analysis of variance (ANOVA). Post-hoc analyses were performed with Fisher's PLSD test. Data were also analyzed by Unpaired or Paired t-test when appropriate. The level of significance was set at p<0.05. All statistical analyses were carried out using the StatView software.

### 3. Results

### 3.1 Compound 1j Prevents Long Lasting Hyperalgesia Induced by Fentanyl in Mice

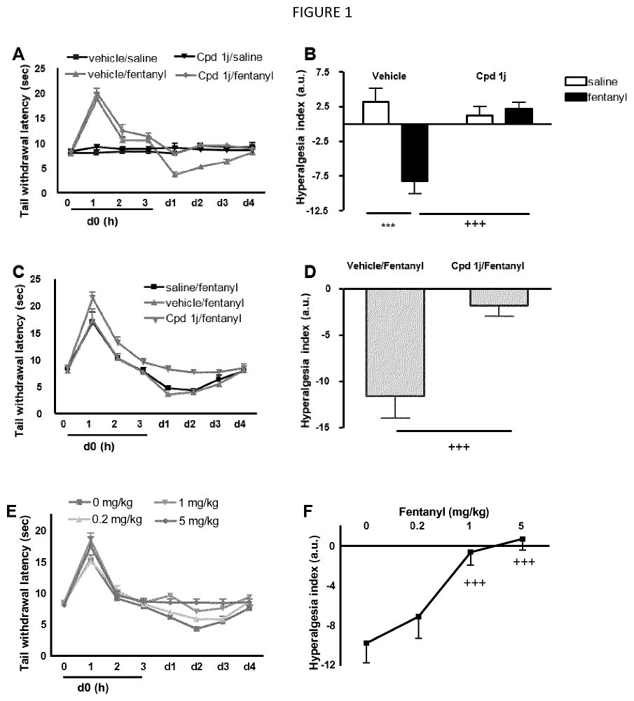

In a previous work, it has been shown that the pharmacological blockade of two NPFF receptors (NPFF1R and NPFF2R) by RF9 prevents the development of fentanyl-induced hyperalgesia (Elhabazi et al. 2012). Here, the capacity of compound 1j, a non peptidic selective antagonist of the NPFF1 receptor subtype, to prevent the development of fentanyl-induced hyperalgesia following oral administration was investigated. First, its activity in our model of fentanyl-hyperalgesia with two different vehicules: Tween 80 (0.5%) and Kolliphor EE (10%) was evaluated. As shown in FIG. 1A, C, fentanyl (4×60 μg/kg, sc.) promoted a short lasting analgesic response in mice, which reached a maximum at 1 h after the last injection of fentanyl and returned to basal level within 3 h. On the next days following fentanyl injection, mice exhibited a delayed hyperalgesic response that lasted for three days. The results showed that RF1359 alone did not have any effect on the basal nociceptive response of mice (FIG. 1.A). Pre-treatment with compound 1j (5 mg/kg, p.o.) before fentanyl slightly (but not significantly) enhanced analgesic effect of fentanyl when compound 1j was solubilised in Kolliphor EL (AUC: 11.4±3 for compound 1j+Fentanyl vs 17.8±1.8 for Vehicle+Fentanyl, p=0.08 by Unpaired t-test, FIG. 1C). Moreover compound 1j completely blocked the development of the long lasting fentanyl-induced hyperalgesia using as vehicle either tween 80 (HI: 2.2±0.3 for compound 1j+fentanyl vs −8.2±1.9 for vehicle+fentanyl, F_{1, 26}=17; p<0.001, two way ANOVA followed by Fisher's PLSD test p<0.001, FIGS. 1A and B) or Kolliphor EL (HI: −1.8±1.2 for compound 1j+fentanyl vs −11.6±2.4 for vehicle+fentanyl, Unpaired t-test p<0.001, FIGS. 1C and D). As compound 1j displayed a better solubility in Kolliphor EL, this vehicle was chosen for the next experiments. The effect of different doses of compound 1j (0.2, 1 and 5 mg/kg, sc) in our model of fentanyl-induced hyperalgesia was further examined. The results show that the development of hyperalgesia induced by fentanyl was dose-dependently prevented by compound 1j (One way ANOVA, F_{3, 30}=14; p<0.001, FIG. 1.E, F) with and ED50 value around 0.5 mg/kg (FIG. 1.E).

3.2 Compound 1j Reduces Hyperalgesia and Tolerance Associated with Chronic Morphine Administration in Mice.

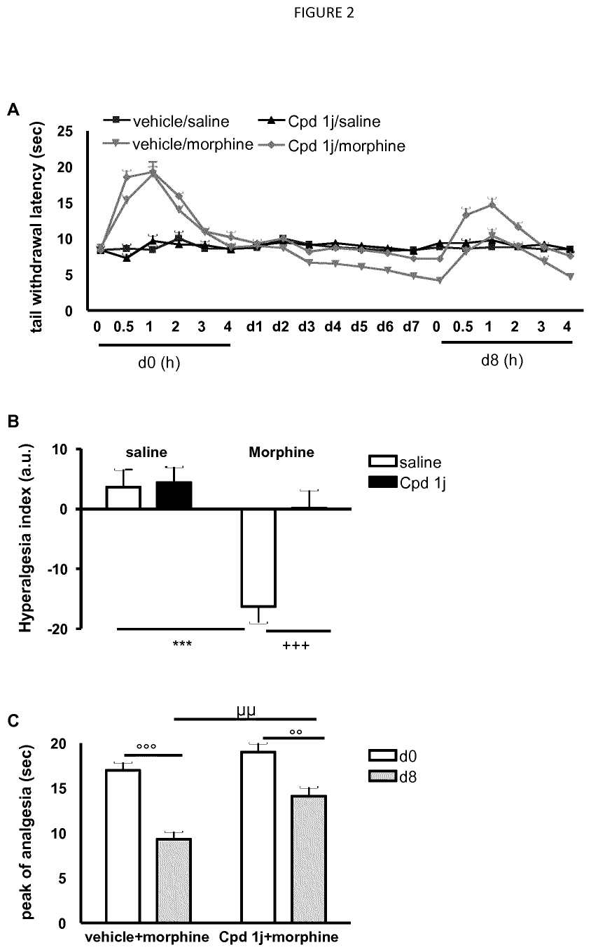

In this experiment, daily administration of morphine (10 mg/kg, s.c.) for 8 consecutive days was performed and the thermal nociceptive response of mice was measured every day before morphine administration. Such a daily morphine treatment led to a progressive decrease of basal nociceptive reaction latency (FIG. 2A), indicative for the development of a robust hyperalgesic state in mice (HI: −16.2±2 for morphine vs 3±3 for saline, F_{1, 32}=17; p<0.001, two way ANOVA followed by Fisher's PLSD test p<0.001, FIG. 2B). Oral treatment of mice with compound 1j (5 mg/kg) before daily morphine administration significantly prevented this hyperalgesia (HI: 0.1±3 for compound 1j+morphine vs −16.2±2 for vehicle+morphine, F_{1, 32}=7; p<0.01, two way ANOVA followed by Fisher's PLSD test p<0.001: FIG. 2B).

In the same experiment, the activity of compound 1j on the development of analgesic tolerance following chronic morphine treatment was assessed. To this end, morphine (5 mg/kg, s.c.) analgesic effect was measured in time-course experiments at day 0 and at day 8 on mice daily pre-treated with either vehicle or compound 1j (5 mg/kg, p.o.). At day 0, morphine analgesia was not significantly modified by co-administration of compound 1j. After 8 days of treatment, the analgesic effect of morphine alone or associated with compound 1j was strongly reduced indicating that tolerance did develop in these animals (FIGS. 2A and C). As shown in FIG. 2C, when compared to day 0, the peak of analgesia was significantly lower at day 8 in both groups treated either with morphine alone (9±0.8 at day 8 vs 17±0.8 at day 0, p<0.001 by paired t-test) or morphine combined with compound 1j (14±0.9 at day 8 vs 19±0.8 at day 0, p<0.01 by paired t-test). However, animals that were pre-treated with compound 1j displayed a significantly higher peak of analgesia than animals treated with morphine alone (14±0.9 for compound 1j+morphine vs 9±0.8 for Vehicle+morphine, p<0.01 by Unpaired t-test) indicating that compound 1j partially maintained morphine analgesia over chronic administration. Altogether, these results indicate that compound 1j can attenuate the development of both hyperalgesia and analgesic tolerance following chronic morphine administration.

### 3.3 Compound 1j Improves the Morphine Analgesia in Incisional Pain Model

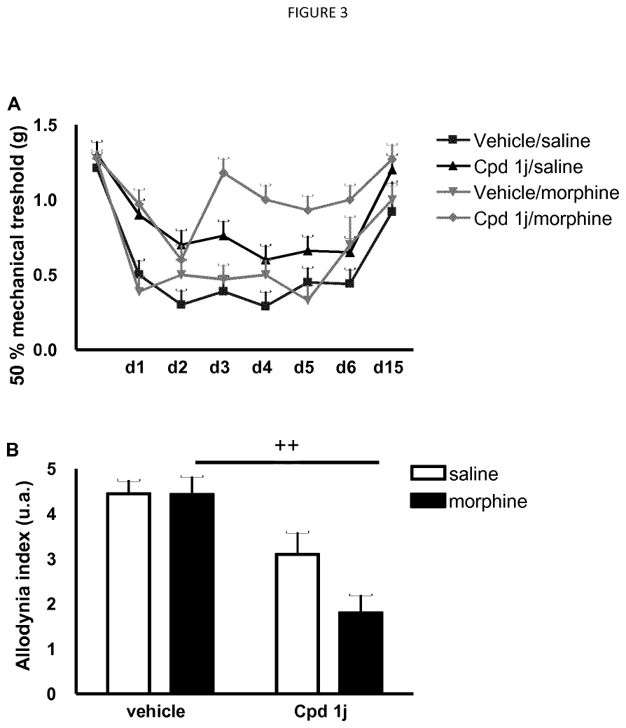

The impact of compound 1j alone on the mechanical nociceptive hypersensitivity induced in animals by paw incision and its ability to improve morphine analgesia in this model was then investigated. As described in material and methods, all tested mice were subjected to a plantar incision under isoflurane anaesthesia followed by daily treatments with morphine in combination or not with compound 1j during six days after the incision. As shown in FIG. 3, in vehicle-treated animals, mechanical nociceptive threshold of the operated paw was strongly reduced after paw incision indicating the development of mechanical allodynia which reached a maximum two days after the incision and persisted until day6 (F_{6, 36}=9.4; p<0.001, repeated measures ANOVA; FIG. 3A). Analyses of allodynia index (d1-d6) revealed that treatment with morphine alone at the dose of 2.5 mg/kg (sc.) failed to prevent significantly the mechanical allodynia (FIG. 3B). However, pre-treatment of animals with compound 1j before morphine significantly enhanced morphine effect at preventing the mechanical allodynia (F_{3, 30}=7; p<0.01, one way ANOVA followed by Fisher's PLSD test p<0.001; FIG. 3B). When it was administered alone, compound 1j slightly attenuated but not significantly the mechanical allodynia developed after paw incision. These results show that compound 1j can significantly improve morphine analgesia in a model of post-operative pain.

### 3.4 Compound 1j Significantly Improves Morphine Analgesia in Neuropathic Pain Model

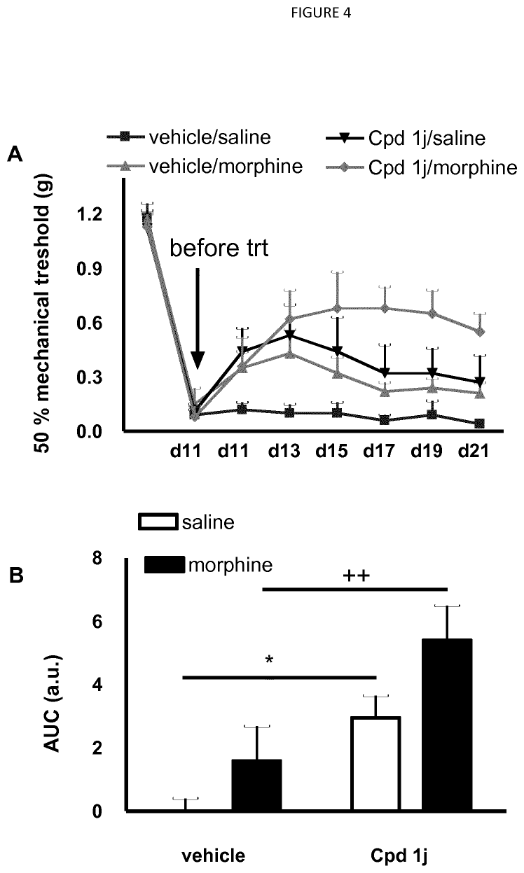

The activity of compound 1j, either alone or in combination with morphine, in a model of neuropathic pain (chronic constriction injury (CCI)-induced neuropathic pain) was next examined. All tested mice were subjected to CCI under deep anaesthesia followed by daily treatments with morphine in combination or not with compound 1j, which started at d11 post CCI and continued for 11 consecutive days. In vehicle-treated animals, mechanical threshold of the ipsilateral paw was strongly reduced after CCI indicating the development of mechanical allodynia that persists until the end of experiment (F_{7, 70}=75, p<0.001, repeated measures ANOVA; FIG. 4A). Analyses of the allodynia index data (d11-d21) by one way ANOVA revealed significant differences between groups (F_{3, 36}=6, p<0.001; FIG. 4B). Post hoc analyses with Fisher's PLSD test indicated that treatment with morphine alone at the dose of 3 mg/kg (sc.) slightly but not significantly prevented the mechanical allodynia associated with neuropathic pain (p>0.05, FIG. 4B). However, pre-treatment of animals with compound 1j before morphine significantly enhanced morphine analgesia effect on neuropathic pain (p<0.01). Moreover, when it was administered alone, compound 1j significantly attenuated the mechanical allodynia developed after CCI (p<0.05). Altogether, these results show that compound 1j significantly improves morphine analgesia and display anti-hyperalgesic effect per se in this model of neuropathic pain. These data suggest that endogenous NPFF system is activated following nerve injury and that pharmacological blockade of NPFF1 receptor subtype can partially restore normal pain sensitivity of injured animals.

### 3.5 Compound 1j Exerts its Anti-Hyperalgesic Effects Via NPFF1 Receptor

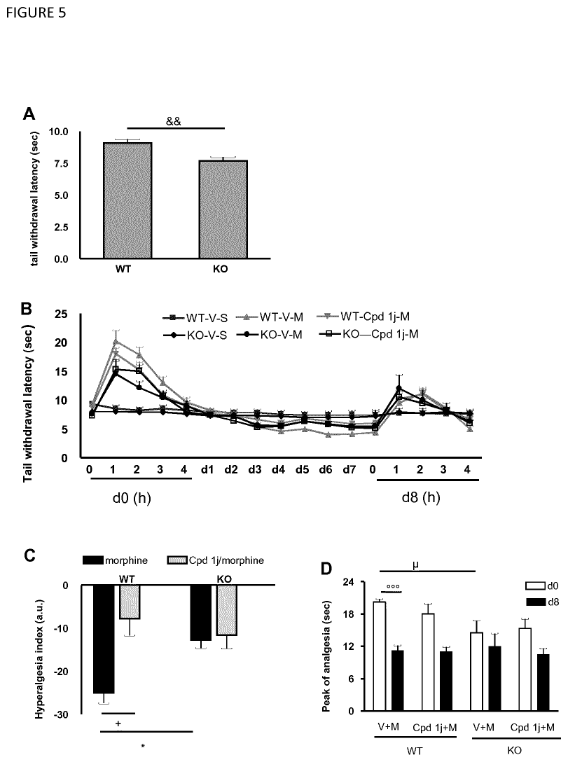

Whether the observed anti-hyperalgesic effects of compound 1j is specific to NPFF1 receptor was then studied. To this end, the effect of compound 1j on morphine-induced hyperalgesia and tolerance in NPFF1R knockout animals were assessed. This experiment was performed in similar experimental conditions than the previous procedure conducted in C57 BL6N WT mice. Both NPFF1R KO and their WT littermates were subjected to daily injections of morphine (10 mg/kg, s.c.) for 8 consecutive days and the thermal nociceptive response of mice was measured every day before morphine administration. First, it was observed that NPFF1R KO animals displayed a slight but significant lower level of nociceptive baseline than their WT littermates (Unpaired t-test, p<0.01; FIG. 5A). Next, the data showed that daily morphine treatment led to a progressive decrease of basal nociceptive reaction latency in NPFF1R KO (F_{8, 72}=8.5; p<0.001, repeated measures ANOVA) and WT mice (F₈, n=35; p<0.001, repeated measures ANOVA), indicative for the development of a hyperalgesic state in all tested animals (FIG. 5 B). However, in agreement with the previous results, the amplitude of the hyperalgesia was significantly lower in NPFF1R KO mice (F_{1, 34}=7; p<0.05, two way ANOVA followed by Fisher's PLSD test p<0.05; FIG. 5 B, C), confirming that NPFF1R is involved in the modulation of opioid-induced hyperalgesia. As expected, oral treatment with compound 1j (5 mg/kg) before daily morphine administration significantly attenuated this hyperalgesia in WT mice (F_{1, 38}=5; p<0.05, two way ANOVA followed by Fisher's PLSD test, p<0.05; FIG. 5 B, C) while in KO animals compound 1j did not show any significant effect. This result clearly indicates that compound 1j exert its anti-hyperalgesic effect through NPFF1 receptor blockade. Similarly to the previous experiment, morphine (5 mg/kg, s.c.) analgesic effect was measured in time-course assays at day 0 and at day 8 on KO and WT mice. At day 0, morphine analgesia was significantly decreased in NPFF1R KO as compared with their WT littermates (p<0.05 by Unpaired t-test; FIG. 5B, D). After 8 days of treatment, the analgesic effect of morphine alone was strongly reduced in WT mice indicating that tolerance did develop in these animals (p<0.001 by Paired t-test). In NPFF1R KO mice, morphine analgesia at d8 did not change significantly compared to d0 (FIG. 5D). Conversely to the previous data from experiment conducted on C57 B6N WT, compound 1j failed to restore morphine analgesia at day 8 in WT mice. Altogether, these results clearly show that compound 1j attenuates the development of hyperalgesia induced by chronic morphine through NPFF1 receptor blockade.

### 3.6 Compound 1c Prevents Hyperalgesia-Induced by Fentanyl in Mice

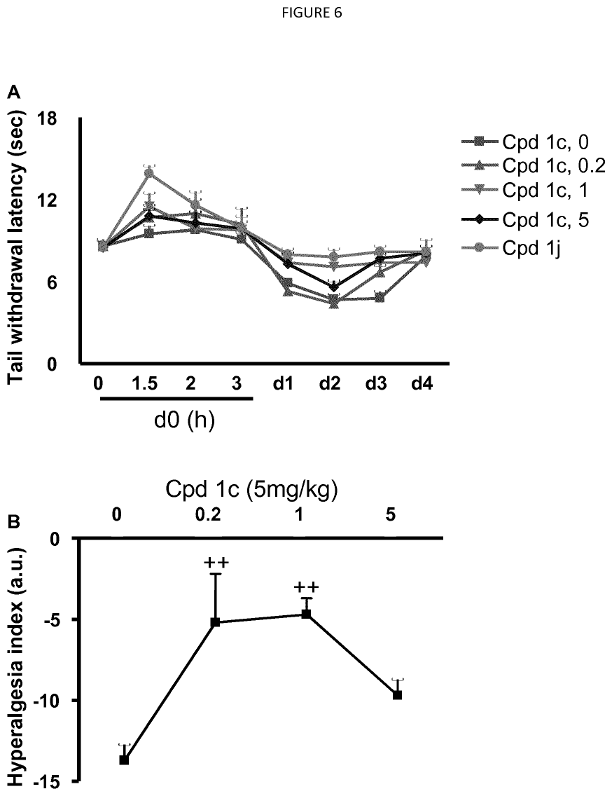

In this assay, it was assessed whether another compound, compound 1c, that is structurally related to compound 1j but with a better bioavailability can also prevent the development of hyperalgesia upon administration of an opiate. To this purpose, fentanyl-induced hyperalgesia experiment was conducted using compound 1c at three doses 0.2, 1 and 5 mg/kg (sc.) and compound 1j (5 mg/kg, sc.) as a reference. As expected, fentanyl (4×60 μg/kg, sc.) promoted a short lasting analgesic response in mice followed on the next days by a delayed hyperalgesic response lasting for three days (FIG. 6 A). Data analyses with one way ANOVA revealed that there are significant differences between the tested groups (F_{4, 34}=5; p<0.001). Post hoc Fisher's PLSD test showed that compound 1c prevented fentanyl hyperalgesia at 0.2 (p<0.01) and 1 mg/kg (p<0.01) but not at 5 mg/kg (FIG. 6 B). Similarly to the previous experiments, pre-treatment of mice with compound 1j (5 mg/kg, sc.) exhibited a significant anti-hyperalgesic effect (p<0.001).

1. A compound for use in the treatment of pain, where the compound has the following formula (I): 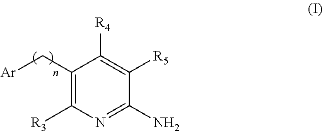
Ar is a carbocyclyl, heterocyclyl, aryl or heteroaryl ring, said ring can optionally be substituted by one or more groups selected from a halogen atom, a (C₁-C₁₀)alkyl group, a cyano group, a carbocycle, aryl, heterocycle, —C(O)R, —C(O)₂R, —C(O)NRR′, —CONHOR, —CONHSO₂R, —NRR′, —N(R)C(O)R′, —N(R)NR′R″, —N(R)C(O)₂R′, —N(R)C(O)NR′R″, —N(R)S(O)₂R′, —OR, —SR, —S(O)R, —S(O₂)R, —S(O)NRR′, or —S(O)₂NRR′, R, R′, and R″ being independently H, (C₁-C₁₀)alkyl, carbocycle, aryl, heterocycle, heteroaryl, (C₁-C₁₀)alkylcarbocycle, (C₁-C₁₀)alkylaryl, (C₁-C₁₀)alkylheterocycle, (C₁-C₁₀)alkylheteroaryl, (C₁-C₁₀)alkoxycarbocycle, (C₁-C₁₀)alkoxyaryl, (C₁-C₁₀)alkoxyheterocycle), or (C₁-C₁₀)alkoxyheteroaryl group, or R and R′ or R′ and R″ may form a 5-10 membered ring, said 5-10 membered ring is optionally substituted by at least one —OH, halogen, (C₁-C₁₀)alkyl, or (C₁-C₁₀)alkyloxy; said substituent can be further substituted by at least one group selected from a halogen atom, an hydroxyl group, a (C₁-C₁₀)alkyl group, a (C₁-C₁₀)alkoxy group and aryl group;
n is 0, 1, 2, or 3;
R₃ represents an hydrogen atom, halogen atom, NRR′, (C₁-C₁₀)alkyl, or (C₁-C₁₀)alkoxy group;
R₄ represents an hydrogen atom, halogen, NRR′, (C₁-C₁₀)alkyl, or (C₁-C₁₀)alkoxy group;
R₅ represents an hydrogen atom, halogen, NRR′, (C₁-C₁₀)alkyl, or (C₁-C₁₀)alkoxy group;
where R and R′, identical or different, are as defined above;
or any salt thereof.

2. The compound for use according to claim 1, where the pain is a chronic pain.

3. The compound for use according to claim 1 or 2, to decrease or block hyperalgesia and/or tolerance effects related to the use of an analgesic compound, in particular an opiate analgesic compound.

4. The compound for use according to claim 3, wherein the analgesic compound is selected from morphine, fentanyl, sufentanil, alfentanyl, heroin, oxycodone, hydromorphone, levorphanol, methadone, buprenorphine, butorphanol, meperidine and a mixture thereof.

5. The compound for use according to anyone of claims 1-4, for the treatment of hyperalgesia occurring or associated with acute or chronic pain in response to surgery, trauma or pathology of a mammal.

6. The compound for use according to anyone of claims 1-5, where the compound is of formula (I) with Ar is a carbocyclyl or an heteroaryl, preferably a furanyl, benzofuranyl, a pyrazolyl, or a pyridinyl group, said Ar group can optionally be substituted by one or more groups selected from a halogen atom, a (C₁-C₁₀)alkyl group, an aryl group, or a —OR, R being as defined above, preferably R being H, (C₁-C₁₀)alkyl, or a —NRR′ group, R and R′ being as defined above, preferably R and R′ are independently H, (C₁-C₁₀)alkyl, or heterocycle.

7. The compound for use according to anyone of claims 1-5, where the compound is of formula (I) with Ar is a phenyl group, preferably substituted by one or more groups selected from a halogen atom, a (C₁-C₁₀)alkyl group, a cyano group, an aryl group, or a —OR, R being as defined above, preferably R being H or (C₁-C₁₀)alkyl.

8. The compound for use according to anyone of claims 1-5, where the compound is of formula (I) with Ar is 1-naphtyl.

9. The compound for use according to anyone of claims 1-8, where the compound is of formula (I) with R4 and R5 both represent a hydrogen atom.

10. The compound for use according to anyone of claims 1-9, where the compound is of formula (I) with R3 is NH2, an halogen atom, such as Cl or F, a (C₁-C₄)alkyl (such as methyl or ethyl), CF3, (C₁-C₄)alkoxy group (such as methoxy, ethoxy, OCH₂CF₃, O(CH₂)₂NH₂), an ether group (such as methoxymethyl), NRR′, where R and R′ are as defined above, preferably R is H and R′ is (C₁-C₁₀)alkyl (more particularly methyl, n-butyl, ethyl, isopropyl), optionally substituted by an aryl (such as phenyl), by an alkoxy (such as methoxy), or by an heterocycle (such as piperidine), R′ can also be an an heterocycle (such as piperidine), or alternatively R and R′ can form together an heterocycle with the nitrogen to which they are attached, such as piperidine.

11. The compound for use according to anyone of claims 1-10, where n is 0 and preferably Ar is a phenyl substituted at least on position 2.

12. The compound for use according to anyone of claims 1-11, where the compound is a compound of formula (II): 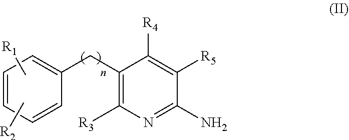
where:
n is 0, 1 or 2, and preferably n is 0;
R3, R4 and R5 are as defined in anyone of the previous claims 1, 9 and 10, and
R1 and R2 are independently hydrogen atoms or the substituents of Ar as defined in claim 1.

13. The compound for use according to claim 12, where R₁ represents a halogen atom, a (C₁-C₁₀)alkyl group, a cyano group (—CN), an aryl(C₁-C₁₀)alkyl group, carbocycle, aryl, heterocycle, —C(O)R, —C(O)₂R, —C(O)NRR′, —CONHOR, —CONHSO₂R, —NRR′, —N(R)C(O)R′, —N(R)NR′R″, —N(R)C(O)₂R, —N(R)C(O)NR′R″, —N(R)S(O)₂R, —OR, —SR, —S(O)R, —S(O₂)R, —S(O)NRR′, or —S(O)₂NRR′, R, R′, and R″ being independently H, (C₁-C₁₀)alkyl, carbocycle, aryl, aralkyl, heterocycle, heteroaryl, (C₁-C₁₀)alkylcarbocycle, (C₁-C₁₀)alkylaryl, (C₁-C₁₀)alkylheterocycle), (C₁-C₁₀)alkylheteroaryl, (C₁-C₁₀)alkoxycarbocycle, (C₁-C₁₀)alkoxyaryl, (C₁-C₁₀)alkoxyheterocycle), or (C₁-C₁₀)alkoxyheteroaryl group, or R and R′ or R′ and R″ may form a 5-10 membered ring, said 5-10 membered ring is optionally substituted by at least one —OH, halogen, (C₁-C₁₀)alkyl, or (C₁-C₁₀)alkyloxy; said R₁ group can be further substituted by at least one group selected from a halogen atom, an hydroxyl group, a (C₁-C₁₀)alkyl group, a (C₁-C₁₀)alkoxy group, and aryl group; and/or
R₂ represents an hydrogen atom, a halogen atom, a (C₁-C₁₀)alkyl group, —C(O)R, —C(O)₂R, —C(O)NRR′, —CONHOR, —CONHSO₂R, —NRR′, —N(R)C(O)R′, —N(R)NR′R″, —N(R)C(O)₂R′, —N(R)C(O)NR′R″, —N(R)S(O)₂R′, —OR, —SR, —S(O)R, —S(O₂)R, —S(O)NRR′, or —S(O)₂NRR′, R, R′, and R″ being independently H, (C₁-C₁₀)alkyl, carbocycle, aryl, heterocycle, heteroaryl, (C₁-C₁₀)alkylcarbocycle, (C₁-C₁₀)alkylaryl, (C₁-C₁₀)alkylheterocycle), (C₁-C₁₀)alkylheteroaryl, (C₁-C₁₀)alkoxycarbocycle, (C₁-C₁₀)alkoxyaryl, (C₁-C₁₀)alkoxyheterocycle), or (C₁-C₁₀)alkoxyheteroaryl group, or R and R′ or R′ and R″ may form a 5-10 membered ring, said 5-10 membered ring is optionally substituted by at least one —OH, halogen, (C₁-C₁₀)alkyl, or (C₁-C₁₀)alkyloxy; said R₂ group can be further substituted by at least one group selected from a halogen atom, an hydroxyl group, a (C₁-C₁₀)alkyl group, and a (C₁-C₁₀)alkoxy group.

14. The compound for use according to claim 13, where the compound is of the following formula (III): 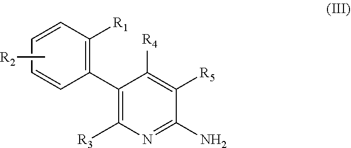
where R3, R4 and R5 are as defined in anyone of the previous claims, and
R₁ represents a halogen atom, a (C₁-C₁₀)alkyl group, a cyano group (—CN), an aryl(C₁-C₁₀)alkyl group, carbocycle, aryl, heterocycle, —C(O)R, —C(O)₂R, —C(O)NRR′, —CONHOR, —CONHSO₂R, —NRR′, —N(R)C(O)R′, —N(R)NR′R″, —N(R)C(O)₂R′, —N(R)C(O)NR′R″, —N(R)S(O)₂R′, —OR, —SR, —S(O)R, —S(O₂)R, —S(O)NRR′, or —S(O)₂NRR′, R, R′, and R″ being independently H, (C₁-C₁₀)alkyl, carbocycle, aryl, aralkyl, heterocycle, heteroaryl, (C₁-C₁₀)alkylcarbocycle, (C₁-C₁₀)alkylaryl, (C₁-C₁₀)alkylheterocycle), (C₁-C₁₀)alkylheteroaryl, (C₁-C₁₀)alkoxycarbocycle, (C₁-C₁₀)alkoxyaryl, (C₁-C₁₀)alkoxyheterocycle), or (C₁-C₁₀)alkoxyheteroaryl group, or R and R′ or R′ and R″ may form a 5-10 membered ring, said 5-10 membered ring is optionally substituted by at least one —OH, halogen, (C₁-C₁₀)alkyl, or (C₁-C₁₀)alkyloxy; said R₁ group can be further substituted by at least one group selected from a halogen atom, an hydroxyl group, a (C₁-C₁₀)alkyl group, a (C₁-C₁₀)alkoxy group, and an aryl group;
R₂ represents an hydrogen atom, a halogen atom, a (C₁-C₁₀)alkyl group, —C(O)R, —C(O)₂R, —C(O)NRR′, —CONHOR, —CONHSO₂R, —NRR′, —N(R)C(O)R′, —N(R)NR′R″, —N(R)C(O)₂R′, —N(R)C(O)NR′R″, —N(R)S(O)₂R′, —OR, —SR, —S(O)R, —S(O₂)R, —S(O)NRR′, or —S(O)₂NRR′, R, R′, and R″ being independently H, (C₁-C₁₀)alkyl, carbocycle, aryl, heterocycle, heteroaryl, (C₁-C₁₀)alkylcarbocycle, (C₁-C₁₀)alkylaryl, (C₁-C₁₀)alkylheterocycle), (C₁-C₁₀)alkylheteroaryl, (C₁-C₁₀)alkoxycarbocycle, (C₁-C₁₀)alkoxyaryl, (C₁-C₁₀)alkoxyheterocycle), or (C₁-C₁₀)alkoxyheteroaryl group, or R and R′ or R′ and R″ may form a 5-10 membered ring, said 5-10 membered ring is optionally substituted by at least one —OH, halogen, (C₁-C₁₀)alkyl, or (C₁-C₁₀)alkyloxy; said R₂ group can be further substituted by at least one group selected from a halogen atom, an hydroxyl group, a (C₁-C₁₀)alkyl group, a (C₁-C₁₀)alkoxy group.

15. The compound for use according to claim 14, where the compound is of the following formula (III) with one or more of the following features:
R₂ is H, a halogen atom, a (C₁-C₁₀)alkyl group (preferably (C₁-C₄)alkyl), or —OR, R is as defined above, and more preferably R is H or (C₁-C₁₀)alkyl; and/or
R₁ represents a halogen atom, a (C₁-C₁₀)alkyl group (preferably (C₁-C₄)alkyl), carbocycle (such as cyclopropyl, cyclopentyl), aryl (such as phenyl), or —OR, with R is as defined above, and more preferably R is H, (C₁-C₁₀)alkyl (such as methyl, ethyl, iso-propyl, or —CH₃(C₃H₅)), (C₁-C₁₀)alkylheterocycle (such as 1-piperidinylethyl) or carbocyclyl (such as cyclopropyl, cyclopentyl); and/or
R₃ is a (C₁-C₄)alkyl (such as methyl or ethyl), NRR′, with R is H and R′ is H, (C₁-C₁₀)alkyl (more particularly methyl, n-butyl, ethyl, isopropyl), optionally substituted by an aryl (such as phenyl), by an alkoxy (such as methoxy), or by an heterocycle (such as piperidine), R′ can also be an heterocycle (such as piperidine), or alternatively R and R′ can form together an heterocycle with the nitrogen to which they are attached, such as piperidine; and/or
R₄ and R₅ are independently an hydrogen atom, an halogen atom or an (C₁-C₁₀)alkyl group, preferably R₄ and R₅ are both hydrogen atoms.

16. The compound for use according to anyone of claims 1-15, where the compound is selected in the group consisting of:
3-phenylpyridine-2,6-diamine, 1a,
3-(2-chlorophenyl)pyridine-2,6-diamine (trifluoroacetate), 1b,
3-(o-tolyl)pyridine-2,6-diamine (trifluoroacetate), 1c,
3-(2-ethylphenyl)pyridine-2,6-diamine (hydrochloride), 1d,
3-(2-isopropylphenyl) pyridine-2,6-diamine (hydrochloride), 1e,
3-(2-(trifluoromethyl) phenyl) pyridine-2,6-diamine (trifluoroacetate), 1f,
3-(2-(methoxymethyl) phenyl) pyridine-2,6-diamine (hydrochloride), 1g,
3-(3-chlorophenyl)pyridine-2,6-diamine (trifluoroacetate), 1h,
3-(4-chlorophenyl)pyridine-2,6-diamine (trifluoroacetate), 1i,
3-(2,3-dichlorophenyl)pyridine-2,6-diamine, 1j,
3-(2,4-dichlorophenyl) pyridine-2,6-diamine (trifluoroacetate), 1k,
3-(2,5-dichlorophenyl) pyridine-2,6-diamine (trifluoroacetate), 1l,
3-(2,6-dichlorophenyl) pyridine-2,6-diamine, 1m,
3-(3,4-dichlorophenyl) pyridine-2,6-diamine (trifluoroacetate), 1n,
3-(3,5-dichlorophenyl) pyridine-2,6-diamine (trifluoroacetate), 1o,
3-(2-chloro-3-(trifluoromethyl) phenyl) pyridine-2,6-diamine (trifluoroacetate), 1p,
3-(3-chloro-2-methylphenyl) pyridine-2,6-diamine (trifluoroacetate), 1q,
3-([1,1′-biphenyl]-2-yl)pyridine-2,6-diamine (hydrochloride), 1r,
2-(2,6-diaminopyridin-3-yl)phenol, 2a,
3-(2-methoxyphenyl) pyridine-2,6-diamine (trifluoroacetate), 2b,
3-(2-(trifluoromethoxy) phenyl) pyridine-2,6-diamine (hydrochloride), 2c,
3-(2-ethoxyphenyl)pyridine-2,6-diamine (hydrochloride), 2d,
3-(2-butoxyphenyl)pyridine-2,6-diamine, 2e,
3-(2-isopropoxyphenyl)pyridine-2,6-diamine 2f,
3-(2-isobutoxyphenyl) pyridine-2,6-diamine (hydrochloride), 2g,
3-(2-methoxyethoxyphenyl)pyridine-2,6-diamine, 2h,
3-(2-(cyclopentyloxy)phenyl)pyridine-2,6-diamine 2i,
3-(2-(piperidin-1-yl)ethoxy)pyridine-2,6-diamine 2j,
3-(4-fluoro-2-methoxyphenyl) pyridine-2,6-diamine (hydrochloride), 2k,
3-(2,3-dimethoxyphenyl) pyridine-2,6-diamine (hydrochloride), 2l,
3-(2,4-dimethoxyphenyl) pyridine-2,6-diamine (hydrochloride), 2m,
3-(furan-2-yl)pyridine-2,6-diamine (trifluoroacetate), 3a,
3-(furan-3-yl)pyridine-2,6-diamine (trifluoroacetate), 3b,
3-(benzofuran-2-yl) pyridine-2,6-diamine (hydrochloride), 3c,
[3,4′-bipyridine]-2,6-diamine, 3d,
[3,3′-bipyridine]-2,6-diamine, 3e,
3′-methyl-[3,4′-bipyridine]-2,6-diamine, 3f,
N-(6-amino-5-(2,3-dichlorophenyl)pyridin-2-yl)acetamide, 4a,
3-(2,3-dichlorophenyl)-N2-methylpyridine-2,6-diamine, 5a,
3-(2,3-dichlorophenyl)-N2-ethylpyridine-2,6-diamine, 5b,
N2-butyl-3-(2,3-dichlorophenyl)pyridine-2,6-diamine, 5c,
N2-benzyl-3-(2,3-dichlorophenyl)pyridine-2,6-diamine, 5d,
3-(2,3-dichlorophenyl)-N2-isopropylpyridine-2,6-diamine, 5e,
3-(2,3-dichlorophenyl)-N2-phenethylpyridine-2,6-diamine, 5f,
3-(2,3-dichlorophenyl)-N2-(2-methoxyethyl)pyridine-2,6-diamine, 5g,
3-(2,3-dichlorophenyl)-N2-(2-(piperidin-1-yl)ethyl)pyridine-2,6-diamine, 5h,
5-(2,3-dichlorophenyl)-6-(piperidin-1-yl)pyridin-2-amine, 5i,
5-(2,3-dichlorophenyl)pyridin-2-amine, 6a,
5-(2,3-dichlorophenyl)-6-methylpyridin-2-amine, 6b,
5-(2,3-dichlorophenyl)-6-ethylpyridin-2-amine, 6c,
6-ethyl-5-(2-methoxyphenyl)pyridin-2-amine, 6d,
6-(methoxymethyl)-5-(2-methoxyphenyl)pyridin-2-amine, 6e,
5-(2-methoxyphenyl)-6-(trifluoromethoxy)pyridin-2-amine, 6f,
5-(2-methoxyphenyl)-6-propylpyridin-2-amine, 6g,
6-isopropyl-5-(2-methoxyphenyl)pyridin-2-amine, 6h,
6-cyclopropyl-5-(2-methoxyphenyl)pyridin-2-amine, 6i,
5-(2,3-dichlorophenyl)-6-methoxypyridin-2-amine, 7a,
3-(2,3-dichlorophenyl)-4-methoxypyridine-2,6-diamine, 8a,
4-methyl-3-(o-tolyl)pyridine-2,6-diamine, 8b,
3-(2,3-dichlorophenyl)-5-fluoropyridine-2,6-diamine 9a,
3-(2,3-dichlorophenyl)-5-ethylpyridine-2,6-diamine, 9b,
3-benzylpyridine-2,6-diamine hydrochloride, 10a,
3-(4-fluorobenzyl)pyridine-2,6-diamine, 10b,
3-(4-chlorobenzyl)pyridine-2,6-diamine, 10c,
3-(3-chlorobenzyl)pyridine-2,6-diamine, 10d,
3-(2-chlorobenzyl)pyridine-2,6-diamine, 10e,
3-(2,4-dichlorobenzyl)pyridine-2,6-diamine (hydrochloride), 10f,
3-phenethylpyridine-2,6-diamine, 11a,
5-(2-Methoxy-phenyl)-3-methyl-pyridin-2-ylamine, 13
5-(2-Methoxy-phenyl)-3-trifluoromethyl-pyridin-2-ylamine, 14
3-Fluoro-5-(2-methoxy-phenyl)-pyridin-2-ylamine, 15
5-(2,3-Dichloro-phenyl)-3-fluoro-pyridin-2-ylamine, 16
5-(2,3-Dichloro-phenyl)-4-fluoro-pyridin-2-ylamine, 17
4-Fluoro-5-(2-methoxy-phenyl)-pyridin-2-ylamine, 18
5-(2,3-Dichloro-phenyl)-4-methoxy-pyridin-2-ylamine (hydrochloride), 19
4-Methoxy-5-(2-methoxy-phenyl)-pyridin-2-ylamine, 20
5-(2-Methoxy-phenyl)-4-methyl-pyridin-2-ylamine, 21
5-(2,3-Dichloro-phenyl)-4-methyl-pyridin-2-ylamine, 22
5-(2-Methoxy-phenyl)-4-(2,2,2-trifluoro-ethoxy)-pyridin-2-ylamine (hydrochloride), 23
5-(2,3-Dichloro-phenyl)-4-(2,2,2-trifluoro-ethoxy)-pyridin-2-ylamine (hydrochloride), 24
4-(2-aminoethoxy)-5-(2-methoxyphenyl)pyridin-2-amine (dihydrochloride), 25
5-(2-Methoxy-phenyl)-6-methyl-pyridin-2-ylamine, 26
5-(2-Methoxy-phenyl)-4,6-dimethyl-pyridin-2-ylamine, 27
5-(2,3-Dichloro-phenyl)-4,6-dimethyl-pyridin-2-ylamine, 28
5-(3-chloro-2-methyl-phenyl)-6-ethyl-pyridin-2-ylamine (hydrochloride), 29
5-(2-cyclopropylphenyl)-6-ethyl-pyridin-2-amine (hydrochloride), 30
5-[2-(cyclopropoxy)phenyl]-6-ethyl-pyridin-2-amine (hydrochloride), 31
6-Fluoro-5-(2-methoxy-phenyl)-pyridin-2-ylamine, 32
5-(2,3-Dichloro-phenyl)-6-fluoro-pyridin-2-ylamine, 33
5-(2,3-Dichloro-phenyl)-6-trifluoromethyl-pyridin-2-ylamine, 34
6-Methoxy-5-(2-methoxy-phenyl)-pyridin-2-ylamine, 35
5-(2-Methoxy-phenyl)-6-(2,2,2-trifluoro-ethoxy)-pyridin-2-ylamine, 36
6-(2-Amino-ethoxy)-5-(2-methoxy-phenyl)-pyridin-2-ylamine, 37
6-(2-Amino-ethoxy)-5-(2,3-dichloro-phenyl)-pyridin-2-ylamine (dihydrochloride), 38
3-(2-Isopropoxy-6-methoxy-phenyl)-pyridine-2,6-diamine, 39
3-(4-Methoxy-2-methyl-phenyl)-pyridine-2,6-diamine, 40
3-(4-Chloro-2-fluoro-phenyl)-pyridine-2,6-diamine, 41
3-(2-Cyclopropyl-phenyl)-pyridine-2,6-diamine (hydrochloride), 42
3-(2-Phenoxy-phenyl)-pyridine-2,6-diamine (hydrochloride), 43
3-(2-Benzyl-phenyl)-pyridine-2,6-diamine, 44
3-(2-Chloro-4-fluoro-phenyl)-pyridine-2,6-diamine, 45
3-(2-Isopropoxy-4-methyl-phenyl)-pyridine-2,6-diamine, 46
3-(4-Chloro-2-cyclopentyloxy-phenyl)-pyridine-2,6-diamine, 47
3-(2-Cyclopropoxy-phenyl)-pyridine-2,6-diamine, 48
3-(2-Isopropoxy-5-methyl-phenyl)-pyridine-2,6-diamine, 49
3-(5-Fluoro-2-isopropoxy-phenyl)-pyridine-2,6-diamine, 50
3-(2,6-Dimethyl-phenyl)-pyridine-2,6-diamine, 51
3-(2-Isopropoxy-5-trifluoromethyl-phenyl)-pyridine-2,6-diamine, 52
3-(4-Fluoro-2-isopropoxy-phenyl)-pyridine-2,6-diamine, 53
3-(4-Chloro-2-methyl-phenyl)-pyridine-2,6-diamine, 54
3-(5-Chloro-2-cyclopropyl-phenyl)-pyridine-2,6-diamine, 55
3-(5-Chloro-2-methyl-phenyl)-pyridine-2,6-diamine, 56
3-(2-Methyl-4-trifluoromethyl-phenyl)-pyridine-2,6-diamine, 57
3-(2-chloro-3-methyl-phenyl)-pyridine-2,6-diamine, 58
3-(2-Methylsulfanyl-phenyl)-pyridine-2,6-diamine (hydrochloride), 59
2-(2,6-Diamino-pyridin-3-yl)-N,N-diethyl-benzamide (hydrochloride), 60
3-(2-Dimethylamino-phenyl)-pyridine-2,6-diamine (hydrochloride), 61
N-[2-(2,6-Diamino-pyridin-3-yl)-phenyl]-acetamide, 62
3-(2-methylsulfonylphenyl)pyridine-2,6-diamine (hydrochloride), 63
3-(2-benzyloxyphenyl)pyridine-2,6-diamine (hydrochloride), 64
3-[2-(cyclopropylmethoxy)phenyl]pyridine-2,6-diamine (hydrochloride), 65
3-(3-Chloro-2-methyl-phenyl)-5-fluoro-pyridine-2,6-diamine, 66
6-ethyl-5-(1-naphthyl)pyridin-2-amine (hydrochloride), 67
3-(1-naphthyl)pyridine-2,6-diamine, 68
3-(2-methoxy-1-naphthyl)pyridine-2,6-diamine, 69
3-(2-isopropoxy-1-naphthyl)pyridine-2,6-diamine, 70
3-(4-methyl-1-naphthyl)pyridine-2,6-diamine (hydrochloride), 71
3-(4-fluoro-1-naphthyl)pyridine-2,6-diamine (hydrochloride), 72
3-(4-chloro-1-naphthyl)pyridine-2,6-diamine (hydrochloride), 73
4-(2,6-diamino-3-pyridyl)naphthalen-1-ol (hydrochloride), 74
3-[4-(dimethylamino)-1-naphthyl]pyridine-2,6-diamine (hydrochloride), 75
5-[2-(cyclopentoxy)phenyl]-6-ethyl-pyridin-2-amine (hydrochloride), 76
3-(4-bromophenyl)pyridine-2,6-diamine, 77
3-(6-morpholino-3-pyridyl)pyridine-2,6-diamine, 78
3-[6-(1-piperidyl)-3-pyridyl]pyridine-2,6-diamine, 79
3-[6-(methylamino)-3-pyridyl]pyridine-2,6-diamine, 80
3-(6-pyrrolidin-1-yl-3-pyridyl)pyridine-2,6-diamine, 81
3-(6-amino-3-pyridyl)pyridine-2,6-diamine, 82
3-[6-amino-5-(trifluoromethyl)-3-pyridyl]pyridine-2,6-diamine, 83
3-(2-methyl-3-pyridyl)pyridine-2,6-diamine, 84
3-(6-fluoro-2-methyl-3-pyridyl)pyridine-2,6-diamine, 85
3-(6-fluoro-3-pyridyl)pyridine-2,6-diamine, 86
3-(2-fluoro-3-pyridyl)pyridine-2,6-diamine, 87
3-(4-methoxy-3-pyridyl)pyridine-2,6-diamine, 88
3-(2-methoxy-3-pyridyl)pyridine-2,6-diamine, 89
3-(3,5-dimethyl-1H-pyrazol-4-yl)pyridine-2,6-diamine, 90
3-(5-methyl-1H-pyrazol-4-yl)pyridine-2,6-diamine, 91
2-(2,6-diamino-3-pyridyl)benzonitrile (hydrochloride), 96
and one of a salt thereof.

17. A compound of formula (III): 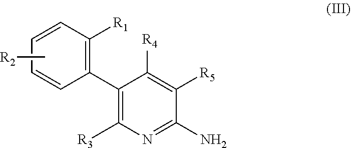
where R₃, R₄ and R₅ are as defined in anyone of claims 1, 9, 10, and
R₁ represents a halogen atom, a (C₁-C₁₀)alkyl group, a cyano group (—CN), an aryl(C₁-C₁₀)alkyl group, carbocycle, aryl, heterocycle, —C(O)R, —C(O)₂R, —C(O)NRR′, —CONHOR, —CONHSO₂R, —NRR′, —N(R)C(O)R′, —N(R)NR′R″, —N(R)C(O)₂R′, —N(R)C(O)NR′R″, —N(R)S(O)₂R′, —OR, —SR, —S(O)R, —S(O₂)R, —S(O)NRR′, or —S(O)₂NRR′, R, R′, and R″ being independently H, (C₁-C₁₀)alkyl, carbocycle, aryl, aralkyl, heterocycle, heteroaryl, (C₁-C₁₀)alkylcarbocycle, (C₁-C₁₀)alkylaryl, (C₁-C₁₀)alkylheterocycle), (C₁-C₁₀)alkylheteroaryl, (C₁-C₁₀)alkoxycarbocycle, (C₁-C₁₀)alkoxyaryl, (C₁-C₁₀)alkoxyheterocycle), or (C₁-C₁₀)alkoxyheteroaryl group, or R and R′ or R′ and R″ may form a 5-10 membered ring, said 5-10 membered ring is optionally substituted by at least one —OH, halogen, (C₁-C₁₀)alkyl, or (C₁-C₁₀)alkyloxy; said R₁ group can be further substituted by at least one group selected from a halogen atom, an hydroxyl group, a (C₁-C₁₀)alkyl group, a (C₁-C₁₀)alkoxy group and aryl group;
R₂ represents an hydrogen atom, a halogen atom, a (C₁-C₁₀)alkyl group, —C(O)R, —C(O)₂R, —C(O)NRR′, —CONHOR, —CONHSO₂R, —NRR′, —N(R)C(O)R′, —N(R)NR′R″, —N(R)C(O)₂R′, —N(R)C(O)NR′R″, —N(R)S(O)₂R′, —OR, —SR, —S(O)R, —S(O₂)R, —S(O)NRR′, or —S(O)₂NRR′, R, R′, and R″ being independently H, (C₁-C₁₀)alkyl, carbocycle, aryl, heterocycle, heteroaryl, (C₁-C₁₀)alkylcarbocycle, (C₁-C₁₀)alkylaryl, (C₁-C₁₀)alkylheterocycle), (C₁-C₁₀)alkylheteroaryl, (C₁-C₁₀)alkoxycarbocycle, (C₁-C₁₀)alkoxyaryl, (C₁-C₁₀)alkoxyheterocycle), or (C₁-C₁₀)alkoxyheteroaryl group, or R and R′ or R′ and R″ may form a 5-10 membered ring, said 5-10 membered ring is optionally substituted by at least one —OH, halogen, (C₁-C₁₀)alkyl, or (C₁-C₁₀)alkyloxy; said R₂ group can be further substituted by at least one group selected from a halogen atom, an hydroxyl group, a (C₁-C₁₀)alkyl group, a (C₁-C₁₀)alkoxy group.

18. The compound according to claim 17, where the compound presents one or more of the following features:
R₂ is H, a halogen atom, a (C₁-C₁₀)alkyl group (preferably (C₁-C₄)alkyl), or —OR, R is as defined above, and more preferably R is H or (C₁-C₁₀)alkyl; and/or
R₁ represents a halogen atom, a (C₁-C₁₀)alkyl group (preferably (C₁-C₄)alkyl), carbocycle (such as cyclopropyl, cyclopentyl), aryl (such as phenyl), or —OR, with R is as defined above, and more preferably R is H, (C₁-C₁₀)alkyl (such as methyl, ethyl, iso-propyl, or —CH₃(C₃H₅)), (C₁-C₁₀)alkylheterocycle (such as 1-piperidinylethyl) or carbocyclyl (such as cyclopropyl, cyclopentyl) as defined above; and/or
R₃ is a (C₁-C₄)alkyl (such as methyl or ethyl), NRR′, with R is H and R′ is H, (C₁-C₁₀)alkyl (more particularly methyl, n-butyl, ethyl, isopropyl), optionally substituted by an aryl (such as phenyl), by an alkoxy (such as methoxy), or by an heterocycle (such as piperidine), R′ can also be an heterocycle (such as piperidine), or alternatively R and R′ can form together an heterocycle with the nitrogen to which they are attached, such as piperidine; and/or
R₄ and R₅ are independently an hydrogen atom, an halogen atom, an (C₁-C₁₀)alkyl group, or an (C₁-C₁₀)alkoxy group, preferably R₄ and R₅ are both hydrogen atoms.

19. The compound according to claim 17 or 18, where the compound is selected in the group consisting of:
3-(2-chlorophenyl)pyridine-2,6-diamine (trifluoroacetate), 1b,
3-(2-isopropylphenyl) pyridine-2,6-diamine (hydrochloride), 1e,
3-(2-(trifluoromethyl) phenyl) pyridine-2,6-diamine (trifluoroacetate), 1f,
3-(2-(methoxymethyl) phenyl) pyridine-2,6-diamine (hydrochloride), 1g,
3-(2,3-dichlorophenyl)pyridine-2,6-diamine, 1j,
3-(2,4-dichlorophenyl) pyridine-2,6-diamine (trifluoroacetate), 1k,
3-(2,5-dichlorophenyl) pyridine-2,6-diamine (trifluoroacetate), 1l,
3-(2,6-dichlorophenyl) pyridine-2,6-diamine, 1m,
3-(2-chloro-3-(trifluoromethyl) phenyl) pyridine-2,6-diamine (trifluoroacetate), 1p,
3-(3-chloro-2-methylphenyl) pyridine-2,6-diamine (trifluoroacetate), 1q,
3-([1,1′-biphenyl]-2-yl)pyridine-2,6-diamine (hydrochloride), 1r,
3-(2-methoxyphenyl) pyridine-2,6-diamine (trifluoroacetate), 2b,
3-(2-(trifluoromethoxy) phenyl) pyridine-2,6-diamine (hydrochloride), 2c,
3-(2-ethoxyphenyl)pyridine-2,6-diamine (hydrochloride), 2d,
3-(2-butoxyphenyl)pyridine-2,6-diamine, 2e,
3-(2-isopropoxyphenyl)pyridine-2,6-diamine 2f,
3-(2-isobutoxyphenyl) pyridine-2,6-diamine (hydrochloride), 2g,
3-(2-methoxyethoxyphenyl)pyridine-2,6-diamine, 2h,
3-(2-(cyclopentyloxy)phenyl)pyridine-2,6-diamine 2i,
3-(4-fluoro-2-methoxyphenyl) pyridine-2,6-diamine (hydrochloride), 2k,
3-(2,3-dimethoxyphenyl) pyridine-2,6-diamine (hydrochloride), 2l,
3-(2,4-dimethoxyphenyl) pyridine-2,6-diamine (hydrochloride), 2m,
3-(2,3-dichlorophenyl)-N2-methylpyridine-2,6-diamine, 5a,
3-(2,3-dichlorophenyl)-N2-ethylpyridine-2,6-diamine, 5b,
N2-butyl-3-(2,3-dichlorophenyl)pyridine-2,6-diamine, 5c,
N2-benzyl-3-(2,3-dichlorophenyl)pyridine-2,6-diamine, 5d,
3-(2,3-dichlorophenyl)-N2-isopropylpyridine-2,6-diamine, 5e,
3-(2,3-dichlorophenyl)-N2-phenethylpyridine-2,6-diamine, 5f,
3-(2,3-dichlorophenyl)-N2-(2-methoxyethyl)pyridine-2,6-diamine, 5g,
3-(2,3-dichlorophenyl)-N2-(2-(piperidin-1-yl)ethyl)pyridine-2,6-diamine, 5h,
5-(2,3-dichlorophenyl)-6-(piperidin-1-yl)pyridin-2-amine, 5i,
5-(2,3-dichlorophenyl)pyridin-2-amine, 6a,
5-(2,3-dichlorophenyl)-6-methylpyridin-2-amine, 6b,
5-(2,3-dichlorophenyl)-6-ethylpyridin-2-amine, 6c,
6-ethyl-5-(2-methoxyphenyl)pyridin-2-amine, 6d,
6-(methoxymethyl)-5-(2-methoxyphenyl)pyridin-2-amine, 6e,
5-(2-methoxyphenyl)-6-(trifluoromethoxy)pyridin-2-amine, 6f,
5-(2-methoxyphenyl)-6-propylpyridin-2-amine, 6g,
6-isopropyl-5-(2-methoxyphenyl)pyridin-2-amine, 6h,
6-cyclopropyl-5-(2-methoxyphenyl)pyridin-2-amine, 6i,
5-(2,3-dichlorophenyl)-6-methoxypyridin-2-amine, 7a,
3-(2,3-dichlorophenyl)-4-methoxypyridine-2,6-diamine, 8a,
3-(2,3-dichlorophenyl)-5-fluoropyridine-2,6-diamine 9a,
3-(2,3-dichlorophenyl)-5-ethylpyridine-2,6-diamine, 9b,
and one of a salt thereof (such as hydrochloride or trifluoroacetate).
More particularly, compounds of formula (III) are selected in the group consisting of:
3-(2-chlorophenyl)pyridine-2,6-diamine, 1b,
3-(2-isopropylphenyl) pyridine-2,6-diamine, 1e,
3-(2-(trifluoromethyl) phenyl) pyridine-2,6-diamine, 1f,
3-(2-(methoxymethyl) phenyl) pyridine-2,6-diamine, 1g,
3-(2,3-dichlorophenyl)pyridine-2,6-diamine, 1j,
5-(2-Methoxy-phenyl)-3-methyl-pyridin-2-ylamine, 13
5-(2-Methoxy-phenyl)-3-trifluoromethyl-pyridin-2-ylamine, 14
3-Fluoro-5-(2-methoxy-phenyl)-pyridin-2-ylamine, 15
5-(2,3-Dichloro-phenyl)-3-fluoro-pyridin-2-ylamine, 16
5-(2,3-Dichloro-phenyl)-4-fluoro-pyridin-2-ylamine, 17
4-Fluoro-5-(2-methoxy-phenyl)-pyridin-2-ylamine, 18
5-(2,3-Dichloro-phenyl)-4-methoxy-pyridin-2-ylamine (hydrochloride), 19
4-Methoxy-5-(2-methoxy-phenyl)-pyridin-2-ylamine, 20
5-(2-Methoxy-phenyl)-4-methyl-pyridin-2-ylamine, 21
5-(2,3-Dichloro-phenyl)-4-methyl-pyridin-2-ylamine, 22
5-(2-Methoxy-phenyl)-4-(2,2,2-trifluoro-ethoxy)-pyridin-2-ylamine (hydrochloride), 23
5-(2,3-Dichloro-phenyl)-4-(2,2,2-trifluoro-ethoxy)-pyridin-2-ylamine (hydrochloride), 24
4-(2-aminoethoxy)-5-(2-methoxyphenyl)pyridin-2-amine (dihydrochloride), 25
5-(2-Methoxy-phenyl)-6-methyl-pyridin-2-ylamine, 26
5-(2-Methoxy-phenyl)-4,6-dimethyl-pyridin-2-ylamine, 27
5-(2,3-Dichloro-phenyl)-4,6-dimethyl-pyridin-2-ylamine, 28
5-(3-chloro-2-methyl-phenyl)-6-ethyl-pyridin-2-ylamine (hydrochloride), 29
5-(2-cyclopropylphenyl)-6-ethyl-pyridin-2-amine (hydrochloride), 30
5-[2-(cyclopropoxy)phenyl]-6-ethyl-pyridin-2-amine (hydrochloride), 31
6-Fluoro-5-(2-methoxy-phenyl)-pyridin-2-ylamine, 32
5-(2,3-Dichloro-phenyl)-6-fluoro-pyridin-2-ylamine, 33
5-(2,3-Dichloro-phenyl)-6-trifluoromethyl-pyridin-2-ylamine, 34
6-Methoxy-5-(2-methoxy-phenyl)-pyridin-2-ylamine, 35
5-(2-Methoxy-phenyl)-6-(2,2,2-trifluoro-ethoxy)-pyridin-2-ylamine, 36
6-(2-Amino-ethoxy)-5-(2-methoxy-phenyl)-pyridin-2-ylamine, 37
6-(2-Amino-ethoxy)-5-(2,3-dichloro-phenyl)-pyridin-2-ylamine (dihydrochloride), 38
3-(2-Isopropoxy-6-methoxy-phenyl)-pyridine-2,6-diamine, 39
3-(4-Methoxy-2-methyl-phenyl)-pyridine-2,6-diamine, 40
3-(4-Chloro-2-fluoro-phenyl)-pyridine-2,6-diamine, 41
3-(2-Cyclopropyl-phenyl)-pyridine-2,6-diamine (hydrochloride), 42
3-(2-Phenoxy-phenyl)-pyridine-2,6-diamine (hydrochloride), 43
3-(2-Benzyl-phenyl)-pyridine-2,6-diamine, 44
3-(2-Chloro-4-fluoro-phenyl)-pyridine-2,6-diamine, 45
3-(2-Isopropoxy-4-methyl-phenyl)-pyridine-2,6-diamine, 46
3-(4-Chloro-2-cyclopentyloxy-phenyl)-pyridine-2,6-diamine, 47
3-(2-Cyclopropoxy-phenyl)-pyridine-2,6-diamine, 48
3-(2-Isopropoxy-5-methyl-phenyl)-pyridine-2,6-diamine, 49
3-(5-Fluoro-2-isopropoxy-phenyl)-pyridine-2,6-diamine, 50
3-(2,6-Dimethyl-phenyl)-pyridine-2,6-diamine, 51
3-(2-Isopropoxy-5-trifluoromethyl-phenyl)-pyridine-2,6-diamine, 52
3-(4-Fluoro-2-isopropoxy-phenyl)-pyridine-2,6-diamine, 53
3-(4-Chloro-2-methyl-phenyl)-pyridine-2,6-diamine, 54
3-(5-Chloro-2-cyclopropyl-phenyl)-pyridine-2,6-diamine, 55
3-(5-Chloro-2-methyl-phenyl)-pyridine-2,6-diamine, 56
3-(2-Methyl-4-trifluoromethyl-phenyl)-pyridine-2,6-diamine, 57
3-(2-chloro-3-methyl-phenyl)-pyridine-2,6-diamine, 58
3-(2-Methylsulfanyl-phenyl)-pyridine-2,6-diamine (hydrochloride), 59
2-(2,6-Diamino-pyridin-3-yl)-N,N-diethyl-benzamide (hydrochloride), 60
3-(2-Dimethylamino-phenyl)-pyridine-2,6-diamine (hydrochloride), 61
N-[2-(2,6-Diamino-pyridin-3-yl)-phenyl]-acetamide, 62
3-(2-methylsulfonylphenyl)pyridine-2,6-diamine (hydrochloride), 63
3-(2-benzyloxyphenyl)pyridine-2,6-diamine (hydrochloride), 64
3-[2-(cyclopropylmethoxy)phenyl]pyridine-2,6-diamine (hydrochloride), 65
3-(3-Chloro-2-methyl-phenyl)-5-fluoro-pyridine-2,6-diamine, 66
5-[2-(cyclopentoxy)phenyl]-6-ethyl-pyridin-2-amine (hydrochloride), 76
and one of a salt thereof (such as hydrochloride or trifluoroacetate).

20. A compound represented by formula (I): 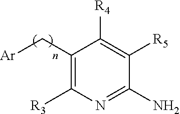
Where:
n, R3, R4, and R5 are as defined in any one of claims 1-19, and
Ar is 1-naphtyl, said naphtyl being optionally substituted as defined in claim 1.

21. The compound according to claim 20, where the compound presents at least one of, or more particularly all, the following features are fulfilled:
n is 0,
R₃ is an (C₁-C₁₀)alkyl group, such as ethyl, or NRR′, such as NH2,
the 1-naphtyl is unsubstituted or substituted by at least one group selected from a halogen atom, a cyano group, a (C₁-C₁₀)alkyl group, —OR, or —NRR′, where R and R′ are as defined above,
R₄ represents an hydrogen atom, and
R₅ represents an hydrogen atom.

22. The compound according to claim 20 or 21, where the compound is selected in the group consisting of:
6-ethyl-5-(1-naphthyl)pyridin-2-amine (hydrochloride), 67
3-(1-naphthyl)pyridine-2,6-diamine, 68
3-(2-methoxy-1-naphthyl)pyridine-2,6-diamine, 69
3-(2-isopropoxy-1-naphthyl)pyridine-2,6-diamine, 70
3-(4-methyl-1-naphthyl)pyridine-2,6-diamine (hydrochloride), 71
3-(4-fluoro-1-naphthyl)pyridine-2,6-diamine (hydrochloride), 72
3-(4-chloro-1-naphthyl)pyridine-2,6-diamine (hydrochloride), 73
4-(2,6-diamino-3-pyridyl)naphthalen-1-ol (hydrochloride), 74
3-[4-(dimethylamino)-1-naphthyl]pyridine-2,6-diamine (hydrochloride), 75

23. A compound represented by formula (I): 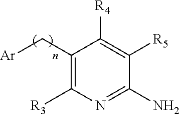
Where:
n, R3, R4, and R5 are as defined in any one of claims 1-19, and
Ar is a carbocyclyl or an heteroaryl, preferably a furanyl, benzofuranyl, a pyrazolyl (preferably 4-pyrazolyl) or a pyridinyl (preferably 3-pyridyl or 4-pyridyl) group, said Ar group can optionally be substituted as specified above, more specifically by one or more groups selected from a halogen atom, a (C₁-C₁₀)alkyl group, an aryl group, a —OR, R being as defined above, preferably R being H, (C₁-C₁₀)alkyl, or a —NRR′ group, R and R′ being as defined above, preferably R and R′ are independently H, (C₁-C₁₀)alkyl, or heterocycle.

24. The compound according to claim 23, where the compound presents at least one of, or more particularly all, the following features are fulfilled:
n is 0,
R₃ is an (C₁-C₁₀)alkyl group, such as ethyl, or NRR′, such as NH2,
R₄ represents an hydrogen atom, and
R₅ represents an hydrogen atom.

25. The compound according to claim 23 or 24, where the compound is selected in the group consisting of:
3-(benzofuran-2-yl) pyridine-2,6-diamine (hydrochloride), 3c
[3,4′-bipyridine]-2,6-diamine, 3d
[3,3′-bipyridine]-2,6-diamine, 3e,
3-(6-morpholino-3-pyridyl)pyridine-2,6-diamine, 78
3-[6-(1-piperidyl)-3-pyridyl]pyridine-2,6-diamine, 79
3-[6-(methylamino)-3-pyridyl]pyridine-2,6-diamine, 80
3-(6-pyrrolidin-1-yl-3-pyridyl)pyridine-2,6-diamine, 81
3-(6-amino-3-pyridyl)pyridine-2,6-diamine, 82
3-[6-amino-5-(trifluoromethyl)-3-pyridyl]pyridine-2,6-diamine, 83
3-(2-methyl-3-pyridyl)pyridine-2,6-diamine, 84
3-(6-fluoro-2-methyl-3-pyridyl)pyridine-2,6-diamine, 85
3-(6-fluoro-3-pyridyl)pyridine-2,6-diamine, 86
3-(2-fluoro-3-pyridyl)pyridine-2,6-diamine, 87
3-(4-methoxy-3-pyridyl)pyridine-2,6-diamine, 88
3-(2-methoxy-3-pyridyl)pyridine-2,6-diamine, 89
3-(3,5-dimethyl-1H-pyrazol-4-yl)pyridine-2,6-diamine, 90
3-(5-methyl-1H-pyrazol-4-yl)pyridine-2,6-diamine, 91

26. A pharmaceutical composition comprising at least one compound as defined in anyone of claims 17-25, in a pharmaceutically acceptable vehicle or support.

27. A compound or a composition according to anyone of claims 17-26, for use in the treatment of pain, preferably chronic pain.

28. The compound or a composition for use according to claim 27, to decrease or block hyperalgesia and/or tolerance effects related to the use of an analgesic compound, in particular an opiate analgesic compound.

29. The compound or a composition for use according to claim 27 or 28, for the treatment of hyperalgesia occurring or associated with acute or chronic pain in response to surgery, trauma or pathology of a mammal.
